(19) United States
(12) Patent Application Publication
(10) Pub. No.: US 2014/0086839 A1
(43) Pub. Date: Mar. 27, 2014

(54) QUINOLONE ANALOGS FOR TREATING AUTOIMMUNE DISEASES

(71) Applicants: Anat Achiron, Tel-Aviv (IL); Michael Gurevich, Rechovot (IL)

(72) Inventors: Anat Achiron, Tel-Aviv (IL); Michael Gurevich, Rechovot (IL)

(73) Assignee: Tel HaShomer Medical Research Infrastructure and Services Ltd., Ramat-Gan (IL)

(21) Appl. No.: 14/005,583

(22) PCT Filed: Feb. 26, 2012
(86) PCT No.: PCT/IL12/50061

§ 371 Date: Sep. 17, 2013

Related U.S. Application Data
(60) Provisional application No. 61/457,396, filed on Mar. 17, 2011.

(51) Int. Cl. C07D 513/14

(52) U.S. Cl. CPC C07D 513/14
USPC 424/9.2; 514/218

(57) ABSTRACT

Provided are methods of treating autoimmune diseases by administering to the subject a therapeutically effective amount of a quinolone compound such as 2-(4-methyl-1,4-diazepan-1-yl)-N-((5-methylpyrazin-2-yl)methyl)-5-oxo-5H-benzo[4,5]thiazolo[3,2-a][1,8]naphthyridine-6-carboxamide (CX-5461). Also provided are methods of monitoring efficiency of treatment of the quinolone compound by determining the expression level of at least one gene of the RNA polymerase I pathway.

### FIELD AND BACKGROUND OF THE INVENTION

The present invention, in some embodiments thereof, relates to methods of treating autoimmune diseases and, more particularly, but not exclusively, to method of treating multiple sclerosis using a quinolone compound such as CX-5461.

Autoimmune diseases are caused by an autoimmune response, i.e., an immune response directed to a substance in the body of the subject. The characteristics of the autoimmune diseases vary and depend on the site affected by the autoimmune response.

Multiple sclerosis (MS) is the most common demyelinating disease of the central nervous system (CNS) affecting young adults (disease onset between 20 to 40 years of age) and is the third leading cause for disability after trauma and rheumatic diseases, with an estimated annual cost 34,000 USD per patient (total life time cost of 2.2 million USD per patient).

The disease is characterized by destruction of myelin, associated with death of oligodendrocytes and axonal loss. The main pathologic finding in MS is the presence of infiltrating mononuclear cells, predominantly T lymphocytes and macrophages, which surpass the blood brain barrier and induce an active inflammation within the brain and spinal cord. The neurological symptoms that characterize MS include complete or partial vision loss, diplopia, sensory symptoms, motor weakness that can worsen to complete paralysis, bladder dysfunction and cognitive deficits, which eventually may lead to a significant disability. The associated multiple inflammatory foci lead to myelin destruction, plaques of demyelination, gliosis and axonal loss within the brain and spinal cord and are the reasons contribute to the clinical manifestations of neurological disability.

The etiology of MS is not fully understood. The disease develops in genetically predisposed subjects exposed to yet undefined environmental factors and the pathogenesis involves autoimmune mechanisms associated with autoreactive T cells against myelin antigens. It is well established that not one dominant gene determines genetic susceptibility to develop MS, but rather many genes, each with different influence, are involved.

Clinically, in 85% of MS patients the illness is initiated with a relapsing-remitting course (RRMS), and in about 10-15% of MS patients have an a-priori primary progressive course (PPMS) without relapses. RRMS is characterized by inflammatory attacks associated with neurological deficits with periods of remissions between the relapses that vary in time. After a period of 10 years, about 50% of RRMS patients will progress to a secondary progressive MS (SPMS) course, characterized by permanent neurological dysfunction, with or without relapses and progressive disability.

Benign MS (BMS) is a clinical variant of RRMS in which the patients develop low neurological disability if at all after a disease duration of at least 10 years. Accordingly, this group of MS patients do not experience devastating accumulating disability over-time and when these patients are examined neurologically and scored by the Expanded Disability Status Scale (EDSS) they receive a score that is equal to or lower than 3.0. This low EDSS score signifies mild disability and when this low disability occurs more than 10 years after disease onset, the course of MS is defined as benign (Pittock S J and Rodriguez M, 2008; Costelloe, L., et al., 2008). Prediction of patients that will have BMS is currently impossible and the definition of these patients is retrospective. The molecular events accountable for the BMS variant of disease are not understood.

WO 2008/081435 publication discloses methods and kits for predicting the prognosis of a subject diagnosed with multiple sclerosis and methods of selecting a treatment regimen of a subject diagnosed with multiple sclerosis.

Achiron A, et al., 2007 (Clinical and Experimental Immunology, 149: 235-242) describe genes of the zinc-ion binding and cytokine activity regulation pathways which predict outcome in relapsing-remitting multiple sclerosis.

WO 2010/113096 discloses methods of predicting clinical course and treating multiple sclerosis.

Current approved drugs for the treatment of MS are either general anti-inflammatory agents or immunomodulators and consequently result only in moderate beneficial effects suppressing disease activity.

CX-5461 (FIG. 1) is a small molecule that was designed to selectively inhibit rRNA synthesis by inhibiting RNA Polymerase I (Pol I), without affecting mRNA synthesis by RNA Polymerase II (Pol II), and without inhibiting DNA replication or protein synthesis (Russell J, Zomerdijk J C. RNA-polymerase-1-directed rDNA transcription, life and works. Trends Biochem Sci 30:87-96, 2005; Drygin D, et al. The RNA polymerase I transcription machinery: an emerging target for the treatment of cancer. Annu Rev Pharmacol Toxicol 50:131-156, 2010). The inhibition of Pol I results in nucleolar stress which causes the release of ribosomal proteins (RP) from the nucleolus and subsequent activation of p53, resulting in cell apoptosis (Kalita K, et al. Inhibition of nucleolar transcription as a trigger for neuronal apoptosis. J Neurochem 105:2286-2299, 2008). In a previous study (Drygin D, et al. Targeting RNA polymerase I with an oral small molecule CX-5461 inhibits ribosomal RNA synthesis and solid tumor growth. Cancer Res 71:1418-1430, 2011) the antiproliferative activity of CX-5461 was studied in cell lines and it was shown that CX-5461 inhibited Pol I activity in human cancer cell lines.

Recent studies indicate that disruption of the SL1/rDNA complex by CX-5461 results from the interference between SL1 and rDNA. SL1, a protein complex containing TATA binding protein-associated factors, is responsible for Pol I promoter specificity. SL1 performs important tasks in the transcription complex assembly, mediating specific interactions between the rDNA promoter region and the Pol I enzyme complex, thereby recruiting Pol I, together with a collection of Pol I-associated factors like RRN3 to rDNA (Cavanaugh A, et al. Mammalian Rrn3 is required for the formation of a transcription competent preinitiation complex containing RNA polymerase I. Gene Expr 14:131-147, 2008).

Additional background art include Leuenroth S J and Crews C M (Triptolide-induced transcriptional arrest is associated with changes in nuclear substructure. Cancer Res. 2008; 68:5257-5266); Liu Y, et al. (Triptolide, a component of Chinese herbal medicine, modulates the functional phenotype of dendritic cells. Transplantation. 2007; 84:1517-1526); Wang Y, et al. (Triptolide modulates T-cell inflammatory responses and ameliorates experimental autoimmune encephalomyelitis. J Neurosci Res. 2008; 86:2441-2449; EP 0983256; PCT/US1998/008562; WO9852933A1; Alice H. Cavanaugh, et al., 2002 (Rrn3 Phosphorylation is a regulatory checkpoint for ribosome biogenesis J. Biol. Chem., 2002; 277: 27423-27432); PCT Pub. No. WO03081201A2.

### SUMMARY OF THE INVENTION

According to an aspect of some embodiments of the present invention there is provided a method of treating an autoimmune disease in a subject, comprising:

administering to the subject a therapeutically effective amount of a compound of Formula (I),

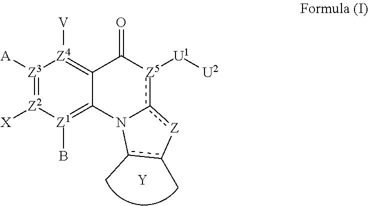

or a pharmaceutically acceptable salt or ester thereof;
wherein [symbol] indicates an optionally unsaturated bond;
each of B, X, A or V is absent if Z¹, Z², Z³ and Z⁴, respectively, is N; and
each of B, X, A and V is independently H, halo, azido, —CN, —CF₃, —CONR¹R², —NR¹R², —SR², —OR², —R³, —W, -L-W, —W⁰, -L-N(R)—W⁰, A² or A³, when each of Z¹, Z², Z³ and Z⁴, respectively, is C;

### Z is O, S, CR⁴₂, NR⁴CR⁴, CR⁴NR⁴, CR⁴, NR⁴ or N;

each of Z¹, Z², Z³ and Z⁴ is independently C or N, provided any three N are non-adjacent;
Z⁵ is C; or Z⁵ may be N when Z is N;
Y is an optionally substituted 5-6 membered carbocyclic or heterocyclic ring;
U¹ is —C(=T)N(R)—, —C(=T)N(R)O—, —C(=T)-, —SO₂N(R)—, —SO₂N(R)N(R⁰)—, —SO₂—, or —SO₃—, where T is O, S, or NH; or U¹ may be a bond when Z⁵ is N or U² is H;
U² is H, or C3-C7 cycloalkyl, C1-C10 alkyl, C1-C10 heteroalkyl, C2-C10 alkenyl or C2-C10 heteroalkenyl group, each of which may be optionally substituted with one or more halogens, ═O, or an optionally substituted 3-7 membered carbocyclic or heterocyclic ring; or U² is —W, -L-W, -L-N(R)—W⁰, A² or A³;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R⁴ may be —W, -L-W or -L-N(R)—W⁰;
each R and R⁰ is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member;
W⁰ is an optionally substituted 3-4 membered carbocyclic ring, or a C1-C6 alkyl group substituted with from 1 to 4 fluorine atoms;
provided one of U², V, A, X and B is a secondary amine A² or a tertiary amine A³, wherein
the secondary amine A² is —NH—W⁰, and
the tertiary amine A³ is a fully saturated and optionally substituted six-membered or seven-membered azacyclic ring optionally containing an additional heteroatom selected from N, O or S as a ring member, or the tertiary amine A³ is a partially unsaturated or aromatic optionally substituted five-membered azacyclic ring, optionally containing an additional heteroatom selected from N, O or S as a ring member;

thereby treating the autoimmune disease in the subject.

According to some embodiments of the invention, the Z¹ is N, and each of Z², Z³ and Z⁴ is C.

According to some embodiments of the invention, the U is —W or -L-W, where W is an optionally substituted 5-6 membered unsaturated or aromatic azacyclic ring, optionally containing an additional heteroatom selected from N, O and S; or W is an optionally substituted 5-7 membered saturated azacyclic ring containing an additional heteroatom selected from N and S.

According to some embodiments of the invention, the U² is -L-N(R)—W⁰.

According to some embodiments of the invention, the Y is an optionally substituted phenyl ring.

According to some embodiments of the invention, with the proviso that when Z¹ is N, Z² and Z⁴ are C, Z⁵ is C, U¹ is —C(O)NH—, U² is -L-W, and L is an ethylene linker, one of V, A, and X is independently an optionally substituted aryl, heteroaryl, or 7-membered azacyclic ring, optionally containing an additional heteroatom selected from N, O and S as a ring member, if W is pyrrolidin-1-yl, N-methyl-pyrrolidin-2-yl, piperidin-1-yl or morpholin-1-yl

According to an aspect of some embodiments of the present invention there is provided a method of treating an autoimmune disease in a subject, comprising:

administering to the subject a therapeutically effective amount of a compound of Formula (II),

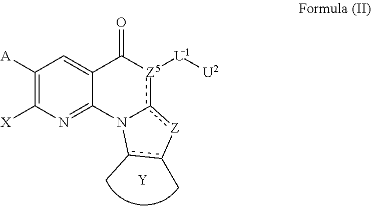

or a pharmaceutically acceptable salt or ester thereof;
wherein [symbol] indicates an optionally unsaturated bond;
each of A and X is independently H, halo, azido, —CN, —CF₃, —CONR¹R², —NR¹R², —SR², —OR², —R³, —W, -L-W, —W⁰, -L-N(R)—W⁰, A² or A³;

### Z is O, S, CR⁴₂, NR⁴CR⁴, CR⁴NR⁴ or NR⁴;

Y is an optionally substituted 5-6 membered carbocyclic or heterocyclic ring;
U¹ is —C(=T)N(R)—, —C(=T)N(R)O—, —C(=T)-, —SO₂N(R)—, —SO₂N(R)N(R⁰)—, —SO₂—, or —SO₃—, where T is O, S, or NH; or U¹ may be a bond when U2 is H;
U² is H, or C3-C7 cycloalkyl, C1-C10 alkyl, C1-C10 heteroalkyl, C2-C10 alkenyl or C2-C10 heteroalkenyl group, each of which may be optionally substituted with one or more halogens, ═O, or an optionally substituted 3-7 membered carbocyclic or heterocyclic ring; or U² is —W, -L-W, -L-N(R)—W⁰, A² or A³;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R⁴ may be —W, -L-W or -L-N(R)—W⁰;
each R and R⁰ is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member;
W⁰ is an optionally substituted 3-4 membered carbocyclic ring, or a C1-C6 alkyl group substituted with from 1 to 4 fluorine atoms;
provided that one of U², A, and X is a secondary amine A² or a tertiary amine A³, wherein
the secondary amine A² is —NH—W⁰, and
the tertiary amine A³ is a fully saturated and optionally substituted six-membered or seven-membered azacyclic ring optionally containing an additional heteroatom selected from N, O or S as a ring member, or the tertiary amine A³ is a partially unsaturated or aromatic optionally substituted five-membered azacyclic ring optionally containing an additional heteroatom selected from N, O or S as a ring member;

thereby treating the autoimmune disease in the subject.

According to some embodiments of the invention, the at least one of A and X is a tertiary amine A³.

According to some embodiments of the invention, the A³ is selected from the group consisting of imidazole, imidazoline, pyrroline, piperidine, piperazine, morpholine, thiomorpholine and homopiperazine.

According to some embodiments of the invention, the U¹ is a —C(=T)N(R)—, T is O, and U² is -L-W or -L-N(R)—W⁰.

According to some embodiments of the invention, with the proviso that when U¹ is —C(O)NH—, U² is -L-W, and L is an ethylene linker, one of A and X is independently an optionally substituted aryl, heteroaryl, or 7-membered azacyclic ring, optionally containing an additional heteroatom selected from N, O and S as a ring member, if W is pyrrolidin-1-yl, N-methyl-pyrrolidin-2-yl, piperidin-1-yl or morpholin-1-yl.

According to an aspect of some embodiments of the present invention there is provided a method of treating an autoimmune disease in a subject, comprising:

administering to the subject a therapeutically effective amount of a compound of Formula (III),

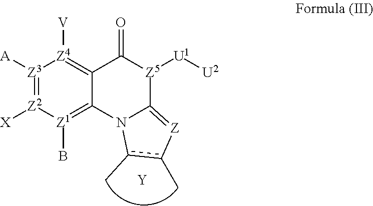

or a pharmaceutically acceptable salt or ester thereof;
wherein [symbol] indicates an optionally unsaturated bond; and
each of B, X, A or V is absent if Z¹, Z², Z³ and Z⁴, respectively, is N; and
each of B, X, A and V is independently H, halo, azido, —CN, —CF₃, —CONR¹R², —NR¹R², —SR², —OR², —R³, —W, -L-W, —W⁰, -L-N(R)—W⁰, A² or A³, when each of Z¹, Z², Z³ and Z⁴, respectively, is C;
each of Z¹, Z², Z³ and Z⁴ is independently C or N, provided any three N are non-adjacent;
Y is an optionally substituted 5-6 membered carbocyclic or heterocyclic ring;
U¹ is —C(=T)N(R)—, —C(=T)N(R)O—, —C(=T)-, —SO₂N(R)—, —SO₂N(R)N(R⁰)—, —SO₂—, or —SO₃—, where T is O, S, or NH; or U¹ may be a bond when Z⁵ is N or U² is H;
U² is H, or C3-C7 cycloalkyl, C1-C10 alkyl, C1-C10 heteroalkyl, C2-C10 alkenyl or C2-C10 heteroalkenyl group, each of which may be optionally substituted with one or more halogens, ═O, or an optionally substituted 3-7 membered carbocyclic or heterocyclic ring; or U² is —W, -L-W, -L-N(R)—W⁰, A² or A³;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R⁴ may be —W, -L-W or -L-N(R)—W⁰;
each R and R⁰ is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member;
W⁰ is an optionally substituted 3-4 membered carbocyclic ring, or a C1-C6 alkyl group substituted with from 1 to 4 fluorine atoms;
provided that one of U², V, A, X and B is a secondary amine A² or a tertiary amine A3, wherein
the secondary amine A² is —NH—W⁰, and
the tertiary amine A³ is a fully saturated and optionally substituted six-membered or seven-membered azacyclic ring optionally containing an additional heteroatom selected from N, O or S as a ring member, or the tertiary amine A³ is a partially unsaturated or aromatic optionally substituted five-membered azacyclic ring, optionally containing an additional heteroatom selected from N, O or S as a ring member, thereby treating the autoimmune disease in the subject.

According to an aspect of some embodiments of the present invention there is provided a method of treating an autoimmune disease in a subject, comprising:

administering to the subject a therapeutically effective amount of a compound of Formula (IV),

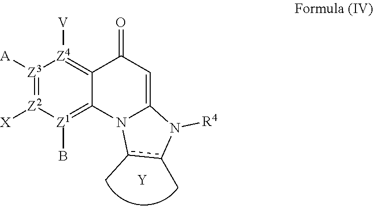

or a pharmaceutically acceptable salt or ester thereof;
wherein [symbol] indicates an optionally unsaturated bond;
each of B, X, A or V is absent if Z¹, Z², Z³ and Z⁴ respectively, is N; and
each of B, X, A and V is independently H, halo, azido, —CN, —CF₃, —CONR¹R², —NR¹R², —SR², —OR², —R³, —W, -L-W, —W⁰, -L-N(R)—W⁰, A² or A³, when each of Z¹, Z², Z³ and Z⁴, respectively, is C;
each of Z¹, Z², Z³ and Z⁴ is independently C or N, provided any three N are non-adjacent;
Y is an optionally substituted 5-6 membered carbocyclic or heterocyclic ring;
in each NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;

### R⁴ is —W, -L-W or -L-N(R)—WO; and

each R is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member; and
W⁰ is an optionally substituted 3-4 membered carbocyclic ring, or a C1-C6 alkyl group substituted with from 1 to 4 fluorine atoms,

thereby treating the autoimmune disease in the subject.

According to an aspect of some embodiments of the present invention there is provided a method of treating an autoimmune disease in a subject, comprising:

administering to the subject a therapeutically effective amount of a compound of Formula (V),

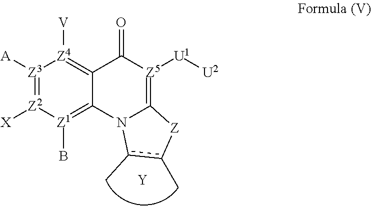

or a pharmaceutically acceptable salt or ester thereof; [symbol]
indicates an optionally unsaturated bond;
A and V independently are H, halo, azido, —CN, —CF₃, —CONR¹R², —NR¹R², —SR², —OR², —R³, —W, -L-W, —W⁰, -L-N(R)—W⁰, A² or A³;

### Z is O, S, CR⁴₂, NR⁴CR⁴, CR⁴NR⁴ or NR⁴;

Y is an optionally substituted 5-6 membered carbocyclic or heterocyclic ring;
U¹ is —C(=T)N(R)—, —C(=T)N(R)O—, —C(=T)-, —SO₂N(R)—, —SO₂N(R)N(R⁰)—, —SO₂—, or —SO₃—, where T is O, S, or NH; or U¹ may be a bond when U2 is H;
U² is H, or C3-C7 cycloalkyl, C1-C10 alkyl, C1-C10 heteroalkyl, C2-C10 alkenyl or C2-C10 heteroalkenyl group, each of which may be optionally substituted with one or more halogens, ═O, or an optionally substituted 3-7 membered carbocyclic or heterocyclic ring; or U² is —W, -L-W or -L-N(R)—W⁰, A² or A³;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R4 may be —W, -L-W or -L-N(R)—W⁰;
each R and R⁰ is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member;
W⁰ is an optionally substituted 3-4 membered carbocyclic ring, or a C1-C6 alkyl group substituted with from 1 to 4 fluorine atoms;
provided one of U², A, and V is a secondary amine A² or a tertiary amine A3, wherein
the secondary amine A² is —NH—WO, and
the tertiary amine A³ is a fully saturated and optionally substituted six-membered or seven-membered azacyclic ring optionally containing an additional heteroatom selected from N, O or S as a ring member, or the tertiary amine A³ is a partially unsaturated or aromatic optionally substituted five-membered azacyclic ring optionally containing an additional heteroatom selected from N, O or S as a ring member,

thereby treating the autoimmune disease in the subject.

According to an aspect of some embodiments of the present invention there is provided a method of treating an autoimmune disease in a subject, comprising:

administering to the subject a therapeutically effective amount of a compound of Formula (VI),

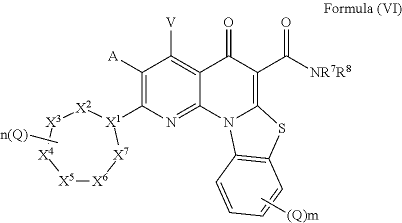

or a pharmaceutically acceptable salt or ester thereof;
wherein:

### X¹ is CH or N;

X², X³, X⁴, X⁵, X⁶ and X⁷ independently are NR⁴, CH₂, CHQ or C(O)₂, provided that: (i) zero, one or two of X², X³, X⁴, X⁵, X⁶ and X⁷ are NR⁴; (ii) when X¹ is N, both of X² and X⁷ are not NR⁴; (iii) when X¹ is N, X³ and X⁶ are not NR⁴; and (iv) when X¹ is CH and two of X², X³, X⁴, X⁵, X⁶ and X⁷ are NR⁴, the two NR⁴ are located at adjacent ring positions or are separated by two or more other ring positions;
A and V independently are H, halo, azido, —CN, —CF₃, —CONR¹R², —NR¹R², —SR², —OR², —R³, —W, -L-W, —W⁰, or -L-N(R)—W⁰;
each Q is independently halo, azido, —CN, —CF₃, —CONR¹R², —NR¹R², —SR², —OR², —R³, —W, -L-W, —W⁰, or -L-N(R)—W⁰;
in each —NR¹R², R¹ and R² together with N may form an optionally substituted azacyclic ring, optionally containing one additional heteroatom selected from N, O and S as a ring member;
R¹ is H or C1-C6 alkyl, optionally substituted with one or more halogens, or ═O;
R is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R4 may be —W, -L-W or -L-N(R)—W⁰;
each R is independently H or C1-C6 alkyl;
R⁷ is H and R⁸ is C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring; or in NR⁷R⁸, R⁷ and R⁸ together with N may form an optionally substituted azacyclic ring, optionally containing an additional heteroatom selected from N, O and S as a ring member;
m is 0, 1, 2, 3 or 4;
n is 0, 1, 2, 3, 4, or 5;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member; and
W⁰ is an optionally substituted 3-4 membered carbocyclic ring, or a C1-C6 alkyl group substituted with from 1 to 4 fluorine atoms,

thereby treating the autoimmune disease in the subject.

According to some embodiments of the invention, the X¹ is CH and two of X², X³, X⁴, X⁵, X⁶ and X⁷ are NR⁴.

According to some embodiments of the invention, the X¹ is CH and one of X², X³, X⁴, X⁵, X⁶ and X⁷ are NR⁴.

According to some embodiments of the invention, the X¹ is CH and none of X², X³, X⁴, X⁵, X⁶ and X⁷ are NR⁴.

According to some embodiments of the invention, the X¹ is N and none of X², X³, X⁴, X⁵, X⁶ and X⁷ are NR⁴.

According to some embodiments of the invention, the X¹ is N and one of X⁴ or X⁵ is NR⁴.

According to an aspect of some embodiments of the present invention there is provided a method of treating an autoimmune disease in a subject, comprising:

administering to the subject a therapeutically effective amount of a compound of Formula (VIII),

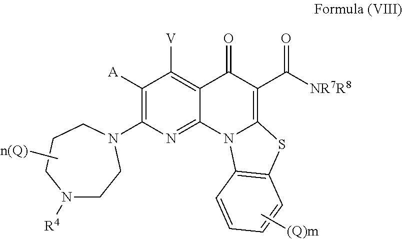

or a pharmaceutically acceptable salt or ester thereof;
wherein:
A and V independently are H, halo, azido, —CN, —CF₃, —CONR¹R², —NR¹R², —SR², —OR², —R³, —W, -L-W, —W⁰, or -L-N(R)—W⁰;
each Q is independently halo, azido, —CN, —CF₃, —CONR¹R², —NR¹R², —SR², —OR², —R³, —W, -L-W, —W⁰, or -L-N(R)—W⁰;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R4 may be —W, -L-W or -L-N(R)—W⁰;
each R is independently H or C1-C6 alkyl;
R⁷ is H and R⁸ is C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring; or in NR⁷R⁸, R⁷ and R⁸ together with N may form an optionally substituted azacyclic ring, optionally containing an additional heteroatom selected from N, O and S as a ring member;
m is 0, 1, 2, 3 or 4;
n is 0, 1, 2, 3, 4 or 5;
p is 0, 1, 2 or 3;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member; and
W⁰ is an optionally substituted 3-4 membered carbocyclic ring, or a C1-C6 alkyl group substituted with from 1 to 4 fluorine atoms,

thereby treating the autoimmune disease in the subject.

According to some embodiments of the invention, the R⁷ is H and R⁸ is a C₁₋₄ alkyl substituted with an optionally substituted aromatic heterocyclic ring.

According to some embodiments of the invention, the optionally substituted aromatic heterocyclic ring is selected from pyridine, pyrimidine, pyrazine, imidazole, pyrrolidine, and thiazole.

According to some embodiments of the invention, the R⁷ and R⁸ together with N in —NR⁷R⁸ form an optionally substituted azacyclic ring selected from the group consisting of morpholine, thiomorpholine, piperidine or piperazine ring.

According to an aspect of some embodiments of the present invention there is provided a method of treating an autoimmune disease in a subject, comprising:

administering to the subject a therapeutically effective amount of a compound of Formula (VII)

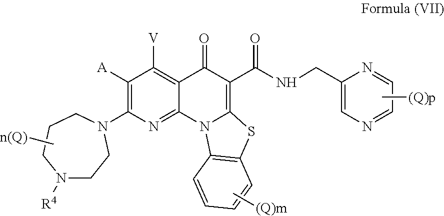

or a pharmaceutically acceptable salt or ester thereof;
A and V independently are H, halo, azido, —CN, —CF₃, —CONR¹R², —NR¹R², —SR², —OR², —R³, —W, -L-W, —W⁰, or -L-N(R)—W⁰;
each Q is independently halo, azido, —CN, —CF₃, —CONR¹R², —NR¹R², —SR², —OR², —R³, —W, -L-W, —W⁰, or -L-N(R)—W⁰;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 hetero alkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R⁴ may be —W, -L-W or -L-N(R)—W⁰;
each R is independently H or C1-C6 alkyl;
m is 0, 1, 2, 3 or 4;
n is 0, 1, 2, 3, 4 or 5;
p is 0, 1, 2 or 3;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member; and
W⁰ is an optionally substituted 3-4 membered carbocyclic ring, or a C1-C6 alkyl group substituted with from 1 to 4 fluorine atoms,

thereby treating the autoimmune disease in the subject.

According to some embodiments of the invention, the A and V are independently H or halo.

According to some embodiments of the invention, the R⁴ is H or C1-4 alkyl.

According to some embodiments of the invention, the m and n are each 0.

According to some embodiments of the invention, the p is 0 or 1.

According to some embodiments of the invention, the compound having the structure:

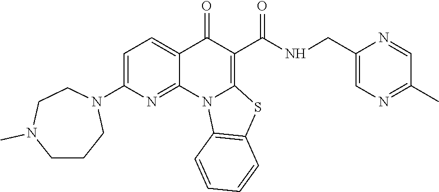

or a pharmaceutically acceptable salt or ester thereof.

According to an aspect of some embodiments of the present invention there is provided a method of treating an autoimmune disease in a subject, comprising:

administering to the subject a therapeutically effective amount of a compound selected from the group consisting of:

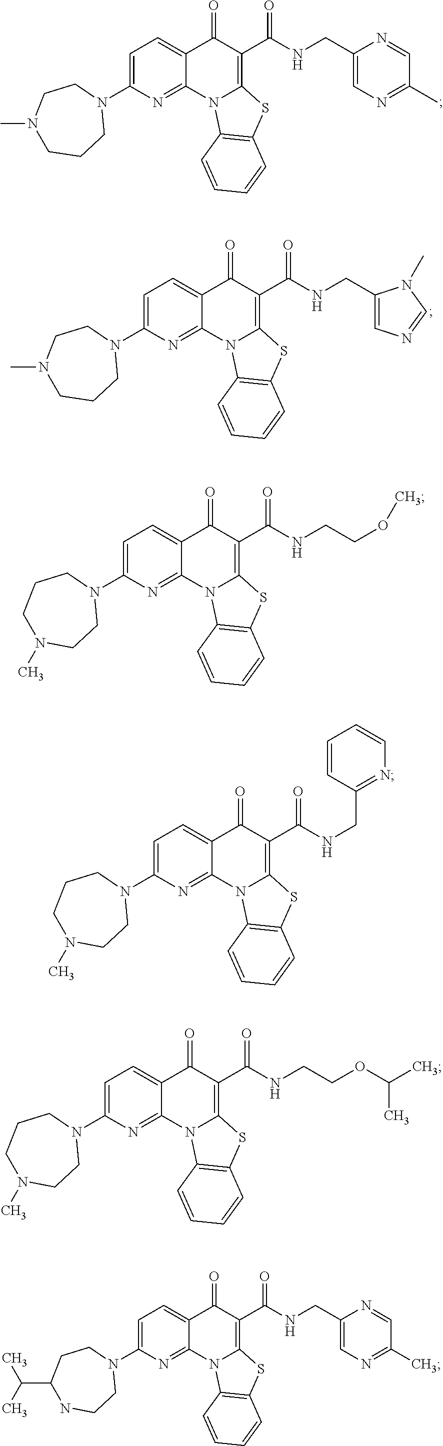

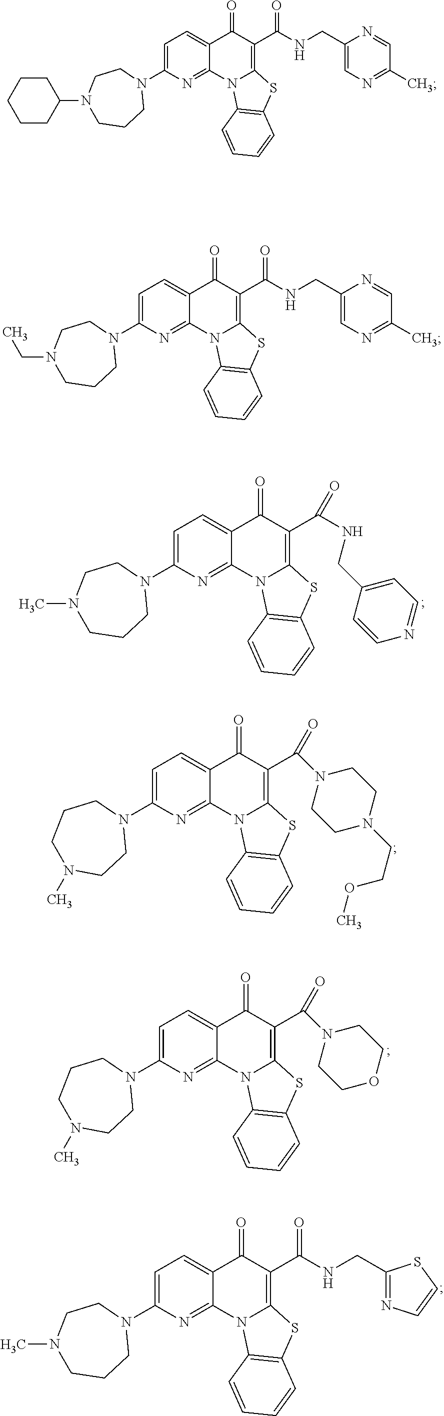 [SMILES: Cc1cnc(CNC(=O)c2c(=O)c3ccc(N4CCCN(C5CCCCC5)CC4)nc3n3c2sc2ccccc23)cn1 ; CCN1CCCN(c2ccc3c(=O)c(C(=O)NCc4cnc(C)cn4)c4sc5ccccc5n4c3n2)CC1 ; CN1CCCN(c2ccc3c(=O)c(C(=O)NCc4ccncc4)c4sc5ccccc5n4c3n2)CC1 ; COCCN1CCN(C(=O)c2c(=O)c3ccc(N4CCCN(C)CC4)nc3n3c2sc2ccccc23)CC1 ; CN1CCCN(c2ccc3c(=O)c(C(=O)N4CCOCC4)c4sc5ccccc5n4c3n2)CC1 ; CN1CCCN(c2ccc3c(=O)c(C(=O)NCc4nccs4)c4sc5ccccc5n4c3n2)CC1]

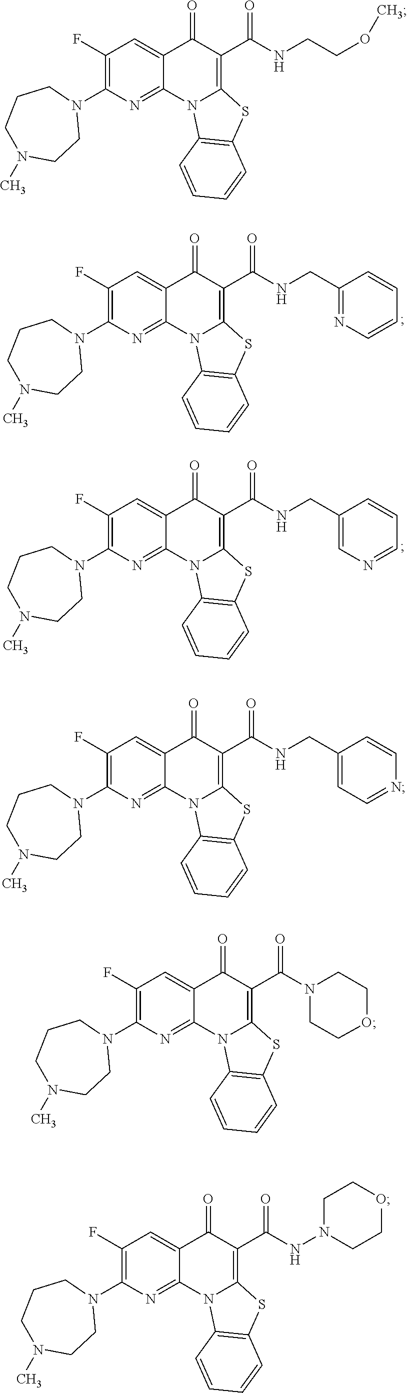

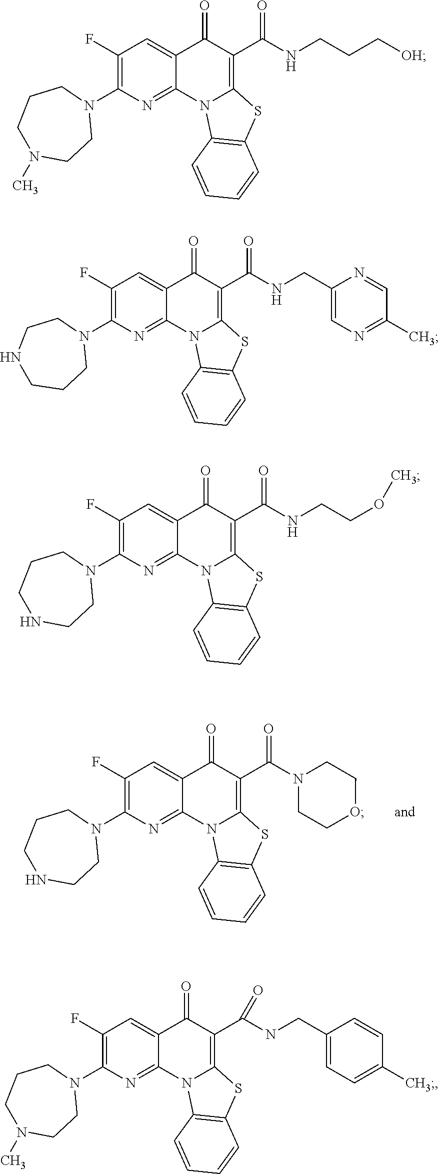

or a pharmaceutically acceptable salt or ester thereof,

thereby treating the autoimmune disease in the subject.

According to an aspect of some embodiments of the present invention there is provided a method of monitoring treatment efficiency of a subject having an autoimmune disease, the method comprising:

(a) treating the subject with the compound according to the method of some embodiments of the invention, and

(b) comparing a level of expression of least one gene involved in the RNA polymerase I pathway in a cell of the subject following the treating with the compound to a level of expression of the at least one gene in a cell of the subject prior to the treating the subject with the compound,

(i) wherein a decrease above a predetermined threshold in the level of expression of the at least one gene following the treating with the compound relative to the level of expression of the at least one gene prior to the treating with the compound indicates that the compound is efficient for treating the subject;

(ii) wherein an increase above a predetermined threshold in the level of expression of the at least one gene following the treating with the compound relative to the level of expression of the at least one gene prior to the treating with the compound indicates that the compound is not efficient for treating the subject; or

(iii) wherein when a level of expression of the at least one gene following the treating with the compound is identical or changed below a predetermined threshold as compared to prior to the treating with the compound then the treatment is not efficient for treating the subject

thereby monitoring treatment efficiency of the subject having an autoimmune disease.

According to some embodiments of the invention, the compound is 2-(4-methyl-1,4-diazepan-1-yl)-N-((5-methylpyrazin-2-yl)methyl)-5-oxo-5H-benzo[4,5]thiazolo[3,2-a][1,8]naphthyridine-6-carboxamide as depicted in Formula

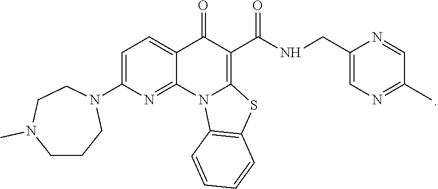

According to some embodiments of the invention, the autoimmune disease is multiple sclerosis.

According to some embodiments of the invention, the compound is 2-(4-methyl-1,4-diazepan-1-yl)-N-((5-methylpyrazin-2-yl)methyl)-5-oxo-5H-benzo[4,5]thiazolo[3,2-a][1,8]naphthyridine-6-carboxamide as depicted in Formula

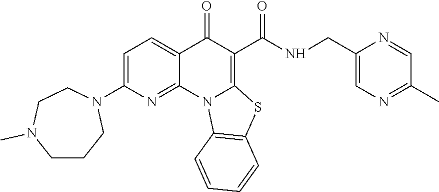

and wherein the autoimmune disease is multiple sclerosis.

According to some embodiments of the invention, the multiple sclerosis is a relapsing-remitting course of multiple sclerosis.

According to some embodiments of the invention, the multiple sclerosis is a progressive course of multiple sclerosis.

According to some embodiments of the invention, the multiple sclerosis is a benign multiple sclerosis.

According to some embodiments of the invention, the multiple sclerosis is a progressive-relapsing course.

According to some embodiments of the invention, administering the compound is performed following diagnosis of the autoimmune disease.

According to some embodiments of the invention, the autoimmune disease is multiple sclerosis and whereas the diagnosis comprises appearance of brain lesions characteristics of the multiple sclerosis.

According to some embodiments of the invention, the compound is provided at a concentration range of about 0.081-1.62 mg/kg/day.

According to some embodiments of the invention, the compound is provided at a concentration range of about 0.81-1.215 mg/kg/day.

According to some embodiments of the invention, the compound is provided at a concentration of about 1.01 mg/kg/day.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of the chemical structure of CX-5461;

FIG. 2 is a schematic illustration of the chemical formation of ethyl 2-chloro-5-oxo-5H-benzo[4,5]thiazolo[3,2-a][1,8]naphthyridine-6-carboxylate (2);

FIG. 3 is a schematic illustration depicting the chemical conversion of ethyl 2-chloro-5-oxo-5H-benzo[4,5]thiazolo[3,2-a][1,8]naphthyridine-6-carboxylate (2) to ethyl 2-(4-methyl-1,4-diazepan-1-yl)-5-oxo-5H-benzo[4,5]thiazolo[3,2-a][1,8]naphthyridine-6-carboxylate (3);

FIG. 4 is a is a schematic illustration depicting the chemical conversion of ethyl 2-(4-methyl-1,4-diazepan-1-yl)-5-oxo-5H-benzo[4,5]thiazolo[3,2-a][1,8]naphthyridine-6-carboxylate (3) to 2-(4-methyl-1,4-diazepan-1-yl)-N-((5-methylpyrazin-2-yl)methyl)-5-oxo-5H-benzo[4,5]thiazolo[3,2-a][1,8]naphthyridine-6-carboxamide(4);

FIG. 5 is a histogram depicting the effect of CX-5461 on viability of mouse splenocytes. Mouse splenocytes were plated in DMEM+10% FCS+P/S+Q and 10 μg/ml PHA (250,000 cell/well) in the presence of increased concentrations of CX-5461 (50-4000 nM) for 72 hours. Cells cultured with PHA alone served as controls. Following incubation cell viability was determined by XTT assay;

FIG. 6 is a histogram depicting the effect of CX-5461 on viability of human PBMCs. Human PBMCs were plated in DMEM+10% FCS+P/S+Q and 20 μg/ml PHA (400,000 cell/well) in the presence of increased concentrations of CX-5461 (50-500 nM) for 72 hours. Cells cultured with PHA alone served as controls. After incubation cell viability was determined by XTT assay;

FIGS. 7A-D are histograms depicting the effects of CX-5461 on apoptosis of human PBMCs. Human PBMCs were plated in DMEM+10% FCS+P/S+Q (400,000 cell/well) in the presence of elevated concentration of CX-5461 (25-250 nM) for 24 hours. Cells cultured with vehicle alone served as controls. After incubation the percent of cells positive for Annexin V (FIG. 7A), P53 (FIG. 7C), RRN3 (FIG. 7D) and total apoptotic cells was determined by flow cytometry (FIG. 7B);

FIG. 8 is a histogram depicting the effects of CX-5461 on viability of neuronal progenitor cells (NPCs). Mouse NPCs were plated in neurospheres medium containing basic FGF and EGF at 20 ng/ml each (26,000 cell/well) in the presence of increased concentrations of CX-5461 (20-500 nM) for 72 hours. Cells cultured without CX-5461 served as controls. Following incubation cell viability was determined by XTT assay;

FIG. 9 is a graph depicting the EAE clinical score in mice treated with CX-5461 on the same day when EAE was induced in the mice. Note the delayed onset and suppressed severity of MOG induced EAE by various concentrations of CX-5461 administered from EAE induction.

FIG. 10 is a graph depicting the EAE clinical score in mice treated with CX-5461 after EAE onset in the mice. Note the suppression of MOG induced EAE by CX-5461 treatment CX-5461 administered from EAE clinical onset.

FIG. 11 is art accepted conversion Table approved by the FDA for determination of human equivalent dose (HED) based on body surface area (BSA) (Reagan-Shaw S., et al., FASEB J. 22:659-661 (2007)). To convert dose in mg/kg to dose in mg/m², multiply by Kₘ value. The human equivalent dose is calculated as follows: HED (mg/kg)=Animal dose (mg/kg) multiplied by (Animal Kₘ/human Kₘ).

### DESCRIPTION OF SPECIFIC EMBODIMENTS OF THE INVENTION

The present invention, in some embodiments thereof, relates to methods of treating autoimmune diseases and, more particularly, but not exclusively, to method of treating multiple sclerosis using a quinolone compound such as CX-5461.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have uncovered that 2-(4-methyl-1,4-diazepan-1-yl)-N-((5-methylpyrazin-2-yl)methyl)-5-oxo-5H-benzo[4,5]thiazolo[3,2-a][1,8]naphthyridine-6-carboxamide (also known as “CX-5461”), which is a potent RNA Polymerase I (Pol I) inhibitor, can be used to treat autoimmune diseases such as multiple sclerosis.

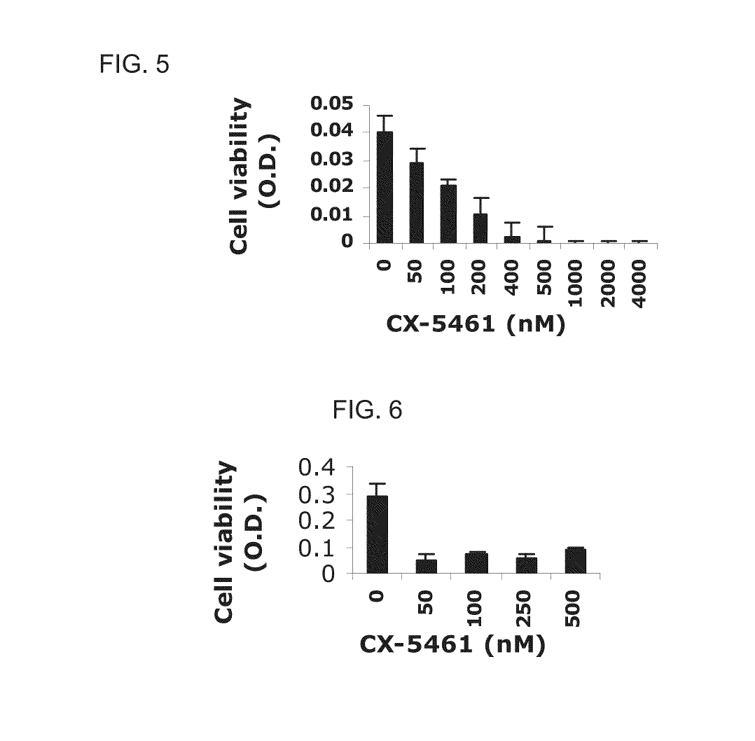

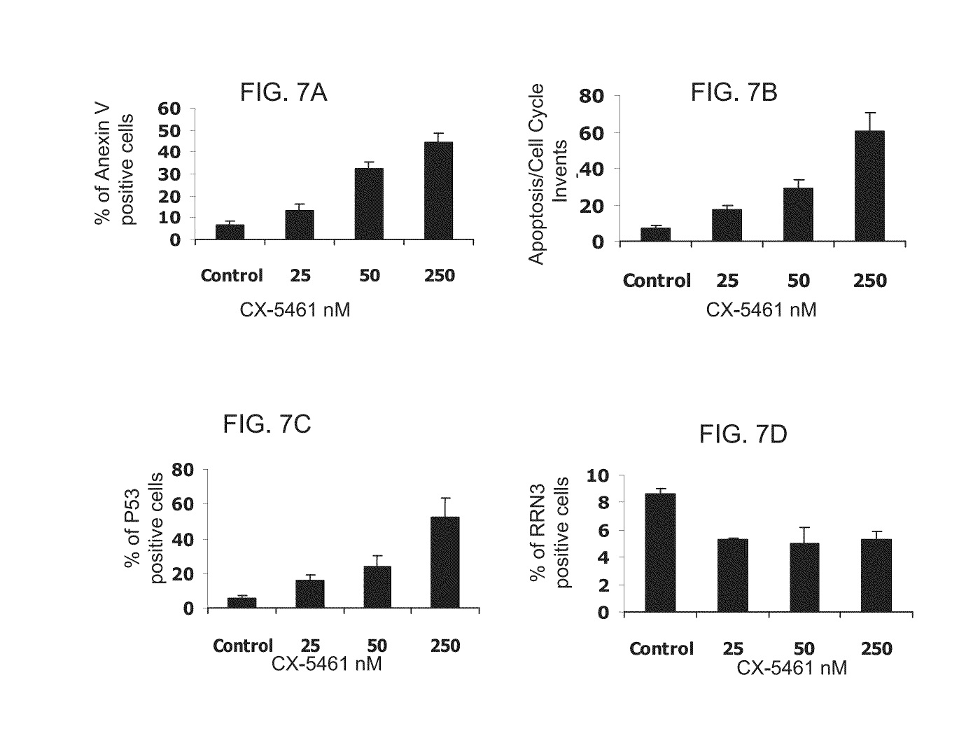

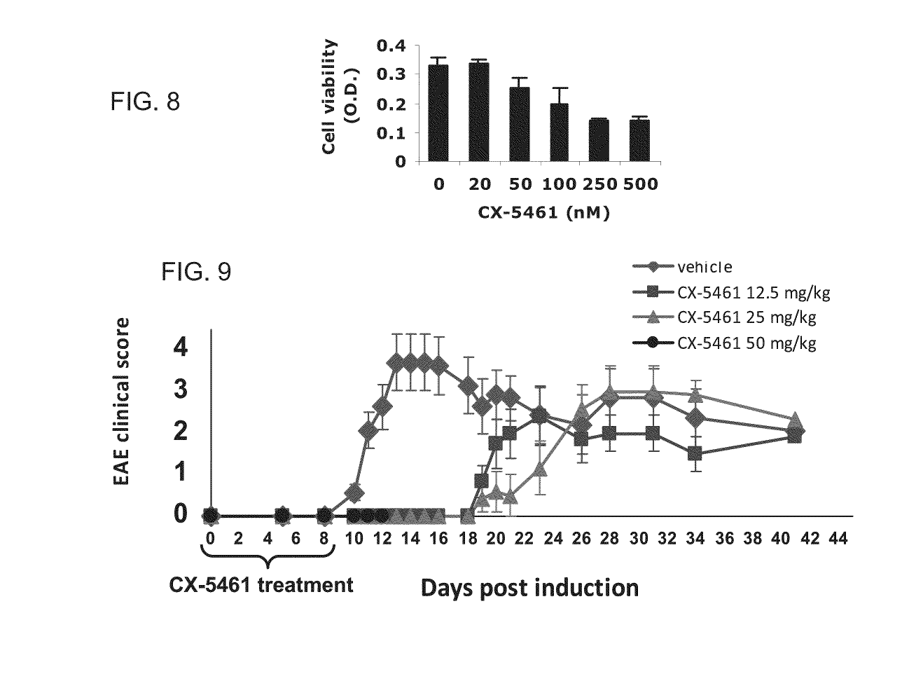

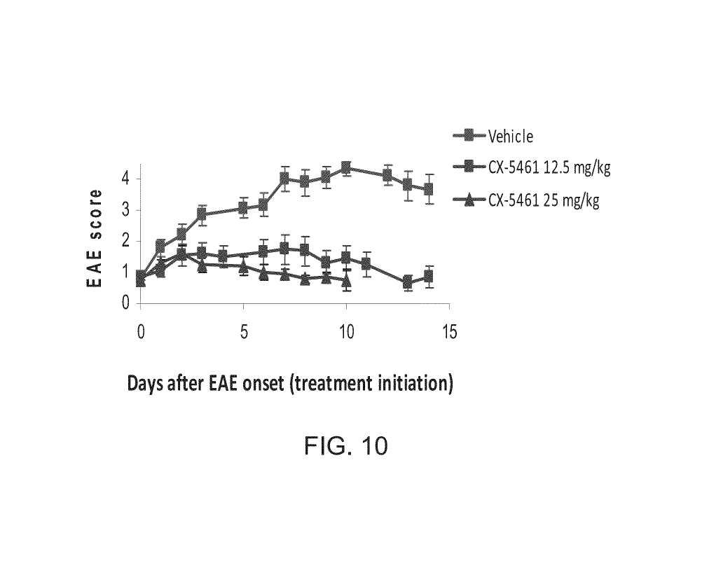

As shown in the Examples section which follows, the present inventors uncovered that the quinolone compound CX-5461 can reduce the viability of immune cells (e.g., splenocytes) in a dose-related manner (Example 2, FIG. 5), reduce the viability of normal (e.g., healthy) PBMCs (Example 3, FIG. 6), and induce apoptosis of PBMCs obtained from multiple sclerosis subjects (Example 4, FIGS. 7A-B). In addition, the present inventors have uncovered that CX-5461 increases the level of p53 in PBMCs obtained from multiple sclerosis subjects (Example 4, FIG. 7C) and decreases the number of RRN3-positive cells in PBMCs obtained from multiple sclerosis subjects (Example 4, FIG. 7D). The present inventors have further tested the viability of neuronal progenitor cells (NPCs) in the presence of the CX-5461 compound, and found that CX-5461 does not affect NPCs' viability at a concentration lower than 20 nM, and does not suppress more than 50% viability at concentrations higher than 50 nM (Example 5, FIG. 8). Moreover, the present inventors performed in-vivo experiments in which they tested the ability of CX-5461 to treat EAE, animal model of multiple sclerosis. Thus, as shown in Example 6 of the Examples section which follows, when CX-5461 was administered to the animals along with MOG-induction, i.e., before development of symptoms of EAE mice (a multiple sclerosis animal model), the animals exhibited delayed onset of multiple sclerosis symptoms as compared to control animals which were not treated with the CX-5461 compound (Example 6, Table 2). In addition, the CX-5461 compound also significantly decreased the maximal and cumulative clinical scores of multiple sclerosis in the EAE mice (Example 6, Table 2, FIG. 9), demonstrating the feasibility of the CX-5461 compound to treat multiple sclerosis patients by reducing severity of disease, and to delay onset or deterioration of disease. Moreover, when CX-5461 was administered in a dose of 12.5 mg/kg/day to mice upon disease onset (when the mice exhibited multiple sclerosis clinical score of 0.5 or 1.0), the treated mice exhibited significantly lower EAE scores as compared to the control vehicle group (Example 7 and FIG. 10), with a significantly reduced cumulative or maximal EAE score (Example 7, Table 3). Furthermore, the present inventors have shown that a dose of 25 mg/kg/day of CX-5461 is toxic to mice, as evidenced by a change in their fur color and brightness and loss of weight. These results demonstrate that treatment with the CX-5461 compound (e.g., in dose of about 1.01 mg/kg/day) upon onset of disease significantly decreases the severity of the disease.

According to an aspect of some embodiments of the invention, there is provided a method of treating an autoimmune disease in a subject, comprising: administering to the subject a therapeutically effective amount of a quinolone compound, thereby treating the autoimmune disease in the subject.

According to an aspect of some embodiments of the invention, the quinolone compound is for use in treating an autoimmune disease in a subject.

According to some embodiments of the invention, the quinolone compound has a general structure of Formula (I),

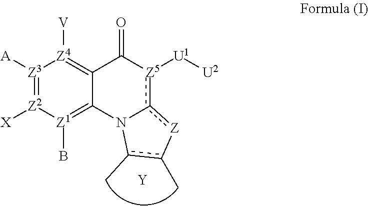

or a pharmaceutically acceptable salt or ester thereof;
wherein [symbol] indicates an optionally unsaturated bond;
each of B, X, A or V is absent if Z¹, Z², Z³ and Z⁴, respectively, is N; and
each of B, X, A and V is independently H, halo, azido, —CN, —CF₃, —CONR¹R², NR¹R², —SR², —OR², —R³, —W, -L-W, —W⁰, -L-N(R)—W⁰, A² or A³, when each of Z¹, Z², Z³ and Z⁴, respectively, is C;

### Z is O, S, CR⁴₂, NR⁴CR⁴, CR⁴NR⁴, CR⁴, NR⁴ or N;

each of Z¹, Z², Z³ and Z⁴ is independently C or N, provided any three N are non-adjacent;
Z⁵ is C; or Z⁵ may be N when Z is N;
Y is an optionally substituted 5-6 membered carbocyclic or heterocyclic ring;
U¹ is —C(=T)N(R)—, —C(=T)N(R)O—, —C(=T)-, —SO₂N(R)—, —SO₂N(R)N(R⁰)—, —SO₂—, or —SO₃—, where T is O, S, or NH; or U¹ may be a bond when Z⁵ is N or U² is H;
U² is H, or C3-C7 cycloalkyl, C1-C10 alkyl, C1-C10 heteroalkyl, C2-C10 alkenyl or C2-C10 heteroalkenyl group, each of which may be optionally substituted with one or more halogens, ═O, or an optionally substituted 3-7 membered carbocyclic or heterocyclic ring; or U² is —W, -L-W, -L-N(R)—W⁰, A² or A³;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R⁴ may be —W, -L-W or -L-N(R)—W⁰;
each R and R⁰ is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member;
W⁰ is an optionally substituted 3-4 membered carbocyclic ring, or a C1-C6 alkyl group substituted with from 1 to 4 fluorine atoms;
provided one of U², V, A, X and B is a secondary amine A² or a tertiary amine A³, wherein
the secondary amine A² is —NH—W⁰, and
the tertiary amine A³ is a fully saturated and optionally substituted six-membered or seven-membered azacyclic ring optionally containing an additional heteroatom selected from N, O or S as a ring member, or the tertiary amine A³ is a partially unsaturated or aromatic optionally substituted five-membered azacyclic ring, optionally containing an additional heteroatom selected from N, O or S as a ring member;

thereby treating the autoimmune disease in the subject.

According to some embodiments of the invention, Z¹ is N, and each of Z², Z³ and Z⁴ is C.

According to some embodiments of the invention, U is —W or -L-W, where W is an optionally substituted 5-6 membered unsaturated or aromatic azacyclic ring, optionally containing an additional heteroatom selected from N, O and S; or W is an optionally substituted 5-7 membered saturated azacyclic ring containing an additional heteroatom selected from N and S.

According to some embodiments of the invention, U² is -L-N(R)—W⁰.

According to some embodiments of the invention, Y is an optionally substituted phenyl ring.

According to some embodiments of the invention, the compound with the proviso that when Z¹ is N, Z² and Z⁴ are C, Z⁵ is C, U¹ is —C(O)NH—, U² is -L-W, and L is an ethylene linker, one of V, A, and X is independently an optionally substituted aryl, heteroaryl, or 7-membered azacyclic ring, optionally containing an additional heteroatom selected from N, O and S as a ring member, if W is pyrrolidin-1-yl, N-methyl-pyrrolidin-2-yl, piperidin-1-yl or morpholin-1-yl

According to some embodiments of the invention, the quinolone compound has a general structure of Formula (II),

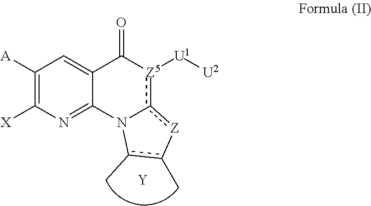

or a pharmaceutically acceptable salt or ester thereof;
wherein [symbol] indicates an optionally unsaturated bond;
each of A and X is independently H, halo, azido, —CN, —CF₃, —CONR¹R², —NR¹R², —SR², —OR², —R³, —W, -L-W, —W⁰, -L-N(R)—W⁰, A² or A³;

### Z is O, S, CR⁴₂, NR⁴CR⁴, CR⁴NR⁴ or NR⁴;

Y is an optionally substituted 5-6 membered carbocyclic or heterocyclic ring;
U¹ is —C(=T)N(R)—, —C(=T)N(R)O—, —C(=T)-, —SO₂N(R)—, —SO₂N(R)N(R⁰)—, —SO₂—, or —SO₃—, where T is O, S, or NH; or U¹ may be a bond when U2 is H;
U² is H, or C3-C7 cycloalkyl, C1-C10 alkyl, C1-C10 heteroalkyl, C2-C10 alkenyl or C2-C10 heteroalkenyl group, each of which may be optionally substituted with one or more halogens, ═O, or an optionally substituted 3-7 membered carbocyclic or heterocyclic ring; or U² is —W, -L-W, -L-N(R)—W⁰, A² or A³;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R⁴ may be —W, -L-W or -L-N(R)—W⁰;
each R and R⁰ is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member;
W⁰ is an optionally substituted 3-4 membered carbocyclic ring, or a C1-C6 alkyl group substituted with from 1 to 4 fluorine atoms;
provided that one of U², A, and X is a secondary amine A² or a tertiary amine A³, wherein
the secondary amine A² is —NH—W⁰, and
the tertiary amine A³ is a fully saturated and optionally substituted six-membered or seven-membered azacyclic ring optionally containing an additional heteroatom selected from N, O or S as a ring member, or the tertiary amine A³ is a partially unsaturated or aromatic optionally substituted five-membered azacyclic ring optionally containing an additional heteroatom selected from N, O or S as a ring member, thereby treating the autoimmune disease in the subject.

According to some embodiments of the invention, with the proviso that when U¹ is —C(O)NH—, U² is -L-W, and L is an ethylene linker, one of A and X is independently an optionally substituted aryl, heteroaryl, or 7-membered azacyclic ring, optionally containing an additional heteroatom selected from N, O and S as a ring member, if W is pyrrolidin-1-yl, N-methyl-pyrrolidin-2-yl, piperidin-1-yl or morpholin-1-yl.

According to some embodiments of the invention, at least one of A and X is a tertiary amine A³.

According to some embodiments of the invention, A³ is selected from the group consisting of imidazole, imidazoline, pyrroline, piperidine, piperazine, morpholine, thiomorpholine and homopiperazine.

According to some embodiments of the invention, U¹ is a —C(=T)N(R)—, T is O, and U² is -L-W or -L-N(R)—W⁰.

According to some embodiments of the invention, the quinolone compound has a general structure of Formula (III),

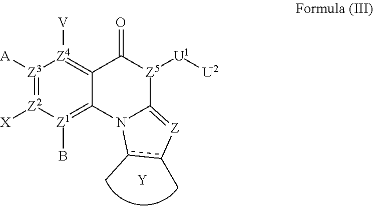

or a pharmaceutically acceptable salt or ester thereof;
wherein [symbol] indicates an optionally unsaturated bond; and
each of B, X, A or V is absent if Z¹, Z², Z³ and Z⁴, respectively, is N; and
each of B, X, A and V is independently H, halo, azido, —CN, —CF₃, —CONR¹R², —NR¹R², —SR², —OR², —R³, —W, -L-W, —W⁰, -L-N(R)—W⁰, A² or A³, when each of Z¹, Z², Z³ and Z⁴, respectively, is C;
each of Z¹, Z², Z³ and Z⁴ is independently C or N, provided any three N are non-adjacent;
Y is an optionally substituted 5-6 membered carbocyclic or heterocyclic ring;
U¹ is —C(=T)N(R)—, —C(=T)N(R)O—, —C(=T)-, —SO₂N(R)—, —SO₂N(R)N(R⁰)—, —SO₂—, or —SO₃—, where T is O, S, or NH; or U¹ may be a bond when Z⁵ is N or U² is H;
U² is H, or C3-C7 cycloalkyl, C1-C10 alkyl, C1-C10 heteroalkyl, C2-C10 alkenyl or C2-C10 heteroalkenyl group, each of which may be optionally substituted with one or more halogens, ═O, or an optionally substituted 3-7 membered carbocyclic or heterocyclic ring; or U² is —W, -L-W, -L-N(R)—W⁰, A² or A³;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R⁴ may be —W, -L-W or -L-N(R)—W⁰;
each R and R⁰ is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member;
W⁰ is an optionally substituted 3-4 membered carbocyclic ring, or a C1-C6 alkyl group substituted with from 1 to 4 fluorine atoms;
provided that one of U², V, A, X and B is a secondary amine A² or a tertiary amine A3, wherein
the secondary amine A² is —NH—W⁰, and
the tertiary amine A³ is a fully saturated and optionally substituted six-membered or seven-membered azacyclic ring optionally containing an additional heteroatom selected from N, O or S as a ring member, or the tertiary amine A³ is a partially unsaturated or aromatic optionally substituted five-membered azacyclic ring, optionally containing an additional heteroatom selected from N, O or S as a ring member, thereby treating the autoimmune disease in the subject.

According to some embodiments of the invention, the quinolone compound has a general structure of Formula (IV),

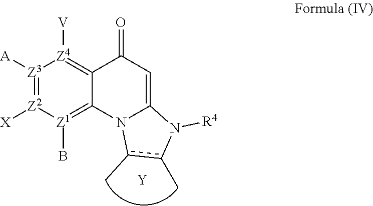

or a pharmaceutically acceptable salt or ester thereof;
wherein [symbol] indicates an optionally unsaturated bond;
each of B, X, A or V is absent if Z¹, Z², Z³ and Z⁴ respectively, is N; and
each of B, X, A and V is independently H, halo, azido, —CN, —CF₃, —CONR¹R², NR¹R², —SR², —OR², —R³, —W, -L-W, —W⁰, -L-N(R)—W⁰, A² or A³, when each of Z¹, Z², Z³ and Z⁴, respectively, is C;
each of Z¹, Z², Z³ and Z⁴ is independently C or N, provided any three N are non-adjacent;
Y is an optionally substituted 5-6 membered carbocyclic or heterocyclic ring;
in each NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;

### R⁴ is —W, -L-W or -L-N(R)—WO; and

each R is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member; and
W⁰ is an optionally substituted 3-4 membered carbocyclic ring, or a C1-C6 alkyl group substituted with from 1 to 4 fluorine atoms,

thereby treating the autoimmune disease in the subject.

According to some embodiments of the invention, the quinolone compound has a general structure of Formula (V),

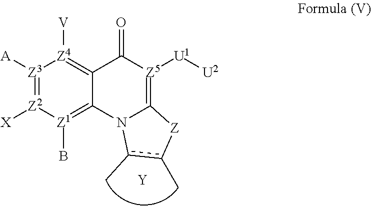

or a pharmaceutically acceptable salt or ester thereof; [symbol]
indicates an optionally unsaturated bond;
A and V independently are H, halo, azido, —CN, —CF₃, —CONR¹R², —NR¹R², —SR², —OR², —R³, —W, -L-W, —W⁰, -L-N(R)—W⁰, A² or A³;

### Z is O, S, CR⁴₂, NR⁴CR⁴, CR⁴NR⁴ or NR⁴;

Y is an optionally substituted 5-6 membered carbocyclic or heterocyclic ring;
U¹ is —C(=T)N(R)—, —C(=T)N(R)O—, —C(=T)-, —SO₂N(R)—, —SO₂N(R)N(R⁰)—, —SO₂—, or —SO₃—, where T is O, S, or NH; or U¹ may be a bond when U2 is H;
U² is H, or C3-C7 cycloalkyl, C1-C10 alkyl, C1-C10 heteroalkyl, C2-C10 alkenyl or C2-C10 heteroalkenyl group, each of which may be optionally substituted with one or more halogens, ═O, or an optionally substituted 3-7 membered carbocyclic or heterocyclic ring; or U² is —W, -L-W or -L-N(R)—W⁰, A² or A³;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R4 may be —W, -L-W or -L-N(R)—W⁰;
each R and R⁰ is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member;
W⁰ is an optionally substituted 3-4 membered carbocyclic ring, or a C1-C6 alkyl group substituted with from 1 to 4 fluorine atoms;
provided one of U², A, and V is a secondary amine A² or a tertiary amine A3, wherein
the secondary amine A² is —NH—WO, and
the tertiary amine A³ is a fully saturated and optionally substituted six-membered or seven-membered azacyclic ring optionally containing an additional heteroatom selected from N, O or S as a ring member, or the tertiary amine A³ is a partially unsaturated or aromatic optionally substituted five-membered azacyclic ring optionally containing an additional heteroatom selected from N, O or S as a ring member,

thereby treating the autoimmune disease in the subject.

According to some embodiments of the invention, the quinolone compound has a general structure of Formula (VI),

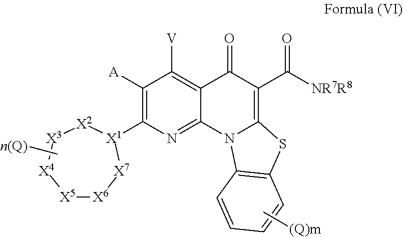

or a pharmaceutically acceptable salt or ester thereof;
wherein:

### X¹ is CH or N;

X², X³, X⁴, X⁵, X⁶ and X¹ independently are NR⁴, CH₂, CHQ or C(O)₂, provided that: (i) zero, one or two of X², X³, X⁴, X⁵, X⁶ and X¹ are NR⁴; (ii) when X¹ is N, both of X² and X⁷ are not NR⁴; (iii) when X¹ is N, X³ and X⁶ are not NR⁴; and (iv) when X¹ is CH and two of X², X³, X⁴, X⁵, X⁶ and X¹ are NR⁴, the two NR⁴ are located at adjacent ring positions or are separated by two or more other ring positions;
A and V independently are H, halo, azido, —CN, —CF₃, —CONR¹R², —NR¹R², —SR², —OR², —R³, —W, -L-W, —W⁰, or -L-N(R)—W⁰;
each Q is independently halo, azido, —CN, —CF₃, —CONR¹R², —NR¹R², —SR², —OR², —R³, —W, -L-W, —W⁰, or -L-N(R)—W⁰;
in each —NR¹R², R¹ and R² together with N may form an optionally substituted azacyclic ring, optionally containing one additional heteroatom selected from N, O and S as a ring member;
R¹ is H or C1-C6 alkyl, optionally substituted with one or more halogens, or ═O;
R is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R4 may be —W, -L-W or -L-N(R)—W⁰;
each R is independently H or C1-C6 alkyl;
R⁷ is H and R⁸ is C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring; or in NR⁷R⁸, R⁷ and R⁸ together with N may form an optionally substituted azacyclic ring, optionally containing an additional heteroatom selected from N, O and S as a ring member;
m is 0, 1, 2, 3 or 4;
n is 0, 1, 2, 3, 4, or 5;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member; and
W⁰ is an optionally substituted 3-4 membered carbocyclic ring, or a C1-C6 alkyl group substituted with from 1 to 4 fluorine atoms,

thereby treating the autoimmune disease in the subject.

According to some embodiments of the invention, X¹ is CH and two of X², X³, X⁴, X⁵, X⁶ and X¹ are NR⁴.

According to some embodiments of the invention, wherein X¹ is CH and one of X², X³, X⁴, X⁵, X⁶ and X⁷ and NR⁴.

According to some embodiments of the invention, X¹ is CH and none of X², X³, X⁴, X⁵, X⁶ and X⁷ are NR⁴.

According to some embodiments of the invention, wherein X¹ is N and none of X², X³, X⁴, X⁵, X⁶ and X⁷ are NR⁴.

According to some embodiments of the invention, X¹ is N and one of X⁴ or X⁵ is NR⁴.

According to some embodiments of the invention, the quinolone compound has a general structure of Formula (VIII),

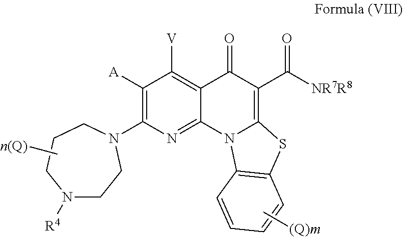

or a pharmaceutically acceptable salt or ester thereof;

wherein:
A and V independently are H, halo, azido, —CN, —CF₃, —CONR¹R², —NR¹R², —SR², —OR², —R³, —W, -L-W, —W⁰, or -L-N(R)—W⁰;
each Q is independently halo, azido, —CN, —CF₃, —CONR¹R², —NR¹R², —SR², —OR², —R³, —W, -L-W, —W⁰, or -L-N(R)—W⁰;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R4 may be —W, -L-W or -L-N(R)—W⁰;
each R is independently H or C1-C6 alkyl;
R⁷ is H and R⁸ is C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring; or in NR⁷R⁸, R⁷ and R⁸ together with N may form an optionally substituted azacyclic ring, optionally containing an additional heteroatom selected from N, O and S as a ring member;
m is 0, 1, 2, 3 or 4;
n is 0, 1, 2, 3, 4 or 5;
p is 0, 1, 2 or 3;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member; and
W⁰ is an optionally substituted 3-4 membered carbocyclic ring, or a C1-C6 alkyl group substituted with from 1 to 4 fluorine atoms,

thereby treating the autoimmune disease in the subject.

According to some embodiments of the invention, R⁷ is H and R⁸ is a C₁₋₄ alkyl substituted with an optionally substituted aromatic heterocyclic ring. According to some embodiments of the invention, the optionally substituted aromatic heterocyclic ring is selected from pyridine, pyrimidine, pyrazine, imidazole, pyrrolidine, and thiazole.

According to some embodiments of the invention, R⁷ and R⁸ together with N in —NR⁷R⁸ form an optionally substituted azacyclic ring selected from the group consisting of morpholine, thiomorpholine, piperidine or piperazine ring.

According to some embodiments of the invention, the quinolone compound has a general structure of Formula (VII)

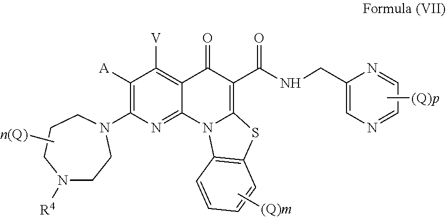

or a pharmaceutically acceptable salt or ester thereof;
A and V independently are H, halo, azido, —CN, —CF₃, —CONR¹R², —NR¹R², —SR², —OR², —R³, —W, -L-W, —W⁰, or -L-N(R)—W⁰;
each Q is independently halo, azido, —CN, —CF₃, —CONR¹R², NR¹R², —SR², —OR², R³, —W, -L-W, —W⁰, or -L-N(R)—W⁰;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R⁴ may be —W, -L-W or -L-N(R)—W⁰;
each R is independently H or C1-C6 alkyl;
m is 0, 1, 2, 3 or 4;
n is 0, 1, 2, 3, 4 or 5;
p is 0, 1, 2 or 3;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member; and
W⁰ is an optionally substituted 3-4 membered carbocyclic ring, or a C1-C6 alkyl group substituted with from 1 to 4 fluorine atoms,

thereby treating the autoimmune disease in the subject.

According to some embodiments of the invention, A and V are independently H or halo.

According to some embodiments of the invention, R⁴ is H or C1-4 alkyl.

According to some embodiments of the invention, m and n are each 0.

According to some embodiments of the invention, p is 0 or 1.

According to some embodiments of the invention, the compound having the structure:

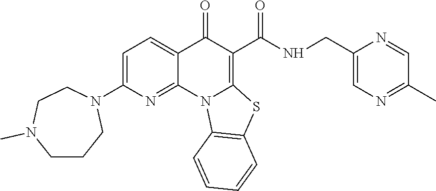

or a pharmaceutically acceptable salt or ester thereof.

According to some embodiments of the invention, the quinolone compound has a structure depicting in a Formula selected from the group consisting of:

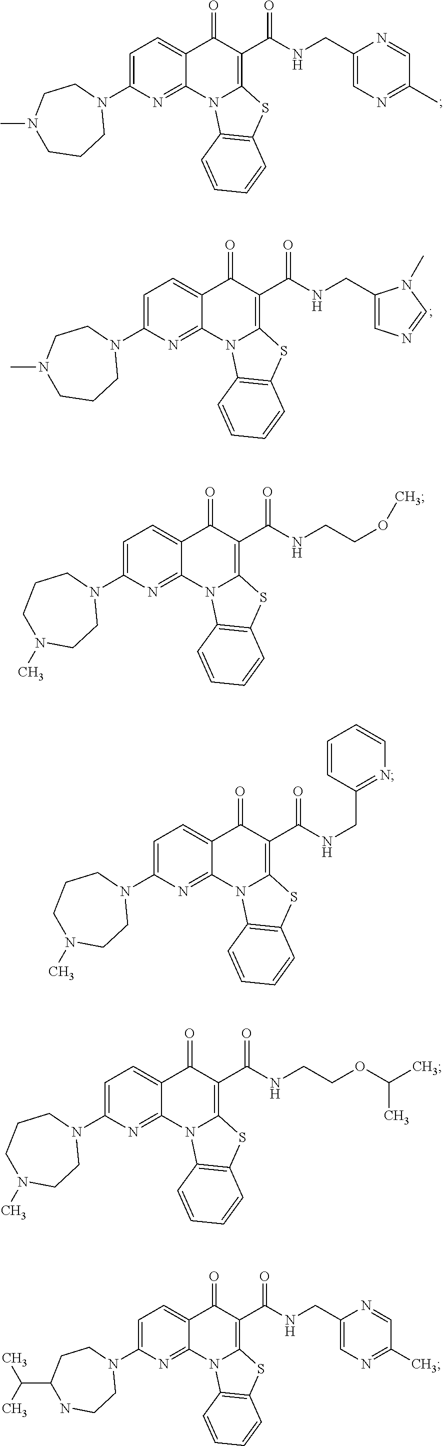

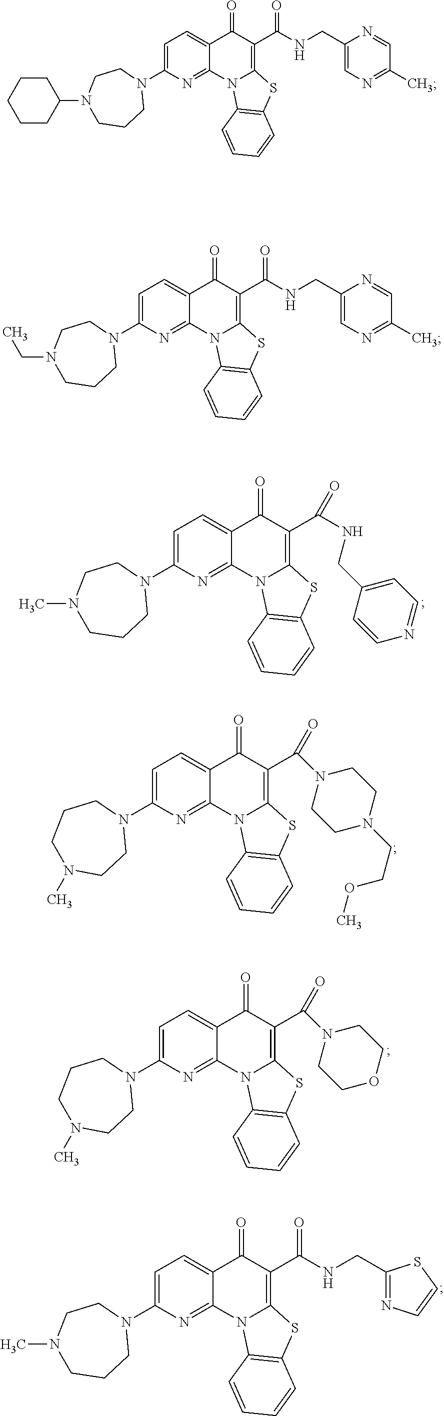 [SMILES: Cc1cnc(CNC(=O)c2c(=O)c3ccc(N4CCCN(C5CCCCC5)CC4)nc3n3c2sc2ccccc23)cn1 ; CCN1CCCN(c2ccc3c(=O)c(C(=O)NCc4cnc(C)cn4)c4sc5ccccc5n4c3n2)CC1 ; CN1CCCN(c2ccc3c(=O)c(C(=O)NCc4ccncc4)c4sc5ccccc5n4c3n2)CC1 ; COCCN1CCN(C(=O)c2c(=O)c3ccc(N4CCCN(C)CC4)nc3n3c2sc2ccccc23)CC1 ; CN1CCCN(c2ccc3c(=O)c(C(=O)N4CCOCC4)c4sc5ccccc5n4c3n2)CC1 ; CN1CCCN(c2ccc3c(=O)c(C(=O)NCc4nccs4)c4sc5ccccc5n4c3n2)CC1]

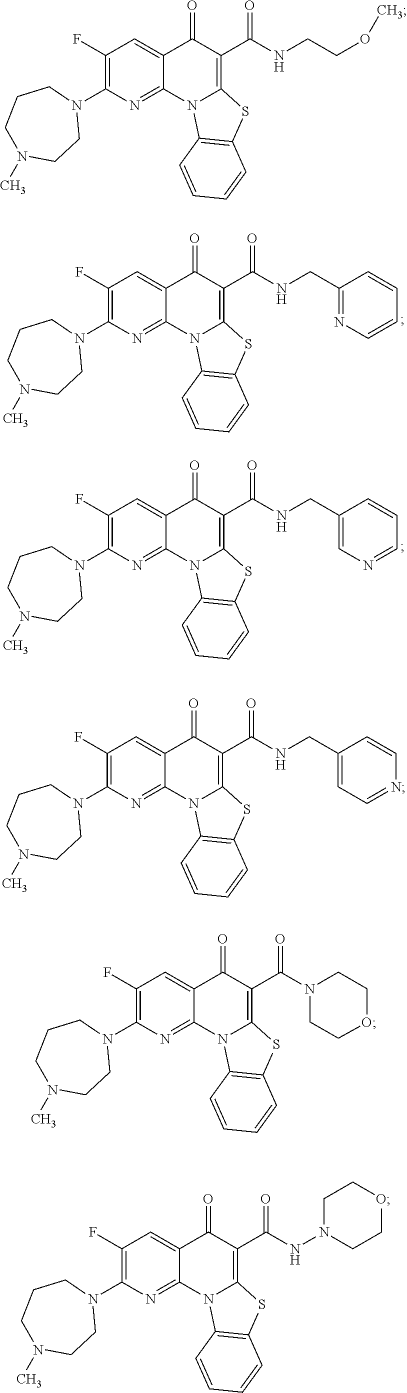

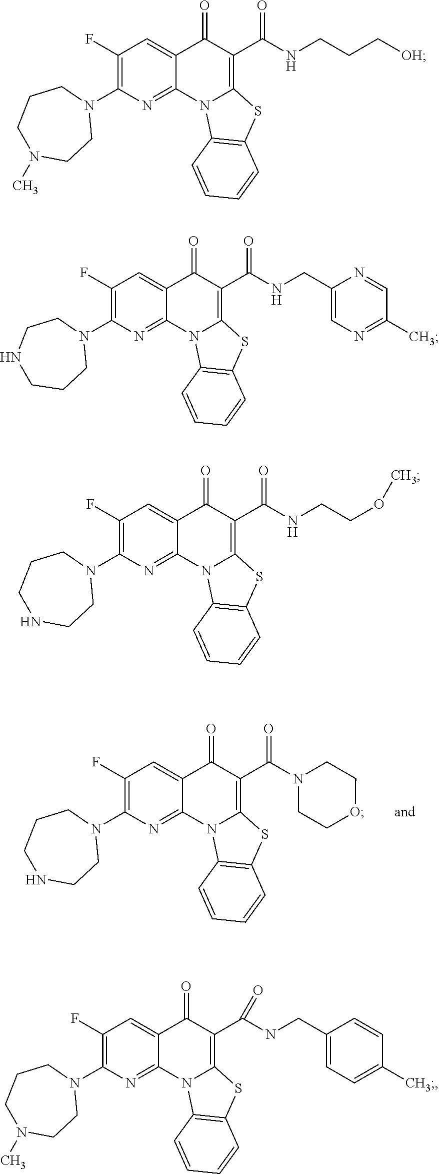

or a pharmaceutically acceptable salt or ester thereof,

thereby treating the autoimmune disease in the subject.

According to some embodiments of the invention, the compound is 2-(4-methyl-1,4-diazepan-1-yl)-N-((5-methylpyrazin-2-yl)methyl)-5-oxo-5H-benzo[4,5]thiazolo[3,2-a][1,8]naphthyridine-6-carboxamide as depicted in Formula

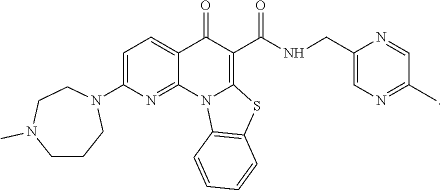

Methods of synthesizing the compound of some embodiments of the invention are known in the art and include for example those described in Nagasawa et al., US Patent Application Publication No. 2009/0093455A1, which is fully incorporated herein by reference. In addition, Example 1 of the Examples section which follows, provides a detailed description of synthesize of the CX-5461 compound.

For use as pharmaceutical agents, the compound of some embodiments of the invention is sterile.

According to some embodiments of the invention, the compound is purified using known methods.

According to some embodiments of the invention, the compound has 95-99.9% purity.

As used herein the phrase “autoimmune disease” refers to any disease caused by an autoimmune response, i.e., an immune response directed to a substance in the body of the subject.

It should be noted that since autoimmunity can affect any organ or tissue of the subject, e.g., the brain, skin, kidney, lungs, liver, heart, or thyroid of the subject, the clinical expression of the disease depends upon the site affected.

Following is a non-limiting list of autoimmune diseases or disorders (including autoimmune-related diseases or disorders) which can be treated by the compound of some embodiments of the invention: Acute Disseminated Encephalomyelitis (ADEM); Acute necrotizing hemorrhagic leukoencephalitis; Addison's disease; Agammaglobulinemia; Alopecia greata; Amyloidosis; Ankylosing spondylitis; Anti-GBM/Anti-TBM nephritis; Antiphospholipid syndrome (APS); Autoimmune angioedema; Autoimmune aplastic anemia; Autoimmune dysautonomia; Autoimmune hepatitis; Autoimmune hyperlipidemia; Autoimmune immunodeficiency; Autoimmune inner ear disease (AIED); Autoimmune myocarditis; Autoimmune pancreatitis; Autoimmune retinopathy; Autoimmune thrombocytopenic purpura (ATP); Autoimmune thyroid disease; Autoimmune urticaria; Axonal & neuronal neuropathies; Balo disease; Behcet's disease; Bullous pemphigoid; Cardiomyopathy; Castleman disease; Celiac disease; Chagas disease; Chronic inflammatory demyelinating polyneuropathy (CIDP); Chronic recurrent multifocal ostomyelitis (CRMO); Churg-Strauss syndrome; Cicatricial pemphigoid/benign mucosal pemphigoid; Crohn's disease; Cogans syndrome; Cold agglutinin disease; Congenital heart block; Coxsackie myocarditis; CREST disease; Essential mixed cryoglobulinemia; Demyelinating neuropathies; Dermatitis herpetiformis; Dermatomyositis; Devic's disease (neuromyelitis optica); Discoid lupus; Dressler's syndrome; Endometriosis; Eosinophilic fasciitis; Erythema nodosum; Experimental allergic encephalomyelitis; Evans syndrome; Fibrosing alveolitis; Giant cell arteritis (temporal arteritis); Glomerulonephritis; Goodpasture's syndrome; Granulomatosis with Polyangiitis (GPA) see Wegener's; Graves' disease; Guillain-Barre syndrome; Hashimoto's encephalitis; Hashimoto's thyroiditis; Hemolytic anemia; Henoch-Schonlein purpura; Herpes gestationis; Hypogammaglobulinemia; Idiopathic thrombocytopenic purpura (ITP); IgA nephropathy; IgG4-related sclerosing disease; Immunoregulatory lipoproteins; Inclusion body myositis; Insulin-dependent diabetes (type1); Interstitial cystitis; Juvenile arthritis; Juvenile diabetes; Kawasaki syndrome; Lambert-Eaton syndrome; Leukocytoclastic vasculitis; Lichen planus; Lichen sclerosus; Ligneous conjunctivitis; Linear IgA disease (LAD); Lupus (SLE); Lyme disease, chronic; Meniere's disease; Microscopic polyangiitis; Mixed connective tissue disease (MCTD); Mooren's ulcer; Mucha-Habermann disease; Multiple sclerosis; Myasthenia gravis; Myositis; Narcolepsy; Neuromyelitis optica (Devic's); Neutropenia; Ocular cicatricial pemphigoid; Optic neuritis; Palindromic rheumatism; PANDAS (Pediatric Autoimmune Neuropsychiatric Disorders Associated with Streptococcus); Paraneoplastic cerebellar degeneration; Paroxysmal nocturnal hemoglobinuria (PNH); Parry Romberg syndrome; Parsonnage-Turner syndrome; Pars planitis (peripheral uveitis); Pemphigus; Peripheral neuropathy; Perivenous encephalomyelitis; Pernicious anemia; POEMS syndrome; Polyarteritis nodosa; Type I, II, & III autoimmune polyglandular syndromes; Polymyalgia rheumatica; Polymyositis; Postmyocardial infarction syndrome; Postpericardiotomy syndrome; Progesterone dermatitis; Primary biliary cirrhosis; Primary sclerosing cholangitis; Psoriasis; Psoriatic arthritis; Idiopathic pulmonary fibrosis; Pyoderma gangrenosum; Pure red cell aplasia; Raynauds phenomenon; Reflex sympathetic dystrophy; Reiter's syndrome; Relapsing polychondritis; Restless legs syndrome; Retroperitoneal fibrosis; Rheumatic fever; Rheumatoid arthritis; Sarcoidosis; Schmidt syndrome; Scleritis; Scleroderma; Sjogren's syndrome; Sperm & testicular autoimmunity; Stiff person syndrome; Subacute bacterial endocarditis (SBE); Susac's syndrome; Sympathetic ophthalmia; Takayasu's arteritis; Temporal arteritis/Giant cell arteritis; Thrombocytopenic purpura (TTP); Tolosa-Hunt syndrome; Transverse myelitis; Ulcerative colitis; Undifferentiated connective tissue disease (UCTD); Uveitis; Vasculitis; Vesiculobullous dermatosis; Vitiligo; Wegener's granulomatosis (now termed Granulomatosis with Polyangiitis (GPA).

According to some embodiments of the invention, the autoimmune disease is multiple sclerosis.

The diagnosis of “multiple sclerosis” can be made when a subject has experienced at least one neurological attack affecting the central nervous system (CNS) accompanied by demyelinating lesions within the brain or spinal cord, which may have, but not necessarily confirmed by magnetic resonance imaging (MRI). The neurological attack can involve acute or sub-acute neurological symptomatology (attack) manifested by various clinical presentations like unilateral loss of vision, vertigo, ataxia, incoordination, gait difficulties, sensory impairment characterized by paresthesia, dysesthesia, sensory loss, urinary disturbances until incontinence, diplopia, dysarthria, various degrees of motor weakness until paralysis, cognitive decline either as a monosymptomatic or in combination. The symptoms usually remain for several days to few weeks, and then partially or completely resolve.

Further details on the diagnosis of multiple sclerosis according to 2010 McDonald Criteria for Diagnosis of MS are provided in Polman C H., et al., 2011 (“Diagnostic criteria for multiple sclerosis: 2010 revisions to the McDonald criteria” Annals of Neurology, vol. 69 (2): pages 292-302) which is fully incorporated herein by reference in its entirety.

For example, the diagnosis of multiple sclerosis can be made upon (I): Clinical presentation of ≧2 attacks, with objective clinical evidence of ≧2 lesions or objective clinical evidence of 1 lesion with reasonable historical evidence of a prior attack; (II): Clinical presentation of ≧2 attacks, with objective clinical evidence of 1 lesion, additional data have to include dissemination in space, demonstrated by: ≧1 T2 lesion in at least 2 of 4 MS-typical regions of the CNS (periventricular, juxtacortical, infratentorial, or spinal cord); (III): Clinical presentation of 1 attack, with objective clinical evidence of ≧2 lesions, additional data have to include dissemination in time, demonstrated by: Simultaneous presence of asymptomatic gadolinium-enhancing and nonenhancing lesions at any time; or A new T2 and/or gadolinium-enhancing lesion(s) on follow-up MRI, irrespective of its timing with reference to a baseline scan; (IV): Clinical presentation of 1 attack, additional data have to include dissemination in space and time, demonstrated by: For DIS: ≧1 T2 lesion in at least 2 of 4 MS-typical regions of the CNS (periventricular, juxtacortical, infratentorial, or spinal cord) and for DIT: Simultaneous presence of asymptomatic gadolinium-enhancing and nonenhancing lesions at any time; or A new T2 and/or gadolinium-enhancing lesion(s) on follow-up MRI, irrespective of its timing with reference to a baseline scan; (V): Clinical presentation of Insidious neurological progression suggestive of MS (PPMS), additional data have to include 1 year of disease progression (retrospectively or prospectively determined) plus 2 of 3 of the following criteria: 1. Evidence for DIS in the brain based on ≧1 T2 lesions in the MS-characteristic (periventricular, juxtacortical, or infratentorial) regions 2. Evidence for DIS in the spinal cord based on ≧2 T2 lesions in the cord 3. Positive CSF (isoelectric focusing evidence of oligoclonal bands and/or elevated IgG index).

According to some embodiments of the invention, the subject has relapsing-remitting multiple sclerosis (RRMS).

According to some embodiments of the invention, the subject has a primary progressive multiple sclerosis (PPMS).

According to some embodiments of the invention, the subject has a secondary progressive MS (SPMS).

According to some embodiments of the invention, the subject has benign multiple sclerosis (BMS).

According to some embodiments of the invention, the subject has a progressive-relapsing course of MS.

According to some embodiments of the invention, treating the subject refers to changing the disease course of the subject from a typical RRMS course to a BMS course.

According to some embodiments of the invention, treating the subject refers to suppressing the activity of typical RRMS course.

According to some embodiments of the invention, the compound is 2-(4-methyl-1,4-diazepan-1-yl)-N-((5-methylpyrazin-2-yl)methyl)-5-oxo-5H-benzo[4,5]thiazolo[3,2-a][1,8]naphthyridine-6-carboxamide as depicted in Formula

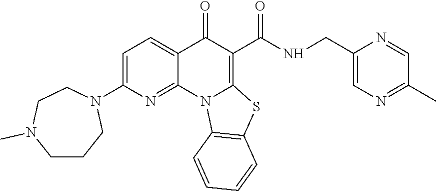

and wherein the autoimmune disease is multiple sclerosis.

According to some embodiments of the invention, administering the compound is performed after diagnosing the subject as having the autoimmune disease.

According to some embodiments of the invention, the autoimmune disease is multiple sclerosis and the diagnosis comprises appearance of brain lesions characteristics of the multiple sclerosis.

According to some embodiments of the invention, the compound prevents the appearance of additional neurological attack(s) and/or brain lesion(s) as compared to the number of neurological attack(s) and/or brain lesion(s) present at time of diagnosing multiple sclerosis.

As used herein the phrase “treating” refers to inhibiting or arresting the development of the autoimmune disease [e.g., multiple sclerosis] and/or causing the reduction, remission, or regression of the autoimmune disease and/or optimally curing the autoimmune disease. Those of skill in the art will understand that various methodologies and assays can be used to assess the development of autoimmune disease, and similarly, various methodologies and assays may be used to assess the reduction, remission or regression of the autoimmune disease.

As used herein, the term “subject” includes mammals, preferably human beings at any age which suffer from the pathology (the autoimmune disease).

According to some embodiments of the invention, the term “subject” encompasses individuals who are at risk to develop the pathology or are suspected of having the pathology.

The compound of some embodiments of the invention can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the compound of some embodiments of the invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

According to some embodiments of the invention, the compound is administered by oral administration.

Conventional approaches for drug delivery to the central nervous system (CNS) include: neurosurgical strategies (e.g., intracerebral injection or intracerebroventricular infusion); pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (the compound of some embodiments of the invention) effective to prevent, alleviate or ameliorate symptoms of a disorder (e.g., an autoimmune disease such as multiple sclerosis) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fing1, et al., 1975, in “The Pharmacological Basis of Therapeutics”, Ch. 1 p. 1).

Dosage amount and interval may be adjusted individually to provide tissue or blood levels of the active ingredient which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

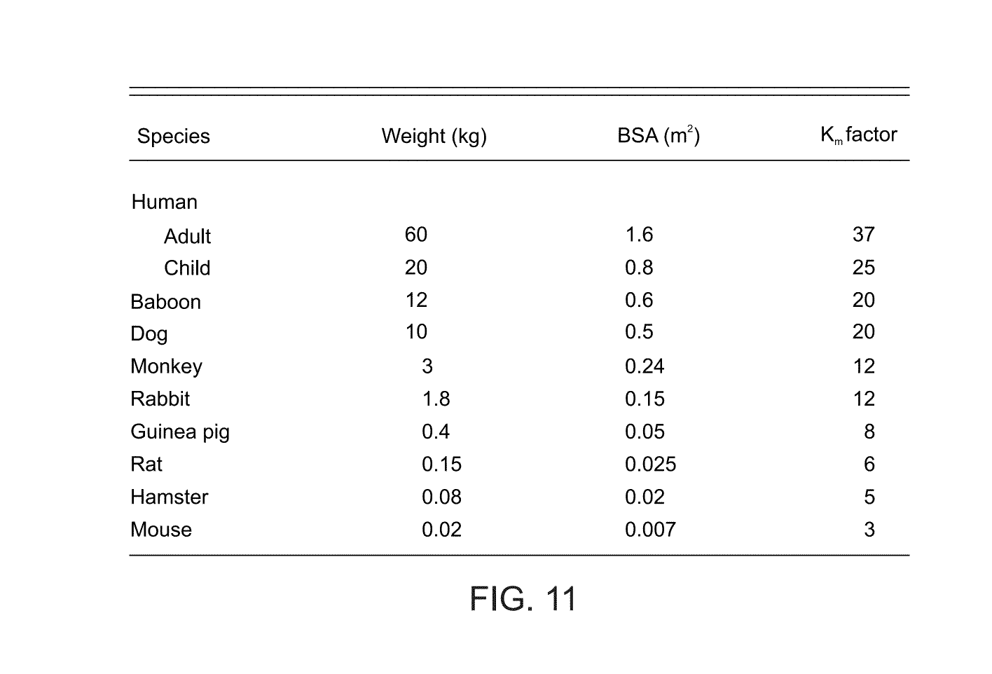

The doses shown herein with respect to the mouse animal model can be converted for the treatment other species such as human and other animals diagnosed with the autoimmune disease. FIG. 11 shows an art accepted conversion Table approved by the FDA (Reagan-Shaw S., et al., FASEB J. 22:659-661 (2007)).

The human equivalent dose is calculated as follows: HED (mg/kg)=Animal dose (mg/kg) multiplied by (Animal Kₘ/human Kₘ).

According to some embodiments of the invention, the compound is provided at a concentration range of about 0.081-1.62 mg/kg/day, e.g., about 0.162-1.539 mg/kg/day, 0.243-1.458 mg/kg/day, 0.324-1.458 mg/kg/day, 0.405-1.377 mg/kg/day, 0.486-1.296 mg/kg/day, 0.567-1.215 mg/kg/day, 0.648-1.134 mg/kg/day, 0.729-1.053 mg/kg/day, 0.81-0.972 mg/kg/day, 0.891-1.215 mg/kg/day, 0.972-1.134 mg/kg/day.

According to some embodiments of the invention, the compound is provided at a concentration range of about 0.81-1.215 mg/kg/day.

According to some embodiments of the invention, the compound is provided at a concentration of about 1.01 mg/kg/day.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

The teachings of the invention can be also used to determine efficiency of the compound of some embodiments of the invention in treating the autoimmune disease (e.g., multiple sclerosis) by determining the effect of the compound on the expression level of the at least one gene of the RNA polymerase I pathway. This can be used to develop a tailored treatment of an autoimmune disease by monitoring drug efficacy. This system is based on measuring the level of genes of the RNA polymerase I pathway during treatment with the compound and the ability to perform an ongoing fine-tuning drug efficacy assessment.

Thus, according to an aspect of some embodiments of the invention, there is provided a method of monitoring treatment efficiency of the compound of some embodiments of the invention, the method comprising:

(a) treating the subject with the compound according to the method of some embodiments of the invention, and

(b) comparing a level of expression of least one gene involved in the RNA polymerase I pathway in a cell of the subject following treating with the compound to a level of expression of the at least one gene in a cell of the subject prior to treating the subject with the compound,

(i) wherein a decrease above a predetermined threshold in the level of expression of the at least one gene following treating with the compound relative to the level of expression of the at least one gene prior to treating with the compound indicates that the compound is efficient for treating the subject;

(ii) wherein an increase above a predetermined threshold in the level of expression of the at least one gene following treating with the compound relative to the level of expression of the at least one gene prior to treating with the compound indicates that the compound is not efficient for treating the subject; or

(iii) wherein when a level of expression of the at least one gene following treating with the compound is identical or changed below a predetermined threshold as compared to prior to treating with the compound then the treatment is not efficient for treating the subject;

thereby monitoring treatment efficiency of the subject having an autoimmune disease.

As used herein, the phrase “level of expression” refers to the degree of gene expression and/or gene product activity in a specific cell. For example, up-regulation or down-regulation of various genes can affect the level of the gene product (i.e., RNA and/or protein) in a specific cell.

It should be noted that the level of expression can be determined in arbitrary absolute units, or in normalized units (relative to known expression levels of a control reference). For example, when using DNA chips, the expression levels are normalized according to the chips' internal controls or by using quantile normalization such as RMA.

As used herein the phrase “a cell of the subject” refers to at least one cell (e.g., an isolated cell), cell culture, cell content and/or cell secreted content which contains RNA and/or proteins of the subject. Examples include a blood cell, a cell obtained from any tissue biopsy [e.g., cerebrospinal fluid, (CSF), brain biopsy], a bone marrow cell, body fluids such as plasma, serum, saliva, spinal fluid, lymph fluid, the external sections of the skin, respiratory, intestinal, and genitourinary tracts, tears, saliva, sputum and milk. According to an embodiment of the invention, the cell is a blood cell (e.g., white blood cells, macrophages, B- and T-lymphocytes, monocytes, neutrophiles, eosinophiles, and basophiles) which can be obtained using a syringe needle from a vein of the subject. It should be noted that the cell may be isolated from the subject (e.g., for in vitro detection) or may optionally comprise a cell that has not been physically removed from the subject (e.g., in vivo detection).

According to some embodiments of the invention, the white blood cell comprises peripheral blood mononuclear cells (PBMC). The phrase, “peripheral blood mononuclear cells (PBMCs)” as used herein, refers to a mixture of monocytes and lymphocytes. Several methods for isolating white blood cells are known in the art. For example, PBMCs can be isolated from whole blood samples using density gradient centrifugation procedures. Typically, anticoagulated whole blood is layered over the separating medium. At the end of the centrifugation step, the following layers are visually observed from top to bottom: plasma/platelets, PBMCs, separating medium and erythrocytes/granulocytes. The PBMC layer is then removed and washed to remove contaminants (e.g., red blood cells) prior to determining the expression level of the polynucleotide(s) therein.

According to some embodiments of the invention, the level of expression of the gene(s) of the invention is determined using an RNA and/or a protein detection method.

According to some embodiments of the invention, the RNA or protein molecules are extracted from the cell of the subject.

Methods of extracting RNA or protein molecules from cells of a subject are well known in the art. Once obtained, the RNA or protein molecules can be characterized for the expression and/or activity level of various RNA and/or protein molecules using methods known in the arts.

According to some embodiments of the invention, detection of the expression level of the RNA of the Pol I pathway is performed using a probe which specifically hybridizes to a polynucleotide expressed from the gene of the Pol I pathway (e.g., including any alternative spliced form which is known in the art).

Non-limiting examples of methods of detecting RNA molecules in a cell sample include Northern blot analysis, RT-PCR, RNA in situ hybridization (using e.g., DNA or RNA probes to hybridize RNA molecules present in the cells or tissue sections), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface [e.g., a glass wafer) with addressable location, such as Affymetrix microarray (Affymetrix®, Santa Clara, Calif.)].

For example, the level of RRN3 in a sample can be determined by RT-PCR using primers available from Santa Cruz Biotechnology Inc. (sc-106866-PR), or Taqman Gene Expression Assay HS00607907_ml (Applied Biosystems, Foster City, Calif., USA), according to manufacturer's recommendation.

According to some embodiments of the invention, detection of the expression level of the protein of the Pol I pathway is performed using an antibody which specifically binds to a polypeptide expressed from the gene of the Pol I pathway (e.g., including any variants thereof which is known in the art).

Non-limiting examples of methods of detecting the level and/or activity of specific protein molecules in a cell sample include Enzyme linked immunosorbent assay (ELISA), Western blot analysis, radio-immunoassay (RIA), Fluorescence activated cell sorting (FACS), immunohistochemical analysis, in situ activity assay (using e.g., a chromogenic substrate applied on the cells containing an active enzyme), in vitro activity assays (in which the activity of a particular enzyme is measured in a protein mixture extracted from the cells). For example, in case the detection of the expression level of a secreted protein is desired, ELISA assay may be performed on a sample of fluid obtained from the subject (e.g., serum), which contains cell-secreted content.

As described above, the level of expression of least one gene involved in the RNA polymerase I pathway in a cell of the subject following treating with the compound is compared to the level of expression of the at least one gene in a cell of the subject prior to treating the subject with the compound.

As used herein the phrase “following treating with the compound” refers to any time period after administering the compound to the subject, e.g., from a few minutes to hours, or from a few days to weeks or months after drug administration.

According to some embodiments of the invention the level of expression is determined following administration of the first dose of the compound.

According to some embodiments of the invention the level of expression is determined following administration of any dose of the compound.

As used herein the phrase “prior to treating with the compound” refers to any time period prior administering the compound to the subject, e.g., from a few minutes to hours, or from a few days to weeks or months prior to drug administration.

According to some embodiments of the invention the level of expression is determined prior any dose of the compound (e.g., when the subject is naïve to treatment).

According to some embodiments of the invention prior to treating refers to when the subject is first diagnosed with autoimmune disease, e.g., multiple sclerosis.

According to some embodiments of the invention prior to treating refers to when the subject is suspected of having the autoimmune disease (e.g., multiple sclerosis), or diagnosed with probable autoimmune disease (e.g., probable multiple sclerosis).

According to some embodiments of the invention prior to treating refers to upon the onset of the autoimmune disease.

According to some embodiments of the invention the effect of the treatment on the subject can be evaluated by monitoring the level of expression of at least one of the polynucleotides described hereinabove. For example, downregulation in the level of RRN3 in the subject following treatment can be indicative of the positive effect of the treatment on the subject, e.g., switching from a typical RRMS to a BMS course of multiple sclerosis.

As described above, a decrease above a predetermined threshold in the level of expression of the at least one gene following treating with the compound relative to the level of expression of the at least one gene prior to treating with the compound indicates that the compound is efficient for treating the subject.

As used herein the phrase “a decrease above a predetermined threshold” refers to a decrease in the level of expression in the cell of the subject following treating with the compound which is higher than a predetermined threshold such as a about 10%, e.g., higher than about 20%, e.g., higher than about 30%, e.g., higher than about 40%, e.g., higher than about 50%, e.g., higher than about 60%, higher than about 70%, higher than about 80%, higher than about 90%, higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times, higher than about 350, higher than about 500 times, higher than about 1000 times, or more relative to the level of expression prior to treating with the compound.

As described, an increase above a predetermined threshold in the level of expression of the at least one gene following treating with the compound relative to the level of expression of the at least one gene prior to treating with the compound indicates that the compound is not efficient for treating the subject.

As used herein the phrase “an increase above a predetermined threshold” refers to an increase in the level of expression in the cell of the subject following treating with the compound, which is higher than a predetermined threshold such as about 10%, e.g., higher than about 20%, e.g., higher than about 30%, e.g., higher than about 40%, e.g., higher than about 50%, e.g., higher than about 60%, higher than about 70%, higher than about 80%, higher than about 90%, higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times, higher than about 350, higher than about 500 times, higher than about 1000 times, or more relative to the level of expression of the at least one gene prior to treating with the compound.

As described, a level of expression of the at least one gene following treating with the compound which is identical or changed below a predetermined threshold as compared to prior to treating with the compound is indicative that the treatment is not efficient for treating the subject.

As used herein the phrase “changed below a predetermined threshold as compared to prior to treating with the compound” refers to an increase or a decrease in the level of expression in the cell of the subject following treating with the compound, which is lower than a predetermined threshold, such as lower than about 10 times, e.g., lower than about 9 times, e.g., lower than about 8 times, e.g., lower than about 7 times, e.g., lower than about 6 times, e.g., lower than about 5 times, e.g., lower than about 4 times, e.g., lower than about 3 times, e.g., lower than about 2 times, e.g., lower than about 90%, e.g., lower than about 80%, e.g., lower than about 70%, e.g., lower than about 60%, e.g., lower than about 50%, e.g., lower than about 40%, e.g., lower than about 30%, e.g., lower than about 20%, e.g., lower than about 10%, e.g., lower than about 9%, e.g., lower than about 8%, e.g., lower than about 7%, e.g., lower than about 6%, e.g., lower than about 5%, e.g., lower than about 4%, e.g., lower than about 3%, e.g., lower than about 2%, e.g., lower than about 1% relative to the level of expression of the at least one gene prior to treating with the compound.

Non-limiting examples of genes involved in the RNA polymerase I pathway which can be used according to the method of the invention include RRN3, LRPPRC, POLR1B, POLR1C, POLR1D, POLR2A, POLR2B, POLR2C, POLR2D, POLR2E, POLR2E, POLR2F, POLR2G, POLR2H, POLR2I, POLR2J, POLR2J2, MGC13098, POLR2K, POLR2L, POLR3B, POLR3C, POLR3D, POLR3E, POLR3F, POLR3G, POLR3K, POLRMT, POLRMT and POLS.

Sequence information regarding gene products (i.e., RNA transcripts and polypeptide sequences) of the genes of RNA polymerase I pathway and of probes which can be used for detection thereof can be found in Table 1 hereinbelow.

TABLE 1
Table 1
Genes involved in the RNA polymerase I pathway

| | | | Representative | | |
|---|---|---|---|---|---|
| | | Representative | polypeptide | | |
| Affymetrix | | Public ID/ | Public ID/ | Gene | |
| ProbSet | SEQ ID NO: | SEQ ID NO: | SEQ ID NO: | Symbol | Gene Title |
| 216902_s_at | 1 | AF001549/40; | NP_060897/81 | RRN3 | RRN3 RNA polymerase I |
| | | NM_018427/41 | | | transcription factor homolog |
| 211971_s_at | 2 | AI653608/42; | NP_573566/82 | LRPPRC | leucine-rich PPR-motif containing |
| | | NM_133259/43 | | | |
| 220113_x_at | 3 | NM_019014/44 | NP_001131076/83/ | POLR1B | polymerase (RNA) I |
| | | | NP_061887/120 | | polypeptide B, 128 kDa |
| 207515_s_at | 4 | NM_004875/45 | NP_976035/84 | POLR1C | polymerase (RNA) I |
| | | | | | polypeptide C, 30 kDa |
| 209317_at | 5 | AF008442/46 | NP_976035/85 | POLR1C | polymerase (RNA) I |
| | | | | | polypeptide C, 30 kDa |
| 218258_at | 6 | NM_015972/47 | NP_057056/86/ | POLR1D | polymerase (RNA) I |
| | | | NP_689918/121 | | polypeptide D, 16 kDa |
| 202725_at | 7 | NM_000937/48 | NP_000928/87 | POLR2A | polymerase (RNA) II (DNA directed) |
| | | | | | polypeptide A, 220 kDa |
| 217420_s_at | 8 | M21610/49 | NP_000928/88 | POLR2A | polymerase (RNA) II (DNA directed) |
| | | | | | polypeptide A, 220 kDa |
| 201803_at | 9 | NM_000938/50 | NP_000929/89 | POLR2B | polymerase (RNA) II (DNA directed) |
| | | | | | polypeptide B, 140 kDa |
| 208996_s_at | 10 | BC000409/51 | NP_116558/90 | POLR2C | polymerase (RNA) II (DNA directed) |
| | | | | | polypeptide C, 33 kDa |
| 214263_x_at | 11 | AI192781/52 | NP_116558/91 | POLR2C | polymerase (RNA) II (DNA directed) |
| | | | | | polypeptide C, 33 kDa |
| 216282_x_at | 12 | AJ224143/53 | NP_116558/92 | POLR2C | polymerase (RNA) II (DNA directed) |
| | | | | | polypeptide C, 33 kDa |
| 203664_s_at | 13 | NM_004805/54 | NP_004796/93 | POLR2D | polymerase (RNA) II (DNA directed) |
| | | | | | polypeptide D |
| 214144_at | 14 | BF432147/55 | NP_004796/94 | POLR2D | polymerase (RNA) II (DNA directed) |
| | | | | | polypeptide D |
| 213887_s_at | 15 | AI554759/56 | NP_002686/95 | POLR2E | polymerase (RNA) II (DNA directed) |
| | | | | | polypeptide E, 25 kDa |
| 217854_s_at | 16 | BC004441/57 | NP_002686/96 | POLR2E | polymerase (RNA) II (DNA directed) |
| | | | | | polypeptide E, 25 kDa |
| 209511_at | 17 | BC003582/58 | NP_068809/97 | POLR2F | polymerase (RNA) II (DNA directed) |
| | | | | | polypeptide F |
| 202306_at | 18 | NM_002696/59 | NP_002687/98 | POLR2G | polymerase (RNA) II (DNA directed) |
| | | | | | polypeptide G |
| 209302_at | 19 | U37689/60 | NP_006223/99 | POLR2H | polymerase (RNA) II (DNA directed) |
| | | | | | polypeptide H |
| 212955_s_at | 20 | AL037557/61 | NP_006224/100 | POLR2I | polymerase (RNA) II (DNA directed) |
| | | | | | polypeptide I, 14.5 kDa |
| 212782_x_at | 21 | BG335629/62 | NP_006225/101 | POLR2J | polymerase (RNA) II (DNA directed) |
| | | | | | polypeptide J, 13.3 kDa |
| 216242_x_at | 22 | AW402635/63 | NP_116581/102 | POLR2J2 | DNA directed RNA polymerase II |
| | | | | | polypeptide J-related gene |
| 214740_at | 23 | BE676209/ | NP_001091084/103/ | POLR2J2 /// | DNA directed RNA polymerase II |
| | | 64 | NP_006225/122/ | MGC13098 | polypeptide J-related gene /// |
| | | | NP_116581/125 | | hypothetical prote |
| 202634_at | 24 | AL558030/65 | NP_005025/104 | POLR2K | polymerase (RNA) II (DNA directed) |
| | | | | | polypeptide K, 7.0 kDa |
| 202635_s_at | 25 | NM_005034/66 | NP_005025/105 | POLR2K | polymerase (RNA) II (DNA directed) |
| | | | | | polypeptide K, 7.0 kDa |
| 202586_at | 26 | AA772747/67 | NP_066951/106 | POLR2L | polymerase (RNA) II (DNA directed) |
| | | | | | polypeptide L, 7.6 kDa |
| 211730_s_at | 27 | BC005903/68 | NP_066951/107 | POLR2L | polymerase (RNA) II (DNA directed) |
| | | | | | polypeptide L, 7.6 kDa /// |
| | | | | | polymerase (RNA) II |
| 219459_at | 28 | NM_018082/69 | NP_001154180/108/ | POLR3B | polymerase (RNA) III (DNA directed) |
| | | | NP_060552/123 | | polypeptide B |
| 209382_at | 29 | U93867/70 | NP_006459/109 | POLR3C | polymerase (RNA) III (DNA directed) |
| | | | | | polypeptide C (62 kD) |
| 210573_s_at | 30 | BC004424/71 | NP_006459/110 | POLR3C | polymerase (RNA) III (DNA directed) |
| | | | | | polypeptide C (62 kD) |
| 208361_s_at | 31 | NM_001722/72 | NP_001713/111 | POLR3D | polymerase (RNA) III (DNA directed) |
| | | | | | polypeptide D, 44 kDa |
| 218016_s_at | 32 | NM_018119/73 | NP_060589/112 | POLR3E | polymerase (RNA) III (DNA directed) |
| | | | | | polypeptide E (80 kD) |
| 205218_at | 33 | NM_006466/74 | NP_006457/113 | POLR3F | polymerase (RNA) III (DNA directed) |
| | | | | | polypeptide F, 39 kDa |
| 206653_at | 34 | BF062139/75 | NP_006458/114 | POLR3G | Polymerase (RNA) III (DNA directed) |
| | | | | | polypeptide G (32 kD) |
| 206654_s_at | 35 | NM_006467/76 | NP_006458/115 | POLR3G | polymerase (RNA) III (DNA directed) |
| | | | | | polypeptide G (32 kD) |
| 218866_s_at | 36 | AF060223/77 | NP_057394/116 | POLR3K | polymerase (RNA) III (DNA directed) |
| | | | | | polypeptide K, 12.3 kDa |
| 203782_s_at | 37 | NM_005035/78 | NP_005026/117 | POLRMT | polymerase (RNA) |
| | | | | | mitochondrial (DNA directed) |
| 203783_x_at | 38 | BF057617/79 | NP_005026/118 | POLRMT | polymerase (RNA) |
| | | | | | mitochondrial (DNA directed) |
| 202466_at | 39 | NM_006999/80 | NP_001165276/119/ | PAPD7 | polymerase (DNA directed) sigma |
| | | | NP_001165277/124/ | | |
| | | | NP_0089307126 | | |

According to some embodiments of the invention, the at least one gene involved in the RNA polymerase 1 pathway is selected from the group consisting of POLR1D, LRPPRC, RRN3 and NCL.

According to some embodiments of the invention, the at least one gene involved in the RNA polymerase 1 pathway is RRN3.

According to some embodiments of the invention, the at least one gene involved in the RNA polymerase 1 pathway is LRPPRC.

According to some embodiments of the invention, the at least one gene involved in the RNA polymerase 1 pathway is POLR1D.

According to some embodiments of the invention, the at least one gene involved in the RNA polymerase 1 pathway comprises RRN3 and POLR1D.

According to some embodiments of the invention, the at least one gene involved in the RNA polymerase 1 pathway comprises RRN3 and LRPPRC.

According to some embodiments of the invention, the at least one gene involved in the RNA polymerase 1 pathway comprises POLR1D and LRPPRC.

According to some embodiments of the invention, the at least one gene involved in the RNA polymerase 1 pathway comprises RRN3, LRPPRC and POLR1D.

According to some embodiments of the invention, the at least one gene involved in the RNA polymerase 1 pathway is RRN3 and NCL.

According to some embodiments of the invention, the at least one gene involved in the RNA polymerase 1 pathway is LRPPRC and NCL.

According to some embodiments of the invention, the at least one gene involved in the RNA polymerase 1 pathway is POLR1D and NCL.

According to some embodiments of the invention, the at least one gene involved in the RNA polymerase 1 pathway comprises RRN3, POLR1D and NCL.

According to some embodiments of the invention, the at least one gene involved in the RNA polymerase 1 pathway comprises RRN3, LRPPRC and NCL.

According to some embodiments of the invention, the at least one gene involved in the RNA polymerase 1 pathway comprises POLR1D, LRPPRC and NCL.

According to some embodiments of the invention, the at least one gene involved in the RNA polymerase 1 pathway comprises RRN3, LRPPRC, POLR1D and NCL.

Qualifying the compound as being suitable for treating the autoimmune disease in the subject can be also performed by an in-vitro method.

Thus, according to an aspect of some embodiments of the invention there is provided an in vitro method of predicting an efficiency of the compound of some embodiments of the invention for treatment of a subject diagnosed with an autoimmune disease, the method is effected by:

(a) contacting cells of the subject with a therapeutically effective amount of the compound; and

(b) comparing a level of expression in the cells of at least one gene involved in the RNA polymerase I pathway following contacting with the compound to a level of expression of the at least one gene in the cells prior to contacting with the compound,

(i) wherein a decrease above a predetermined threshold in the level of expression of the at least one gene following contacting with the compound relative to the level of expression of the at least one gene prior to contacting with the compound indicates that the treatment is efficient for treating the subject;

(ii) wherein an increase above a predetermined threshold in the level of expression of the at least one gene following contacting with the compound relative to the level of expression of the at least one gene prior to contacting with the compound indicates that the treatment is not efficient for treating the subject; or

(iii) wherein when a level of expression of the at least one gene following contacting with the compound is identical or changed below a predetermined threshold as compared to prior to contacting with the compound then the treatment is not efficient treating the subject.

thereby predicting the efficiency of the compound for treatment of the subject diagnosed with the autoimmune disease.

Contacting cells with the compound can be performed by any in vitro conditions including for example, adding the compound to cells derived from a subject (e.g., a primary cell culture, a cell line) or to a biological sample comprising same (e.g., a fluid, liquid which comprises the cells) such that the compound is in direct contact with the cells. According to some embodiments of the invention, the cells of the subject are incubated with the compound. The conditions used for incubating the cells are selected for a time period/concentration of cells/concentration of drug/ratio between cells and the compound and the like which enable the compound to induce cellular changes, such as changes in transcription and/or translation rate of specific genes, proliferation rate, differentiation, cell death, necrosis, apoptosis and the like.

Methods of monitoring cellular changes induced by the drugs are known in the art and include for example, the MTT test which is based on the selective ability of living cells to reduce the yellow salt MTT (3-(4,5-dimethylthiazolyl-2)-2,5-diphenyltetrazolium bromide) (Sigma, Aldrich St Louis, Mo., USA) to a purple-blue insoluble formazan precipitate; the BrDu assay [Cell Proliferation ELISA BrdU colorimetric kit (Roche, Mannheim, Germany]; the TUNEL assay [Roche, Mannheim, Germany]; the Annexin V assay [ApoAlert® Annexin V Apoptosis Kit (Clontech Laboratories, Inc., CA, USA)]; the Senescence associated-β-galactosidase assay (Dimri G P, Lee X, et al. 1995. A biomarker that identifies senescent human cells in culture and in aging skin in vivo. Proc Natl Acad Sci USA 92:9363-9367); as well as various RNA and protein detection methods (which detect level of expression and/or activity) which are further described hereinabove.

According to some embodiments of the invention, the cells are incubated under conditions which enable the effect of the compound on cellular processes such as downregulation of the at least one gene of the RNA polymerase I pathway.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to an aspect of some embodiments of the invention there is provided a kit for monitoring efficacy of a compound for treating an autoimmune disease, the comprising the compound of some embodiments of the invention and at least one agent suitable for detecting the expression level of at least one gene of the RNA polymerase I pathway.

According to some embodiments of the invention, the kit further comprises a reference cell obtained from a subject who is responsive to the compound, i.e., a subject whose clinical symptoms were improved following treatment with the compound.

According to some embodiments of the invention, the agent suitable for detecting the expression level of at least one gene of the RNA polymerase I pathway is an antibody which specifically binds to a protein encoded by the at least one gene of the RNA polymerase I pathway.

According to some embodiments of the invention, the agent suitable for detecting the expression level of at least one gene of the RNA polymerase I pathway is a polynucleotide probe which specifically hybridizes to an RNA of the at least one gene of the RNA polymerase I pathway.

According to some embodiments of the invention, the compound which is included in the kit is CX-5461 and the agent is suitable for detecting the expression level of RRN3 in cells.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example I

CX-5461 Synthesis

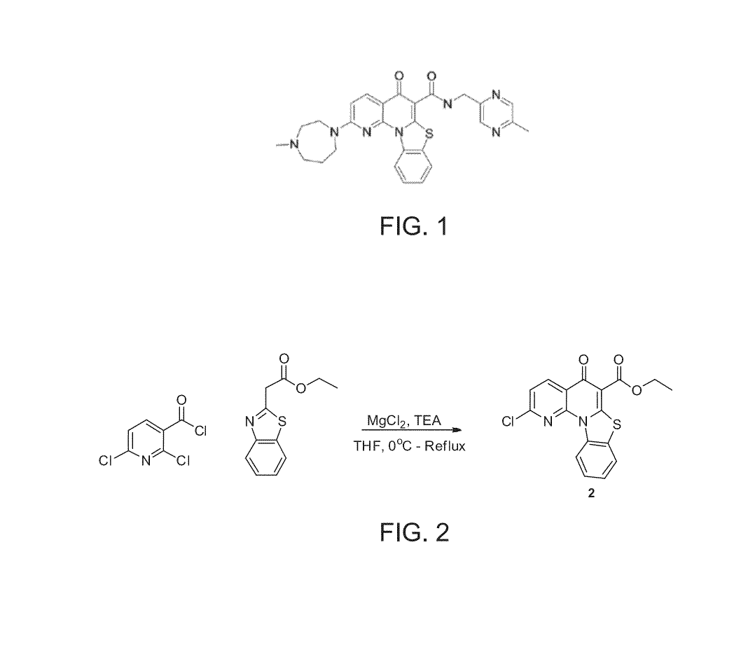

The title compound 2-(4-methyl-1,4-diazepan-1-yl)-N-((5-methylpyrazin-2-yl)methyl)-5-oxo-5H-benzo[4,5]thiazolo[3,2-a][1,8]naphthyridine-6-carboxamide (CX-5461) (schematic illustration of the chemical structure is shown in FIG. 1) was synthesized according to reported procedures (Chua P C, et al. A novel and efficient synthesis of 3-carboxy-4-oxo-1,8-naphthyridines using magnesium chloride. Tetrahedron Letters 49:4437-4442, 2008; Schwaebe M K, et al. Facile and efficient generation of quinolone amides from esters using aluminum chloride. Tetrahedron Letters 52:1096-1100, 2011; Nagasawa J Y, et al. PCT Int. Appl 2009046383, 09, April 2009). THF was distilled over Na and benzophenone, acetonitrile was dried over 4 Å molecular sieves. All other reagents were used with no further purification. 2,6-dichloronicotinoylchloride, N-methylhomopiperazine, triethylamine, magnesium chloride and 1,8-Diazobicyclo[5.4.0]undec-7-ene (DBU) were all purchased from Sigma Aldrich, ethyl 2-(benzothiazol-2-yl)acetate and (5-methylpyrazin-2-yl)methaneamine were purchased from TCI. ¹H NMR analyses were performed using a Bruker Avance DPX-400 Ultra shield or alternatively Bruker Avance DMX-500. All the chemical shifts are referenced to the residual solvent signal. All MS analyses were performed on a Thermo Scientific LCQ Fleet mass spectrometer with an ESI source. All the spectra were recorded in the positive mode (unless mentioned otherwise) and were analyzed by the Thermo Scientific Xcalibur software.

2,6-dichloronicotinoylchloride (2.42 gr.; 11.5 mmol) was slowly added to a solution containing ethyl 2-(benzothiazol-2-yl)acetate (1.89 mL; 10.4 mmol), magnesium chloride (1.48 gr.; 15.6 mmol) and tetrahydrofuran (25 mL). The resulting suspension was cooled to 0° C. and triethylamine (3.50 mL; 25.0 mmol) was added dropwise over 30 minutes. Once the addition was complete, the reaction mixture was allowed to stir while heating to room temperature over 2 hours. The reaction was then warmed to 80° C. for 1 hour and was monitored by LCMS. Once the reaction was over, 200 mL of water were added, and the resulting suspension was filtered by a biichner funnel. The remaining solid, was then dissolved in a (3:1; volumne/volume (v:v)) chloroform:dichloromethane mixture (500 mL), washed with water, dried over Na₂SO₄, filtered and the solvent was removed under reduced pressure (FIG. 2).

The product was then purified by trituration with cold diethylether to afford ethyl 2-chloro-5-oxo-5H-benzo[4,5]thiazolo[3,2-a][1,8]naphthyridine-6-carboxylate (2) as a beige solid (3.34 gr; 89%). ¹H NMR (400 MHz, CDCl₃) δ ppm 1.50 (t, 3H, J=7.2 Hz), 4.52 (q, 2H, J=7.2 Hz), 7.49 (t, 1H, J=7.6 Hz), 7.58 (d, 1H, J=8.4 Hz), 7.61 (t, 1H, J=8.0 Hz), 7.77 (d, 1H, J=8.0 Hz), 8.86 (d, 1H, J=8.0 Hz), 9.55 (d, 1H, J=8.8 Hz); MS (ESI): 359 (M+H)⁺.

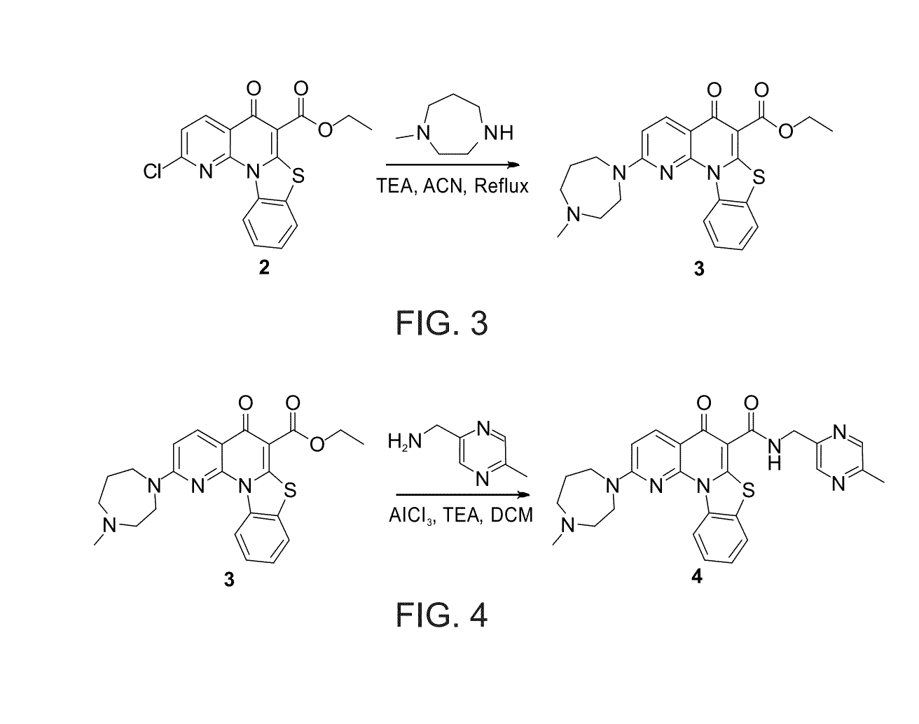

To a slurry of ethyl 2-chloro-5-oxo-5H-benzo[4,5]thiazolo[3,2-a][1,8]naphthyridine-6-carboxylate (2) (3.34 gr; 9.3 mmol) in acetonitrile (48 mL) was added N-methylhomopiperazine (1.21 mL; 9.7) and triethylamine (1.35 mL; 9.7 mmol). The mixture was heated under reflux for 24 hours after which additional 0.2 eq of N-methylhomopiperazine (231 μL) and 0.2 eq triethylamine (260 μL) were added and stirring was continued for additional 24 hours. The reaction was then cooled to RT and the product was collected by filtration via a biichner funnel, the solid was further washed with cold acetonitrile to afford ethyl 2-(4-methyl-1,4-diazepan-1-yl)-5-oxo-5H-benzo[4,5]thiazolo[3,2-a][1,8]naphthyridine-6-carboxylate (3) as a tan solid (3.34 gr; 81%) (FIG. 3). ¹H NMR (500 MHz, CDCl₃) δ ppm 9.33 (d, J=7.57 Hz, 1H), 8.48 (d, J=8.96 Hz, 1H), 7.69 (dd, J=7.58, 1.47 Hz, 1H), 7.47-7.38 (m, 2H), 6.69 (d, J=9.00 Hz, 1H), 4.45 (q, J=7.13 Hz, 2H), 4.22-4.05 (m, 2H), 3.93-3.74 (m, 2H), 3.30-3.10 (m, 2H), 3.08-2.93 (m, 2H), 2.66 (s, 3H), 2.50-2.31 (m, 2H), 1.46 (t, J=7.13 Hz, 3H); MS (ESI): 437 (M+H)⁺.

To a solution of ethyl 2-(4-methyl-1,4-diazepan-1-yl)-5-oxo-5H-benzo[4,5]thiazolo[3,2-a][1,8]naphthyridine-6-carboxylate (3) (3.34 gr; 7.66 mmol), (5-methylpyrazin-2-yl)methaneamine (2.87 mL; 25.3 mmol), and DBU (3.45 mL; 23.0 mmol) in dichloromethane (118 mL) was added AlCl₃ (3.06 gr; 23.0) portionwise. The reaction mixture was then left to stir at RT for 45 minutes after which, the reaction was diluted with dichloromethane (118 mL) and NaOH 4N (118 mL), and stirring was continued for additional 20 minutes. The layers were then separated and the organic layer was washed with H₂O (2×100 mL), NaCl sat. (1×100 mL), dried over Na₂SO₄, filtered and the solvent was removed under vacuum. The resulting solid was then purified by trituration with acetonitrile to afford 2-(4-methyl-1,4-diazepan-1-yl)-N-((5-methylpyrazin-2-yl)methyl)-5-oxo-5H-benzo[4,5]thiazolo[3,2-a][1,8]naphthyridine-6-carboxamide(4) as a white yellowish solid (2.948 gr, 75%) (FIG. 4). ¹H NMR (500 MHz, MeOD) δppm 9.26-9.10 (m, 1H), 8.57 (d, J=1.33 Hz, 1H), 8.52 (d, J=1.33 Hz, 1H), 8.20 (d, J=9.08 Hz, 1H), 7.71-7.64 (m, 1H), 7.37-7.31 (m, 2H), 6.72 (d, J=9.12 Hz, 1H), 4.76 (s, 2H), 3.90-3.77 (m, 2H), 3.76-3.63 (m, 2H), 2.95-2.84 (m, 2H), 2.75-2.68 (m, 2H), 2.57 (s, 3H), 2.45 (s, 3H), 2.10 (m, 2H); MS (ESI): 514 (M+H)⁺.

### Example 2

### In Vitro Studies CX-5461 Effects on Viability of Mice Splenocytes

No data regarding the effects of CX-5461 on immune cells is available. Towards the application of CX-5461 in the EAE mouse model of multiple sclerosis (MS), the present inventors have examined CX-5461 effects on the viability of immune cells and a range of effective doses. These experiments were done to provide applicable data for the in-vivo animal studies.

Experimental Methods

Measurements of Cell Viability—

Cells viability was assessed by the 2,3 bis[2-Methoxy-4-nitro-5-sulfophenyl]-2H-tetrazolium-5-carboxanilide (XTT) as say (Biological Industries, Kibbutz Beit Hemeek, Israel) which measures the reduction of a tetrazolium component (XTT) into soluble formazan product by the mitochondria of viable cells. The intensity of the dye obtained is proportional to the number of metabolic active cells. Mouse splenocytes were plated in presence of PHA (10 mg/ml) and elevated concentrations of CX-5461 (50-4000 nM) for 72 hours. Cells plated in absence of CX-5461 served as controls. Following incubation, cell viability was measured by 2,3-bis-(2-methoxy-4-nitro-5-sulfophenyl)-2H-tetrazolium-5-carboxanilide (XTT) assay according to manufacturer's instructions (Biological Industries, Kibbutz Beit Hemeek, Israel). The intensity of the dye obtained is proportional to the number of metabolic active cells.

Experimental Results

As shown in FIG. 5, CX-5461 decreased cell viability of splenocyte cells in a dose dependent manner, by 27% at 50 nM to maximal 97-100% at 500 nM and higher concentrations. Accordingly, the range between 50 to 500 nM concentrations was chosen for further experiments.

### Example 3

### In Vitro Studies: CX-5461 Effects on Viability of Human Peripheral Mononuclear Cells (PBMCS)—Healthy Subjects

No data regarding the effects of CX-5461 on human blood immune cells is available. Towards the application of CX-5461 in cell cultures of PBMCs obtained from MS patients, the present inventors have examined CX-5461 effects on the viability of human PBMCs obtained from healthy subjects in a range of the effective doses, as follows.

Experimental Procedures

PBMCs (2.0*10⁻⁵ cells/well) from normal, healthy, subjects were plated in the presence of PHA (20 μg/ml) with elevated concentrations of CX-5461 (50-500 nM) for 72 hours. Cells plated in absence of CX-5461 served as controls.

Experimental Results

As shown in FIG. 6, maximal effect of CX-5461 on viability of human PBMCs was detected at a concentration of 50 nM by 35%. Increased concentrations of CX-5461 did not result with further decrease in cell viability.

### Example 4

### In Vitro Studies: CX-5461 Effects on Apoptosis of Human PBMCS from Multiple Sclerosis Patients

Inhibition of Pol I by CX-5461 results in nucleolar stress which causes the release of ribosomal proteins from the nucleolus and subsequent activation of p53, resulting in cell apoptosis of human cancer cells (Drygin D, et al. Targeting RNA polymerase I with an oral small molecule CX-5461 inhibits ribosomal RNA synthesis and solid tumor growth. Cancer Res 71:1418-1430, 2011). To test the effect of CX-5461 on apoptosis in human PBMCs obtained from MS patients, the present inventors used the minimal effective doses of CX-5461 (at a range of 50 to 250 nM and including a lower concentration of 25 nM as well) obtained from experiments on PBMCs from healthy subjects which are shown in Example 3 hereinabove.

Experimental Procedures

Measurement of Apoptosis—

Apoptosis was measured by flow cytometry as follows: (1) percent of Annexin V positive cells; (2) number of cell cycle/apoptotic cells using propidium iodid (PI) staining; (3) percent of P53 positive cells to demonstrate the role of P53 dependent apoptosis mechanism; and (4) percent of RRN3 positive cells used as a reporter for detection of Pol I activity in cells after incubation with CX-5461.

For these purposes the present inventors used mAb anti Annexin V-FITC/propidium iodide (PI, 0.5 mg/ml), anti p53 mAb and anti RRN3 mAb (Sigma-Auldrich, USA), stained and presented as percentage of total cell counts. Fluorescence intensity measurements were done after excitation at 488 nm and detection at 520-530 nm for green fluorescence and at 665-685 nm for PI. Apoptotic level of PBMCs obtained from MS patients was measured at baseline and after 24 hours of incubation with increasing concentrations of CX-5461 (25-250 nM).

Experimental Results

CX-5461 Significantly Stimulated Apoptosis of PBMCs from MS Patients—

The results shown in FIGS. 7A-D demonstrate that CX-5461 significantly stimulated apoptosis of PBMCs from MS patients in a dose dependent manner as was demonstrated by the correlative increase in the percent and number of PI and Annexin V stained cells with increased CX-5461 concentrations. The mechanism of apoptosis was mediated by RRN3 as demonstrated by significant (p=0.00003) reduction in its expression and accordingly by stimulation of P53 expression.

### Example 5

### In Vitro Studies: CX-5461 Effects on Viability of Neural Progenitors Cells in Mice

As MS is a central nervous system disease the present inventors aimed to evaluate the effects of CX-5461 on viability of neuronal cells. The present inventors tested the effective range of CX-5461 concentrations found in the peripheral blood in relation to cell viability found on the regenerative capacity of the neural progenitor cells (NPCs).

Experimental Methods

Cell Viability Assay of Neuronal Progenitor Cells—

NPCs derived from the subventricular zone of the lateral ventricle of adult mice were allowed to proliferate in the presence of increased concentrations of CX-5461 (20 nM to 500 nM) for 72 hours, and cell viability was tested using XTT assay.

Experimental Results

CX-5461 does not Affect NPCs Viability at a Concentration Lower than 20 nM, And does not Suppress More than 50% Viability at Concentrations Higher than 50 nM—

As shown in FIG. 8, CX-5461 at a concentration of 20 nM had no effect on NPCs viability, while a 30% decrease in cell viability was detected at a concentration of 50 nM and ˜50% decrease in cell viability was demonstrated at higher concentrations (100, 250 and 500 nM). These results are similar to the findings shown in Examples 2-4 hereinabove with respect to mice splenocytes and human PBMCs both from healthy subjects and MS patients and suggest that CX-5461 would not result in significant effect (above 50% suppression of viability) at concentration lower than 50 nM and does not affect NPCs viability in concentration lower than 20 nM.

### Example 6

### In Vivo Experiments: CX-5461 Delayes Onset and Reduces Clinical Scores of MOG Induced EAE in Mice

Myelin oligodendrocyte glycoprotein (MOG) induced experimental autoimmune encephalomyelitis (EAE) is a widely accepted model for studying the clinical and pathological features of MS (Miller, S, Karpus W J. Experimental autoimmune encephalomyelitis in the mouse. Curr Protoc Immunol Chapter 15: Unit 15.1, 2007). To test the effect of CX-5461 on multiple sclerosis, the present inventors have tested the effect of blocking RNA Pol-I pathway by CX-5461 treatment at the disease induction phase of MOG-induced EAE. For this experiment, CX-5461 was first administered on the day of EAE induction and continued till the animals in the control untreated group developed clinical signs of EAE.

Experimental Methods

EAE Induction—

EAE was induced in female C57BL/6 mice, 8 weeks of age, by subcutaneous immunization to the flank of emulsion (1:1 ratio volume/volume) containing 200 mg MOG₃₅₋₅₅ peptide dissolved in phosphate-buffered saline (PBS) (at a concentration 2 mg/ml) and 0.5 mg Mycobacterium Tuberculosis H37Ra (Difco, Detroit, Mich.) in 100 mL incomplete Freund's adjuvant (Sigma, St. Louis, Mo.). Pertussis toxin 200 ng/mouse (dissolved in 100 ml PBS (List Biological Laboratories, Campbell, Calif.) was injected intra-peritoneal on the day of immunization and again two days later. Mice were monitored daily for clinical signs of EAE, scored as follows: 1-flaccid tail; 1.5—hind limb weakness; 2—paralyzed hind limbs and poor righting ability; 2.5—forelimb weakness, paralyzed hind limbs, paralyzed tail; 3—four limbs paralysis, inability to right; 4—quadriplegia; 5.0—moribund or death of the animal after preceding clinical disease. Animal reaching a score of 4 were scarified using CO₂. Mice were kept in an SPF (Specific Pathogen Free) environment. Animal breeding and maintenance and all experimental protocols were performed in accordance with the Israeli Council for Animal Care guidelines and all experiments were approved by Sheba IRB Animal Care Ethical Committee.

CX-5461 Prophylactic Treatment Initiated at Induction Phase of MOG-Induced EAE—

EAE was induced in female C57Bl/6J female mice (8 weeks old) as described above. Treatment with CX-5461 was initiated on the day of induction and continued till the clinical signs of EAE appeared in the control animal group (In 2 out of the 7 mice clinical signs of EAE appeared). CX-5461 was administered orally to each animal; the following doses of CX-5461 in 0.2 ml of vehicle were used: 12.5 mg/kg (n=6 mice), 25 mg/kg (n=6 mice) and 50 mg/kg (n=7 mice) in 0.2 ml of vehicle. Vehicle treated mice served as controls (n=7 mice). Treatment continued for 9 days post induction, until the first sign of EAE (grade 1) appeared in the vehicle animal group (in 2 out of 7 mice). Mice were monitored daily for clinical signs of EAE up to 42 days post induction.

Experimental Results

CX-5461 Delayed EAE Onset—

The onset of EAE in all CX-5461 treated mice was significantly delayed and occurred 10 days later than the appearance of the disease in the vehicle-control group (FIG. 9, and Table 2, below). This effect was evident in all experimental groups and was not dependent on CX-5461 concentration.

TABLE 2
Suppression of EAE score by CX-5461

| | Number | Disease onset | Maximal | Cumulative |
|---|---|---|---|---|
| Treatment group | of mice | (days p.i.) | clinical score | disease score |
| Vehicle | 7 | 11.7 ± 1.39 | 4.21 ± 0.32 | 46.43 ± 8.5 |
| 1. CX-5461 12.5 | 6 | 20.83 ± 1.36 | 2.83 ± 0.49 | 16.25 ± 3.94 |
| mg/kg/day | | | | |
| 2. CX-5461 25.0 | 6 | 23.1 ± 1.2 | 3.3 ± 1.0 | 14.1 ± 3.5 |
| mg/kg/day | | | | |
| P-value (group 1 | | 0.0004 | 0.0208 | 0.0059 |
| vs. Vehicle) | | | | |
| P-value (group 2 | | 6.2*10⁻⁵ | 0.05 | 0.0050 |
| vs. Vehicle) | | | | |

Table 2: Provided are the disease onset, maximal clinical score and cumulative disease scores of mice treated with CX-5461 on the day of induction of EAE by MOG.
“days p.i.”—days post induction of EAE.

CX-5461 Decreased EAE Severity—

In addition to the delayed EAE onset, the severity of EAE in CX-5461 treated animals was significantly lower than in the control-vehicle treated animals (FIG. 9, Table 2 hereinabove). The cumulative and maximal EAE score was significantly lower in mice treated with CX-5461 12.5 mg/kg (p=0.006 and p=0.02, respectively) as well as in mice treated with CX-5461 25.0 mg/kg (p=0.005 and p=0.05, respectively) than in the control vehicle treated mice group.

The results of CX-5461 treatment at EAE induction demonstrated that blocking Pol I pathway through treatment with its inhibitor CX-5461 in concentrations of 12.5 and 25.0 mg/kg/day delayed MOG-induced EAE and suppressed its severity. Animals treated with CX-5461 at the concentration of 50 mg/kg/day similarly did not develop EAE. However treatment was toxic to the animals and resulted in death after a mean of 7.6±0.5 days of treatment post-induction. Animals showed change in the color and brightness of their fur, loss of weight and change in behavior (the animals were gathered together and movements were reduced) since day 5 of treatment. This concentration was excluded from further experiments.

### Example 7

### In Vivo Experiments: CX-5461 Treats MOG Induced EAE in Mice

To test the effect of CX-5461 treatment on MOG-induced EAE mice, the present inventors have blocked RNA Pol-I pathway by CX-5461 treatment at the clinical symptomatic phase of MOG-induced EAE. For this experiment, CX-5461 was first administered on the day when the animal developed clinical signs of EAE and continued for the following 14 days.

Experimental Methods

EAE Induction—

EAE was induced in mice as described in Example 6, hereinabove.

CX-5461 Treatment Initiated at Clinical Onset of MOG-Induced EAE—

EAE was induced in female C57/6J female mice as described above. Treatment with CX-5461 was administered orally in 0.2 ml of vehicle with the following concentrations: 12.5 mg/kg/d (n=11 mice) and 25.0 mg/kg/d (n=8 mice). Vehicle treated mice served as controls (n=9 mice). CX-5461 treatment was initiated from the day of disease onset (score 0.5 or 1.0) for the following 14 days. The day of disease onset was evaluated for each animal daily.

Experimental Results

CX-5461 Decreased EAE Severity—

Both groups of mice treated with CX-5461 at a concentration of 12.5 mg/kg/day and 25.0 mg/kg/day had significantly lower EAE scores as compared to the control vehicle group (FIG. 10).

All mice from the 25 mg/kg/day group died after 10.1±0.5 days of CX-5461 treatment as result of toxic effects demonstrated by change in their fur color and brightness and loss of weight. All of these animals (that were treated with 25 mg/kg/day CX-5461) exhibited low EAE score 0.88±0.18 (FIG. 10).

In the group of mice treated with CX-5461 12.5 mg/kg/day all the animals survived and remained with a low EAE score, as demonstrated by lower (p=0.0004) maximal and cumulative (p=1.6*10⁻⁵) score compared to vehicle group (Table 3, hereinbelow).

TABLE 3
EAE score after CX-5461 treatment at EAE onset

| | | EAE Score at | | |
|---|---|---|---|---|
| Experiment | Number | Disease onset | Maximal | Cumulative |
| groups | of mice | (start treatment) | EAE score | disease score |
| Vehicle | 9 | 0.77 ± 0.17 | 4.38 ± 0.2 | 58.16 ± 5.09 |
| CX-5461 12.5 | 11 | 0.86 ± 0.14 | 2 ± 0.47 | 18.59 ± 4.26 |
| mg/kg/day | | | | |
| P-value | | 0.7 | 0.0004 | 1.6*10⁻⁵ |

Table 3: Provided are the EAE scores at disease onset, the maximal clinical score and cumulative disease scores of mice treated with CX-5461 on the day of EAE onset (appearance of clinical symptoms).

The results presented in Table 3 and FIG. 10 demonstrate that treatment of MOG-EAE model with CX-5461 at a concentration of 12.5 mg/kg/day significantly decreased the severity of the disease.

As the current multiple sclerosis treatments are given intramuscularly, subcutaneously or intravenously with uncontrolled plasma peaks and undesired side effects that decrease adherence to treatment, the use of CX-5461 which is orally bio-available is likely to improve patient compliance.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating an autoimmune disease in a subject, comprising:
administering to the subject a therapeutically effective amount of a compound of Formula (I), 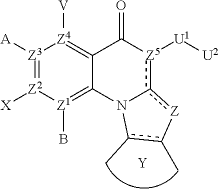
Formula (I)
or a pharmaceutically acceptable salt or ester thereof;
wherein [symbol] indicates an optionally unsaturated bond;
each of B, X, A or V is absent if Z¹, Z², Z³ and Z⁴, respectively, is N; and
each of B, X, A and V is independently H, halo, azido, —CN, —CF₃, —CONR¹R², —NR¹R², —SR², —OR², —R³, —W, -L-W, —W⁰, -L-N(R)—W⁰, A² or A³, when each of Z¹, Z², Z³ and Z⁴, respectively, is C;
Z is O, S, CR⁴₂, NR⁴CR⁴, CR⁴NR⁴, CR⁴, NR⁴ or N;
each of Z¹, Z², Z³ and Z⁴ is independently C or N, provided any three N are non-adjacent;
Z⁵ is C; or Z⁵ may be N when Z is N;
Y is an optionally substituted 5-6 membered carbocyclic or heterocyclic ring;
U¹ is —C(=T)N(R)—, —C(=T)N(R)O—, —C(=T)-, —SO₂N(R)—, —SO₂N(R)N(R⁰)—, —SO₂—, or —SO₃—, where T is O, S, or NH; or U¹ may be a bond when Z⁵ is N or U² is H;
U² is H, or C3-C7 cycloalkyl, C1-C10 alkyl, C1-C10 heteroalkyl, C2-C10 alkenyl or C2-C10 heteroalkenyl group, each of which may be optionally substituted with one or more halogens, ═O, or an optionally substituted 3-7 membered carbocyclic or heterocyclic ring; or U² is —W, -L-W, -L-N(R)—W⁰, A² or A³;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R⁴ may be —W, -L-W or -L-N(R)—W⁰;
each R and R⁰ is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member;
W⁰ is an optionally substituted 3-4 membered carbocyclic ring, or a C1-C6 alkyl group substituted with from 1 to 4 fluorine atoms;
provided one of U², V, A, X and B is a secondary amine A² or a tertiary amine A³, wherein
the secondary amine A² is —NH—W⁰, and
the tertiary amine A³ is a fully saturated and optionally substituted six-membered or seven-membered azacyclic ring optionally containing an additional heteroatom selected from N, O or S as a ring member, or the tertiary amine A³ is a partially unsaturated or aromatic optionally substituted five-membered azacyclic ring, optionally containing an additional heteroatom selected from N, O or S as a ring member;
thereby treating the autoimmune disease in the subject.

2. A method of treating an autoimmune disease in a subject, comprising:
administering to the subject a therapeutically effective amount of a compound of Formula (II), 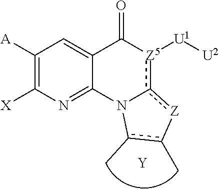
Formula (II)
or a pharmaceutically acceptable salt or ester thereof;
wherein [symbol] indicates an optionally unsaturated bond;
each of A and X is independently H, halo, azido, —CN, —CF₃, —CONR¹R², —NR¹R², —SR², —OR², —R³, —W, -L-W, —W⁰, -L-N(R)—W⁰, A² or A³;
Z is O, S, CR⁴₂, NR⁴CR⁴, CR⁴NR⁴ or NR⁴;
Y is an optionally substituted 5-6 membered carbocyclic or heterocyclic ring;
U¹ is —C(=T)N(R)—, —C(=T)N(R)O—, —C(=T)-, —SO₂N(R)—, —SO₂N(R)N(R⁰)—, —SO₂—, or —SO₃—, where T is O, S, or NH; or U¹ may be a bond when U2 is H;
U² is H, or C3-C7 cycloalkyl, C1-C10 alkyl, C1-C10 heteroalkyl, C2-C10 alkenyl or C2-C10 heteroalkenyl group, each of which may be optionally substituted with one or more halogens, ═O, or an optionally substituted 3-7 membered carbocyclic or heterocyclic ring; or U² is —W, -L-W, -L-N(R)—W⁰, A² or A³;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R⁴ may be —W, -L-W or -L-N(R)—WO;
each R and R⁰ is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member;
W⁰ is an optionally substituted 3-4 membered carbocyclic ring, or a C1-C6 alkyl group substituted with from 1 to 4 fluorine atoms;
provided that one of U², A, and X is a secondary amine A² or a tertiary amine A³, wherein
the secondary amine A² is —NH—W⁰, and
the tertiary amine A³ is a fully saturated and optionally substituted six-membered or seven-membered azacyclic ring optionally containing an additional heteroatom selected from N, O or S as a ring member, or the tertiary amine A³ is a partially unsaturated or aromatic optionally substituted five-membered azacyclic ring optionally containing an additional heteroatom selected from N, O or S as a ring member;
thereby treating the autoimmune disease in the subject.

3. A method of treating an autoimmune disease in a subject, comprising:
administering to the subject a therapeutically effective amount of a compound of Formula (III), 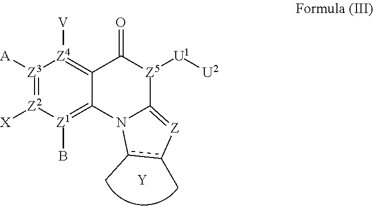
or a pharmaceutically acceptable salt or ester thereof;
wherein [symbol] indicates an optionally unsaturated bond; and
each of B, X, A or V is absent if Z¹, Z², Z³ and Z⁴, respectively, is N; and
each of B, X, A and V is independently H, halo, azido, —CN, —CF₃, —CONR¹R², —NR¹R², —SR², —OR², —R³, —W, -L-W, —W⁰, -L-N(R)—W⁰, A² or A³, when each of Z¹, Z², Z³ and Z⁴, respectively, is C;
each of Z¹, Z², Z³ and Z⁴ is independently C or N, provided any three N are non-adjacent;
Y is an optionally substituted 5-6 membered carbocyclic or heterocyclic ring;
U¹ is —C(=T)N(R)—, —C(=T)N(R)O—, —C(=T)-, —SO₂N(R)—, —SO₂N(R)N(R⁰)—, —SO₂—, or —SO₃—, where T is O, S, or NH; or U¹ may be a bond when Z⁵ is N or U² is H;
U² is H, or C3-C7 cycloalkyl, C1-C10 alkyl, C1-C10 heteroalkyl, C2-C10 alkenyl or C2-C10 heteroalkenyl group, each of which may be optionally substituted with one or more halogens, ═O, or an optionally substituted 3-7 membered carbocyclic or heterocyclic ring; or U² is —W, -L-W, -L-N(R)—W⁰, A² or A³;
in each —NR¹R², R¹ and R² together with N may form an optionally substituted azacyclic ring, optionally containing an additional heteroatom selected from N, O and S as a ring member;
R¹ is H or C1-C6 alkyl, optionally substituted with one or more halogens, or ═O;
R² is H, or C1-C10 alkyl, C1-C10 heteroalkyl, C2-C10 alkenyl, or C2-C10 heteroalkenyl, each of which may be optionally substituted with one or more halogens, ═O, or an optionally substituted 3-7 membered carbocyclic or heterocyclic ring;
R³ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
each R⁴ is independently H, or C1-C6 alkyl; or R⁴ may be —W, -L-W or -L-N(R)—W⁰;
each R and R⁰ is independently H or C1-C6 alkyl;
L is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
W is an optionally substituted 4-7 membered azacyclic ring, optionally containing an additional heteroatom selected from N, O and S as a ring member;
W⁰ is an optionally substituted 3-4 membered carbocyclic ring, or a C1-C6 alkyl group substituted with from 1 to 4 fluorine atoms;
provided that one of U², V, A, X and B is a secondary amine A² or a tertiary amine A3, wherein
the secondary amine A² is —NH—W⁰, and
the tertiary amine A³ is a fully saturated and optionally substituted six-membered or seven-membered azacyclic ring optionally containing an additional heteroatom selected from N, O or S as a ring member, or the tertiary amine A³ is a partially unsaturated or aromatic optionally substituted five-membered azacyclic ring, optionally containing an additional heteroatom selected from N, O or S as a ring member,
thereby treating the autoimmune disease in the subject.

4-9. (canceled)

10. A method of monitoring treatment efficiency of a subject having an autoimmune disease, the method comprising:
(a) treating the subject with said compound according to the method of claim 1, and
(b) comparing a level of expression of least one gene involved in the RNA polymerase I pathway in a cell of the subject following said treating with said compound to a level of expression of said at least one gene in a cell of the subject prior to said treating the subject with said compound,
(i) wherein a decrease above a predetermined threshold in said level of expression of said at least one gene following said treating with said compound relative to said level of expression of said at least one gene prior to said treating with said compound indicates that said compound is efficient for treating the subject;
(ii) wherein an increase above a predetermined threshold in said level of expression of said at least one gene following said treating with said compound relative to said level of expression of said at least one gene prior to said treating with said compound indicates that said compound is not efficient for treating the subject; or
(iii) wherein when a level of expression of said at least one gene following said treating with said compound is identical or changed below a predetermined threshold as compared to prior to said treating with said compound then the treatment is not efficient for treating the subject
thereby monitoring treatment efficiency of the subject having an autoimmune disease.

11. The method of claim 1, wherein said compound is 2-(4-methyl-1,4-diazepan-1-yl)-N-((5-methylpyrazin-2-yl)methyl)-5-oxo-5H-benzo[4,5]thiazolo[3,2-a][1,8]naphthyridine-6-carboxamide as depicted in Formula

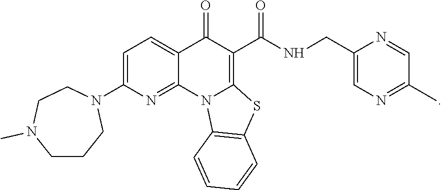

12. The method of claim 1, wherein the autoimmune disease is multiple sclerosis.

13. (canceled)

14. The method of claim 12, wherein said multiple sclerosis is a relapsing-remitting course of multiple sclerosis.

15. The method of claim 12, wherein said multiple sclerosis is a progressive course of multiple sclerosis.

16. The method of claim 12, wherein said multiple sclerosis is a benign multiple sclerosis.

17. The method of claim 1, wherein said administering said compound is performed following diagnosis of said autoimmune disease.

18. The method of claim 17, wherein said autoimmune disease is multiple sclerosis and whereas said diagnosis comprises appearance of brain lesions characteristics of said multiple sclerosis.

19. The method of claim 12, wherein said compound is provided at a concentration range of about 0.081-1.62 mg/kg/day.

20. The method of claim 12, wherein said compound is provided at a concentration range of about 0.81-1.215 mg/kg/day.

21. The method of claim 12, wherein said compound is provided at a concentration of about 1.01 mg/kg/day.

22. The method of claim 1, wherein Z¹ is N, and each of Z², Z³ and Z⁴ is C.

23. The method of claim 1, wherein U is —W or -L-W, where W is an optionally substituted 5-6 membered unsaturated or aromatic azacyclic ring, optionally containing an additional heteroatom selected from N, O and S; or W is an optionally substituted 5-7 membered saturated azacyclic ring containing an additional heteroatom selected from N and S.

24. The method of claim 1, wherein U² is -L-N(R)—W⁰.

25. The method of claim 1, wherein Y is an optionally substituted phenyl ring.

26. The method of claim 2, wherein at least one of A and X is a tertiary amine A³.

27. The method of claim 2, wherein A³ is selected from the group consisting of imidazole, imidazoline, pyrroline, piperidine, piperazine, morpholine, thiomorpholine and homopiperazine.

28-43. (canceled)


SEQUENCE LISTING (126 sequences)

SEQ ID NO: 1 - DNA, 296 bp, Homo sapiens
gatgaccagatcatcaactggctgctagaattccgttcttctgtcatgtacttgacaaaagactttgagcaacttatcagtattatattgagattgccttggttgaatagaagtcaaacagtagtggaagagtatttggcttttcttggtaatcttgtatcagcatagactgttttcctcagaccgtgtctcagcatgattgcttcccattttgtgcctccccgagtgatcattaaggaaggcgatgtagatgtttcagattctgatgatgaagatgataatcttcctgcaaattt

SEQ ID NO: 2 - DNA, 476 bp, Homo sapiens
  misc_feature (58)..(58): n is a, c, g, or t
tttccttcaacttgtggatgcaggcaaggtggatgatgccagagctctcctacagagnatgtggtgcnaattgctgaacaaaccccgattttgttgttgttcctccttaggaattctaggaaacaaggaaaggcatcaactgtgaaatctgtgttagaattgattcctgaattaaatgaaaaggaagaagcatacaattccctcatgaaaagctatgtctcagagaaagatgtcacatctgctaaagcactgtatgaacatttgactgcaaagaatacaaaattggatgatctgtttctaaagcgttacgcatctttgctgaagtatgctggagagcctgtccctttcattgaaccccctgaaagctttgaattttatgcacagcagctaagaaaattgagggaaaactcttcttgaaataaccaggcgatactttgttttgtatatatttgtgattctgtgtctacatgttattt

SEQ ID NO: 3 - DNA, 533 bp, Homo sapiens
aggcttagcaggatgggcgcagtggctcacgccactaatcccaacattttaggaggcctaggcaggagcaatcacttgtgcctgggagttctagactagcctgggcgagacttcatctctacaaaaaaagcaacaacgacaaaaaaaattagccaagcatagtggcacacccctgtagtcccagctacttgggaggctgaggtgggaggattgcttgaacccaagaggtcgaggctgcagtgagccaagattgtgccactgcactccagcctgggtgacagagcaggaccctgtctctattttataaattaaaaaaggctgggtgtggtggctcacacccataatcccaacactttggctcagcagattgcttgaacccaggaattcaagtccaatctgggcaacatggggaaaccccagctctacaaaaaaaattagcctggtgtggtggcacatgcctgtagttccagctactcaggaggctgaggtgggagaatctcctgagcctggaaggtccaggcagtgagccaaga

SEQ ID NO: 4 - DNA, 411 bp, Homo sapiens
ccaggcatatgacatggatccccctggggaaccaggctgatctctttccagagggcactatccgaccagtgcatgatgatatcctcatcgctcagctgcggcctggccaagaaattgacctgctcatgcactgtgtcaagggcattggcaaagatcatgccaagttttcaccagtggcaacagccagttacaggctcctgccagacatcaccctgcttgagcccgtggaaggggaggcagctgaggagttgagcaggtgcttctcacctggtgttattgaggtgcaggaagtccaaggtaaaaaggtggccagagttgccaacccccggctggataccttcagcagagaaatcttccggaatgagaagctaaagaaggttgtgaggcttgcccgggttcgagatcattata

SEQ ID NO: 5 - DNA, 291 bp, Homo sapiens
gttgagtcaacgggggtgttgccaccagatgtgctggtgagtgaagccatcaaagtactgatggggaagtgccggcgcttcttggatgaactagatgcggttcagatggactgagcttggatgcttctgaggcaagctgaagctttgggttctgactgacccaccctacaggactgctgaacagagagcccagtgtgactagggatcctgagttttctgggacaattccagctttaatcaatacattttgttaaatgtgccataaaatgagactttttacgcctttataag

SEQ ID NO: 6 - DNA, 441 bp, Homo sapiens
gcacgaggaagaccataccctaggaaattctctacgttacatgatcatgaagaacccggaagtggaattttgtggttacactacgacccatccttcagagagcaaaattaatttacgcattcagactcgaggtacccttccagctgttgagccatttcagagaggcctgaatgagctcatgaatgtctgccaacatgtgcttgacaagtttgaggccagcataaaggactataaggatcaaaaagcaagcagaaatgaatccacattctagtcctttatgcagtatacaaggagaactgtcctgtaggatattctcttcctgatggtgcagaacccagaattagaagtttgtggttacagcatactctgtccttcagaaaggcgtgattctagctgttgaccccttgcagctgttggaatctctgcaagaacctctgtatt

SEQ ID NO: 7 - DNA, 393 bp, Homo sapiens
gctatcagcccggatgacagtgacgaggagaactgagggcacgtggggtgcggcagcgggctagggcccagggcagcttgcccgtgctgccgtgcagttcttgcctccctcacggggcgtcacccccagcccagctccgttgtacataaataccttgtgacagagctcccggtgaacttctggatcccgtttctgatgcagattcttgtcttgttctccacttgtgctgttagaactcactggcccagtggtgttctacctcctaccccacccaccccctgcctgtccccaaattgaagatccttccttgcctgtggcttgatgcggggcgggtaaagggtattttaacttaggggtagttcctgctgtgagtggttacagctgatcctcggg

SEQ ID NO: 8 - DNA, 238 bp, Homo sapiens
gttctggcctagagctgtatgcggaatggaagcacgttaatgaggactctcaggagaagaagatcctgctgagtccagagcgagtgcatgagatcttcaaacgcatctcagatgaggagtgttttgtgctgggcatcgagccccgctatgcacggccagagtcgatgattgtcacagtgctgcccgtgcccccgctgtccgtgcggcctgctgttgtgatgcagggctctgcccgtaa

SEQ ID NO: 9 - DNA, 545 bp, Homo sapiens
tttattggccccacttattaccagcgtttgaagcatatggtggatgataagattcactctcgtgctaggggacctattcagatcctcaatagacagcccatggagggtagatctcgtgatggtggcctgcgttttggagaaatggaacgagattgtcagattgcccatggagcagcccagtttttaagggaaagattgtttgaggcatcagatccatatcaggttcatgtttgcaatctttgtggaataatggcgattgccaacaccaggacccatacatatgaatgcaggggctgccgcaataaaacccagatttctttggtgcgaatgccttacgcatgcaaactattgtttcaggaacttatgtctatgagtattgcaccgcgaatgatgagtgtttagctattttacaggagtcaacaagataattaaatatcttggtgtcttgtttctattgtgtggctttataaaaatgacaaatatgtactgtgttgtgataaaaagtattttatttgtttaatgatatgcatgcttttcttctgtaa

SEQ ID NO: 10 - DNA, 439 bp, Homo sapiens
ttaactgcagcttgcctgcttcagcaaaaacggagattcaggccagcagctggatatgggggtctctcttcagactcttctcgtttctgagaatctagtctactgttggttgagcttcttggcaggacatcagtaccaactagaagtgggtcatagatagattaccagggatgcagtggtgtttaggcaggataggtctttactggccctgactgctgttaataattggcagcagtgctccccagatcccagaaggtccctgctggagtgtttccagtgcacctgtagggaaccaactagacttctctcctggttagtccagctctttactctaaaccctttctgtccaaaatgagtcattttcagttgtaccttagatgtctggtgttgaggatcaagtgccatagcctttattcagggggcctataaacccttccag

SEQ ID NO: 11 - DNA, 350 bp, Homo sapiens
  misc_feature (55)..(55): n is a, c, g, or t
caattcccagggtgtccctaatttgagaagtaaccttttggaatagcattagacnctggctgtccccntccccncncaaataaacatgatatttcattctctgtccagcagtcatgaaccccttcacctccaatgacntgatcatttagtttggtgggggtgggggtgggggtnngggtnggggnnnanncanccgcaggagcaagggcccctcccacatacacaggaggagtatttcatttctccttaatgaaggctctggccctaacccctcagcactgtctccagataggaacatgcacaaagcagttaattaggcagcctggagaaaaccagagatccagtacaga

SEQ ID NO: 12 - DNA, 554 bp, Homo sapiens
  misc_feature (270)..(270): n is a, c, g, or t
cctgctagcgcttcaaattcccaggtgtccctaatttgagaagtaaccttttggaatagcaggagcaagggcccctcccacatacacaggaggagtatttcatttctccttaatgaaggctctggccctaacccctcagcactgtctccagataggaacatgcacaaagcagttaattaggcagcctggagaaaaccagagatccagtacagaaaggaaaggatatttattgattaacagaagttgtctttttaaaagtgtttatttttngcaataaagagcacaacataatctccttcatgcgtcatcgtgccacttagctgagccagccagctctgcctccctccccaaagacctttgnnnnnnnnngctttanaggatgaatctgttgaggaggtctgtttccgagctggcctgggctgccccatacccctcaccagcatagctgacggcttcggcgatgttctgggaaccagtgatgtggtgccagtaggatcgccgcggcanactgcgtcgtgggtgtgcagggatacgagtaatgttccgtgcccttg

SEQ ID NO: 13 - DNA, 338 bp, Homo sapiens
ggtcgtgccaaagaggtccacaaccaggtgtgcactgttcactgcagcccatttgctgtatgaactgtggttgttgtgtgcccaatgacaaggctactaagaaattcatcatttgaaacgtagaggccgcagcagtcagcgatgtttctgaaatgagcatccttgacgcctgtgtacttcccaggctggatgtgaagctacattaccatgtgagttgtgccattcacagcacagtggtgaggaattgagctcatgaagcaggcaaggaccgaacacctccaccccaacgtagacctgcaggtgctgccccatgacctccaccaaagcccatataagga

SEQ ID NO: 14 - DNA, 349 bp, Homo sapiens
  misc_feature (73)..(74): n is a, c, g, or t
ttttttacttcataggccaggcttgaagttctgaacactttgaagtctccaattatgagagatccagtctaannctctggcctgctaattagcaataagtgctttatttggaaggagggagtcatccactcttgagccactgcagtgaagtcacttgatctcagtctgggggaaaacacttcaatagctaaacattctagctttgatttttctgaagggaatacacttgttttcaattttggggtttttctttggggcacttgcttgactctgtatgaacttgtgatccaaggaaaaaggagaaagaacagtgttggcttttaaaatcaggatggttttatgtttgcta

SEQ ID NO: 15 - DNA, 416 bp, Homo sapiens
  misc_feature (45)..(45): n is a, c, g, or t
accgaccagatgtttgtgttctttccagaggagcccaaggtgggnatcaagaccatcaaggtgtactgccagcgcatgcaggaggagaacatcacacgggctctcatcgtggtgcagcagggcatgacaccctccgccaagcagtccctggtcgacatggcccccaagtacatcctggnagcagtttctgcagcaggagctnntcatcaacatcacggagcacgagctagtccctgagcacgtcgtcatgaccaaggaggaggtgacagagctgctggcccgatannntaagctccgagagaaccagctgcccaggatccaggcgggggacccntgtggcgcgctactttgggataaagcgtgggcaggtggtgaagatcatccggcccagtgagacggctggcaggtacatcacc

SEQ ID NO: 16 - DNA, 339 bp, Homo sapiens
gccccattcacctgtggatttgtgcgagatgcagcctcagaaggaacaaggcccccagagggaggtcacctgggggcaactggtgccgggtcttcacccagaccacgctgggtcccctctgttgggggtttggggtccgggtctcccaccagccactgcttcctcctgggccctcaattccacccctcgtcttccctccctcggggccctgatgcgtggcggcccccacccggcctcggctctttactccattcacagccatgcacgcgctcaagtacaccagggtgcgagatgccagctctggagttctcggttgttgtaggaggttgggtgttttca

SEQ ID NO: 17 - DNA, 485 bp, Homo sapiens
agataagctaggagccgcgcgagtcgtagtgtcgctgtttgcgggtctccgcgcgggaccggggcgcagcggggtcgctgaggcgagggtgtcatgtcagacaacgaggacaattttgatggcgacgactttgatgatgtggaggaggatgaagggctagatgacttggagaatgccgaagaggaaggccaggagaatgtcgagatcctcccctctggggagcgaccgcaggccaaccagaagcgaatcaccacaccatacatgaccaagtacgagcgagcccgcgtgctgggcacccgagcgctccagattgcgatgtgtgcccctgtgatggtggagctggagggggagacagatcctctgctcattgccatgaaggaactcaaggcccgaaagatccccatcatcattcgccgttacctgccagatgggagctatgaagactggggggtggacgagctcatcatcaccgactgagctggagt

SEQ ID NO: 18 - DNA, 543 bp, Homo sapiens
gtatggctttgtaattgctgtcaccaccattgacaatattggtgctggtgtgatccagccaggccgaggctttgtcctttatccagttaagtacaaggccattgttttccggccatttaaaggggaggtcgtggatgctgttgtcactcaggtcaacaaggttggactcttcacagaaattgggcccatgtcttgcttcatctctcgacattccatcccttcagagatggagtttgatcctaactccaacccaccatgttacaagacaatggatgaggatattgtgattcagcaggacgatgagatccgcttaaagattgtggggacccgtgtggacaagaatgacatttttgctattggctccctgatggacgattacttggggcttgtaagctgagcctggtggcctcctacccttggtcctactctaggaagtgtgattgtcacacttatcatgttgtccagaggtccagtctggctgctgttgtggaggcaaggaaggcaactcatcccagaaggcatctggtgcttcttgtagcttaa

SEQ ID NO: 19 - DNA, 377 bp, Homo sapiens
ccttccagggctgaccagtttgagtatgtaatgtatggaaaagtgtacaggattgagggagatgaaacttctactgaagcagcaacacgcctctctgcgtacgtgtcctatgggggcctgctcatgaggctgcagggggatgccaacaacctgcatggattcgaggtggactccagagtttatctcctgatgaagaagctagccttctgaacctcgcctgaagccagcctctctgccaagtcactcaggtcatgggcattgttcaagcctgagtggcagccgctcttgctcacctgttgaggaagggctggctcactgtccaccgtggcggcatctttaactggcctccactcaatgggaaactgactcgcctgtga

SEQ ID NO: 20 - DNA, 305 bp, Homo sapiens
tggagcccgacgggacttacgagccgggcttcgtgggtattcgcttctgccaggaatgtaacaacatgctgtaccccaaggaagacaaggagaaccgcattctgctctacgcgtgccggaactgtgattaccagcaggaggccgacaacagctgcatctatgtcaacaagatcacgcacgaagtggacgaactgacccagattatcgccgacgtgtcccaggaccccacgttgccgcggaccgaggaccacccgtgccaaaagtgcggccacaaggaggctgtgttcttccagtcacacagtgcg

SEQ ID NO: 21 - DNA, 515 bp, Homo sapiens
  misc_feature (173)..(173): n is a, c, g, or t
gttcttgctcttcgagggcgagaagaagatcaccattaacaaggacaccaaggtacccaatgcctgtttattcaccatcaacaaagaagaccacacactgggaaacatcattaaatcacaactcctaaaagacccgcaagtgctatttgctggctacaaagtcccccaccccntngnngnanaagatcatcatccgagtgcagaccacgccggactacagcccccaggaagcctttaccaacgccatcaccgacctcatcagtgagctgtccctgctggaggagcgctttcgggtggnncnataaaagacaagcaggaaggaattgagtaggggccagagggggctctgctcggcctgtgagccccgttcctacctgtgcctgaccctccgctccaggtaccacaccgaggagagcggccggtcccagccatggcccgccttgtggccacccctcaccctgacaccgacgtgttggccacccctcaccctgacaccgacgtgtcctgtacataga

SEQ ID NO: 22 - DNA, 354 bp, Homo sapiens
  misc_feature (252)..(253): n is a, c, g, or t
gttcttgctcttcgagggcgagaagatcaccattaacaaggacaccaaggtacccaatgcctgtttattcaccatcaacaaagaagaccacacactgggaaacatcattaaatcacgtgcctgcttccccttcgccttctgccgtgattgtcagtttcctgaggcctccccagccacgcttcctgtacagcctgcagaactctgccccagagcacatcagctatgtgccccagctctcaaacgacaccttgnncggggaggctcaccctgtccaccttcacgctggagcagcctctaggccagttcagcagccacaacatctctgacttggataccatctggctggtggtggcc

SEQ ID NO: 23 - DNA, 299 bp, Homo sapiens
cagccggtcccctggataaagaggtgagggggtgtgaagtctgggagcccccctcccagggccaggctacagggggacagcagggctataccaggagtgccacagagcttgcatttgggtgtgatctaatccccagtcctgccctgacctcctgggtgctcttagccaagccctcttccctgtgtgtgctagaaagtgagggtcactcccttccacacaagggagtgaattgagggtggggatctgagctctctgagcagcaaagctggacacaggccaaattgccaatcctgagtccc

SEQ ID NO: 24 - DNA, 537 bp, Homo sapiens
  misc_feature (83)..(83): n is a, c, g, or t
ccagtgaactcctgaaacctcaaacagtaccaaacctttatacactgttttttccatatatatatacctatgataaagtatanatgtataanattaagcatagcaagagattaataataatgtaatagaacaatgataacnatactataataanaagttatgtgaatgtggtntggntctctcttgctttncaaaatatcttcttgtacagtactcacctattttagaatgtggttgactacaggtaaccaaaaccacagaaagggaaactttggatgaggggggcactactgtacttaggaatacaactatatacatatgattttatttttaagaccatattatatttgggtatctactaatattttgtataaagcaattttttgttccattacgtgactttttgttttattgtatatgtaatttaacacacaataaagggtaaagttgcttccccaaaccacacttttaatcaaaacctagaatcatctgcagtccttgttaaaaatgcaggtttctagaaccctctgaagttct

SEQ ID NO: 25 - DNA, 253 bp, Homo sapiens
ttccgtgctgtgtaggggctaacaatggacacccagaaggacgttcaacctccaaagcagcaaccaatgatatatatctgtggagagtgtcacacagaaaatgaaataaaatctagggatccaatcagatgcagagaatgtggatacagaataatgtacaagaaaaggactaaaagattggtcgtttttgatgctcgatgaatgctgggaattcagaggaatgtcttcacttatacttggatttgctctcttc

SEQ ID NO: 26 - DNA, 231 bp, Homo sapiens
  misc_feature (63)..(63): n is a, c, g, or t
ctttcacactgtggcagcccagtgaagcagactgggccatgaactctcctagccctggggccnagcctgttccacaggcacccctgcaggaggcgctgccaggagagccttccatctcggggctctttgaggttccctccttctgggtgttcttcaggctgagcagagaggctcctgtaccctctctctcggaatctgaagagccagatttaggccgggcaaaggggctca

SEQ ID NO: 27 - DNA, 349 bp, Homo sapiens
ctgcttcacttgtggcaagatcgtcggcaacaagtgggaggcttacctggggctgctgcaggccgagtacaccgagggggatgcgctggatgccctgggcctgaagcgctactgctgccgccggatgctgctggcccacgtggacctgatcgagaagctgctcaattatgcacccctggagaagtgaccacgctggaacccacccacccgctgtgctgaccatgggccctgagcgtcctgccccgaattcacgaggctgaggcatccgggagctggcgtaatgcctggccgcagtgtgtgtgtatcccataccccactctggaaggaaccatccagtaaaggtctttca

SEQ ID NO: 28 - DNA, 503 bp, Homo sapiens
aagaattcccttgcgtattctctctctaaaacaaccaaaaaaaaatggagaggctttttatatactctaagactggctaaacaaccttgatcattgagcctcgagccatgggagagatgctgaccatgtggactgcaaggctgcttgattcacagatggatgtgacctaaaggataaataagctattacttatgtgctgatctcttgacattcactcattagaagaccttactccttcaagcaaatgtttggggtcaaatttaccatatcttctggctaaccatattcaagattcttctgaaacttggaggatgtaaagaatccatttgatttggtcagcctggcttttgtcgtggtggctggctcggataaattttcccaacaattaaatcttgcctttacacacccaaactttgtaattttagtcttggtgaaatataatgaatttgttcctaccttgtcaagcaagaatgtcgtcttctcctatggactcaattgctatt

SEQ ID NO: 29 - DNA, 276 bp, Homo sapiens
ccccatccaggaccttctatttatatactgtgaacatcctgtcagctgcccgaatgttgttcgacaggtgctacaagagcatagccaacttgatagaaaggaggcaatttgaaaccaaagagaataagcgtctactagaaaaatctcagagggtagaagccatcattgcatctatgcaggctactggtgcagaggaagcacagttacaagaaatagaggagatgatcacagctcctgaacgtcagcagctagagaccctgaaacgtaatgtcaaca

SEQ ID NO: 30 - DNA, 517 bp, Homo sapiens
gagtgagattaccacttcctctagtgctcccttcacccagccattgtcttccaatgagatcttcagatccctacctgttggctataacatctctaagcaagttcttgatcagtatctcactctgctggcagatgatccactagagtttgttggaaagtctggcgacagtggtggaggaatgtatgtcatcaacctccataaggcattagcatccctagccacagccactctgaagtccgtcgtacaggagagatttgggtctcgctgtgctagaatattccgtctagttttgcagaagaaacacatagagcagaagcaagtggaagactttgcaatgattcctgcaaaggaggcaaaggatatgctatataagatgctctcagaaaatttcatgtcactccaggttggatgccagtgagatccaggtggacgaaaccatcttcctgctggagtcttacattgagtgcaccatgaagagacagtgatccagaagaagcatcttcctcagaagatctgg

SEQ ID NO: 31 - DNA, 530 bp, Homo sapiens
cctgctccagacattttgttcttgaatctgtgagacccagaagggcccactgagcccactcactccacctttggcaaccattgttccaggtcccccagggcttcctcccacagcagctgtgaatggcacagtgaccttcctgcagcgtggagatggcacatccttgctgctggggacttggccctgctatttatttttgtatttatgtcttaatctcttccactgatgcatcctccaagggtagatggggagggtctgtgtgaaggggccggcttctcttggtgcctgctgggttgcaggggcaggaagcgtgtggactgcagcttctgctggtgctccccccgtcctcctggaggcagtataggagagagagcaaggattgagtctgagacttaagcactcggtcccagcttgccagttcctggttctgtgtccttggaaaactacctaacctttctgagcctcctatactatccgacacaaatggggatgatacctacctccagggttggcgtgaggattcatgggct

SEQ ID NO: 32 - DNA, 382 bp, Homo sapiens
ggtacgccgaaacatgatccagtctcggttgactcaagagtgtggagaagatctcagtaaacaggaggtggataaagtactaaaggactgctgtgtaagctatggtggcatgtggtaccttaaagggacagtacagtcttgacaatagtagcaaactactaacccagcaaatctaagcccaaggaagaagggcggaaccagaagtagggcctcgacttgcttcagacgacacagagcaagaggaactgaccatctcatgacctgtggcattgcacggtgcagtggacagaagggattatcctcagccagtcgcagggtcagcttaagttagttagatcactcccagaagagaccagctgggaccttctttgcagtacaattt

SEQ ID NO: 33 - DNA, 216 bp, Homo sapiens
  misc_feature (55)..(56): n is a, c, g, or t
agagtggaacatgccttttccgcacaatattaattcctttttgtatcagaaaggnnctnttaggagttatgctaccatacttacttcaaacccaatgactactgtcaaggtcatattttcagtacataaatactatcattttcattctaaagaatattttcactgttccttctttcttaaagtcttatgtttcactctttaactcaaatgtattct

SEQ ID NO: 34 - DNA, 391 bp, Homo sapiens
  misc_feature (295)..(295): n is a, c, g, or t
gataaccacttgaaccaggcaaatacactgaagataaaggttatttctttttttagctttgaatttgtcatgaccattttagtcttgcagatagcagggcagccccttggggcaaggatttactctgggaggtaccgttaagagccttctttccccctttgaaagatccttttacaatgttaaagtatactagttgcaagaacaagcaggatttgcaggttgctttaccagcatgagtctcatttttctggcttaaaatctgggactgtgaaattattccataggaaagtgaatnttattttgcagaattagcctcttacataaaagtatttgttgaagtgtctttaaaattgctatcatgagcaaaactggttgctgtaatgcttgtttt

SEQ ID NO: 35 - DNA, 442 bp, Homo sapiens
ggataccagattggagaagacttccaagagagatgatgccaagaaataaatgtaaaaaagcaggcccaaaacccaaaaaggcaaaagacgcaggcaaaggcacaccactcactaatactgaagatgtgttgaaaaaaatggtggaattggaaaaaagaggtgatggtgaaaaatcagatgaggaaaatgaagagaaagaaggaagcaaagagaaaagtaaagaaggtgatgatgacgatgacgatgatgccgcagaacaggaggaatatgatgaagaagagcaagaagaggaaaatgactacattaattcatactttgaagatggagatgattttggcgcagacgtgatgacaacatggatgaggcaacctattaggcatgaaatttttcaaaaaatatttttatgatgcagcttctgaacatttggacagacttgatttgt

SEQ ID NO: 36 - DNA, 299 bp, Homo sapiens
ctgcgggaacgggctgatcgtggaggagggacaacgctgccaccgcttcgcctgcaacacgtgcccctacgtgcacaacatcacccgcaaggtaacaaatcggaagtacccaaaactgaaagaagtggatgatgtgcttggtggagcagctgcctgggagaatgttgactctactgcagagtcgtgtcccaaatgcgaacatcctcgtgcttacttcatgcagcttcagacccgctctgcagatgagccgatgaccaccttctacaagtgctgcaatgctcagtgtggacaccgctgga

SEQ ID NO: 37 - DNA, 441 bp, Homo sapiens
tatcgcctggactccaaggtcaagcaaataggaggtggaattcagagcatcacctacacccacaacggagacatcagccgaaagcccaacacacgtaagcagaagaacggcttcccgcccaacttcatccactcgctggactcctcccacatgatgctcaccgccctgcactgctacaggaagggcctgaccttcgtctctgtgcacgactgttactggactcacgcagctgatgtctccgtcatgaaccaggtgtgccgggagcagtttgtccgcttgcacagcgagcccatcctgcaggacctgtccagattcctggtcaagcggttctgctctgagccccagaagatcttggaggccagccagctgaaggagacactgcaggcggtgcccaagccaggggccttcgacctggagcaggtgaagcgttccacctacttc

SEQ ID NO: 38 - DNA, 406 bp, Homo sapiens
  misc_feature (73)..(73): n is a, c, g, or t
gctgaccttcgaggcggtggatgtacccatgctttgccccccgctgccctggacatcgccgcactctggtgcnttcctgctcagccccaccaagctgatgcgcacggtggaaggcgccacgcagcaccaggagctgctggaaacctgcccgcccaccgcgctgcatggcgcactggacgccctcacccaactgggcaactgcgcctggcgcgtcaacgggcgcgtgctggacctggtgctgcagctcttccaggccaagggctgcccccagctaggcgtgccggccccgccctccgaggcgccccagccgcccgaggcccacctgccgcacagcgccgcgcccgcccgcaaggccgagctgcgccgtgagctggcgcactgccagaaggtggcccgggagatgcac

SEQ ID NO: 39 - DNA, 449 bp, Homo sapiens
ctctgaatgtgatcgacgcccagcaaggacaagctttaaaatgtctgcggtctgcccttttgaagcaggactggctcactctgtcattgggagctgtcagctgcgactgcaggttctctaggaggcattccagaatagagtagcacactgtgtctgcagttctcgatgaccgaaagttatcaaaaatatttaaaatatttaaattgtgaacctattgataaagaatatttataaaaactgatctgtaggcctgtactaatctctacgcattagcaatattgactgtaaacccacattaaggaaaccactacgggtctggcagtgcgtgtcccgtggggtgtgcattttaaaactcgattcatagacacaggtaccatgttccatttccgtcatggtgaagcaaatgaattggcctggctaccactgtggtcgcgtgctacaggtttgac

SEQ ID NO: 40 - DNA, 201998 bp, Homo sapiens
aagcttgcagtaagccgagatcgcgccactgcactccagcctgggtgacagagggagactccatctcaaaaaaaaaagagagaaagtaaaggaataaaagaatggctaccccatagacggagcagccgtgagggctgctggttgcccatttttatggttatttgttgatgatatgctaaacaaggagtggatttttcatgcctcctctttttagaccatatagggtaacttcttgatgttgccgtggcatttgtaaactgtcatggtgctggtaggagtgtagcagggaggatgatgggaggtcagtcttgtctctattttggttttggtgggttttggccagctccttcactgcaacctgttttatcagcaaggtctttatgactggtattttgtgctgaccttctatgtcatcctgtgacttagaatgccttaaccatcagggaatgcagcccagtagtttcagcctcatttttcccggctcctatttaagatggagttgctctggttcacacacctctgacatgatcattgcccactgcggcttccacctcccgggttcaagagatcctcctgcctcaccctcccaaggtgctgggactacaggtgtgtgccaccagctcagctaatttttgtattttttgtagagatggtgtttttccatgttgcccaggctggtctcaaactcctgggctcaagcaatccttctgtctcagcctcccaaagtactgggattacaggcatgtcccaccatgcccagactaatatttacttttaatcagactaagatagggttactacttgagttgctatggctccagctgaaagaaagcccgtgcagtcatatcacgcgtaaacatttgctttatgctaaaaatatggtggacctggcattacagctattacaaatctcctaagatgtctcgggtagtgtattagttacttttcatactgctatgaagaaatactggaaactgggtaatttataaagaaaaagaggtttaatgtactcacagttccacaaggctggagaggcctcagaatcatggtggaaggcaaagaaggagcaaaaaggtatgtcttccatggcagcaggcaagagagcacgtgcagggaaactgccctttataaaaccatcagatttagtgagatgtattcactatcacgagaacagtatgggaaaaacctgcccccatgattcgattacctcctaccgggtccctcccacgacacatggggattatgggaactacaattcaagatgaaatttgggtggggacgcagccaaaccatatcgggtagcaacaacctagggtcagttttgcaggtggtaaagccatttaccaagatagttgtgggtaaagaagggcagatttattagagaaattgtgaaaatatgttgcagtgggcagctcagcagagaaggggctacctgcaaagaggcaagggctggaggaaagttttatagggtcctgctgaagggtgctacgtgtggaatgaggtcattgtgcccgcaggttgtttgtgattagctgtctctaacaattgttcatacaataattgttcattattgttctcaacttggggctctccccaacctggggacccttccttattgttgcttacttatcaggtctccacataaaggtgtggaaacttcattcattcatatcttcaacacaaattgtaggtagcctgttttttaaaacatttattcaacaaatatttagtccaagccactattacttactaccttctctactattgtatggacttttaactatctctgacactattcactattcttccacattctctattatttatacctatggtaaaatttgccagtttgaccatacaactaatactcacagggaatatatagagtctagaagaaaatatacaggtccttaaaggctgccctgccaacaaaaccataacgcaggaacaaacatcacaactatgccaaataatcaatcctacaatgtccaaaattttactttaaaactggaattaccagacttcctttctgcattaaccagtttaactagacagtaacgaaatattcctactttatgctgtgatagtttgtttgtttgtttgtttgtttatttatttatttatttatttaagacagagtttcgctcttgttgcccaggctggagtgcagtggcacgatctcagctcaccacaacctccgcctcccaggttcaagcgattctcctgcctcagcctcccgagtagctgggattacaggcatgtaccaccacgcccgggtaattttgtatttttagtagagacggggggtttctccatgttggtcaggctggtctagaactccagacctcaggtgatacccctgcctcagcctcccaatgtgctgggattacagctgtgaagccaccgcgcccggctgctgtgatagttgagatgtaaaccaaaaataaaattctaagccacccaatccgactgaatggacccttcctgttgagcaaggacattccaaagtaaactgaaaagaccagcttaggccatgatgggaaggggaggtgtcaacatgcctcattctaccttcctccctctggaatccagacacaactgaccagcattaacattaaaacagagatcttaagctgggcacagtggctcatgcctgtaatcccagcactttgggaggccaaggtgggatcacctgaggtcagaagttcaagaccagcctggccagtatggtgaagccatgtctctactaaaaatacaaaattagccggacattgtggtgcacgtctgtcatcccagcaaggcaggcgaatcacttgaacccaggaagcagaggttgcagtgagccaggatcatgccattgcactccagcctggtcaacagagcgagactccgcctcattaaaaaaaaaaaaaaaaaaaaattagccgggcgtggtggcgggcacatgtagtcccagctactagggaggctgaggcaggagaatggtgtgaaccagggaggcggagcttgcagtgagccgagattgtgccactgcactccagcctggacagaagtgcatttcataatgcattttaattgcattagcagtgatttaatttttttagatgctaaaacttatgggtgaaagtggattaaatgtagccaaatgcaacatcaaaatcttcaggcacaaaaacccattaactttttcatactctcagaaggtgaacctaatttcaaatgaaagctgcctccagaatatattgttaagcgtattctagatataattcattttggcaaacatactgtagaaattcacataacattttactgtactaaaagtaaattgcccatgtaacaaaaaatatcttttcagagcttgaaatgaattttaaaggatgactgatggtccctggaagagaaacagtaaacaaataaggtttgtagcaatgatgtatgagttagaaattgcagttccagatgatctctttattaaagagacgatctacacttaatttgatcaagtgttatgaacatagttcatgttaagtctccatttaaatacaacctgaaataccaaagttaattttcttttctttctttctttttttttttttttagaaggagtcttgctctgttgcccttcctggagtgcagtgacgtgatcttggctcactgcaacctccacctcctgggcttgagcgatcctactgcctcagccccccaagtagctgggaggacaggcgcaagccacggcactcagctaatttttgtatttttcgtagagatagggtttcaccatgttgcccaatttggtctcgaactcctgagctcaagtgatccgcccgccttggcctcccaaagtgctgggattacaggcatgagccaccgtgcctggccagaaaattgtaaacacacacaaactctcaagtggcctaattccctctcaccaaaccaatcacaatacagataaaagagaataacttgtgttcatttttgtacaaacaaaaaagatataaattgtgaatgatgcatgatttttaattacaagtaaactgggcaaatgcttctgcattatttaaagctaaaaggtgatcagtggaaactttcctctgttagtactctaatactttttatatttatcggctcactacaacctgtgcctaccaggttcaagcgattctcctgtctcagccacctgagtagccgagaccacaggcacgcactaccatgtccggctaattttgtatttttaatagagacagggtttcaccgtgttggccatgctggtcttgaactcctgacctcaaccgatccgcctgccttggcctcccaaagttctgggattacaagcgtgagccacagcgcccagccttattataattgttactatttaaatctcttttgctctctccttcaagagagacctcatcccattcagttgcttccatttatttattcatcttctgcctcctgggctcgagagatcctccagcgtgagtctcccaagtagctgggactacaggctcacaccaccaagcttggctaaattttgtaggttttggagagacaggctcttgccacgttgcctaggctggtctcaaactcctgggctcagatgatccacctgccttcgcctcccaaagcactgggacatgagccaccacgcccagccgcaagtacttttacacaaaatgcaaacaccattcttccatcataaaagtgataccacagcttccgtgaagttttgccaggtagtactcataattaccttgggtaaactttttgatgttaaactgtatcttcttattacgagtttttccattgtattaactgcttttacaacaacacaaataacaagttattttacaaaccatttagaaatttctgtactatggtcccagtaatgtaaaatatattaatgcctattacattcagataaattatacacttggaaaccacatacttatgacttacagaaacttacataaacaaattatagaaattacatgctcaatttttaggtatatagtcttaaattaagcttaaatatacattctcaagataaattaacagttcagggcttcacaacttgaaatctgtggaagatgacattggagacaacagaactctggtggaattcttagatggaatttgccgaaacttttttttttttttttttttgagatggagtgtcgctctgtcgcccaggctggagtgcagtggcgcaatctcagctcactgcaagctctgcctcccgggttcacgccattcttctgcctcagcctcccgagtagctgggactacaggcgcccaccgccacgcccggctaattttttatatttttagtagagatggggttttactatgttagccaggatggtctcgatctcttgaccttgtgatctacccgccttggcctcccaaagtgaaacttttctttaaaatagagatgggatcttgctgtattgcccaggctggtctcagactccttgccttaagcagtcctcccacctcagcctcctaaagtgctgggattacaagcgtgaagcattacatccaagtgaaacttcttgagatggttacataatgtctaaatctgctggtgtagaagttaataaagtgtagaactgaataactattaaatattagatcaagtttctcatgtttatcttaacgtataacgatttatcttaaagcactgattttcacaaaataacatcagtgtgaaattggaaaagaagccaaatattttatttcacgtatctgggaaatgaggtgctttagtcaactgaatctgcccaaaactaaaaagcattaattaaaaagtacttaactcagaaattataaaaataggagacatcaataaaatacattctacacagaatacgccaaccatacactactcttttttgataataaaaaatgtatttactgagccagttgtggtggctcacgcctataatcccagcaccttggaaggccaatgagagtggatcagttgaggccaggagtttgagaccagcctggccaacatggtgaaatgccgtctctactaagaatacaaaaatgagccgggcacggtggcacgcacctgtaatcccaggtactccgaaggatgaggcaggataattgtttgaactcaggaggtggaggttgcagtgagccaaaatcatgccactgcactccagcctgggtgacagagtgagtctctgtctcaaaaaaaaaaaaaaaaaaaaagaaaaaaagtcagttgcagtggctcacgcctgtaatcccagcactttgggaggctgaggcaggcggattacaaggtcaggagatcgagaccaccctggccaacatggtgaaacctcctctctactaaaaatgcaaaaattaggctgggcacggtggctcacacctgtaatcccagcactttgggaggccgaggcgcggagatcacgaggtcaggagattgagaccatcctggctaacacagtgaaaccctgtctctactaaaaatacaaaaaattagctggatgtggtggcagcacttgtagtcccagctacttgggtggctgaggcaggagaatggcgtgaacccgggaggcagagtttgcagtgagccgagatcccaccactgcactccagcttaggcgacagagccagactgtgtctcaaaaacaggaaagaaaacaaaagaaaatttggactattgccaattacaaatatttttagagaagaattcaaaacagtaactgtggatgatggaaacaatagttatgataaaagtctgatgaaacttcccagttcacaaggaaatttaattacttatgtgcagcattttaagacagtaatcagaatcatgactgacagcatcatatcagggccagcagacttttataaatttcatacaatcttcagaaataataacttttttttttttttttggatagattctacctttgtcacccaggcgggagtgcagtggcatgatctcggctcactacaacctccgcatcctgggttcaagcagttctcctgtctcagcctcccgagtagctgagattacaggcatgtgccaccaggcatggctaatttttgtatttttagtggagacagggtttcactctattaggctggtctggaactcccgacctcaggtgatccacgtgcctttgtctcccaaagtgctgggattacaggcatgagtgacagtgcccagccattcgtgacatgtttatacaaatataactttagcaaatatttagcataactatcaaaattacaaatcatattaaatttgtataaatgtatgcaattttcggaacacgcatatcaacaacatacccataaatataactgagatgagatctaatgtcacctcacttgacagtgccctcccatgcagtatcgccacatttgacaatgcctgcccatttaatctaccaaataaatcgaatcacttaatacctctacaagatgagagatacattctttagactccccaagggatgcagctgaaaaaaatcccaaagttagttttaagccaaaaagacttgatttaggattttgacactggagaaacccatcaaagatgtcaagtttgaaaacacttgatcaaaacagaatcacaggtcactattaaaagagtattaatttaaccagagacttccaaagcaatacagaaacttacatggatataaaaaccctaacccttttaaaggtcagatttgctaagtgatcaaaaggggtacttgaattgaatcgacacaggaagagtgtgtacagggttatgagtgtaggcaggtggttactttggtcatatctccatttgccacctgattacacatgagaatggcatctttactcaccagaaagccagtattataggaggtgtaggaggcattcttggacttgagacaagaacattgttgtgtagaaatttcattgactgtgttaaaattattctccatgggctggagaacacataacatggcctttagaatgagacgggcattgattggatgcaaggtctccacacttactagctgtgtgacattggacagagtgcttcatcattccgagactcagtttttaaaggaaaaacaactaactaccttgcaagcttgctagcaggtttaagtgtaataatgtgtgggaatgactgcaccgtgactaacatgcagtgacagcttaattaatgttaacccttatcattatcatataagaatgtgagttacataagagaggagtcctgtcagttcgttctctgctgtgtccccaagaccatgaatcatggctggcatgtagtaggcatttaataatatatgttcaacaagtatttggcagtcttggagggcagaaaaggaggtggggaagatttttaaataacattctttaaaaagtcacattgtcctacaataccgatttttcttgcatatttaggaaattgagggtttttttctaaaacatgcggacatatgggaaataggatgcaacatttgcactaatgtttcagacacagttagaggtttccaagagattttgcgctggggaggctgcttgctacaagctcccaaagctctgggaggacatagtattcattcctccctcagcagaagcggtgaggcaagaagctctggggagcacccagcgttggacttttagcatagtgtgtcaggtcttcatagtttgggcccagggcacagagaagtcacagctctccggcatcctgtgacctttaccctctttgccaagggaaaatgtggcccaccaaagcaagaaacttgagggcatgggtcaccccagccctggcatctgcccagagcccgagaaggaaggaacaatgatcctccagctacctcacggggctggcacaggtgaccactgccctggcatcacccagctgtgtccggcagcctgaaccccatctgtggggatgcgaggaggaaaatacaaaagtccttaggtgaacactgagaaggcagatgcagcagaaacctccaggccagaactacccagtcttggacctatggtggagatagagcatagctggcgatcatgtgtacttacactctaaggtcacctggttgcactatggcctcatctgtggctctgaaaatgaagatttggaaggagatcatcacagctaatgtttaacaagcccctcctgtgtgccaaatcattcacccctcaccacaaccgaatgagctaaggattctcattatatatagtttatggagagggaagtgcagacataaagaggtgaattatcttacccagatcacacagctgataagtggtggaggcagaatagaatctaaacagtgtggctccggagcccacatgcattgattcgacaagtgtttattgagcacctgccgcggacaaggccttgtgtgattaaatagggttataattagtaatataaaaatgagaaatcactaatgctttttagacttaacattttgtttttttgtaggtttcaggcacagaactgtatatccaataatagtgaaatggatcccactaattatgacagaaatgatgatacatttaaatgacttggatgttttataggtatgatctcgtgaaatcttgagagaaactgaatgacgaatgaaactattgttcctgtttcacacagaagaaaactgaggttaaaaggggtaaagtaattttgcatggcatgaagtagaaattcaaagtacaggaatttgaacttggttctgtccttttctgaagcccttgaccactatagactcaaacatcaccttgtttttccactcattcaacacttttttttttaaattatctaataggttggcactcatcatgagcccctgttctcattctgcaaatggtgaagctctctattgtcctgaccccacagttcctgtcccatgaccagggccagctcaccaaggagctgcagcagcatgtaaagtcagtgacatgcccatgcgagtacctgaggaaggtgagtgagtgcagacagatggggcctggtgcccttgagcagttcccgggtctcagctgccacacatctcatagccggtgatgctgggggaagcttacgcagtcacagtactggcttcttcctctttttctttccatacaagtggcttagggatggggtagagtagttgacttatttggatgaaaaccactatcttctgtcagaaactcaaaaggaatcattgctggcatggtaacctaaagaaaaacaaccagacaagtgcccaacgacacttaaaaaggtgatttattatcttgccaagtttaggctgggcatggtgactcatgcctctaatcccagcattttgggaggctgaggctggtggatcaccggaggccaggactttgagaccagcctgaccaatatggcaaaacctcgtccctactaaaaatacaaaaattagccgggcatggtggtgtgagcctgtagtcccagctactcaggaggctgagacaggagaattgcttagattcaggaggtgggggttttagtgggccgagatcacgccattgcactccagactgtgcgacagagcgagactctgtcaaaaaaaaaaaaaaaaaattatcctgcaaaatttgaaaaggaaattcaaatcaacagcttctaaactactttttaacatgactcataataatacattctatagtacatatgtatgttctataactttgaataaaagagttaaccacatcacatttattttataacatgtaatacatattttttattctccttcatttgttttgaatgctctgtgcagtctacaaaaagtccaatagtaataattaaattagtcattaagttgaacattatcttgtcttttaaaatgataatctcaaaaatgatcttttatttttgagatttatatagatacacacacacacacacacacacacacacacacacacacacacacacacacacacatattttttgagacagagtttcactctgtcccccaggctggagtgcaatggcacaatcttggctcactgcaacctctgtctcccgggttcaagcaattcctctgcctcagcctctgagtagctgggactacaggtgtgtgccaccatgcccagctaatttttgtattcttagtagagatggggtttcaccatattggccaggctcgtgtcaactcctgacctcgtgatctgcccaccgcggcctcccaaagtgctgggactataggtgtgagccgctgcacccggtccaagtaaaattattttaacaatatactatgaagagaaaaacactggctatgaaagaatatgcatagttttaccctgtttaaaaataaagattgaaagaatacatatgcaaataagtttacttttatttttggtaacactttactgcattgtctgaatattgacaatcagtatgcattatgaagctacctggctaacattgtgtactcactgtgtgtgccaggccctgggttcaatgctctacatgcacttatatttcatttaattctctctgcaacctgagatggtatagccacctcattttacagagttgaaactgaggctcagagactgaaagttaagcctgaggttgcagtcaataagaggcagagctggaactgaaacctacctgtgtctgaccaccagttcgtgttctgacggcaggctagtctgcatcacagagtgtggagtagatggtgcatgcctgctaggatgggctaggtatcactgtaggtaagaaacagccccaaactatggaaatgtacaccactgaaggctcttttcctgcccatgctgcacatcctccatggctctcctgtgccctgtgccccacatgccctcatcctgccacgagaataaaggagcagcctccatatgggagctgtcagctgctctaagagatgaaggagagagtggcccgtctcaatggctcccaactcttttgcctcgaggtgacacgcttcacttccacgcacatctcctgggtcaaagcaaatcccatgggtacatccactttcaagtggcccaggagagaacctgaaatactcggtggactccattaaggccgtcatatggtgtcagcctgcatgggagactgtggaggggcagaggaggagagtggggaactgatgggaaatgacaggaggactaagtcaccgcagatttgctttatcttcagccaggtggagtttgtcccagagccgcacaaaatcatcaccagcatgattaaacggagtagacttcagaaaaagcagtttggtcggatgtaatcagcagtgaactcagaatcaattgagtgacattgagtcagtaaatctctgactgcctcagttaccccatatgatagttttgaggatgggaacattgagagagttgatttggaaggatatcaagagtaaaaattccaacatttttagttcctttaagttaaatccaggcactgtctttcctgcaagtctcctgttcctttcagattgcacaggtgagagtgctcagattagggctggaggttgtaaaccattgctcccacactgacagtgcccccgtgtcgtgcgtgtattctgcgcattttcctgtgctaaacactctcccaaaacatcgtggggcctgattcttcctctttgttccaatggccctgggtgactcaagtgcccattcaatgaccaggacacagaggtcttagagagatgctccttgaggccccaggtgcgagcctgtaccctgccggagcatgaggcaagggacagggcatcgtctgtggggatagtgggggtagtgggggtagtggtcagccagatttggtgactctacttgctcaccagacgatcctacacctgccacctccgatggatccactgcctctgtgcctgcctgtactgctgatgctccagtggataactcagcatcccagcctaggcccaatgccactgaagatggacctgccccctggggacccaggagtcctaccactcagctgtccccaggagtgcccagaccctcattcttatccaggacctaggagccctacccctggccttccctcatcagccgtaaatgatgatttactgctgttaccatcatcactgccttcagtgaccaagggccttccaaggtgccagctctggaacgaaggatgcccttgggaggtgatgacactcaggtacacgggtgctcaacagattgcttcctcctatcctcagacggtctttgcatgcatgcagccattggcactcccattgtgtggaaggaaaccagcccagggtcacacagctggtcagcagcaacatagctggtctcaaatctaaggtgcctgaccatgcctccatgagggaccgcctccaagggaggttgatcctggctttggggagcctttcctgggctgcacgaataacctccattgttcgagaccccaaactctgctcacatcttcctttccctatctctgcttgggctatgatcacggtgactctagcagcccttcatggacattatagtactctctgccattcacttttgctctaatctgacttcaacccccacttacttggtctctccttttacaaccaccacaaccgaaatctagggctgcttttttttttttttttttttgagacagagtctcattccattctgtcacccaggctggagtgcaatggtacgatctcggctcactgcaacctccgcctcccgggtccaagggattgtcctgcctcagcctcctgagtagctgggattacaggcgtgtgccaccatgcctggctaatttttgtatttttagtagagacggggtttcaccatgttggtcaggctggtctcgaactcctaacctcgtgatccgcctgcctcagcctcccaaagtgctgggattacaggcgtgagccaccatgcccagccaaatctagggcaggaacatggctgcagcatataaaaagaattgaattccatacttttgttaaccctgttttttgtttgtttgtagttgttgctgtttttgagacagagtctcgctctgtcgcctaggctggagtgcagtggtgcaatctcggctcactgcagactctgcctcccgggttcaaactattctcctgcctcagcctcccaagtaggtgggactacaggcgcccaccaccacacccggctaatttttgtattttattagagacagggtttcaccatattggccaggctggtctggaactcctgaccttgtgatccgcccacctcggcctcccaaagtgctgggattacaggcgtgagccaccacacccagcccctgttttgtttttgttttgcttgcttcttagggttgtttttctatttatggtaaaggcattggctttccatttgtagcatcaatagaatatttcctgtttacaataaccttatgtcatagtaaatggtaaagggatttaaagcagtggttttcagctgccagaggcctgagagagtttgggcatactctgtgtgatcgggcagaaggcctgtgggaagtttagcagaggacagggccaggaaaggtgatggacagtgggggtctgtcctggtcaccaggcccctgggtcctgcccacctgcttggagctccccacccatcacacatgatgctgccaagccctctgggtattgtgggcaaataccttaggagagaagctgatgaactttgtttcttgaaatgcacagattccttggacgtccctgagagctcagtcatgaaagtcaacttggttttctccccctcatttgggttcagaatttaaagtccacacacacaggcagtaagatgatatagataaggacgtcatcactcggtttcggatgttaaaatgtctaggtgggttagcggtgatttgagatcacacaaccttgtgccacaaagaggaattcccaggccagagggagacattttattgccatgttatgatctcatcattgagttgaaaggcaatcttgtttcattttggattctttcttatgtttatgtcttataagggcactttgaatttccaagcaaataataattttgaattagcttttaatcattgacttctagcacagttatatgatcagaaacatgctgtgtgatttgattgctctcaaatatattgagatttgctggaacaaaataagtcaggttaatttttgtaaatgtaccaggcatgcttaaaatgaatgtatctacatttgttcccgagatacaggttgatggacggatggctacatggatgtgatggagatggtttactatcgggaccttccgcaccctgctgatgttttgttgcttaggatatgaatggctgagcggaggctgtaaaacctggcactctgcttgggtatgaggttcttcctgccatcctgccatcatttgttttttatgttttgtcgccataagtgaccttgaggaaccctgggagctcaggaaggaaggagcgcccagaagcagggacagggagctggttggggaggaccagaaatcaggtttgtgaaggttccagagaggacctgtctttgggaggagtgtgggagactgagatgggggaggggtcattggaatgatgcgggcgctacttggcattgtccattgtgaggcactgtccattgtgaggcaccaccggggtcatcagggattggtggagagggagtataaagccccagggttggtaagggagggcccagaccgaagaaggtttggtggatagcagaacctttttgtctccctctgattgctcctaagcctcacgctcccttgccccgcgtgtcctgttgcttccctgatcttctccgtgacctgtagctaaaccttccaccagcgcttgagaacttaatttgaaccggatcctttcccagacccctttcttcttctcctcctcctcctccacctcctccaggtgcccaacagcccccttctcctcctttcccttcccttacttccccccttcccctccccttcccctccccctcccctccccctcccctccccctcccctccccaactcagatccggccccggtccccgtccccttccctcccccctgccctaagccacctccacctctgtcctggccgcctcagggcgccctgaaaggaccaggacatgcgggtgcggtggatgctcttttggctcctctttgggctcctactggaatttatcagccatcagtgcatctctgtgagtagacgctggacccgtggggtttcttcctttttactgggctgtatcacgtggcatgaaattacacagctcaggcctgtaatcccagcactttagggggccgaggtgggcagatcacttgagtccaggagttgaagactagccagggcatcatagcgaaaccccatctctacaaaaaattccaataaagattagtcgggcctggtggtgcgtacctgttatcccagttactggagaggctgaggtgggaggatcgcttgggcccaggagctggacgttgcagtgagccgagatggccccgctgcactcttgtttttaacaaagaaaatggaccaaaacaaagtgaaatgtcatttgatttgtgtcatctggtttgatgacttttttttttttttttttttttttagacagagtctcactctgtcgcccaggctggagtgcagtggcaagatctcggctcactgcaacctccgcttctggggttcaagcaattgtcctgcctcagcctcctgagtagctcagattacaacgcctggctaatttttgtatttttagtagaccaccacgcctggctaatttttgtgtttttagtagagatggggtttcaccatgttcgccaggatagtctccatctcttgacctcgtgatctgcctgcctcagcctcccagtgctgggattacaggcgtgagccaccgcgcctggccaaaatatataaccttaagtgtaagtttactaactttggaaagtacatacaccagcataaaccaaccccctttcaagatctacattattttatttatttatttatttattttgagacagtttctcccttgttgcccaggctggagtgcaatggggcaatatcagctcaccgcaacctctgcttcccaggttcgagcgattctcctgcctcagcctcccgagtggctgggattacagacatgtggcaccactcccagctaattttgtatttttagtagagatagggtttctccatgttggtcaggctggttttgaactcccgacctcaggtgatccgcccgcctcggcctcccaaagcgttgggattacaggcgtgaaccaccatgcccagccaagatctacactattatgtcaccccagaaagtgaactctcagtcttcccagccagtctctttcttatcataggttagcttgcttattctggaatttcgcgtatacagatgcatgccatgccataggtactcttttgtgtctgctttgttctgctcaacaccatgtttctgaaatcattaccattgttgtatggttctctaacttcatcatttccatttcagactcagcatatgctgagttcaacctgttgaagggctatctctgtttaattcaccatcttgaaagaaacatttaaaattgagatgttttcaagaatatatagttaaatcctgaggaatcgacgtagaaatgttatcacaagctgtctgaacttactcaggggaagtcttcgtcttcactcacataagagtctaatggaattaatatcaacaatcttagagaaatcccacactattcatgccattttcatgatctccaccttgataatttttttttttttttttttttttttttttttttttttttgagacagagtctcgctctgtcacccaggctgaagtgcagtggtgcgatctcggctcactgcaacctctgcctcccgggttcaagtgattcttctgcctcagcctcccaagtagctggaactataggcatgtgccaccatgccctgctaattttttgtatttttagtagagacgggtttcaccgtgttagctaggatggtctcaatctcctgatctcgtggtccacccacctcggcttcccaaagtgctgggattgcaggcgtgagccaccacgcccagcccaccttgttaatttttaagcactaaaatttgatacttatttgtgaatgaagtaatctcttcattgtatttttttttttttttacttatgctgagatttaaatgacaaagattcatataatccaagagagaagtattatttagagggattcttttaccatgtgatatataataaatgcatccaatgttatacatcaatttaaaaaacaagtaaataactttaaagaaaagataactactggccaggtgcagtggctcacacctgtattcccagcactttgggaggccaaggcaggtggatcatgaggtcaggagttggagaccagcctggccaagatggtgaaaccctgtttctactaaaaatacaaaaattagccgagcgtggtggcaggcgcctgtaatcccagttactcagtagctgaggcaggagaatcgcttgaacccgggaggcggaggttgcagtgagttgagatcatgccactgcaatctagcctgggtgacagagcaaaactttgtctcaaaacaaaaagaaaagaaaagataagataattactttatacttagcttgtcttacccatgagtgacgggctgcatgtggcccaggacagttttgaatgcagttcaacacaaatttgtaaactttcttaaaacattaggagattttggccaggtacagtggctcatgcgtgtaatcccagcactttgggaggctgaggcgggcagattacctgaggtcaggagttcgagaccaccctgaccaacatggcaaaaccccatctccacaaaaaatacaaaaatttgctgagtgcactgtcaggcacctgtactcccagctactcaggaggctgaggcaggagaatcacttgaacctgagaggcagaggttgcagtgagccgggagcacaccactgcactccagcctgggtgacagagtgagaccccatctcaaaaacaacaaacaaaaacaaaaacaaaaaaatggctgggcacggtggctcacacctgtaatcccagcactttgggaggccgaggcaggcagatcgcctgtcaggagttcaaggccagactggccaacatggtgaaacctcatctctactaaaaatacaaaaatgagtcgggcatggtggcagagacctgtaatctcagctactcgggaggctgaggcaggagaatggcttgagcccaggagctggaggttgcagtgagccgagattgcaccactgcactccagcctgggcgactgagtggagcggaactctgtctcaaaaaaaaaaaagaggttttttttagatcatcagctattgttagtgttagtgtatgttatgtgtggctcaagacaactttgcttcttttaatataggcagggaagtcaaaagattggatatccctgctttataccaagaaagacaacaccccacatttgcaatgcctgaaaacactaccagccatctgaaaaacatgtgacttctaacttctgttcttttttgtagcagtggaatcccacggtgatatctgagggatgtggttaccttttggaggaggttgacggtttctaaggatgattctttctgagtgaaatattgtcagtgtcattgaccttttcattatttcaactattattattccaggttatcaatactctggctgaccatcatcatcgtgggactgactttggtggaagtccttggttacatgtcattattgcatttccgacaagttataaagttgtcattaccctctggatagtttacctttgggtgagtatactaactttctgtagaggtatacttgtaatcacaaataagaataaattatataaaacaattcacatttctggacttcattatgaatatgtggttttacccaaaaaatcagggaaatgatttattagtataagaattatgaaaacatctgccatttgcattatgaaaattaaataggtcggtgtttgtttaatagaatgtcaacagagcttttggtcaaaaataagtttttttaacctttgtgctatttatcacaaatggagtatgaggtttcgtcacttaaataggaaattctttctaaactcttctgctttatagttctatcgtatgggtggaaggaaagcttccaatctcctctctgaagattcactgcagaaatgagctgacaacagacagcttaacaggaaaagaaaaacatagaacaggcataaacatgggaaccagctgaaaaatgagactgctagaagggccggatggctgatgcttaaagagcaccctcttctgaggggagagggagatagatggagatgtaggccatttagaggggcagcaaatgatttttaggggaaatgaaagaggccaaggaacaaacaattggcctgagacaaagttcctgtgaggtcatagggacgaggtgacaaactgccggaaggtgaagggcagaactgcactgcgtctcatgatgcagagaaagccccagagactcttagaactgccctccaagagaatcaatgaaaagtgtgtctgggcagggtaattttgaatgacatcattcaaagtgcatgttccgacttggaactggagagagatcagtatgtcaaaagtctgtacttggtaagaatttggctgctaagttgtgccataatttgtcttttgagccttttttcctttgggtaagttgagctctacattttgtcttgccattcatgacagtaaaaatgtggttgtctgggggctgaacctccttctgaacaatgatccaagataaaagtactaataccacaatgcttttttatattcaagggaagaggaagtatgtttcagttttaccacctagataattacacgtcatttggcactgcctttcaagatatgtagaaaacagaaaatatatgagttatgaagatatctaggcacatttaacattctctatgccacttagtcctgaacagagaattttcggtataaattggaggaagcttttttcttttttttttttcttttctcaccccgaagacgagtctccttctgttgcccaggctggagtataatggtgtgacctcggctcactgcaacctccacctcctggcttcaagtgattcccctgcctcagcctctcaagtagctgggattgcaggtgcccaccaccatgcccagctaatttttgtatttttagtagagtcggggttttaccatgttggccaggctagtctcaaaacccgacctcaaatgatccaccaacctcagcctcccaaagtgctgggattacaagcgtgagccaccacgtgagccaggggaagtttttaaatttaccactttttaacaattccacttaggaaagttcagttgagctgttggacttggacaacttcgcacctctcatctttgtccttgtcatctagtcatctataccattacctccttagcagggacatcatgggtgccatgaagcattcatgcgtgatggcatttcttggcttctcatttcttcatgtgtttgacatttcccctagctccaaactgggccagctacctttcctatgaaatctagcagtagctgtgggattgacgtggttgctcttttcatctttttagattacccattgcttctctcgaaatcctagtacatgattttttttttatcctatgtgcagaaatcaggaaaaaacaaattctacaaagaatttgaaagatattatttcaggccaggtgtggtggctcatgcctgtaatcccagcactttgggaggctgaagcagatggatcatttgaggtcaggagttcaagaccagatgggccaacatggtgacaccccatctctactaaaaagacaaaaattagccaggcatggtagcaggcacctgtaatcccagctacttgggaggctgaggcacaagaatcgcttgaatctgggaggtggaggttgccgtgagccaaggtagcgccactgcacttcagcacggttgagagtgacactctgtctcaagaaaaaagtcatttcaatgaccacctcaggagattcataggtatctgacccacatctgagatgggatttgcattgcattttagctatgatgagaagaaatatttaatatcttagaagattaaaagcatactgtgataatatggaaatcttggtgggaattcagtcattagtgagaatgttttgcgttaagttcaaaccagcctcaatgaagctgatgtgagggaagggaaagtgaactctgagtagagcagggacagaaggaagatgctccagtgcagatcaggaaggagcaggggatgaaatgttacaaattctagaactcagagagctgaaggtaattacttccttttcaagttgtgaaacatgttaacctgtggtaaaatacttataagatgataattaccatctaaccgtgttgaagtgtacagttcagttgtgtgaagtatattcatgtcatttttttttttttttttttttttgagacgaagtctcactctgtcaccaggctggagtgcagtggtgggatcttggctcactgcaacctctgcctcctgggttcaagcagttctcctgcctcagcctcccgagtagctgggactacaggcgtgcatcaccatgctcagctaatttttgtatttttagtagagacggggtttcaccatgttgcccaggatggtctccatctcttgaccttgtgattcacccgcctcggcctcccaaagtgctgggattacaggcgtgagctaccgcatctggcctatttttttttttttttttttttttttttttgagacagagtttcaattttgttgcccaggttggagtgcaatggcacaatctcagctcaccacaagcttttcctgctgggttcaagtgattctcctgcctcagcctcccgactagctgggattacaggcatgcaccaccatgcctggctaattttgtatttttagcagagacagcgtttctccatgttggtgaggctggtctcaaactcccgacctcaggtgatccgcctgcctcggcctcccaaagtgctgggattacaggagtgagccaccgtgccagcctcatgtcattcttgtgtgtgtgtgtgtgtatgtgacagagtctcattctgtcgctcaggctggagtgcagtggtgtgatctcggctcactgcaacctccgcctcccagcttcaaacggttctctgcctcagcctcccgagtagcttggattacaggcgcccgctgccatgcccggctaatttttgtatttttagtagagacggggtttcaccatcttggccaggctggtcttgaactcctgaccccgtgatccacctgcctcggcctcccgaagtactgggattatacgcatgagccaccgtgcccagccgtcattcttatattattatttcctaggtgtctctcctgaagactatcttctggtctcgaaatggacatgatggatccacggatgtacagcagagagcctggaggtccaaccgccgtagacaggaaggtatggctctgttggaatccgcatagtgtggaaatgagtttgccctggaaagggaaagaacagcttcttgccctcaggtttctcaccttctcctctcctcactctcaccaagggctgaggtccatttgtatgcacacaaagaaaagagtttcttcctttcgaggaaataaaattgtcctgaaagacgtcattactctacggagacatgtggaaacaaaagttagagctaaaatccgtaagaggaaggtgacaacgaaaatcaaccatcatgacaaaatcaatggaaagaggaagaccgccagaaaacagtaagatgtgccttgacacaaatactgttgtatgaaccatgtgccaatcaaagtagacaactgtaaagtccttgagaatattttctacaatatttgtggcaaattcagtgggttcaaaattgagtttgtcctttctgcttcattagtttaagctgtataattcctttcccttcctacattcttgttttcattttttcggaggaagaggagttgctagtactggcattggttttcctttctctttttttttttttttttttcctgagatggagctttgctgttgttgcccaggctgtagtgcaatggcacaatctcagctcactgccttttgggttcaagcaattctcctgcctcagcctcccaagtagctgggattacaggtgcccaccaccacgcccagctaatttttgtatttttactagagatggggtttcaccatgttgtccaggctggtctcgaacttctgacctcaggtaatccacctgcctcagcctcccaaagtgctgggattagaggcgtgagccaccacagccagccttttttttttttttttttttttttaattttgcgatagagtctcgctctgtcgcccaggctggagtgctatggtgcaatcttggctcactgcaacctctgcctcccagtttgaagcaattctgcctcagcttcccgagtagcttggattacaggtgtgtgccaccacatttgaccaatttttttttttttttttttttttgagacagagtctcactctgtcacccaggctagagtgcagtggcatgatcttggctcactgcaacctccacctcccaggttcaagcgattcttatccctcagcctcttgagtagctgggactacaggcatatgccaccatgcccggataatttttgtatttttagtagaggcggggtttcaccatattggccaagctggtctagaactcctgacatgatccgcacacctcggcctcccaatgtgctgggattacaggcgtgagccaccgtgcccggcccaatttttgtatttttagtagagacaggggttcaccatgttggccaggctagtcttgaactcctgacctcaggtgatctgcctacctcagcctcccagtgtgagccaccgcacccagcctggattgttgaattcaatgcttgggtcacctccagattcattttcacagtctttcatgttttggtcatatgacattgtattttgctgccatatgactgatctttttttgttaaatgtgagatacttgttaaaaaatgtttagcaatgaattgaggcctagtagcatgttatcttgctgcagaagagatgggagtctacttctgggggatggtcaggggtcctccatacaagctgcaattgaagtcgtcggtgcaggctcagtccctacaaaggccagggtatttcctgtccacctttattctgatgcatgactcttctgggtctcaaccagagccagcggacttcagtatgggtcgctttcattggcagaccctcaatccacttgttttccatctaatcccacgcatgtgtgcaaaagctgctgtgcttctttgcatctcagtagttccttctggaattcagcaatgaaactcagggaaatgggttccaaatgcgaggctgactttcgtcctgggtttccttcttctccatcttcacctcatgtctgtttactgccatgttagcaatttgatgtattcaatcatgggttttatattctgtttggtgtcccccattgttctcattggagatcagaagcttcagatgcacttatgtcaactcaagagtagaatgcttccttagcttccctccagagtcaggttttgtgtttctagttcccaagtgcacagcaggagtagtgatgtcctcactggcttctcatttgcattaagctgtgagcttctttagcgtggggacaggaccctgctcccattgcattctcagcaccacaccacacactccttgtttgaggccactccagacagcatgtgctgaaggatgccttgtggtcagaaacaagttcattaactttctctttgaagtgttttcgcccctgtttcctagcgttctgggaattttacacatccttcctataaagccaagtatcaggtgagatccttaggatcaggaccatgaatcaagtggtgtgagggcaacacagcaaacttaccctttttaggccgtttcctttttctgccctcaatctctgtgaactgaaccttgttaaagtcagtcaacaccagggtggatggtttgccgttgtcacctattttcaggacataacaccctgacttaggagccattccgatcatttctaattcaatagatgcgcccagcattcagattgccttttctctcaaccaggatctttaaagttgatgacaagagttccagtcctgaatcatggcaaagtgcagtagtgaactgcggggttattctggaaggatctctctatggctgatggtctcagttccggcatcagcctctgactgagaatcaggtctcacacaggaggagtcagatgaggagcaatcctctgcttccgatggagttagttgtgatgaattggtgaggtctggtttttcacactgaactaaaatgagctttcgctgtgtcaagcacaagactgaccccagagacacacatagtgcacctcatagaagcttttaatagtctttatatttactaaagaataggactaactatggaactatgaagatgagctggaaatgacaggtgacttgccagcaggccagagtgtgacttttttttgtccctcaatgggaggtgtcaattctcccttcggttgtgagaatcagttggttcatttgtgggaaggttgcaggggggaatctttgaatcacagccttcagatgccagaagggcagagggaatcccacacgggctggtggatcatgtgtgtgcatttctctcccttctaatctgaggaaactaagcgtgaaagaatgtgagcatgcagaaaaggagaggcaggtatcagaggcagaggaaaatgggaaattggatatgaaagaaatacacacctacaagtgagttcagaaactgtaccccaccctcttgggaaacgcccattggagtgttgtttttaacctttgtacagtatttagacccagtaaatgcagaaatagaaacaaacggtcagaagacatatcgtgagagagagcgagagagagttcacaaaacagaaaacaaagtaccttaatatttaccagtgaccaaaagatgtgaagtagcaaaacgtctcctgaccccattgccagctagactgtgtggaaactcggttcataccagccattctaggggtggggtgagttgttgtcatccttaggaaagtgtgttgttgtaggatcaaccacatccttcaaaaggactatgcctgtttataagcccagctgtttctgccctgtgaaacacggtaaggatattaatacaaagagaatacagctttatgataaaagatgctcaatgaaggatgaattagggatgtactgagaatggggaaggaaactatcatctcagaagtcagcaggcagtaagcaagaggaggaatcaatacagcaacagtttggatcagactgtacagtttttttgtttttgtttttgtttttctgagatggagtctcgctgtgtcacccaggctggagtgcaatgacgtgatcttggctcactgcaacctccgcctcccaggttcaagtgattcccctgcctcagcctcccgagtagctgggattacaggtgcctgccaccacgcctggctaattttttgtatttttagtagagaaggggtttcaccatattagccacaatggtctcaatctcctgacctcgtgatccatccgccccgccctcccagagtgctgggattacaggcgtcagccaccgtgaccggctcagactgtactcttctagccatctgaaatacgttttctaggtagagatagattgtgtaagggtacagttgtgaggataacagaaacatggcagattatttaaaatcatcctgaaagtggtgctttatctgatgaaagtgattgtaatccataggaaaatgtttcaacgtgcgcaagagttgcggcggcgagcagaggactaccacaaatgcaaagtaaggagcttcctccctgcagttgcaggatagttcagtgctgatgcagatgatgccacggcccttagactctctcaacattcaatttctcatgtgttggctttttcagatccccccttctgcaagaaaggctctttgcaactgggtaagtttgcttgttttccttgcttttggacatagtctgccaggtcaggacatggatacatttttctccctacagctctgtgctcaagccctgcagagggagatggcagagagaaaggctgcctacaagcatcacagtcccatccctgttggtaaccgtgttgcgcaaaaacaccttcatccccacccagtggggcccctgatctaatattctaagtgtcagaggttccgtatttgtaatagcagatgggccctgactgtaaactagtgaagagtgaatgtaacttattacccacagggacaattccaaatgaaggccttaaatgatgctcagctaagctggttcttgtgtggcctctgtaccttcaaaagctgccgagtcctatgattacacgtgatgggacttgtacacttgaagtgaaacacagttttaaaacttgctttgtttagaattcccacctcatttttccatggacaaaagtattctttatgtcctagtgcacttacaatttggtattacctgggagtgaaaagaaatattacagccatgcctaagtgacttcttgaggtgagattgttctgtcagaaaaccctctcccagttcccctgcagctcttcaggaatccacatctctccagagctctttgttctcatgggtggcacctccagagtgaagaagatcctttgtcaagaagggaaacagaggggaaatgagagggtcctgcaggcagagctggaatcaacttccactctgcctcttgcaagctgtgtgaccctgggcacaatttctccttcctctggaaacctctgttttcttagatttggagcagggtggtcacactgaccttgcagagttctgagaatcagagacagaacataaaaggcctggaaaacattctccaaaaagaagctgcaacatgtgtggacagtgggcttttcatgcctctcttactgtctcttactgtctgttgacctggtgcaagaaacatgctctggtgatggctgtgagggaggaatgaggatagacatagacactcctgtgtctcaaacatgcttctttattactctgttatgactctgtcttccctggggcaggaccccagcctgcctacatttgcagacagacacagtggcatgtggagacaacagtgtgtcccaatgactttcctttaccctccagctgtcggcagtactcagtggaagggtgatattatgacactgatacttctattttgaaacctggaggatggaaaggtgcaaaaatctatcaccagcaacagaaggtgcagactgtgttggtggcggtaattttgtccatcaaatgaatatgtgtgaaaacattccctcctttggccctacaggtcagaatggcggcagcggagcatcgtcattcttcaggattgccctactggccctacctcacagctgaaactttaaaaaacaggatgggccaccagccacctcctccaactcaacaacattctataactgataactccctgagcctcaagacacctcccgagtgtctgctcactccccttccaccctcagcggatgataatctcaagacacctcccgagtgtgtgctcactccccttccaccctcagcggatgataatctcaagacacctcccgagtgtgtgctcactccccttccaccctcagcggatgataatctcaagacacctcctgagtgtctcctcactccccttccaccctcagcggatgataaactcaagacacctcccgagtgtctgctcactccccttccaccctcagctctaccctcagctccaccctcagcggatgataatctcaagacacgtgccgagtgtctgctccatccccttccaccctcagcggatgataatctcaagacaccttccgagcgtcagctcactccccttccaccctcagctccaccctcagcagatgataatatcaagacacctgccgagcgtctgcgggggccgcttccaccctcagcggatgataatctcaagacaccttccgagcgtcagctcactccccttccaccctcagctccaccctcagcagatgataatatcaagacacctgccgagcgtctgcgggggccgcttccaccctcagcggatgataatctcaagacaccttccgagcgtcagctcactccccttccaccctcagctccaccctcagcagatgataacatcaagacacctgccttccaccctcagcggatgataatctcaagacaccttccgagcgtcagctcactccccttccaccctcagctccaccctcagcagatgataatatcaagatacctgctgagcgtctgcggattccgcttccaccatcagccgatgataatctcaagacaccttccgagcgtcagctcactccccttccaccctcagctccaccctcagcagatgataatatcaagacacctgccgagcgtctgcgggggccgcttccaccctcagcggatgataatctcaagacaccttccgagcgtcagctcactccccttccaccctcagctccaccctcagcagatgataatatcaagacacctgccgagcgtctgcgggggccgcttccaccctcagcggatgataatctcaagacaccttccgagcgtcagctcactccccttccaccctcagctccaccctcagcagatgataatatcaagacacctgccgagcgtctgcgggggccgcttccaccctcagcggatgataatctcaagacaccttccgagcgtcagctcactgcccttccaccctcagctccaccctcagcagatgataatatcaagacacctgccgagcgtctgcgggggccgcttccaccctcagccgatgataatctcaagacacctcccttagctactcaggaggctgaggcagaaaaaccacgcaaacccaagaggcagagggcggctgagatggaaccacctcccgaacccaagaggcggagggtcggtgacgtggaaccgtcacgcaaacccaagaggcggagggccgctgacgtggaaccatcatcacccgaacccaagaggcggagggtcggtgatgtggaaccgtcacgcaaacccaagaggcggagggccgctgacgtggaaccatcatcacccgaacccaagaggcggagggtcggtgacgtggaaccgtcacgcaaacccaagaggcggagggccgctgacgtggaaccatcattacccgaacccaagaggcggaggttgagctgagaagaggccagtgcactcaagcctgagcaataagaataaaaccgagtagaacaaaataaaaaattcaaaaaacaaaacaaaacccacactccaaaaactaacaaagaataaataaataatataaaaataaaataaatactgcagtccttatgttattgctttgtttcgatatctggtatgattgcctgagggacctgaggtttttaatcataggggtttttttttaatctttagaagtggttggttatgtaaaatattattatttttttttttgagactggattttgctgtgtcacccaggctggagtgcagtggctcgatcacagctcactgcagcctcaacctcctgggcttcaagcaatcctcctgcctcagcctcccaagtagctgggatcacagatatgtgccaccacgcctggccaatgttaaaaaatcctttaacttttttgtagagatgcactcctggactcaagcgatcctcctacttgtcccgaccaccagcccctttctgataaacaaacatttacactgtttattatctgatgccatttctatcttcttccttgtcgtccagacatcgaataattaggtttcttcagggttttctttttcaagtgctcagtgttaaagatcactcacattagggccagacaccatggctcatgcctgtaatcccagcactttgggaggccgaggcgggcagagcacttgaggtggggagtttgagaccagcccggccaacttggggaaaccccacctctactgaaaaaaatacaaaaattagctgggcgtgatggtgcatgtctgtagtcctagccacttgggaggctgaggcatgagaatcgcttgaacccaggaggcagaggttgtagtgagccaagatcacgtcagcacactctagcctgggtgacagagtgagactctgactcaaaaaataaataaaataaatatcacttacatgagatatacccaaggggtggtctacagagacttggaagcagtggttattgcaacaggggcacggaagtcatctggctatgccagggtgcccaggggatactcggggtgggtggcatggtgctgctggggactcatcgcacaggacgctctgattgacgcactgccaggagtagcgctctgtcttggggctgcagccggcctcctcagctcgagtgtaacatcagtcgtggccatggcagcacctgcggatgtcacatgggcaggacagcaggtgggtgaagctctctcctggccctcctctcttgccaggaccatgggtgactgaagacccccagggaggcacagcatcctcttatctaagatttttttttttttaagagacagggtctttaaaaaagtcctgcagtctgcagtcgcccaggctggactgcagaggcacaatcatagctcacggcagccttgaactcctgggctcagcgatcctcccacttcagtgtcccaagtagctgagactacaggcacacgccagcatgtccggctggttttttagtttgtatttcctttgagacagcatatctctctgtcgctcaggctggggtgcaatggctcaatcagctcactttagccttgaactcccgggctcaagtgatactgccacctcaacttcccaagtatgctactacaggaacacaaactccttttttaaattttttgtggatatggggtctcactatgttgcctaggctggtcttgaactcccaggctcaagcagtcctacctcagcctccccaaatgctgggattacaggtgggagctactgtacgcctggccttatctaagctgtttccctgaaaatgcccgtcttgggtaatgattccattggccccaccatgccctgtcctgccttcctggctgtgcccaagcttggtccctgcctgcctgcctcactctctgggtctcgagctcctgtgacacatgactcctctctcttcctggagtgatccaagccctgccacttcctgactttgcccacactgtaccctctgcctggggcaacttcatgtctgcccattgtcccttaggcctcagcccaggcacaagcccctgcctccggaggtcatccaggcctcaccaggctacaccctctcgtaaaattggattccctcccttcagggcaggtttataatgaaatcctcctcagaggccaggtgcggtgacacccatctgtaatcccagcactttgggaggctgaggtgggaggatcacttgaggccagggggtcgagaccagcctgggcaacataagagagactcttgtctctcttgtctctataacaaatttaaaaattagctcaccaggccaggctcagtggctcatgcctgtaatcccaacactttgagaggccgaggcaggtggatcacgaggtcaggagttcgagagcagcctgaccaacacggcgaaaccctgtctctactaaacatacaaaattagccaggcatggtggcacgcacctgtaatcccagctactcgggaggctgaggtaggagaattgcttgaaccccggaggtggaggttgcggtgagccaagatcacgccattgcagtccagcctgagcaacagagcaagactctgtctcgagagaataaaaacacacaaaaaattaactcgccaggatggcacatgcctatagtcctaactacttgggaggctgaggtgggaggattcccttcagcccaggagtttgaggctgcagtgagccactgtgattgtgccactgcactctaacctgggcaaaagcgagaccccaggctagagtgcatgattttgggtcactgcaacctccacctcccaggttcaagtgattctcctgcctcagcctcttgagtagctgggactacaggcatgtgccaccacgtctgggtaatttttgtatttttagtagagacagggtttagtagagaccatggtgaaaccccatctctattaaacaaatctctactaaccccatctctacaaaaaacagctgggcgtggtagtgcacacctgtaattccagctacttgggaggctgaggcacgagaatcatttgcatcttggaggcagagtttgcagtgagctgagatcgcaccactgcactccagccgggatgacagagcaagaccctgtctcaaaaaaaaaaaaagggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgtggtggcgggcgcctgtagtcccagctactcgggaggctgaggcaggagaatggcgtgaacctgggaggcggagcttgcagtgagccgagatcgcgccactgcactccagcctgggcgacagagcgagactccgtctcaaaaaaaaaaaaaaaaaaaaaaaaagaaaaaagaacaaacaacagcaacaacaacaaaaaaacctctgtgtcaatcacagccttcgagctaggggagaggcggccgaattctgccctccgctaacgagctatagctttgtggaaatgggcgagtggcgtgcccttgtgagcctcagggccgcatctgtaaaatgggcataactgtcatgcctgtctttaagaacagccttgggggtaaatgagtggaactaatggaaagatctcagcccacaaccttccacagaacaggcgcttctcacacagtaagtagcaggagtgcagaggctgcaggcatgaatccagtcagactgcagactgcctgggttcaagtcccagctcccacgtcttggtaactaagtggcctcagacaagttacttagtatttcttcttcttctttttttttttttttcagacggagttttgctctgtcacccaggctggagtgcagtggtgtgatctcggctcactgcaacctccgcctcccgggttcaagcaattctcctgcctcagcttcctgagtagctggaattacaggcacctgccaccacatccagctaatttttgtatttttagtagagacagggtttcaccatattggccaggatggtctcgaactcctgacctcgtgatctgcctgcctcagcctcccaaagtactgggattataggcgtgagccaccgcacctggacacattacttaatatttctgtgccttggtttcttcatctgtgaaatgggattgttgtgagaatgcaaagggattcccagggcagttcctagtgcatagtctggctgcctttgtgtgtgtgtgtgtgtgtgtgcatgtgtgtgtgtgtttaatatagagacagggtctcactatgttgcctaggctggtttcaaactcctgggctccagtgatcctcctgcttccacccaaagtggtgggataacaggtgtgagtcaccacacctggtcactttatattatttttttcttttgagacagggtctcgcactgttgccgaggttggaatgcagtggtgcaatctcaactcactgcaaactccgcctcccgggttcaagtgtttctcctgcatcagcctcttgagtagctggtactatagtcaccgggctccttgcctggctaagttttgtatttttagtagagatgcggtttagtgattctcctgcatcagcctcttgagtagctggtactataatcacttagctccttgcccagctaatttttgtattgttagtagagatgcggtttccttttttttttttttttttgagatggagtttcgctcttgttgcctaggctggagtgcagtggtgctatctcggttcaccacagcctccgcctcctgggttcaagcgattctcctcctcagcctcccgagtagctgggattacaggcatgtgccaccgcacctggctaattttgtatttttagtagagacggggtttccccatgttggtcaggctagtctcgaactcctgatgtcaggtgatctgccttccttggcctcccaaagtgctgggattacaggcatgagccagcatgccaggctggcccttttttttttttttttaatcacttaacatatatcttggagaactttccattttgggagttaaagagatttgtttgtttgtttgttttgagacagggtctccctctgtggcccaggctggagctggaccttggctcagtccaacttccaccccccgggctcaagcaatcctcccacttcagccttccaagtagctgggtctatgggcacatgccaccacatcccgctactttttatagtttttgtagagataggattttaccatgttgcccaggctggtcttgaactcctgagctcaagtgatccacctgcttcagcctcccaaattactgggattacaggcatgagccatcttgcccagcctgttttttatttaatatctactaagtgccaactaccatagaggacataaagatgattcagtctctgcagaagtcattttctttctctttcctgttgtacagcacaaaattaatggactaaatagtctgtcactagataaagaagccctaagtaatcaggcacttgctgcagtttttacaaagtttaaaaagccatatgaaacacagtatactccaagtaataagaggcaaaatatgtgaagtgttactgctggggaatttacggactattcttttctacaatatatctgctgtggtctgaatgtgttccccaagattcatatgttaaaacttaaccaccagtctggtagtattaagaggtggggcatcattaagtcatgagggcatgggattagtaacctcataaaaaggttggcgggaacgagctaggccctttcattgcccttacatccctctgtcacttgaagacacagcactggtccccccgggaggacatagcagcaaggtgccgtattggaaatggccaccatgccctcaccagataccaacctgctggtaacttaatcttggccttcctagcctccagaactgtgagaaagaaatttctgggttttgtttgtttgtatgagacagggtctctgtcacctaggctggagtgcagtggcacgatctcggctcactgcaacctctgcctcctgggttcaagtgattctcccacctcagcctcccgagtagctgggattacaggtatgcaccaccacacccagctttttttttttttcatagcgttgtacagatagggtttcgtcatgttgcccaggctgatctcgaactcctaaggtcacacgatccacctgccctggcctcccagcatgctgggattaaaggcgtgagccactgtgcctggccaaaatttccgttttttataaataacccagtctctggtactttcttatagccgcacgaacagataaagactgtacctatctgttgactgggcgcagtggctcacgtctgtaatcccagcacttcgggaggcttagacaggtggatcacgaggtgaggagatcgagaccatcctggctaatatggtgaaaccccgtctctactaaaaatacaaaaaatttagctgggctcggtggcgggcgcctgtagtcccagctactcaggaggctgaggcacgagaatggcatgaacccgggaggcggagcttgcagtgagccgagatcatgccactgcagtccgtcctgggcgaaagagcgagactccgtctcaaaaaacaaacaaacaaacaaacaaacaaaaaagaccgtgcctttctgtctgtctccctcataggtcagtttccacctgattgtaaccacatcaagtatcctagtatatttcatatttacagaaaaataaatgggcaaatactgtcatttacagagaacctgccctgtcctgtacactgtgacatattttgtggtttgtgattatgtgctctgatccttacgatagctctaaaatagctcaaaaagttattcccattttgtacataagaaaattgaagttctggaaacataaggcaattgcccaaagtaatagagtaaatgacaaagctaggatttctttctttcttccattaattaattaattatttgagatagggtccctgttgtgggatgcagtggggagatcatagctcactgcagtctcgacctcctgggctcaactgatcctcacgtctcagcctcctgagtagccgggactacaggtgcacaccaacactatggctaattcttgtatttttctgtagaggtctgcctaggctaggcagagattctggctaggctggtctcaaactcctgggctcaagcaattctcctgcctcaccctcccatatagctgggaccgcaggtgtgtgccaccactcccagctgacttgtttataccagtggttcttaattggaagagttttgtaccccggggacatttggcaacatctggagatgtttttgaatgttacagtggggaggaggggaatgctactgtcacctactgcgtagaggcaagggatgctgctgaacatcccacaatgcacagaacagcctcctccccacccaacagagaagtatctggttggcccaaaatctcaatgatgccaagtctaagaaactgctttgtatttctctgagatactgggatgaggaacgctctaaattagttgtctttgaggataatgtatgcatacatacacatatatgtgtatacatgtatataactaaagatatatagttgagatcttgcatttgtgttaatgaatgtagtttttataaagataattgactcatacattaattgttgactctttggaaaggaaaaaggacaaatgagatacatgctattggttttgttatttagaaattgttcgtgtggatcctccctaccattcagccatttgcaaaccattagttgagcataacttaatcccttctagttcacagcaaagattcatcaaaagccatttggtatactgtcacctcatctctctaccagtgttgattagattacaggagggttacaggagggcaccttatccaaattggccaatgataaattcttttttggtaccttactatatatttgtatcctcagaacaaattctcctttctgcttaagctggcttgacttcatttctttcactagcaagctaagaggctttggccaacagaactactctaggggtagctgtaaaatttatctctaaggaaaggctgctcttttgaaaacacagtttaatgggtcttctgggtatatgacgatagtaaagcaaaattttgttctggaatgaagaagctgattacattgttgtattacaaacaatatattactcagtgaagtgaaaggccacactgtgggcagggtggtgactttgcagatagcatgttaatccttgcatgctttttgttgtttctctttttggagggggagggtcttaattttctttggccctcccccaccccctttattttctctggggaaggcaagactggataaggaggcttttatcctgccaagatgagttggcccacaggacaatttgactgaatagaggctccacaaagaacggacatggccaaagaattacaacaggaaattatgcatttagttttaaggcttcctttttctcctttttttttttttttttttgagaccgagtcttgctctgttgcccaggctggagtgcagtgttgcgatctcagctcactgcaacctctgcctcccgggtccaagcgattctccagcctcagcctcccgagtagctgggattacaggtacctgccaccacacctggctaattcgtatttttggtagagatggggtttcaccatgttggcccggctggtcttgaactcctggtctcaagtgatccacccgcctcagcctcccaaagtacagggattacaggcatgagccacagggcctggccagtatttttcttgtataatgctcagagtttcctttaaatctttttaaaaacttggcagccttagtcttagctgtgctttgggaaaataaggagctgcactcaagcttaagaagatattgctggaagtgtacattggcacagccttcgaagcttcttggtgccctcctgtaacccaatcgacactggctagagagatgaggtcatattgcatcaaatggcttccagtgattctttgctgtggacaagaaagttgctctttttctttgagaaagttgctctttttctttgagagggagttttgatgcctaggctggagtgcaatggcagtgatgtcggctcactgcaacctctgcctcccaggttcaagtgattctcctgcctcagcctcccgagtagctgggattacaggaatgtgccaccatgccaggctaattttgtatttttagtagagacagggtttttccatgttggtcaggctggtctcgaactcccgacctcaggtgatccacccatgttggcctccaaaagtgctgggattacaggcgtgagccaccacgcccagccaagaaggtcactctttatgctcaacaaatgtattgcaaatggctgaaaggtagggaggatccagatgaacaatttctaaataggaaaactaatagccagacacgatggctcacacctgtaatcccagcattttaggaggtcgaggcaggcagatcacctgatgtcaggcatttgagaccagcctgggcaacacggagaaaccccatgtctactaaaaatacaaaaattagccaggtgtggtggcgggcatgtgtagtcccagctactcaggaggctgaggcaggagaatcacttaaacccgggaggcagaggttgcagtgagctgagcttgtgccactgtactccagcctgggcaacagagagagactctgtctcaaaaaaaaaaaaaaaaaatcgtagatatccaatttgttcctttctcctttcctaaaagctttttgtgccccctccaacccaacagttccaccctagaaaaacacttgtacacatacatcaggaaacgatacaagaatattcaggctggtcgcggtggcttgcctataatctcagcattttgggaggccgaggtgggaggacggcgagagatcatgccactgcgctccagcctgggcaacagagggagactccgtctcaaaaaaaaaaaaaaaaaaaagcataatgttgaaaacaaacagcagtataatacagcatggagataactttataaaatgcaaagacaaataaaagtaaatatagtgcattgtttaggagtataaatatggtaattatgcaaagaaaactaaaatgacttttaaaaatcatgatggtgactatttttgggaggaaagcaggtagaaggtgggatctgagagcagcacacagtaagtgtcaattgtcctagttaatgttcggcttcttaaattgggtgatatgttcacaggtgttcattatgttcttttttttttttttttttttgagacggagtctgactctgttgcccaggctggggtgcagtggtgcgatctcggctcactgcaagctccacctcttgggttcatgccattctcctgcctcagcttcccaagtagctgggactacaggtgcccgccaccacacccggctaattttttttttttttgtatttttagtagagacggggtttcaccgtgttagccaggatggtctcgatctcctgacctcgtgatccacctgcctcggcctcccaaagtgctgggattacaggcgggagccgctgcgcccagcccattatgttcttataacttacattaacatattattttatatatgtcaagctttttttcccttttttttttttaagagatgagattttgctttgtcacccaggccagagagtgtcgtggtgcagtcatagctcacagcagcctccaactcctgggttcaagtgattctcccacctctgtctgccaaataattggaactacaggcatgtgccaccatgcctgggtaagtttttttttttaattttttatttattattatactttaagatttagggtacatgtgcacaatgtgcaggttagttacatatgtatacatgtgccatgctggtgcgctgcacccactaactcgtcatctagcattaggtatatctccgaatgctatccctcccccctccccccaccccacaacagtccccagagtgtgatgttccccttcctgtgtccatgtgttctcattgttcaattcccacctatgagtgagaatatgcagtgtttggttttttgttcttgcgatagtttactgagaatgatgatttccaatttcatccatgtccctacaaaggatgtgaactcatcattttttatggctgcatagtattccatggcatatatgtgccacattttcttaatccagtctatcattgttggacatttgggttggttccaagtctttgctattgtgaataatgccgcaataaacatatgtgtgcatgtgtctttaaaacagcatgatttatagtcctttgggtatcctgggtaagttttttaaaagtgttatttgtagagacggagtctggctgtgttgcctgggctggtctcaaactcctggcttcaaggtatcctcctgcctcagcctcccaaagctctggcattacaggtgtgagccactgcacccagactcagactttttttaagggaaagaatgggagtgtaggtggggagacacactttgggaggccaaggtgggaggatcacttgggccgggggttcaagaccagtctgggcaacaaagtgagacctcgtatttaccaaaaatacaaaaaattagctgggcttggtggtgtgggtttgggaggctgaggtgagaggattgcataagctgtaggagcccgaggctgcaacgagctgtgatcgcgccattgcgctctaacttgggctagacaatgagatcctgtctcaaaccaaaacaaaacaaaacagataattgtcaaattgctgttttgctattgttgctttttgtttttgcttcgctttgccttggaagtgaagaagagattctcatttaaacagttatcttgaagtatctttgtgaactagggtgcaattatttcctctgtccttgagacacagatgattcctgtccaacattcccaaggaactcagtaaggaccaaatagagactcaggaaagacagttactgattttacactgttgcaaaacagagctatggtttatgtttaacaaactgctggtggggcgtggtggctcatgtctgtaatcccagcactttgggatgccaaggtgggtggatcacttgaggtcaagagttcgagaccagcctggccaacatggtgaaaccctgtctctactaaaaatacaaaaattagcagggcatggtggtgcatgcctgtaatcctagctactggggagggtgaggcacaagaatcgcttgaacctgggaatcggaggatgcagtgagccgagatcacgacactgtactccatcctgggtgacggagcgagactgtctcaaaaaaaacacaaaaaacaaaaaaaccaaattgctgtattttattttgtgaaatagggtctagctctgttgtccaggctggagtgcaggggtgcaatcacagctcactgcagccttgacctccagggctcaatcgatcctccctcctcagttttcaagtagctgagactacaggtatgcaccaccatatgctgcccaggctggtcttgaactcctggagagagatacatacacacacacacacacacacacacacacacacacacacactttttttttttttttgagacacagtttcgctcgtcacccaggctggagtgcaatggcacaatcttggctcattgcaacctctgcctcctgggttcaagctattatcctgcctcggcctcccaagtagctgggattagtaaggcactgccaccatgcctggctaattttgtatttttagtagagacagggttttgtcatgttggccaggctggtctcaaacttctggcctcaggtgatccacttgcctcggcctcccaaagtgttgggataacaggcatgagccactgcgccgggcccatacatatgcattttaaaaaatttatttatttatttcgagacagggtctcactctgttgcccaagcaggagtgcagtggtgctatctcccaggctcaagcaatcctcagcctcccgagtagctgggactacaggtgtgtgccatcacacccagataatttttattatttttattttttaaattttttgtagagatggagtttcaccgtgtcacccaggctggatatttttgtatttttgataggcctgtacagtttccaaagttgcaacctttccccctccctgagagtaggggcagccccggctctccctctacatcctccacagtcccgaggttttggcctctgtttcctctgtttcctatgcttggaacaccagtcgctcttttgttggtctggctgacttctgttcctcttttaaaaatttaagtttggccgggtgcggtggctcacgcttgtaatcccagcactttgggagtccgaggcgggtggatgacctgaggtcatgagttcaagaccagcctggccaacacagtgcaaccccgtctccactaaaaatacgaaaattagccgggtgtggtggcatgcgcctgtaatcccagctacttgggaggctgaggcaagagaattgcttgaactggggaggcggaggttgcagtgagctgagatcacgccactgcactccacctgggcaacagggcaagactcggtctcaaaagaaaataaataaataaataaataaataaataaataaataaataaagtcaagggggtaacacctcttggtaactctcctgttgtttctcatgccagcatcatcacagccttgaggctctggggtaggtcacttcgtcgagctcgtttccatgaggataacgttatcttgggtgtctgtgagaatgctgcactgagtatagagcccaggctcctgggtcagccgggttcgaatcccctttcctccgtgaagatctgggtcagtcacaagtgcttcagtttcttcgatctgactgagggaggctttgactccaaaaaattaacacttgagtgtacctggccacagcttagcacatccagggtgtttccaccctttctttgggatcctcagggctggatggagccgatccttcccgtctctccttacactcgcgcactcacgctggctggaacaagtcctccaagtagaacgaagagcgcgttttagcggcgctctagcccgccgagagcatacgccctccccacacggggcccctgattgtctgaaggttgcgctggcacgcgcaacttccgggacagaggctgtggctggaaggagctgggcatccggcctgaggcgcagcggtcgcgttagttcggcccaatggcggcaccgctgcttcacacgttgtttgtcgggagatgcggccgcttcgtcctctgcagtcaagacgctgggcgcgtcgaggactgggtaagattcaggccgcttccttctgcgcgtctgggaccaaagctcaggaccgcgcttagaggagcggattgaaaggatgtgggacaaagctaatggcgtgtgataggagcacggggtcgagggtcatctcacgttcacagaaatgagctcattcctcctaactgggtaatagacatgggtggggcctggaaaagtgagtatgttctctgttctggaggcccactttcccgactgtgtctcttcgtgatttcccaggcctgggtactgccttctgcgccttgacccctcttccttccctcttcttcgtccaaatttggaagggatttccctgggctatgtgggttatcagccgaacgttgtcactcatggcaaattgaatattacatcttttttgtttgaaatttgtttcgacacacgtatttgtttcgcagtctttattttgctccacttttaaaatccctaacccccatagcactcttggcgtttaactttcagagtcattaggatgctatgttttttcattaatttactacgtgtaagtgaagcaaaccttgtaaaacaattagcgtaatatgattcctaatatttatcgagctcctgcttactgtgttaaacactggggacagtggtttatccaaagacactaatgtccctgctttctacagagcttacagcatagggtgggaaggcagtacacaggccaataaataaacgaacacgatgatttcagttatacaacaaggtaatgggggagggagaagggagagaaggagtgttcgagatttctcattgggaagacatctgtcatttcagcttctacttgaatgaagacaaaaatctaggccgggcgcggttgctcacgcctgtaatcccagcactttgggaggccgaggtgggcggatcacctgaagtcaggagttcgagaccagcctggccaacatgcgaaacctcgtctctactgaaaatacaaaaattagccaggcgtggtggcagggtgcctgtaatcccagctactcaggaggctgaggcaggagaattgcttgaacccggagatggaggttgcagttaagctgagatcacaccactgcactccagcctgggcagcagagcaagactccatctcaaaaaaaaaaaaaaatccagccacgtagagatttgggaaaagagtatttccaatagagtgaacagtaagtgaaatgagaaacagcttggcttgtttgaagagcagaaaagacatgatggctgtagtaaaacaagttgttggagatgaggtgagagaggtaggcgggggccagattaatgtaggattttaaagaccacactgagagatttggattgttactgtaagtgcagtgggaagacagttattggttgctgagcaaaggagggattgttggttaagagtggaagcagggagaccagtaaagaggccttaacaatagtcccactgaattatgttggttcaataaaggttggctattataatttttattattttcatgaacttaatagcttgttaatcttggttccacaggatttcaaatatgcgtgcattagagaatgattttttcaattctcccccaagaaaaactgttcggtttggtggaactgtgacagaagtcttgctgaagtacaaaaaggtaagaggagataatgtgtgaggtttgcttttggtcaggtcagaatacaactattgctgttatactaaagaccaatagaaatagcaagattaattaagataccagttgaaatcaaatatttaataatagcatgatgccgtcagtgcaaaattagagtaatagtgtcctttttttcccccaccttggcccatttcacaggtaataatgagagagtaataatgtctttactgaggtttcacctcttcaaatgctttatttacaaagcatcttttaactttagcaagtgctagaattaaaaacaattacagcattttatttatttatttatttgtgctggagtcttcctctgtcacccaggctggagtgcagtggcgtgacctcagctcactgcatcctccacctcccaggttcaagcaattctcctgcctcagtcttctgaatagctggggttacaggcacgcaccaccacacctggctaattttttaatttttttaatagagacggggtttcaccatgttggcctggctggtctcaaactcctgagcttgtgatccacccgccttggcctcccaaagtgctgggattacagatgtgagccaccatgcctggcctacagcattttattttttgaggaacttacctaagcattactttgggacagtaaaccggttctctgaatagggatttttgtttttgtggtagtttagaagcatttctactatatctcagcagtagagggaaaatgttaagtaaccgtatgtttatatgaaatatccatttgtatccatatttgagtgaatacttttttagatcctcctgaattagatcattagagctggctgttttttcccctcatgctttttgagaattcgcaggagtatcaactattatattcaaatgtcaatacagaagtatagctaaatgtagtttatcattttcctttttccaagccctctggctgcactaacatgagtgtttaaatttttgtagtcatgattttataatccgcaattgacatgtgaaagttagtgttccttttataatttcatctgatgttaaagtacggttaaaagtcttgctgttgatactaaacaggaaacaaaagcataacttaattctttccccttcttgttaagggtgaaacaaatgactttgagttgttgaagaaccagctgttagatccagacataaaggtaattaattttgtgtttgatcattagcaaaattattgccactttatacagacatagtttgctctttgggtcccattctgttctgcagaacttgctctctccatggtcctcccttactttaatctggtggttctcaaccagggacagttttaccccctagaagacatttggtgatggctgcagatatttttgtcacaactgggaggaaagggtgctactggcatctagtgggtgaatgacagagatgctgctaaacatctcacagtgcacagggcagcctcccataaccaagagtgatccagccccaaatgacaacagtgttgaggctgggaaaccctgctctaatgcttcctttctattagattactacctctttcctccatgctgcatgcaactcttttgtctctttaaagctaaaacaaaccaaaaaaaaaaaacaaaccactgtttcagcatttccaggttcgagatacacctatcatgtagtaaaaccttaatacattttgtttcaccattcttcctttactgcccagttttgaagagaatggtttattaccatggcagtggtagttagattgcctggaatgaaattccaattttattatccagtgtgtgatcttgagcaaattgttttaacctctctgcctctattttccactgtgtgaaaccaagaaaacaatagagatttaaaaaatatggagtgttttgttttttaagagatgtggtcttgctgtgttgctctagctattcacaggtgtgatcatagtgcactacagccttgaactcctggcctcaaatgatcctttctcttcagccttctaaaaagctgggactataggtgcatgccactgtgcctggctttaaacatggaaatacttaacaaggattcaatgagctaatatgcaagaagcacttagaacagtctctgactcaaagtaagggcagtaattgtcatctgttgtttttgttccagctgactgtgctgtatcatttctcactcacatttaagtccactgttcttatcactgtagtaattaccctgacagattacccatgttttttttttacatgctgatttcagtggactttttttgagacaaagtctccttcttgtcacccaggctggagtgcagtggtgtgatatgggctccctgcaacctttgcctcctgggttcaagcaattctcctgcctcagcctcccaaatagctgagattacaggcacccgccaccatgcctggttaatttttttatttttagtagaaacggggtttcaccatgttggccaggctggtcttgaactcctgacctcaggtgacctgcctgcctcggcctcccaaagtgctgggattacaagtgtgagccactgagcccagcctcagtggacttacttttttaagccttgtattccttgtatcagccgacactgttggccacccacttcttaaaacttcagtgtttctgatcctcctgtcttctgatcctttaatctctctctttttttttttttttttttttttgctctgtcgcccaggctggagtgcaatgacgcaatcttggctcactgcaagctccacctcccgagttcaagtgattctcctacctcagcctcccgagtagctgggactacacgcgcccgccaccacccccagctaattttttgtatttttagtagagatggggtttcaccatgttagccaggatggtatcgatcttctgacctcgtgatccacccgccttggactcccaaagtgctaggattagaggtgtgagccaccacacccggccagtgatcctttaagctctagtatctctcgataggttcttgatcttaaatttggtgttgattgggcttcaaaacttgactcttttctcactctgttgattcttctgtgtgatctcctcatctcccttcatggctttgaaatctacctgtgtcctaatatatttgtgtctgtagccaagattgctcttgtgggctccagacttatttcattttcatttttggggatgggcagaacagagtcttgctctgtcacctaggctgtagtgtagtgggatgatcttggctcactgcaacctctgcctcctgggttcaagccatcctcccacctcagcctcctgagtagctgtgccaccacgcccagctaatttttttgtattttcagtagatttggggtttcaccatgctggccaggctggtctcgaactcctgacctcaagtgatccacccgcctcagcctcccaaagtgctgggattatagacgtgagccactgcacccggcctagacttgtttcttaactgtctgttagatgcatttacccagaatcatcatagatgctccaaacttatcatgtccactcttggctgggctccatctttcatggagctttccctggttctctctaagcacatggttgttccttcattgagtctatttcccacacttccagatctctctagttacagatctggtttacaaggccctccatggtctatttggtgcttctttgttccccagatttattatctgttggcttggtcactatacatgccagccatactgaacatttttcagttttctgaaaacatacttttccttctgtaagaagcagaacttccagaaaagactcaactgtgttactgtttaaagacagctgaagcatcactttctctttaaagcttttcctgacccctgcctcctttcccagatacaaagggacattttctttgtgttccactgtattttgtatcagcagttctcattcttggtattttgacataccaagaattgcactagttgtgtggagtgttgcaagtagaacttttttcgtcttgagacagggtctcgctgtgtcacccaggctggagtgtaatgggcccgatcatggctcactgcagcctcaacctcccaggctcaagcaatcttcccacctcaggctcccgagcacctgggaccacaagcatgtgctaccatgcctggctaatttttctagagacgaggtctccctatgttgcccaggctggtctcaaattcctgggctcaagcagtttcctcctgccttggcctccgaaaagtgctgggattacaggcatgagccactgttcctgcctgctagtagaaataataatagttcagtactaaagcatcaaagtctgcaactgatttacttttttttttttctttttgagacatagttttgctcttgttgcccaggctggagtgcagtggcatgatcttggctcactgcaacctctgcctccgagttttttaagtaattctcctgcctcagcctcccgagtagctgggattatgggcatgcaccaccacgcccagctaattttgtatttttagtagagacggggtttctccatgttggttaggctcgtctcgaactcccaacctcaggtgatccacccacctcggcctcccaaagtgttgggattacaggtgtgagccaccacccccagcctgatttacttttaaaaatggtacagtttaaatgttatccttatagttttgttgcagtctttttagtggaaaagagataggatagattattttatttacacactaacttagcttgttttctacatgcctttggccttagtgagctaccgttaatgttatccttaacagttttggacatatgaaattaccatagtacaaatgagttgtggttttactttattttactgccaggctacttgggatttcatcagaaaatggttgatctgtgggagtttgacacatggatatggcatagtaagcactcagtagctgaattaaggtggggaaaaggggacagcttcttctccgcatatagggaggcatgtgggatggtggacagaggatagccttgaccgagacagatgggtttggacctgcttctttactggcctcttggttgggcagattgcttattaatcgttcttagcctcagcttcctgaacagcaaaatgggaataactaaacatcttgcagagttcttaggattagaagaagatatatatgcggagtgtcaggcaccatgcctggcatatggtgtattctcactaaatgataactccatatgaatatccctgtaggtatgatgaccttgtgttgcttttatttatatgtctaagccttccacaaattaggggctttttcttaatggtttttgtcctgtgcagtatatatgcatgaatataattaatatagtaatattttacataattgacactgtattttataccttgcgtttcaaatttagcagttcttctcatgtcactaacaattactatgaacagtaatttgattgcctgaaaaatatttcatggaggaatggggctatcatttatacagaacaaacacatcataatatatttaaactcagccacagatttggtttagaaaagttatgtttattcatgaccccaattgatcagcctagactgaattttatcagcatgcttcctggtcagcttgaatatagaggaaatagaggtagctattgttcctttgtgatcttctaatatttcaatctgctagaattctgcagtttttaaaagtcccaggtgtcaacatttgaggtgatttcactttttcagggcaaacaaaagtgatcaggctgaagtattgcatttaagtctttctcctgtgtattagagttactagattactttcttaaaacgattaagtttattgtgacatctttttctgttttaatacccagttttgatttccttccaagtttgtgaccttttccccccaacctattcttgataaatgattgatataagatagctgtaaatttctgttatcttagaggatttgtgattttgaaagtactctttgtttaacttaaggatgaccagatcatcaactggctgctagaattccgttcttctgtcatgtacttgacaaaagactttgagcaacttatcagtattatattggtaagttcaccatttattttactgtcaagtatgtaattcagaactttggtaatagtatatgttatattaataacatgctgcttttatctttcttcccccactctagagattgccttggttgaatagaagtcaaacagtagtggaagagtatttggcttttcttggtaatcttgtatcagcatagactgttttcctcagaccgtgtctcagcatgattgcttcccattttgtgcctcgtaagtcattactctttgcttgcttggaattttcttttcttttctttttaatacttctttgttaaaataccaccttccccttatatatgagagactgctaccatggaagattccagatgcatattggcaccaggtctggtagacatatattccccgtaatgacccctatggaggtgtctagattcatttgttgctgtgagtttgatgaattataacttgctttattgaactcctggtgaaatctaggaatttttagccatttaaaaactataaagttgcattactttttttcagattgtgcatttaattaatcattgggctaactttggattatggaaaaataactttttttatagctgttcattgtctaggtcaataactttttttgtatagccattcattgtctagatcaatgacagaacaacatattttctttttccctcaaaagcccgagtgatcattaaggaaggcgatgtagatgtttcagattctgatgatgaagatgatagtaagtataaaaaggtttaaagcctgggcacagtagcttacacccataatcccagcattttgggaggccaagatgggaggatcacttgaggccaagagtttgagaccagcctgggcaacatagtgagaccttgtctctgcaaaaaaacattttttttcaaatattttcttaaaaaaggcttaaagtagaactaggcagggtagtgtgtgtctttagtcacagctacctgggaggcttaagtgggtggattgcttgagcccaggagttcaagctctgcctggtggcaagactctgtcttctttaaaaaaaaaaaagtaaagcacagaatacctggcatctattctaataagtagactgcaacaaatgacaacttttgatgtaatctttttgttatatttaccattgatatgcagtcagttgtcctgaatgcattatttatataattagtccatttaattttcattgatgctggtggagaaaagtcttgaaattattatttctctgataaattattccgttttggttagcatgtgtttttagcttcaagtatgtcactttttgtttgtttgtttgttttttgagacagagtctcgctctgttgcccaggctggagtacagtggtgtgatcttggttcgctgcagccttcacctcctaggttcaagtgattctcctgcttcagcctcctgagtagatgggactacaggcgtttgccaccatgcccggctaatttttgtatttttagtagagatggggtttcaccatattggtcaggctggtctcgaactcctgacctcaggtgatccacccacctcggcctcccagagtgctgggattacaggcgtgagccactgtgcccggccagcatttatttttagtttcaagcatgtcgcccttcagttttgttttgatgctcatactctgaacttttcttctttcagatcttcctgcaaattttgacacatatcacagagccttgcaaataatagcaagatatgtaccatcatgagtatacttttccttattttgaatgtttaattctcaagaaaattgtaatcaattagtaaaaattataaaatgttaatagtattaaagcttgagtcttacattgcattttttttttttgtatccacttgaggaaacattacattctacaaaaagtggcatttccattttctatttattctctttaattgtttttcaaagttcgtatgcagattctcccccaattttgtatggtggttggaattttgtttttatcttcaacagatatgctatccaaaatttttcagtgagaaacccctgggtgtgtttgtgtcatgccatatgaataaaaattgcacttctaagaaaagcttttcaggtttgtgggtttcttttggaggggtggatttctagttccctctgtctgttgattatttgttaacttaaaaaaatccaacttgattattttttcttcttttaaaaataatatacatgtgtagtgggaaatgtcagcaaaagtgctgttatgtttctgtgggagagaagctccctctttgatttgctgttgatatcagagttaacagaagcttattttctctaagttgttatagactttctcagaagctatacattgtaagttccagttctggccgggcgcggtggctcacacctgtaatcccagcactttgggaggctgaggcgggcggatcacctgaggtcgggagttcgagactagcctgaccaacatggagaaaccccgtctctactaaaaatacaaaattaggtgggcgtggtggcgcatgcctgtaatcccagctgtttggggggctgaggcaggagaatcgcttgaacccgggaggcagaggttacagtgagccgagattgtaccactgcactccagcctgggcaacaagagcgaaactccgtctcaaaaaacaaaaaaagttccagttctttgaggtaagggttcctgtttgcctcctatgtctatcgatatttgcttttagaatggtagttttcctttttattccttttctagaaagtaaagtcaacatggattgatttaattttttaaaaatagggcaccgtggtttctcatgccaatactggtggaaaaatttccatttgttcgaaaatcagagagaacactggtaagaaatcttttcattgagaacatcatggaaaagttgtttgtatgatttcattttagatgatattaggtctttttctttctttttctgtctttatttttatttttcttttttgagaccgagtctcactctatcgcccaagctggagtgcaatggcgtcatcttggctcactgcaacctctgcctctcgggttcaagcaattctcctgcctcagcctccccattagctgggactgcaggcacctaccaccatgcccagctaatttttgtatttttagtagagacaaggtttcaccatattggccacactgttatcgaacgcctgaccttgtgatctgcctgcctcggcctcccaaagtgctgggattggtgaaccaccgtgcccggctgatgttaggtctttttcttaaaggttactttgtcttctagactttaaactgatgtctaagaatttgactcagattcctttcttataaagcggctattggggattcgcagtgcctttttctgttattactatgtgcaagtcaaggtctgagttcatttccggaatatctgtagtggctttatgctcatacgggcaagaattactagaagataatagttcatgtattactaattgtgaacatgccttattttaacctgaaaacaaagccttccatagaagaattctgcttaagtttttgtacaatgttcagatcatctgtgcagtttttaataattaatagtggttgccttagtagaaaaccgaatctagtagcatacaaaaagaattacgtaccatgaccaagtgcgactgatgttaggaatgcaagattgattttttttttcgggggtggggggacagtctctgtctgtcacccaggctggagtgcagtggcaccatctcagctcactgcagcctctgcctccagggttcaagtgactctcccacctcagcttcccaagtaggtgggactatagacatggggcaccacaccccactaatttttgtgtttttggtagagatggggttttgccatgttggccagactggtcttgaactcctgacctcaagcgatctacccgtctccacctcgcaaagtgttgggattagaggcgtgaaccaccgtgaccggctgagattgagttagtacctgaaaatgaattaataaaatattttgtagcaatagaacaaaggacaaaaaccacataatcatctcagtagatgcagaagtgtgtgacaaacaccaatatccctttatgagaaaaacagaaggaaattttctcaacctgataaagggcatctgaaaaacccacagctaacatcatattcagtggtgaaagaccaaaagttttttcctaagacaaagaacaaaacaaggatttccgctcttgctgcttgtctagccaaggcagttaggcaagaaaaagaattaaaagcatccagatggaaaggaaggcgtaaactctcttttgcatggtgattttatatgtcattctaagaagtttacacacacacaagaaattttagagataataaatgagttcagcatggttacgggacagaagactaacatacactaaccagttgttcaagacaattgaataggggagaatagtcatttcaacaaatgctgctggcagaagtggatatgaacatgcaaaagagtgaagcatatggatatccatatacaaaaatgaactcaataaaagccctacatgaagtgtaaaaactgtaaaactctgagaagaaaacgagtacattttcataatgttggattaggcagtaatttccagatttgatgcctaagcacaagcaaccaaagaaaaaatgcatcaattgtacttcaaaattaaacgttgttatgcttcataggacatcttcaagaagatgaaaagaatccccaaataatgggaggaaatatttctaaattttatgtctggtaatggacttgtatatgtaaagaactcttataattgaataataaaagggcaaatagcccaactgaagagggcaaaggatctgaataggcatttctgcaaaacacatgaaaagaagctcaacatcattagccatcagggaaatgatttcacttaatgcccacaaggatggctataatcagaacgagaagacagtaacaagtgttcacaaggatatggagaaatgggaacgttggaactgtcatatgttgctgtgagaatgtaaaatggtgcagccgttttggaaaatagcctggcatttcttcaaggttaaatgtagaattaacacgtgactcagcagttccatttctgggtttatacccaagagaaatgaaaatatatgtccacagaaaaacttgtacatggatgttcatagcagcagcatccataatagcctcaagtagaagcaactcaaatgtctgtcaactgatgaacagatgacaaaacatggtacaatggaatattactcagcaatgaaaaggaatgctttatatgttacaacatgattggaccctaaaaacatgccaaaaggctgtgtattatatgactccattgataggaaaggaatggtttacatgttacaacatgattgaaccttaaaaacatgccacaaactgtgtatgactccattgatatgagaggaatggtttacatgttacaacatgattgaaccctaaaaacatgtattatatgactccatttatatgaaatgtctcaaagaggcagattcatagaaagactagtggttgccaaggtcttcattttttaggggtgcactaatggatgtaggatttctttttagagtgattaaaatgttacaaaattgctggctgggtgcagtggcttatgcccataatcacagcacttcgggaggctgaagtgggaagatccaggagttgaagaccagcctgggcaacatagtgagaaaatgtctccctaaaaggaagaattaacctcatgtggtggtgtgcacctgtagttctagctactagggaggctgaggaggaaggattgcttatcccgggaattcaaggttgcagtgagctatgattgcacccactgtacctcatcctgagagagagagcaagaccctgtctctaaaagaaaaataaatgttctgaaattgattatgttgacggtcacataactgaatatattaaaaacttaaattgtatactttaagttggtgattgtatgatatatgagttttatcaatacagctacttaaaaacctatagttatgcaaattaaaaatttcatttactggggataattgaaatgattataccgaacataatacatgtagaaacagtatagtttttgtattgctggatagtctgtttttttctttttcaatatttgaaactaaaggtcatgtaattgatgtttttcttacataactgtgaaatatttattctctgttgaaatgttttatcttacgttttctcctttaggaatgttacgttcataacttactaaggattagtgtatattttccaaccttgaggcatgaaattctggagcttattattgaaaaactactcaagctggatgtaagtattgagtaatctatttttattttcttttttttattttttatttttttattttcatttactgacttgaatttgttataatcacagtatgtggaaacaatagtcagtgatagaaaagaatccacttggccaggcatggtggctcatgcctttattcccagcactttgggaggccgaggcaggcagatcacctgaggtcaggagttcaagaccagcctggccaacatggcgaaaccccgtctctataaaaaaaaaaaaaaaaaattagccaggcatgatggtgggtgcctgtaatcccagctactcaggaggctgaggtgggagaattgcttgaatccgggaggcagatcttgcagtgagctgagatcgtgccactgcactccagcctgggcgacagagcgagactccatctcaaaaaaaaaaaaagaaaagaaaagaaaagaaaccactagcaccattctttgcttcctttctttgaatgtgtcttgaactccatctgtgcatgtgctgggagttgtagacagttccttctcatgattggagaacaaggcgttaaatacatagttatccaaatgtaaaagtatggttgtggaaaatgccatgaatgaaacatacattatgagttagagaacctgatagaatcacagtggggtcaggaagggattcctacggaagtgatttttcctgtttggcctttcttaagggcagattataattataaacagttaaaactttgtttaaggaggcccgcactaaggtgcagtgggaatgaaaggaagtggtagattctagtgacattgtgaggaaaggtgaactggtccttgagactggtttggaggaggggaggcagacagtaagggaaaggaatccttcaatagttgctccctgtggaatcgaatcttggtgttgccattaatggtagttagaaatatgaagaggaggctgggtgtggtggctcacgcatgtaatcccagcactttgggaggccgaggcgggcggatcacgaggtcaggagatcgagaccatcctggctaacatggtgaaactctgtctcactaaaaatataaaaaattggccgggtatggtggtgggcacctatagtcccagctactcgggaggctgaggcaggagaatggtgtgaacctgggaggtggagcttgcagtgagcccagattgtgccactctgctccagcctgggtgacagagcaagactctgtctcaaaaaaaaagaaaaaaaaaatatgaagaggaggcagttggaagagtagttccatcttggccaggttcagttgctggtgggcagcctaccagagaatactcacaggcagtcgtggctgcagatggggacctgagcataaacctttggaaagatgcagtttaggacaggggaggagaagggtgatcagaagtatggggaaaaccaagagtctggatgctcaggaagaatccgctggaaggaggagtttggtcagcagcatcagatactgctgtcattttttagaaagatgaaaagagcaacagtccttggatttagtggttagaaggtagtctttgttgctttctggaggaccatgtcagtgaagacgcagaaactgcatttcgggagaggatgtggatggtggggaagcagaattggggctgttagagaccttggtgcagggttgtggtggaaggaggggatggagcagggctaagaggcgtggtttaggagtggggagatgtgagcaggtttgtggactgaggggagaggagctttggtggaggaaaacattgatgctataggaaagcaggaagatggaacaaggtcttagaagagctggagcttgggctcactggtgcagtgctcccttggagttgcacctctctggccaactgtatatgtactctttatagtctttctctggtatatacttaaggaacattttagaatgtttacaaagaaggtcaggcatagttaataaaaaatggcatggtttgagtggtatgttaagatatttgaatggtgatataccaaaataaatattgcatcatgcacatttggcttgcagttcatcatttttctgctcaattgattgacgatatgtttattacacaatgtgtctgtgagtgtcttgtgcatagagattgtattagtccattttcacactgctgataaagacatagctgagcctgggaagaaaaagagatgtttttgtttgtttgtttgagatggtgtctcgctctgttgcccaggctggagtgcagtggtgcgatctcggctcactgcaacctccacctcccgggttcaagcagttctcctgcctcagcctcctgattagctgggattacaggcacgtgccaccatgcccggctaattttttgtatttttagtagagatggggtttcactgtgttagccaggatggtctcaatctcctgacctcatgatccatccacctcggcctcccaaagtgctgggattacaggcgtgagccactgcacctggccaaaaaagaggtttaattggacttacagttccacatggctggggagccctcagaatcatggcgggaggtgaaaggcacttcttacatggtggcggcaagagaaaatgaggaagatgtaaaagtggaaacccctgataaaaccatcagatctcgtgagacttattcactatcatgagaacagtatgggggaaacctaccctatgattcaaattatctcccaccagtcccccccccaacaacatgtgggacttacaggagtacaattcaagatgagatttggggccaggcgtggtggctcatgcctgtaattccagcactttgggaagctgaggccggtggatcacctgaggtcaggagttcgagaccagcctgactaacatggagtaaccccatctctactaaaaatacaaaattagctgggcacagtggcacatgcctgtaatcccagctactcaggaggctgaggcaggagaatctcttgaacctggggggcggagtttgcggtgagccaagatcttgccattgtattccagcctgggcaacaagagcaaaactctgcctcaaaaaaaaaaaaaaaaaaaagtgagatttgggtggggacacagagccagaccatatcagagctagaataaatgttgaatttgttgaggctacctgacatagagcatcatgtgatagttgtcgattttatataagtatgtagtaaaaggggcttggtttattatatttaaattccttcatgacctaggtcagtttacaggcttgcaccataattgtgtattgtgttggggtgtgatataaggcactaatctggacaccttgaacgtgtgtatatcagatgaatttccatcccaaaataacatagttgtattttttaaatccttttattcttttttcccccccctttgttataggtgaatgcatcccggcagggtattgaagatgctgaagaaacagcaaatcaaacttgtggtgggacagattccacggaaggattgtttaatatggttagcagtttattaatgaaagtggagatgaagtttatcataatcaaagggtggaaacagctagtgctgctcatctttgttaaggctttagattgaaagaattaaaatagtttagcaaacttgaaaacgattccttatatgagtaatttgctgccatgtcatttagcacttagcataattggtctatttccaaggctttgaatttgggtttggtgaagtatgtttcacttttgttgttgtaactttcagtgtttgtttttgtaagccagatgctgtctgtgagggcgtggctaatggaaaagcataactgtttaatttctgcattttaccacttgtacactttatagcattacttcttacgagtagctgggaccacagattaccatgcctggctaatttttgtgtttttagaagaggcggggtttcaccatgttggccagactggttgtgaactcctgaccgcaagtgatctgcccgcctcctgccccgcaaagcgttgggattacaggtgtgaacaactgtgcctggcccacgttcccttctcagtacacttggagagaaaacagattgctgcctgccagcccagctaggtgctctgaaaatgtcatcctgcctttggtcactaggtggtggtcttcccttaagcctttctctattaaaatctcatatggggtaattaacagtatttcctttattctttccaagggttgagttgtaactagcccaaaccaacttattaatctagaattttaaaaactttaggctttgacttttcttcttcttcttctttttttttttggtgggggaaagaatatagaaggcttttccttctctgcaacgattttgtggcttcctagaggtcaggagagtgttggtcatgggaaagaaggttgaattcagtctgcccacatgggcgtgcctagctttagaacagcgctatttaggagaagttggaagttacaccctttggtgagaagctgtgtctgtttttttccatgattggcataattaactcaaataccagctgtacattagtccgtatttctgttcatggttgagttcagtgtgtccagagaccggaaggtgctttgcactcacaggagtgcccatgtggagctccatgggatgtgaattattgttggtcactagttctggctgacattggaatcacttgaagagtttttataatatgtggattccaaagccctgtcacaaacctattgaatttgtacctcccaggttgaattttttgttgttttttgtttgtttgttttttgagatggagtctcactctgtcacccaggctggagtgtagtggcatgatctcagctcactgcaacctctgcctcctgggttcaagtgattctcctgcctcaacctcccaagtagctgggattacaggcacctgccaccacgcctggctaatttttgtatttttagtagagagagggtttctccatgttggccagtctggtctcgagctcctgaccttgggtgatccacccgcctcggcctcccaaagtgctgggattacatttacaggcgtgagccactgtgcctggccctgggttgattttgatgcttagctaggtttgggatccattggattatttaacacccgaggtgccttttgtttctaatgatattctctcaacgtgttttaaaaatgaagcccatgagatagttatgagatagtagaacttttccctacattggtgaagtaaaaatcttgggattttgatagtcagattatcttaggcattaaaaaatatcacaccgatgccctctctttttatagggattcgcagaggcatttttggaacatctttggaaaaacttgcaggatccaagtaatcctgccatcatcaggcaggctgctggaaattatattggaagctttttggcaagagctaaatttatttctcttatgtaagtagcctaatttgccgagtactttttaatatcatgctttaaaaagagtatagcattgtctcaagtcagaaatatctcccatatgtttttggcatgtttttaaagtgaataaaattcctactctgtgcaagatgtttatatttctaagtggtgattttagaataaagtgtctccttttttattataaaaccctgtatgtaaggcttttgtcatctcttttgtgtggttgcacttaaagatccatttgttttgtggatagaggacagtgttgtatactgttttgattctttttgtaggtttgtcatttttttcatttgcattccaaatctattgtatctgttaaagctgaagaaaaacccttttaaaggtaatagacctatctaggaggccagtttctttcagtggcccatgaagatatctttggacaaggatgctgttgaaacccttccccaagaacaaaattattcacccataggacttgactggatgcatcagggaatactgaagtccaccagactgtctttctcttgagacgtgttggtgaacatgtcctatttggccaatcaccctaagaggggtgcctttgagatggttaggagaacctgctttccatcccttgggacgttcttaggggctcacctgttcctagaaggtcagagctactctgccttgtaattggaaggttgtcttcctacgcacccatccttatccttcctttctttgcttttcctctgtacccatgggtattatttaaagaaacctatgaacttacttagcatggtttgtaatgaaaggcagttgtgtgtttttatgttattctggtttttttatgaagtgtaaagttgacttgaatttttcttttctctagtactgtaaaaccatgcctagatcttttggttaactggctgcacatataccttaataaccaggattcgggaacaaaggcattctgcgatgttgctctccatggaccattttactcagcctgccaagctgtgttctacacctttgtttttagacacaagcagcttttgagcggaaacctgaaagaaggtcagtgttgtgggagtgctggactggattttccttgtgttcttgtcacccttcagaatggtgattcattacttttttgagatttttataaaaactggattcagaaaactgcatgtacactcaaacttctaataataatttcaagcagctcataggcccctacaaaccccttaagatagatttgagcttgagaaccctacaaaccccttaagatggatttgaggttaagaaagaggtttctgcctttgaaggtttgaaatgtgaagatgtctccagaggtgaggctgagccctgggctgtgccagcgccctgtacaaagcttcagttggatgcaccttctctttgttgtccttgtaacagcccagtaaatggcaggtattctccctttacagacagcaccaaagcacggggaagtcattttcccaagatcacatggttactggcaggattagaaaactgaagccaggttcagctgaccctaaagtttgagtttatatagattacactctgcctgaagccttgagacttaattgaccagtattgttttgctaatttctaagagttacttataattcaaatctatcagttgaaacttactagattagcgtattttagttgaagagagtctccaagaacagtgtttataagtcattgtaaattgttctgtttatgtttatgaataattcatatggttttgtgggtcacttcctctaaaccagggtctgtcaaccctgcatgattgccatttgggctgcatcatccttcatcgtcgggggctgtcctctgcactgtaggatgtttaacagcctccacctactagctgccaacagcagtccctgaccacccccagctgtgacaactaaaagtgtctccagacactgccaggtgtcctctgtgggggttgcagtctccttaggttaacagccacagctctaaactgacagttgtacgtgttgcattatatatgtttacctacatcctacatgcttctaaaagatgttgtatgaactagtaggatgaggttttatcacaaggtaagtaaatacaagctctgcttttctttgtataaattaatgccaggaatctgaattaaatatcttgtttttgtaagcagtgacatcccatttaggtaatttttattgaaatatgcatcaaagaaactcctaagaaaatatacttaagtacaagttggtcagcttgcctcttaaaataaatgtgatgtctttattttactcatgtaggaaagaattgtattcactaagtctaagaaagtggcttctgtctaaatttgccgtccgttgaggtagaaggcaaatttggagttttcttgtttagaaaaaaaactacagatgactactgtgcacctgaaaacagcactcagcttcactaacgagacatgcaagctagaatcaaattgctgttttgttttgttgcctgtcgtgattgttagctgaaaccaaatcacaaggtctttttctccctctgtattagctcagcatacactgagcttacaaacgtatgaacttcacgttgtcgtggaatcttacagcctgctacttcctaagtttcctttagagaagctgccttggtgaccaatgaatgtggttagcctagtgatactcttctgggccatatactgtgtgactatctgcatggacctttattgaaagcatttctgcaaataatttttttaagttttttttaaatgtgtgataatttgtgcttttaaagatatcttacacttttcacttatttgtacctttaaaaatcttttttttttttaaaccaaaggtttgcagtatcctcagagtctgaattttgagcggatagtgatgagccagctaaatcccctgaagatttgcctgccctcagtggttaacttttttgctgcaatcacaaagtaagttatttatgctttcttgatgggagttatttaaaatatttttatttatgtttctctagtattgtaagagtctgttaaatttctatgaaattagtaacattataaaaggccaggcgtggtggctgacgcctgtaatctcaacattttgggaggctgaggtgggaggattgcttgaggccaggagttaaaagaccagcttgagcaacatagtgagaccctatctctataaaaaaattttaaaaattagctgggtgtggttgcctgtgcctgtagtcccaactactcaggaggctgaggtaggagaatcacttgaacccaggaggcagaggttgcagtgagctgagatcgtgccattgcactccagcctgggtgacaagggcaagactccatcaaaaaaaaaaaaaaaaaaaggaatttctgcaatacgctacaacatgaatgattttgtaggacattacgctaagtaaaataagccagtcacagaaaaacaaatactgtatggttctacttaaaggaagcccatagagttgtcaaaattagagagacagaaagtagaatggtggtccccagcggctgcagaaagacagagtggggaaattattgtttaatgggtacagagttttcattttacaggatgaagacttgtggatatggatggtggtgatggttgcacaacaatatcaatttattttataccactgaaccgtgcacttcaaaatggttaagatggtaagttttatgttgtgtattttaccataataaaaaaaattgtagagggaaaaacagtctgcctccacttttgatatgggactgctaacatcttccaccctccctctccccctctgccccacatctgggcaagctaagaaagcctgctgctctctcctctggcaccagctggaaattcatacccaacaagccctagccctcccaccagacccacatttcatccccatccccatcgcatccccatccccatccccatccctaaccaccataaatgctaagggagtttccttgcctggttttctgaaaccatttttggacctgcttgggaatctgccctgctctctcagaaagcttcattatatgagcaataaaccttttcctaccctcttggtgcatgtggtgtatcatcagtcttgacatctaaaacaaattttgggtggtggggtccatgtctttgcagggtgaccacaatagtacctggcacattatgtgtttaataaacagagattactgtcatatttattttattttattttttgagatgaaatttcactcttttttcctaggctggggtgtagtggtgcgatcttggctcactgcaacctccacctcccgggttcaagtgattctcctgcctcagcctcccaaggagctgagattacaggcatgcgccaccacacctggctaattttgtatttttagtagggatggggtttcaccacgttagccagactggtctcgaactcctgacctcagatgatccacccaccttgacctcacttacaggcgtgagccaccgcgccttgtctctgttatatttatttctctatttaaattgatggatatatgcaaacctgatcattatcatacttatgccttgacacaagagaggcaataaactaatctaagtgatgcttgtgatgccaaagatgtcagaacactttctgggccaatggcagatacctcatgtcaccagatgctaagggtccacaataaaaagcgttgaatgaaaattttgaggataaatatctccaggttgaggaagaaggttgcacatatcgggtgctcaataaatatttgtcgaatgaatgaatgagtgaatggccccagtgtgtggggcttgggaagtgattggatataggcagagaaaaggaacaagtcaaaaataattcagaaatcaagaacaagcaagttgccttgatatacttcattcctacacttggcaaactttagtgattaaggaaacaatgttttaaaaaaagttttggtgatgagacattcaggaagatctatcaataaatagcaaacctggtcctttttaagacactgtgtataaaaaaattccaaaaagattaaaatcagtgcagaaaccaaagaaccatttttttctatatcatattgatcatttcaagtggaactgttagctatcttagaaaaattgtggttctcaattggttttgcctctatctctgaaccaccattcctaaaggaaaacattcaaccagaaaatttcagcacatcacaatctctctgaagattaagaagtctctgtgaaggactgaatgtataaattgaaaaatttttgctgtcacatttaggtaaaagagaaatcgttcttcatatccccttcctttccatctgtagctcacctatgtcaactttcctctcagaagtgaaataaaattaaagctatgacactgagtgtcagtcatggagggacacgttccccacttagcctttgctgaagtgtttccagagacaactgtctaatgccaggtccccacttagtgggctgcacttttctactcatttgcacataatccagaagtcacattttgggttcacagtttccactggggtaacctcattaggcctggccacctctgtgcttcttgtgaagtctctgatttgaaggattagtatcctttccagactgtgtgggttgactttcacccatctggagttgcttggaacaaagataactacctcaactccttgtcatgaaagcaaaaaaaaaatagcattagagttctgagaacaggatgtcattacgggttgtgccttgtcagctacaggtaagatatttgagtggctcctcaagcctccccaactccctctccaacctcgctacacagtcttcccaggtctggtatttgacctccagactgccgcccagaaagcaactcagagctcagcaacaccatgatgatgaaacacaaatatgtgggcccccaactttggagactccactggttgcttttgtgcagctctttctatatatgactttcaaaatggcagccaaccctctcaactgttggttgatctgaatatgtaaagttcagccttcaaacccagcaaacagtcagctataggatagagttcaggtgctggggaatgaccacggttggctgctacgttgggaaccctggtgacatcatatatggacctgggaatgtaaagaaacgtaggaaatctgaatttatgacttttctaatctctctatcgggatccttttttggaatcaagatgattttccttctaaaaggtcattttattacagtaatgggcagggtagacatacctcactagcgtactctcaaaatttcttgcatgcatatgctttctccggcatgcatatgctttctactacaatgtgaacaagcctacgaaagctagcctaatggaggatgagagaccacgtggaggagagctgaagtgccccagccagcagctagcctacacccacagctattttgcttgtgttccactcgtcctatttgcataatattgttctttaagttaacccattcttaatttattgttttaatcttaagcaataatattcaagaactaatgagttttgtgtattcattatatttttccataatatatattatactaataaatgcccattcaaatttttgtttgagtgctcaagtgggtatcatttaaaataatcttatatactatatataacagccaaggtttgggcaacacaacagaaactgcatgagtttattttatcagaattttttaaacggtatgggagaactaaaaaagtaaaaaagggaacccttgagttaacaaggagataagaactacgtaaagcagttactgtcctgaaggaataaagggaagagcatggggttattagaacctagaagtttggaaccaccaggagctgggaccctatgaggagagggttggcccctgacaatgctggtgtctctaagggagctcctgaggctgattctagcagtgtagggaaagaaactggaaactggaacaatttcctctgtaatcaatgacccttgccagggtaaagaatcactgctgaagagatgctactggaaaagcaagcaaacaaaaaggagggtgtcccttccccttcttccttccttccagtctccctcatgacagagcatctggctggtgacgggaaaaggtgctccacagagcaccaccccaacatcacacgggcatctgctgacccacagctgactgcagatgaggaagctcagcctaggtcacacaaacgcctcccagctgagtctagcctaaattgccaacctgccaaatcatgaactaataagtgactattgttttaagccactacctttaagggaattcagtatgcagcaatagctatctgatccatataggcctacaaggctatagaaaaactatcacatgtaatcccagcactttgggaggccagggcgggtggatcaactgaggtcaggagttcgagaccagcctgaccaacatggagaaacctcgtccctactaaaaacacaaaattagccaggcatggtggtgcatgcctgtaatcccagctacttgggaggctgaggcaggagaatcacttgaacctgggaggcagaggtttcagtgagccaagatcgcaccattgcactccagcctgggcaaaaagagcgaaactctgtcaaaaaaaagaaaaagaaagaaagagagagagagagagagaaagcagagaggctactgcagagaaaagtctagaaggatgggttcatgggttcatcgagagacaatagcttaacaaccagcacaccatagttggcaaaacactatcattgaaaaaaaaacatgctcaaaaggggaaatgccagtttgggtaaatatgcttttgtgttggagagaaagaatttggaacaggcttttcagacccccttaaggcccaacaaacaaattataatttagacaagtctgggattcttcacagctcagcttgtggtgatggtattagcttcacaactccaaacaagttaagctgtctgtgtgaaatctcctcaacaacacctcactggcaaacctggaggtgctgaaaacagagctttcaattcttgtttgcaaccaagggagttgagttggcagatgggcactgtgtccagccttgggaaaggacatcgcagactttgcatcctaagaactcataaccacaacggcaaggtaagacacaagctcttgaaagtttccatcacagtgcagcacaaatgaccttggctatgtgccctgttattgctggtccctgcttaaaaatctcctgtgacttccaaccacacaaatttcctacctggttgcaaaaatgcccttgataattcacccctccctctatcttgccccctttacaatgtggcttggcagctcctcccatcaagagttaaaatctatttcctcaccccttgaatctaggctggccatgggacttgctttggccaatagatgtggcagaaattatggcgtgacagttctaagcatgagtctcaagaggctttgcatgcagcaactttctcttagaaccctgccaccatgtgaacaatcctgcctgggctagcctaatggaggatgagagaccatgtggaagagagctgaggtgccccagccaacaaccagcctaccccagaagcagaggcatctgctgacccacagctgactccagatgcataagggagctcagcctagatccagaacgcctcccagctgagtctagcctagactgccagcctgccaaatcatgaagtaataagggactattgttttaagtcactaccttttgggggaatttgatatgcaacaatagctctctgatacatataggcctacaagtctatagaaaaactatgctgcctctctctccagccacacaatttctttctcttctcatttactattctaattcctctgtgttatagtctgtgtcccaaaattcatgtcaaaatcctaatctccaaggtaatggtattagaaggtaaatctttggtaggtgatcaggtcatgagggtggagccctcatgaatgggattagtaaccttataaaagagaacccagagagctcatttgctgcttctgccatgtgaagatacagtgaaaaaagaagcaggcccttgccagatacgagtttgccaatgccttgatcttggaattcccagcctccagaactgtgagcagtaagtttctattgtttataagctacccagcctatggcattttgttacggcagcctgaatggactaagacagtctacctagaccattatttccctttcatcatccaccagccaattccagcacatcttttagatctcagcttaaatactccctccaagacctccctctatctctaatatgaatgaaatccatatctcaagttcttcacagaatcctctactctttccttcatggcatttgtcataatttgtaattatatatctagcaaagttctttgttgttaaacatctacctcctccactctcctagaaactccacaaggacatccctgcacccagtgcctaggcaatgccagacacatagcagatgctccattaattatctgtcgaatgactgaatggcttccaagttagttaactgggcacccttgataacagattctggcctatttgaaggatcaaagaagaaagtggtgctaccttctcccctgccactatcttgcccacttgtggtgccagttcaggaggtttggaatggatgtggctaatgatagacgtagacctattgcctttcttggatcataattctgccaggctctgagtccatgtggcatcgatggctaattgtcctccaaaatttatcctctcttcttccatttataccctcccatggagttttaacagggcatgtggtcaccctactgggatctcacttctcagcttcccttgcaactggatgtggccttgtgactaaattctcatgaacagaatgtgagtgcaagtgatgtgtcagtatcttcatcactttcctaaaaagggaactgctggtcctccacttcctctctttcacccttccaatgagccagaacatgcatgtgatgctggtgagtcagcttcagtcacatgaataaaaacaaactccaggagatgactaagcaataagacagaaggaacccaagtccctagacgagttcacagaaccaagctacctatccaaccctgggcccacctggattataacatgagaaaaacataagtcctaatcatatttttgaagcactgcattttagggcttctttgtgacagcagcctaccctctagtctaatcaatatacctcaccaagtctcctgctcctaagggagacaaagaagcaaaatgagtctcaaaacatcatccaaatggaatagatacagacctgtaatcccaacactgtgggtgcccaaggcgggtggatcacttgaggtcaggagtttgagaccaacctggccaacatggcaaaaccctgtctccactaaaaatacaaaaattagccggacgtggtgttgtgcacctgtaatcccacctacccacgaggctaagccgggagaattgcttgaacccaggagggggaggttgcagtgagccgagatcatgccactgcactccagcctgggtaacagagtgagactctgtctcaaaaaaataaataaaaataaaaataaatagaccattaattaatagatatagccttggtctgtgaccaaagctcagaatgttatgatattcctttcctatgtcacctcaacttgcccctgtcatcagacaggacaaattccccactggtcctttgcactcacagctgttacatttgaaatgggagcttagccttccctgccctggttcctccttagactcatttgggaaaacaggaaacgtaattatttctgccattacctttatctcatggagcctgacagagtgtaaccaatggtaggaattaaaacattctaattgccaactcacaacaactcccgaaaaaaatcattttaactcattatacatattaaattatgacatgcttaatgtccaaacctaatagattcagtactcaggaaatcccttatacaggtagacacctttcctcctgtactttaagaaaatcttacatcaatatgcgggacttctcaaatttttctatcacagttttcttaataggaaggagaatttgtgccaaaagatgtatggaaatttagcacaaagtagccctctacaagcggaggatttctttaaagcattgtgttttatctcaagattccatggcaaatgtttatcttctctgcttgttttagtatgaaatagttttcatttgcttgtcatcatttttaaggagttgaaaatacaatcaacctcactcatcataaaataaaagcaaattaaaactactatgacatattttcacctacaaaattgagaacattttaaaatgtgataatagttctattaacaagggtgtgagaagacaattctcaggcagggcatggtggctcacacctgtcatcccagagctttgggaggctaaagtgggaggatcgcttgagctcaggaattcaagatccacctaggcaacatagtgagactctgtctctacaaaaagtaaaatataaattagctgggtgtggtggcatgcggctatcatcccagctactggggaggctgaggcgggaggattgcttgaactggggaagaccaggttgccatgagccatgatcacgccactgtattctagcctgggtggcaggaccggaccctgtcccaaaaaaaaaaaaaaaaaaaaaaaaatccctcttccctgattggtgggactgtaagctggtgcaacctcttgggaggagcaggtggttagaaaacatgtatcaaatttttatgcaaaatttaaatagacccacaattagaccctaggaatttattctccagatattctcatgcatgcgtgcaaggtatatttgcaaagatttgtatccagcactatctgtaattgcaaaaatccagaagcaacctcagtgtcaatccttagaagactgtgtacatgagatactgtacatgttggttgggttctccgagaagcgcatgcccggatagaatgcaaaagattcattagggagtaacacctctgccagaaaagcagaagagataggattgggcaggaggggccattagagcacaatttagagctaccaccatttcagaaggcagcaaagattgcctgttagagaaatggtcaggcccttgtaccctcagtcactggatgggagccactccaagaagagcatgaccatgacttaacagctaaggggaccctgaaagagctgccaggttaaggctatcagctcctcactccccacagctggacagagagcctttgtttaaggaggatctgagcagctcatctccaggtctggcacaagggttacccataggatgaagatggaagacatgaatctatatgtattagtatggaacaatctccatgatagatttttaagtgaaagagcaatagtaaattgcaaaagagaacataacgttttgggactaggcagaagtgataaccgaacaacagtgtgaatggagtaaaatgtcaccgaattgtacactttaaagtggctaattttgctatgtgaatttcacctcaatttttttttttttttttgagatggtgtttcactcttgttgtccaagctggagtgcaatggcaaagtctcggctcaccacaacctccgcctcctgggttcaagcaattctcctgcctcagcctcttgagtagctgggattacaggtgcgtgccaccacgcccggctaattttgtatttttagtagagacggggtttctccatgttagtcaggctggtcttgaactcccgatctcaggagatctgcctgcctcggcctcccaaagtgtgggattacaggcgtgagccaccgcgcctgaccccatctcaatgttttaaaagagagagaatacagcatgctgtcatttgtgttaattttaaaaaggaaatgaatttatgtgcatgtataaatgctagacatggaatctctctgaaaggagccatgaaacactcatactatgatctccagtcaggaaagagacttaattttcactgtacgcccttggtgctgcttaaatttttatcatgtgcatgtaattacaccctgtctttaaaaatcattaaagatgtttaattgttctgatgaaggaatacattacttgccatcaagaaaaatgaatgaaaaattttctgcgagacaatttttagcaagacactgttgtattgatcattcaagttcagaaaattcagcctccgtcaaggggcacaaacatcatatatcaggttcagtttgtcctctctctcagagtcaaagtgctttaggaacatagacacaataagtttctggaaccaaatggcaaatatcaaaacttgctagaacaggagaaaagtgtatcttattgaaaattcaccagctgctataccattcagcattgggaaaatcagcataccttcttagacttcattattttaaagatggcaaaatagccaagtcatggatgtctccccctttcatcaaaatgtaaagaactagctgcctctgggactctccaccaattttcaagcacgtcttttgaacccatttgatggtgtcactcaataagggcacctttttcaacttggctgcctctttttgacccaaaataatttcaacccttttctgcagctccgggcttcaccaggctttctattattgcatatacctttcatagtgattctaaaccgacctcgaatgaagagacaaatgattttttatctattggtttgattgcacttctccttgtactgctccaagacaaggcttgtcttttgagttgcaaaaaatactagcgctctatttccaaagtcaaacaagtggcttttcaatgtctctgcaagtgttttgtttcatgcagtcagtgctgacttttctcgatggagagacatatggtttgggcccattttagcaactctataataaaactgattataaaaataagcatctaagaatatcttaggcttttaagattgacaccactgcttgctactcaattgctagttgtggttggcagtgcacgcagtgtctttgtggtcaagttcattgtggcaagctcagaggtcatgtcagatcacaacacagggactttgaattgggtggacatccattcattacgtggcacacgtcacgagcttcaaggttttgcttcaaaaactcttctgtcttctaggtgaaagtataagtttaaacttactgctctttaagaaagtaaatgaaaaatgacactaaagtcccaaaagccagaattgtcagcaatcctaggtgcagttcattcattcattcattcacgtattcattcaataaatatttatggagggcctatttacctggcagcacttcatgaggccctggaaatacaatagtgagcaataaaaacacactccttcaccaggtggagctatagtctactagggagatatagatgttaaacaaattatcacacaggccgggcatggtggctcacacctgtaatcccagcactttgggaggccgaggcaggtggatcacctgaggtcaggagtaggagttcaagaccaggctgactaacatggtgaaaccccatctttactaaaaatacaaaattagctaggcatggtggtgcatgcctgtgatccaagctacttgggaggctgaggcaggagaattgcttgaaccggggaggcagaggttgcaatgagctgagatcgcaccattgcactccagcctgggcaacaagagcgaaactccaactcaaaaaaaaaaaaaaattatcacacaaacaagtacataattctatattgtgaagtgtcccaaagaaaaatatgccactcttaataagtacaggaggcttaatttggaaagttagaaaagtgatgtttaaagtgagaactgtaagacaagtcactttgtcagtgcaaagtggaaagaaagtattttaagtagcaaggagagcatgagtaaagaccacggagaaggaaagcgaggatgcagttagagacatgaaaggactgcattggtggggcaccgggatgaaggagatgatgaagaagatgtaaccagaaaggctggcagggacaaggtcatgctgggtcttgcaggccagcatgagcacttgagattcttaaagtaattgcaagggagcctttgagggttttaatgagggcagtatctttatcagatgtgcacttgtttggggtttctctggattttgttgaaagaacaatttaagcagaaggcagattaggaaataggagaatcgagaggctgtatatgttgagacgcagtggtagtctagggtgacaatgcaggaaatgggaagcagtggatgaactggggatatgttttgaaggtagaatagatgatggctgggaagacagtgactattcaagggcaggggggttgggggaggtatcaagaatgtttagaatatagctaaccacatacccttttagaaacagtcttctctgaacatttctcctgtctccaagcctcagtttcctcatctatgaaataagaacactactacttcctttttaaggttgttaaactggttaaatgagattatgcagggaaagcatcactagtcagtgctcaaaaatgtgctttttaaatttcctccctttgcctcttattctcaactttgtcctttgtaatattattgttctttaagtgggcttggttttgtcctatctttgcccattcactcactgctccccatccacccaaatcccctctgtattctgtttatgcaagactgagtttaccccttctcagtccattgacttatctctcctcactcattgacttgtcccaggcaatttattctgcaatcttggacaaaaatctggattttcagccaggtgcagtggctcacacctgtaaacccagcactttgggaggccaaggcgggtagatcatttgaggtcaggagtttgagaccagcctgaccaacatgacgaaaccctgtctctactagaaatacaaaaattagctgggtgtggtgacgtgtgcctgtaattccaggcatgcctgtaatcccagctactcgggaggctgaggcaggagaatcgcttgaacccaggagacggaggttgcagtcaggcgagctcacgccactgtattccagcctgggcaacaaagcgagacttcatctcaaaaaaaaataataataattcattatgtaatccagctttgaaacactctttggctacacttttgtatgctttaaggaggaacaaaacacagatggtctccaacttacattggttaaatctacaatttttcagctttacaatggtgcaaaaacaatgtgcattcagtagaaactgtacttcaagtacccatacaaccattctggtttgccccttcagtacaatgttcaatgaattatgtgagatattcaacactttattataaaacaggctttattttagatgattttgcccaaccttaggctaatgtaagtgttctaagcatgtttaaggtaggctaggctaagctatgatgttcagtaggttaggtgtattaaagcaagttttacttaagatattttcaagttacagtgggtttattgagatgcaacctcattgtaagtcaaggaacatctgtacttcagaagtcatcaaagctgcatgagcaggacacaagtcatatgaaaagccaggtagacataatgctataaaaaatccctccattgggccgggcacggtggctcatgcctgtaatcccagcactttgggaggccggggagggtggatcacgaggtcaggagattgagaccatcctgactaacacagtgaaaccccgtctctactaaaaatacaaaaaattagccaggcatggtggcgggcacctgcagtcccagctactcgggaagctgaggcaggagaatggcaagaacccgggaggcggagcttgcagtgagccgagatcacgccactgcactccagcctgggtgacagagcaagactccatcaaaaaaaaaaaaaaatccctccattgtcagagtgtgagcttccagctcattatcccagaagcccgagatagcagcagttctcagatcttgtgataaaggtcatctcctatcctggggctctcaggaccataatgcaagagtctccctctaaacctgccagccccagggcttttcccgccttcctcatcctaagtcctgaaaagttcactgggccaaatggtgaaccacgcacttattgccccataacccttggtacaaatgtctccaaatatatccatcaagcctacaggtagtactgagaataacaacagtagctaacattgattggacacttctaagccccttaaatccattatcttacttaattctcacaacactgatcaagagttggataaaataatccactctcaagccagcaaatctaaaccagccactcttccgtatggattcctgctcttatggtaacaagggcttgccttccccacctttattcttaacccttctggaaaacctctgctcctccttttctgagatggaaaaatttataagtgaaaaaccattccatctttcgaggtgtggagggaggaaaacaatcactcctgccttcaactaagagtgtgaaaaataagcttaactaaacctgaaatacattttcaaatgcctttgaaaagacttataaatcaaatcacatttgtccatctctctgctcttcaaaattatcatgcatgcacctgaagtttaagcaaagaaatccattaaacaaacaaacctaaaatcataaaacccagatttagagatttatccgctcagtctaatgaatgcccaattcagaatacaattttgtcttcaaagagccctgaaggttcttatctttcttatctttctatagtgttaacagaaatattacatctttgaaaagaagaaaaacattattcccagagctaaaacagaaaaggctttgaactattttagggataaatcaactcacagttaccaataaaccaaaaagaataaaaaagactgtttcaaaccaagttgactactcttacatatattcaagtgtcaacttacaaatcagtctttaaatatacacgtacactttctaactctcctgaaatgtcacccaagcccccattcaatcagctaaaaacaatttaattctttctctagggaggaaatcaggttatcagataagtaaaccttaaataccatttctaggcctgatgtggtggctcatgcctgtaatcccagcactttgggaggctaaggcaggtggatcacttgaggtcaggagtttgagaacagcttggccacatggtgaaaccctatctctactaaaaatacaaaacttagccaggcatggtgacaggcacctgtaatcccaactactcaagataatctgcataccaattgtgggtagacataggtttttgtccagagccctccacagacccatcccttacctaccattgtctctcgggcttcaaccttatttgaaagtcttaatttgcagttccacatactgcaaacacaagacccagtctttctggttcttattttacctggagattaaaatacaggctgggcgtggtggctcatgcctgtaatcctagcactttgggaggccaaggtgggtggattgcttgaggccaggagtttaagaccagcctgggcaacatggtgaaacccctgtctctactaaaaatacaaaaatcatccgggtgtggtggtgtgtgccgacagtcccagctactcaggtagctgaggcataagaatcgcttgaaaccaggaggcagaggttgcagtgagctgaaatctcaccactgcactccagcctgggcaacaaggcaagactgtctccaaaaaaaaaattaagtttctgtcttacaatatcataagaaaatggctggacaggttttcaccaaagttggagggtacttttgtgatgggtttggtttaaattggtttaaaatataagacacatagtccatagagaattcacctatggactatgctgctaagagaatctcaaagagatgcactgttatgctccagagttttgtgagaggccactaaggtcaggagacacatgccatatatatcaagatgctgtcaacagagaaaaccagtgaggtttcaaacagaagccccgctccattcaaccaggcagccactcctcattgcaggtgctgacctgggctttggctgcttctcacatgggcaactctatacactctattcctgggagaagggcagcaaagacccacttattaaatgatgtttaacaatcctcggccgggcgcggtggctcacgcctataatcccagcactttgggaggccgaggtgggcggatcatgaggtcaggagatcgagaccatcctggctaacaaggtgaaaccctctctctactaaaaatacaaaaaaattagccgggcgtggtgtcgggcacctgtagtcccagctacccaggaggctgaggcaggagaatggtgtgaacctgggaggtggagtttgcagtgagccgagatcatgccactgcactccagcctgggtgacggccgtctcaaaaaaaaaaaaaaaatatcctccaggcaattgtgtgacagctggaatgaaaaatcaggggcaaattgtacatataagggaacaattgttcatatttgtgtaagctaccctccggagtctacaagttaaaaggcacactttaatcaatttggcaacttgcatggcattttcctccactatttgtaggatgctggtatctccttaacagctactgttttcctatgcaacacacaatgacttcttgaacacatggcagcttttcatttgttcatttaacaaatacttattgagttactactatgtgccaaacaccattataaaggtactgaggatacagcggtgaacaagatggacaaaaatccctgcctttgtactacattcttgagtgggtgtgaggagacaatgaaccaaagaatgaacaaactgtgtattgtgcaggtgtgtcatgggaaaaaaatgaaggagggaagaaaagcggaaaagcagaaaatgtcaggaatgcacttccgtggcgggtggccagacaggtggccaagaagtgacatttgaactaaagaaggtataagtgagcaagctatgagggaatttggcaaaacaatttcggaggcggaggtcacagccagcaggtgcaaaggcctggggcaggagtgggtccagggcatgggacggatggggagaaggtcagcatggctgaaggaagtagggggtaagctcaaacaagtcgcaggtgggaaactgagtgtattggaccttgtaggcaattttaagaactttagtttccactcattaacatggaaaaccactggaaggttttgagcaaaggaataacataatctgcctttttttcttcaaatgctgtgaaacaaatacttatttgaccctatcaccatttctacctttggaaaggctatggtgtgttactggatgttgaggatagcttactcttcaatgtgcagtaaccaaactgaattcattctttctcaagatgagagaaagataagccaggtatggtggctttcttagataagccaggtatggtggctcacgcctgtgagctgaggcgagaggatcacttgaggccaggagttgaagaccagcctgggcaacatagtgagacccctcatctcttaaaaatttttttttagttagccagacattgtggcatccgcctgtaatcccagctctttgggaagctgaagtgagctatgatcacgccactgcaccccagcctgggtgacagagtgagacccccatctctaattttgaaaaaaagactggatagggcctggttaatacaactaactccccaaaattcaagtttttcatataggtcttttttaaaaaatagctttaattgacataaaattcacccatttaaagtttacaattcaatggatttttatatattcacaatgtagtacaaccatcattataattttttttttttttttagacagagtctagctctattgtccagagctggagtgcagtggcgtgatctcagctcactgcaacctccatctcctgagttcaagcgatcctcccactttggcctcccaagtaactaggattacaggcatgtgccatcacgcctggctaagttttgtatttttagtagagatggggtttcaccacgttggccacgcttgtctcaaactcctggcttcaagtgatccacctgcctcgggtccaaagtgctgggattacaggcatgagccacagcacctgcccgtaatctacgttagaactttttatcatcccatcacccatttaagtctttacccattagccatcactccccattttctcaacccttcccgcaaaacccctacccagccttgggcaaccactaatctactttctatacatttgcctattctgaacatttcatatcaatggaatcatactacatgtgacattttgcatctggcttcttagaataagattttcaaggttcgtctatgttgtagcatatatcagcacttcagtccttttgagtttttttttaacaatctttaccattttcaagtgtatcgtttcatggcagtaagtatattcacactgttgtgtaaccatacccaccatccatctccagaactcttgtcacctctccaaactgaaactctgtacccattaaacaacaattcccattccccaccccaccccagtccctggtatggcaaccaccattgtactttctgtctctataaatatgactactcatacttcattttttaaattgccaagtaatgtttcattgtatggatatactgtacaacaatttaactatccattcagctaatggacatttggggtttttttaaccttttggctatcatgaataatgttctgatacttcgtgtgtgtgtatagatagatagataaattaaatagaagatagaagagagagagagagattggagacagggtctcactctgtcactcaggctggaatgcagtggcagggacacatctccctgcagcctcaacctcccaggctcaagtgatcctcccacctcagcctcccaagcagctgggactacacgcatgtgccaccacacctgactaatgtatgtatttacttatttatttatgtattgtaaagacagagtctagctatgttgcccaggctggtctcaaactcctgacctcaagtgatccttcctacctcagcctcccaaagtaccaagattacaggcatgagcccccgtgcccagcctgatacttttttttttaagtattattccagttgccttgttgaaaatagaccccaagaaagcaaatctcaaacagagaaaactgctaggaagttcttgctggaatccaggtgagaacggatagaggctcacatttaaatgaagtagtcagaaatagccacatttggatgtatttttatacaattcctgctcctgaagtcttccccactcctttttttttttttttaaccattactacaattgctttgctgcctttttgctgatttattggatcacgtgtttaaaaccctgatgtgaacacctacatttatccttcttactgggtatgtgttaggtatttaacaaagtcttagttctcctggagtctgcctgcatgaaccaaccaaatataaatctgcaaaatgggaactctacagtgtctcttcagttttgctgtcaagatttcacagcctcagcttctaaaattatttcatcaagttcaatggatacatattcttgaactcttttctagcctatattttccaacaatgttgctaactatatttccataccagccttcttatctaacatactggttaaaatgtcaaaaagcagagggtttaaaaagcttttctcggtggaatgtgcttctccttcatacatgatataacttgatttgaacaatgtcacaaagatattttctctgttagattaaaattttgtttgcatgaatttttcaatagctttaagcagttgaatagcaatatatgcaggaagaagctgagagacttatgtaatagatatttcatgtatctataacccacactgctgcccaggaaatgtgcgctgcattaatagagaggattttttcctgctgaataccttgaggagttggccaacacgtttgggagtagaagtagaaagggccaggtgtgatggctcatgcctgtaatcccagcactctgggaggccaagtggggaggattgcttaagcccaggactttgaggccagcctgggcaacagagtgagactccatctctaaagaaaaaaaatcataaaaaactaaaattctctgccaaaatggacacagaaaaaactgacaatccagagaaagataatatgcaatgaagctagacatggccaaattagaaaatgatattgagagagaacaagagcaagaaagaggagccctcagcattgagagggctgaggaagcacagaaatgactgatgggttggttagttagttactttttgtgaagtgtgcaatgtaaatttcactttggtctccccaccggaatcatcaactaaagtctacactgctatatcggctatctattgctgtgtaacaaattattccaaaactcagtggcttaaaacaacacatttattatctcacagtttctgtgggttaggaattcgaagatgggccccctgcttcagggtctcccatgggttgctatctaggtgtaagctaggtctcatctcaagactcaactggggcaggatccacttccaagtgcacccacatgattattggcaggattcgtttcccatgaactgttgtcagaagccgctttcagatccttgccacgtgggcctctccgaagggcagctcacaacacaacagcttgatttatcagagcaagcaggcaatgaggcagaacagggacctctcttagggacatgcagcactcccaccctcaaacatagaaataaagaaaaatcttaagttcctttaagaaaaattccaggcacttagctagcccttaaaaaataaataaggccgggcacgggggctcatgcctgtaatcctagcactttgggaagccgaggcagttggatcacttgaagtcaggagttcaagaacagcttggccaacatgacaaaaccccatctctactgaaaatacaaaattagccaggagtggtggcgcatgcctgtagtcccagctactcaggaggctgaagcaggagaattacttgaacccaggaggtggaggttgcagtgagccgagatcatgccattgcactccagcctgggcaatggagaaagactctgtctcagaaataaataaatacatcaataaacaacttaataagcaagaagataatagtagcttagaataatgggcaaaaaagttaaaatcatgggatgtttggcttccctataaaaactaatgttcatagattgtttttcaaaaatgaggactccccactaaatgggtccagcaacacacaaggtccagcaacacaactcagataagggggacctgaaggctaaactcttaacacttttctcagttctaaatttcttcctaaggggagtagaggaagtcacaccccaggccagaactaacattccactgatctcaaatttttagacaaggcttctcctcctaagccaattacaaatcaaaacatctttaaatctacctttgacccatgggttcccactttgagacgtcctgcctttttaggtcaaaccaatgtagagcctcccatatattgatttataactttgcatgtaacctctgccttcctgcaattacaaatccttacctataagccatccgggagcttgggacttaagcattaactaattatctttgcttggtgcccctccaataaataccccacttcctcttgctacaatcccaatatcaatgtttggttttgctgtgctgggcagggggacccaagttaggttcagtatcagcaagaaggcaagacagagtgtgtgctagcaagacagaagtccgtgtgtttggtaacctaatctcaaactgaaatgccatcacctttgctgtgttctactgattaaaagctagtcacccatatgttcattgcagcactattcacaaaagcaaagacattgaatcaacctaggtgcccatcaatggagaattggaaaaagaaaatgtggtacatatataccatggaatactacacatccataaaaaggaacaaaatcataccctttgcagctacatagatgcagctggcagccattttcctaggtgaattaacgtaggaatggaaaaccaaatactgcatgttctcatttataagcaagagctaaacgttgggtacacatggatataaacatatgaacaataaaaactggggactacagctgggcatggtggctcacacctataatcccagcactttgggaggccaagctgggcggatcacttgaggtcaggagttcaagaccagcctggccaacatggcaaaaccctgtctctactaaaaatacaaaaattagcagggcgtggtggcaagcacctgtaatcccagctactcaggaggctgaggcatgaggatcacttgaatttggaaggtggaggttgcagtgagccaagatcatgccactgcactccagcctgggcaacagagcaagactctgtctcaaaaaaaaaaaaaaaaaaaaaaaaaaaaaaaaaaacggggactgctagatgagagagaaagggagagacaaaagggctgacaaactatgctcactatttggatgacagaaccagtcataccccaaacatcagcaacagacaacatacccatgtaacaaacctgcacatgtatcccctacatctaaaataaaagttgaaattataggctgttcgcagtggctcatgccagtaaccccagcactttgggaggctggggcagaaggatcacttgagcctaggaaatcaagaccagcctgggcaacatagggagacttcctctctacaataaaattaaaaattagccaggcattgtgatgtgcacctgtggtcccaactacttggaaggctgaggtgggaggattgcttgagcccaggagttcaaggctgcagtgagccgtgattgtgtcactgtactcatcctgggccacagagcaagaccctgtctcaaaaaaagagacagctcactgtcagctcactgcaacccccacctcctggattcaagcgattctcctgcctcagcctcccaagtagctgggactacaagagcgcaccaccatacccagctaatttttgcattttttttttttagtagagacaaggtttcactatgttggccaggctggtcttgaactcctgacctcgtgatccgcccgcctcagcctcccatagtgctaggattacaggcgtgagccaccgtacctggccgagaaattattttttaagtgaaaataaaaaaataaaagctagtcactaggtccagcccacagtcagggcaaggggtcacaaaaggacgtgaatatgaggtggcagggatcatttggggtcatcttagaagctgtctaccacaatgttccactatgaattatttcagaggattcacacctggggcaaggaggtacatcgatagcatgcaaccagaaggagtcctgagacagtcatttgcagaaaccctggtgaagttttggttttccttggccatgcaatcaggggctactcactggctgatggactcagctgacacccagattaatttgagaactgtatccacagtcattaactacggggcagtgcccaactgtccccaagccagaagtaagaggtatgcatcttcacgacctcaatgacaaacttgatcatataaaataggcagcattaattgacctggttcaggctacgccggaggtggactaccacctggaaacagagagacattgcagaagcttccctgaggttccccttgcagccttcaggggtgaattctttgacactgggtgtttgaaatggagcaatcaaggccccagcaaaaaacagatgcacactccaatggggtaattgaggacagtttaggaaagagattatttacagaagtgtggacaggattaagagaaaacaaggatgtggagcacgctggtattatcaacagtggggtctaaaagggaaggagagagagagagagaatcctggaacccagagagagctgtcgctgtaagacagaactgcccaacaggaactgtggcctttagggagaaactgagctactgcaaactctcagcccggaaggaaagaagccaaaggaataaataccctgacctccccttccaccctccaatctcctactgggtccccactggccagtctggctccctgggcacaccagagttgagaagaacggacagcggatttggaggcgcaagaagaaagtatccaaaacctagacagaaagccaggcagcttcacatgagctttttaaaagtgtagaagtcataaaaattctcctggaaatgagcaaacacatgggctttcagaagatgaacacaaaatagcttttaaaatgaaagtctgctctggaagggaaaaggtggttcctggcaatgtgcagaggaggattaaagtccccaccctcaactccagcctgctgcgtcttccaagagagaattccccagagcctggactagggaagcattggcgccactgggaaaagctgctacagcccttgttgggcggctctttctcacagctactgaagctgcctttgcaaagattatgacagtgagagaatctggtgttgctgactccatcttgtttctagcctcacaggctaactgtcctcactcattcctgggcataggccaagttaatcatgagatgaatttttttattttatttatttgctttttgagacaaggcctcactctgtcgcccaggatggagtgcagtggcatgatcacggctcaccacagcctcgaactccctggtctcagggtggtcctcccacctcagcctccagagtagctaggactgcaggcgcatgttaatttttttgtattttttgtggagatggggcttcgccatgttgcccagtctggtcttgaactcctgggctcaagcgatctgcctgccttggcatcccaaagtgctgagattgcaggtgtgagctactgcacctggataggtatttagtttatagtttaatttgaaagcaaggatgataatagtgttccactaaaactgattccctcattgtttcagggctgaaaccacctttgtaaaactaaggaaaggccacaagattagggaggggcctgaattctgctaaaatggaggcatagtcttaggcatagttttagaaccagccattgttctataagtcacaagatttgtgacttccccaattgctcctatagataacatcactattatagaacctaggattggtcttttgagatgtttttcagactttgcattctggcaaataactgaccccacctggacttgtaactcatgactcaactggtcctgtggcccctacccagaggtggactcagagcaccaggaccatttcccacacctctattgcatccccaactaatcagcagcatccattgcctagtcctctgcccaccaaactatttttgaaaaaccacaggctgggtgcggtggctcatacctgtaatctcagcactttgggaggccaaggtgaggggatcacctgaggtcaggagttcgagaccagcctgaccaacatggagaaaacctgtctctaccgaaaatacaaaattagccgggcatggtggcacatgcctgtaatcccagctacttgggaggctgaagcaggagaatcacttgaacccggggggcggaggttgtggtgagccaagagcacaccattgcacaccagcccaggcaacaagagcaaaactccacctcaaaaagaaaagaaaagaaaaaccacaacctccaagtttttggggagactgatttgagtgataactccagttcttccacatggccagcctcaagttaattaaactctttcttcactgcaataccacagtctcagcaaactggttttgtctatgcagtgggtaggaaggttgagtaatcacactacagttctttctggattccaggagtctctctccccttcccttttagacctggttggtaagggctctgtgctgttgacagttccagagtacctcacatcctttgtttcatttaacccagtccatgcttctgtaaatactctctttgctaaactctcctcaagtaattgattttaatgtgcatccattgcctgccaggacctgattagtccatttcaagcatccaatatcaaagaatgcacccaaaacaaagacgctgttaggaaacacagaactgggcactgcaaaggtctttgagcatgacacaaggatgttgcaagagcggcaggcccagaaggcaagtggggattgaagaaccttaacagcatattcacggggctcagtcctaggattcagcatcatgacatatcacctccaagcttccatgctttcatttgtatagctaagacttgacaagactagtgagggtgtccgatgatgaggacagtcaaggatgttaaattcctgactatacattaacaaggaggaaaaaaactaacaaacctgcatcagtgtgaagaccagaaaggcctgtccacagagccagatgacaaggtacaacaggatgctgttgaaaaaaatcagactagataagagcaggttgctgtgtgagcaaactcagaccatacaagagaactataggcccctctcctccagcccagaattcctgccctgagtgatgctaaatcagaaggaagaaaagcaggagatgatattggctaccctccggggacaggaagcatcacctggttggactaccagactgccattcacacttatatacatggagtcagtcgggtatgactcagtcccctgaggccaaatgtcagcgaggagagtgggaggagcaaatctctctcctccattagagttgtctccggaacaaacccaggtctcaaggcaaaggcctcacacactattttgctattttgtcacccacgtaaagttttcagaaacaggatccctgtagctcatcaggcactcaggtgcatcaaagctgaattcagggtaaagattgatgctgtggcccagtgaagctgagatgcccatactctctctgttcaggttatagagaaaatgggcactttgtgatcacttatacccataataaaaaacaatttgtgtgcatctcatgagcaagaaaaataaacaggaaaaaagaaagcaacccaactacttgtaagtataaggaaatccaacccatatttgttcatataactgaaaggtccaggggcaaacctgcaggtatggtttgatgcaggtgccaacatcttttgccaggacccagtgtttctcacaccctttcttttttctttttttctttccttctttctttctttttctttttgtctttccttctttctcttttttttttttaacaggatctcactctgtcacccaagctggagtgcaatggtgcaatctcagctcactatagcctcaacctcccaggctcaagcaatcctcccacctcagcctcctgagtggctaggaccacaggcatgcaccacaatgcccagctaattcttttcattatttatagaaacagggagtctcactatgttgccagggctggtctcgaactcctgggctcatgtaatcctcccacccacctaagcctcccaaagttctgggattacaggtgtcagccaccatgcctgacctcacaccatttcttaactccattcttctcactccatttcttaatttcattcctttataaagcttctcttcttactatttcaagatggctgcccaattcatgtgcagaggaaagagaagttctttctctttactctgacagttgaataaaaaatccaaagcctggctctctttggtccatccctgaatcagtcattatggcctggggaatggagtatgctaattgacttaagggaatcagggcccagcactggagtgaaggtggggctaatgccacctaatccactggagagtaccaaaagtgtgcttccccaaaggaaattcacaatactgtgggaaaggatgaattgatgctgagtcactatgaatgacaaatgcaaaagataaacataccaggccccactccttgcaggaagcaaaagatcctagagggagaggctgacatggaacaggatgtctgaccaataaaacttcttccaatgaggattcacagacatagtcataccttccaggttaagtaaggctcaattccaggcagctgtctgtctcagctcctcatgcacatccgtcgcttctgtctacccagcatttgtttctcccttattcagttctcattgctgtgtaacaaattgacagaagtgcatcaactaaagcaacacaaatgtattatctcacagctctataggtcaaaatccaagcacggctcaaccggattctctgctcagggtctcatggggctgaaatcaaggtgtcagctggagctgtagtcttatctaaagctcagggtcttcttccaggatgattggttgttttcagacttccgctccttctgattatcttgagataggaggcaggacttgactctggaggtggggcttggacaccggaccaagttcaggactaactaaaacagagctgggagggaagcagctttccctaagacacacccaccagtgtgccaggtcagtttaccattgacacggcaatacctgggagttaccacccctttccatggcaatgacctgatgacctaaagttactaccccttctctagaaagttctgcagaaaccacccttgaatctgcatataattaaaagcaggtataaatatgactgcaaaactgcccagagatgccactctctggttacagggtagccctgctctgcaggagccgtcatggagctgtaacactgcaggagctgtaacaccaccgcttcagtaaagctgttttcttctacctccagcttgcccttgaattctttcctgggcaaggccaagaaccctcacaggctaagccccagtttggagttcatctaccctgcatcaatatgactgaggtcttttttcttgctggctatcgaccagagacctctctcacctcctaaagacaaacctaggtccttgccctgtggcctctccataggctttctcacactttgagcatctctgacttcaggaagggcctagtcccttttaaaggtgcacctgattaggtcaggcccacccagatgctctcccttttgattaactcaaaatcaactaactagtagcccagtcagggcagggctattccatcacatcctctaattgtgcagcactggagaggaggagattgcacagattgtgcacaccagggagcagggatcttgggggccatctcagaattctgcctaccacatgttagtaattgatctcttctggaatcagtcctttcctgctcagtcaatgttatttgtggtaggatttagggtaatcagacctagccttggccaattggtgtaaccattgtctgggccacagtggtcagatcagaaatgggcacctgcctcaagcaagcccaatcagacctaacctggggacttttgctgaagtaactaggaaagatctctctctctctctctctctctctctctctctctctctctttcttgttattgcggaagtgttaggctgtaaacttagagctgatggtggacaccatgtgctttgattcaaatgaccccccaaaactcatgttgaaactgaatcccaaaagtgggagcattgagaggtgggacctttaagaagcgattgtatccctcttgagggcagagcctttactgaccacctaatgttttattaaagcttgtctgcaccacccctatccctaccatcaccatctggagctctcgatcctttcctccagatttctttgctccataatatttatcactatctgatacaggggtgtgtggatgtgaacatgtatatgtgtgcatgtgtgtgtttatggtccgcctctcccagtccccggtagactgtaagcttcatgaaagcagggctttgtgtactgttgtttctgtagcatgaggtctgccaaaaaagtaggttccccataaatgtttgttaaatgaatatattctgattctatctccccctcctccagaatggatctaaagcttcccaatctttgcctgagtgagtgtcctactgagttcaattttgtttcagaaaggacgtgtgtatgttcacccatatgctgcacaaatatgtaatggacaccagccatgaccagcctactggggactgcttggcagtagcaacatggcagcgaacaagacagatgagcctcctgcccacagaagcttacattcgggtggaatacaggctggtgagttgtcatgccgattcctccaacccaaaacccttgccagtatggcaggtcctggagctggactggccaccattgagaatccacccaactgcccatcagcatctcaagttccctgttgcaagatgccagggagcatgagagaggaccgtgggggagagtgagctctcaatgaacctggtgtggcagggagaagacgaagatggggcagatccccagggggaagacaacttaagggcagatttgttgaactaagttacttcaaagggtcttccatcttccagcatgagcagagaagatgaccacacacctgggggagagtcagatggcttcttcaattggaacgttcttccctaaggatgagctaatatagcacatgacctgaaaatccttctgggagactccaggtaaaagggccttgactgcaccagggagagaagctaagttgtagatttgggggaaatgagtgttagaacagtgagtgctcaagggaagtagttaagtgagccatgagatacaaagtctgccatggcaaagtaggtggaacaaccatgttatgagtataagttctgaccccagctttcctgtgatttctacgacccttggtcttgaagatgggaaataaatgggtgcatgacagaagttaaagactattatcgggtatcaagtaccatagcactttttggaggcagaagagcttgacaaatgggcatagtgtacatagtaaatgatggtaagggtaaaggataaaatgtatcatcttcctgtcatcagaatgggaccagcactgtgtaaggtgggcatgttaatgacatgatatataagacagcttttgctacaataacaaggaactccctaggcctggcacggtggctcatgcctataaccccagcactttgggaggctaaggcgggtggattacctgaggtcaggagtttgagaccagcctggccatcatggcgaaaccctgtctctaccaaaaaatacaaaaattagccaagcaaggtggcactgcctgtaatcccagatactcgggaggctgaggcactagtggtgcttgaacccaggaggcagaggttgcagtgagccgagatcactccactgcactccagcctgggtgacaaagtgagactccatctcaaaaaataacaataacttaaaaaaagagaaatagctctcaaatcccaatagatccctataacaaatatttgtttcttgcttctgagtcttcagattggctgtgattacactgggtttggcttggctgagctcagctaggttctgctgagctcagcttggctccaaggtttgggttgagttcaggtctgctccacatgtcccttcacatgagaagcaattgccaagaggtcaagtcaaatcatgcagcacatttaaaacttctgctgggggaaaatgtgtctgctcacattccaatgaccaaagcccaaagttagaagggcaaggaattagactctacctattgagatgtactacaaagtcacatggcaaaaagcttataattctaataaaggaactaagcagaatcactaggagcaatcacctagtccgccacacatggagatgtgccacagggactcagagactggaaggaattccccagggccagagttctcctgggaaactacagcctccaccattgcctcccagatttcatcagcatctctgtagtctggctcatcagaggccacaacggagataaaggcaaataaagacttcagctgctggcaagctgcagatatctccatggatcagccaagcccatgtctctttctgaaacaatcagtaatcggggaagcgaccacagaaaagcgtaatacaaactacctatggtattggaagaatcccaggaatcgttggaggtcttgaatgaatttgaagagggtactcagttcaagactactttaagacacacactttgtagatgtcccaactagatactgtgtggcctgggaattctgcaatgtttactttttttcaattatattatgttataatatatttataagaaatatatatcatatataagattatatatagcttatatatctatatataatctagatatattatatatctaaatataatctttgatatatatctcatatattatcttatacatatgatatattatcatatacatgagttatacgtatctcatatatattattttcttatatatatgaaatatatctcatatatataagatatgagagctatatatctcatatatagatagatatagatatctatctacatctatattcccaggccacacagtgtctagttgggacatctacaaaatgtgtgtcttaaactagtcttgaacgagtaccctcttcaaattcattcaagacctccaacgattcctgggattcttccaataccataggtagtttgtattacgcttttctgtatatgagatatatatctcatacacagatagatatagatcattccatcacccaggctggagtgcagtggcacaatcatagttcattgcagcctcaaaattctgggctcaaccgatcctcccacttcagcctcctgagtggctgggactacaggtgtgtgccaccaggcgggggctaatttttctttttttggagacagagtctcactctgttgcccagactcagatgtagtggtgcaatctcagctcactgcaacctccacctcccaggttcaagcagttctcctgcttagcctcccaagtagctgggactacaggcacgtgcaaccactcccagctaattttttgtattttactagagacgaggtttcaccatgttgcccaggctggtcttgaactcctgagctcagatacctgcccgcctcagcctcctaaaatactgggattacaggagtgacccacccactgcccccagcctttttttttttttttttttttttttttttttttggtagggacaggatttccctatgttgcccatgctggacatgaactcctggcctcaagtgatcctcctgcctcagcctcccaaagtgctaagattataggtatgagccaccacgcctggcccaatatttctaaaggcccctcaagaggcaaaagtgggcaaaggacttttaaggacaaaaaatgcctagtattgaatattaagttgtttactgtgttgtaactctcttccatgacttcagtaaagcagagtaagcacacacatgccccaagacacagtaacctctgtttggtagtacagcccatcacaagcagctgtggctgaacccctgggtaccaggactagagaggataacttcattgtaagttccactggatgttaggctgaagttgcagcccccatcaagccaggcttggtgtctggagcgggcctgaaggtagagattctgcaaaaggatctaactggggtgatattcaaaaaatttagaagccagaatggcacaggtcctgactgataagaaccagagccaacatgctgtgatctgcatacaagtgccagtgatccctgcctccagctgaggagggctgtgaaggatggaggctagaaatccatcaggagagctgacattgaaaggtgaatgggagcagcacctgccagcgtgacctgctgctttcctgcatgcaaaggactgtgcatttcaacctgagaccctggggttcctgcagggcatcagctagatttgtcttcctgcctggggcgatcaccaattatatgtgctgtgaggcaaactgtcctcccaccagaggaggaggtgaagggatttacagaatctctctctgatctgacttattagagaagttaaaggacacctgtccaggaggcagcacagtgcagagataaagaagtcaggattcaaatttgtcctatctgggaagcttagacaagagctccttaacctctctcacactcagtcccctcatctggaaaatgggaatcctaacagtgcctacctggcaggagcatggccacaatgaaatgagctaatgacacagtgaaagcccttagacagcctgacccagagtcagccctctgtaatgggagtcattattcaagatgggaggaaagaaacaggaattagatcagaaacaaatgaccagaaagagagatgaaatacaaaagctacatgaagtcggcagtgtgaagcctgactcgcaggagagtggattttgttactgtttgctttttcttgtccaggataatccaagactggcagaaagtgagaataccgattgagatccaaggacatcctcatgggagtctgtgcagacacgttttcatcaagaacccctctccaagcagctggtccagcccagctaaactgggggctgctgtttgtaagaaaattaatgctctgggccaagcacggtggctcatgcctgtaatctcagcactttaggaggccaaggcaggcggatcacttgaggccaggagttcaagaccagcctggccaacgtggtgaaatcccgtctccaccaaaaatgcaaaacttagctgggcatggtggcgcatgcctgtaatcccagctactcaggaggctgaggcaggagaatcacttgaacccgggaggtggatgttgcagcgaaccaagatcatgccactgcactccagcctgggtgacagagtgaggctccatctcaaaaacaaaaacaaaaaaaattaatgttctgtacctgaggagcacccatttgctgctacttccctgccaggatgaaggaaaaccaagtcagacattaaataacacgtgcaggatcacaggatcacacctttcacactgcagtcttagttttctataaaaccacgtgacctctgaaaatacactccagcctctgcaacacctactcatgacgctttgtaaaatccaccgccttttagggacaccaagattttcggaaacatgcagttttcttacctccagatgagatgtctactgtggataggtgactaggaggagaacccaaggtgtgctgatggcagaacagaagcacctaggatgccaccagggaaacgccctgacaagcagctgtgatgctgtcttcgagaaggtgtttgcaaacaccactgctgccccccttccctgagccctgacttcctaaacctaaggctaaaagcatcctggcaattcccggaagtaacttcattctagcaggatcgagccagtgggtggagttttctcagcccgccaggatcacatcagtgactgacttacccaatgtacttttattttttatttcaaccaattccccaaagcccagagcaacttaaaaccagaagagccaccaccatctccaccaaaaacagggaatattttgagagtttaatgataacttcacagccatccattcagctgagtcccaaggaaatggaagacacctgaaaaatgtatttttaaattgatttaacgttgagccatgttaaatgttttaacttccaaccaatgcaatgcccaccaaaacatcaattagtcccagcaatcaggcaagaatattggcttctgctcagggattcatttatttcagctctgctaaatatcgtagaggagaaatccaatgtagcctgtagcttcaagaaagtgaaattaagcccaagtagaaactagatgaggccctggctaaaatggtgccttttcttgcactgcctctctctggtatctctctaaagtgagggcagcaacttgattgtaagggtctcgtgctgttaacagaaaacacccttccttccccacaggttctctgtctccaagctggaaaacaaaagaggctgcaatgggacagagaagggcttcctgctgaggagatgagtgggcttaagaatttgcaagatcaaaaatcaaaacaattaaacccatggagatagagtagaaggagagtgaacagaggctgggaagggtaatgggagagttggtggggaggaggcagatggttaacgggtacaaaaaaaacagaaagatgagtaagacctacttttccatagcacaacaggatgactatagtcaacagtaattttttttttttttgagataacgtcactctgtcacccaggctagagagcagtggcatggtctctgcccactgcaacttctggctcctcagttcaagcgattctcctgcctcagcctcccgagtagctggaattacaggcgcacaccaccatgtccagctaatttttgtatttttatagagatggggtttcaccatgttggccaggctggtcttgaactcctgacctcaggtgatccacctgcttcggcctcccacagtgctgggattacaggcatgagccactgcacccgaccctatagtcaataataatttaatcgcacattttaaaataactaaaagagtacaattggattgtttgtaacacaaaggataaaggtttgaggtaatggataccccattttacatgatgtgattattacacatcgcatgcctgtatcaaaacatctcatgtaccccataaatatatatacctactatgtactaatgaaaattaaaatttaaaaaaattaaaaataatttgcaagatcatttttaataattatttttcatttggggtgcattttcaccatcaaggccccagggtttccctgccctagatgagggtctggatccagggaagggtgtcccaccatctggagcatcctgtctctacacatgttggccagctgcttggggcaacacaggacgatgcgctcaggccactcaccacaggagcttggccattagaaccagcaggggacaaccaagccaaagaccccacgactggagagctcctaaaagcaaatacaaaataagagtacacatgaagttctggcagcaagcagaaggtagacacatgatgtccaactccccgagtctctctctggggccacttgatgcagagccttgatttgttcgtctcttaaatgggaacaatgatccccaaagaggaggaggggtgggggagtgagaattaaatgagattgtgtcagtcagcatgtactagggagcttgttagaaacaccaattccaggatcccctggagattctgatttagcaagtctgccaggagacccaggaatttgtattttaagcatctctcccaggtgattctgataattaagcaactttaggaaagaaaagattagggctctggtccataggaagggttcatccaatggcagccctcacttaggcccttggggtgaatgaaggaggggcatccaggatcttgcccagccagggacatgcaggaaacatctttttaaggggccctcatcactttctgacactgctgtttcaagggaaaactggtcacacctttctgcaggggatttgacaacatccattaaaaaaaaaaatttaagacagagtctcgccctgtcacccaggatggagtgcaatggcgccatctcggctcactgtagcctctgtctcccaggttcaagcgattcctctgcctcagcttccctagtaactgggactaaggcatgtgccaccacacctggctaatttttgcatttttagtagagacggggtttcaccatattggccaggctcatctcaaatttctaaccccaagtgatccacctgcctcagcctcccaaagtgctgggattacaggcataagctgccacatccgaccaacatccatcaaaattataaatgcagaccctgtgatctagcaatctcactttgaagagtttatcctacagacatacttgcacagatgcaaattatatcaggtcaggagctgcagcattgttcataacagcaaaaaaaaaaaaaaaatagaaacaactgcaaggtccatctacagacctctggttgaatacagttacggcatagccataaaaccaacaatggcacagctcctgtgcacagatacagaactccctccaaagtatattgttaggtgaaaaatacaaggtgcagaataaggtgtataataagcttttgtgaaaagtgcagagatcatctatatttgatttgctgtgtatgcatagaatatctctggacaaatacacaagaagctgctaacattggttgcctctggggagggaacagggtggctgggggacaggggctggaggaaaagatttcaccacatgccttgcagactttatatattcggaaccctgtgcattatctattgcacatattatctattcatgcaaaacgctataacatttacaaataactttcaaatatatgcatatcatatttcaatggaaagttttttttgttgctgtttgtttgtttgtgatggagttttgctcttttgcccaggctggagtgaactggcatgatctcagctcactgcaacctctgcccccagtgttcaagcaactctcctgccttagcctcccgaatagctgggattacaggtgccccccaccacacccggctaatttttgtatttttagtagagacggggttttgccatgttggccaggctggtctcaaaactcctgacctcaggtgatccgcccacctcggcctcccaaagtgctaggattacaggcatgagccaatgtgcccagcctcaatgaaaagtgttttaaagtaataatataatacatcaaaaggcatgatttatgttttcaagtgccacctctggccacttgtggctgtagaaatggggaatactccaattcaccatccctatgtgaaggcaggacagggagacattcaagtcaggaaaaggtggagtggaggttttagtcaagcaccaagtcctctgacttctctgggcacttgcccatgccggtacctactcctgagacactgtctacctcgctaagcccatcttcttctcaccttgtgtctcacctgagacatcactgactttaggaggtttcctgacccatgtgtgttccccatctcagcacccagcacctgcctcatgttcaccagggtacatgtcttccctccccgccaggctgcactgagagctcaatgagggcagggaacatctctgttccccatcttgttagaagctatctccccagagcatggtccaaatgtggtgttaagaacagaagttatcacaaagaagtgagaaagtgaaggggaaatggggagagctgtaagaaaggcatgggggagaaaaaggaaagaaggccatgttcccaggatccaatgccactccctggggaggcttctctatcttagctcagccctcagtcactatcaccctccctggtcttgtcagatttgaaatatcttcttcatttacaagagggccttatctatcttggtcacctctgtattcccagcccctactgcggacttggaacatagtaggtgcccagtaaacatgggttgaattaattaatattggacaggccaggcttggtggctcgcgcctgtaatcccaacactttgggaggctgaggtgggcagatgacctgaggtcaggagttcaagaccagcttggccaacatggtgaagccccatctctactaaaaatacaaaaattaaccaggtgtggtggcacacacctgtgatcctagctactgggaggctgaggtgggaggatcgcttgagcctgggaggtggaggttgcagtgagccgagatcgcggcactgcactccagcctgggcgacagggcgagactctgagtcaaaataataataataataatagttcattattattggatagtgaggagggagtttgcaaaggagcggctaaaatgaaattagtcctgagtttctgatgttatgcgttctgggtaggggcgggagaactggaagatgtttaatgccccccacccaagggcagccatagttgagaatcactaattctgaagcttggtagggaagcttgatagggactttgggtttttcactgagggcagaatccccagtgactgtcagagacctcgctctgaatcttaagaacctccccaccccaggcctgacagggaggacaaggaaggagagtggggctgggacgcggggcagagggcggctctgctcacctgcgggagaacgcgaggatgaggttctagtgggaccggcgctagcgggcgcgtcctgccaggagtgagtcttggcgccatctagcgcctgctggaggctctgcagctgcagtggactgagccgccggcgctgggcgtgggtcaccgccgcggcgttctccggacccagggaggcgatctgctccgcggacagctcctgcaggaggcaaaggggcagcaccctcacacggggcccctccccggatttcccacatctcgggcgtccgcgcaggatgcacacaactttgccatccccttgtgtgtatcatgtgtggtcctctttaagatatttccttaaaattagatcactcttgcactaccaaagtaaatgcatttaaaaatagaaaatttaaaatggaagtttccaaaaatggaaagccggcagacttgccataagcaagcattaaataggagccgcaaaataaaaataaaacaatattgttgaatcctagctagatttgtccgccaacaatatctgaacccctggcctcttggaaaaaaaagagagagagagagaatataaaatgtactatacagggttaaattgacacttccttcttgaagtatttagaagtactaatggagagttgaaaagggaagcatgatttcctccctatgtggcaatgttgtttaatgcaatgcaggacagcttcccagtgcttcaagtcttccacctcctgaaacactgatgtggagggggaaacacaggccttaaagatcagaggcctgaattcgagcccctgctctgccacatacttgctgtgtcaccttgaacaaattacacagcctccgtgggctttggggataaatgtgagacggcatagagaactcatctcctctgctgactgattctgatcctttggtttgactgcctgagcaccatgtgatgagctctgtgagggctccatggagggaaaatgcagtcatctattggtgatatctgctatggacaacatgagttggaaattctgccagccagactatgtcttcaaggactgtgaacaaggtgtcttctgaagtcacttccagatcaaaggacttggtgactcgttccaatgggactggaatacgaaggggactctatatcatcatgtttattttctaaaggccctgaagaatctgggaaagatgcttatgctccctcatcccttcctctcctgtcagtcaccctctcctcctcttttgtccaggtagatacttccctggaatcatatccctctgtgtaaagcccttccaggaaccccactccagaacacagttcaggctcctggaaaaaactctgcccacctctccagcctcacacatggcattcttcactccaggagagtgggtgccaatatttacagttccctagaggcagcatcttcctgtgcacctctttctgcctggaacactgtttcttcttcatcgccagctgttctccaagattcagcatctggtcggtcaagcgccatggctcatgcctacaatcccagcactttaggaggctgaggcagaatgattaacggagctcaggagttcgagaccagcctgggcaacacagtgagaccccccttctctacaaaaagtttttttaattagccaagtgtgtagtcctagttacttaggctgacgtgagaggattgcttgagcccaggggatcaagggtgcagtgagccatcattgtgccactgcactcccttgggcaacagagtgagactttgtctcaaaaaaattaaaattcagcatcacctcctctcctctcactacacacacacacacacacacacacacacacacacacacacggtggggtggtcagagaggtacttttttggcttccatataccattaacacagagtctgtattctaaaaattatctcttttatcaatgttgctgacttgcgacccacaggccagattgaatctctaggcctacggcactgtgttttcaaaaataaattcagattggctgccaacatttaattatcaagcaattccatatgaaaatgaagatttctgacttcttctcgaaaaaagtgaaaaggaaaattcagcaatcttgggcccacatggccacactgcatgaatgtgctaggaatgagaagcagcctccacatttagacaaggctctccagttcacttcggtctctacccagcctactgcaccttatgttaccttttaggcccctgaaggcactatgtgataaccccttcacctgtgcatccttaggtgcacctaccctggagcctgccacaatgtaggtgatcagtagggatttctggaatagataaatacctgtacaaggaagcagcacaacagcactccaaacaagaccaagagaaagtgcttagcaaaatgccagcatccaagaccaagctgcttgtcccatgcagagtgccccagaggggaagcaggtatttgctcaatggctttgaggactgctatttattgccctcaaacagctattatgcctgcaatgtgctcggaactgtccaggatgctgaagtgattgaaggttggctttctttttttttttttgagacagagtctcgctctgtcgcccaggctagagtgtagtggagcgatcttggctcactgcaagctccgccccccgggttcacaccattctcctgcctcagccttctgagtagctgggactacaggcacatgtgaccaccccagctaatttttttgtatttttagtagagacagggttttaccatgttagccacgatggtctcaatctcctgacctggtgatccaccctcctcggcctcccaaagtgctgggattacaggtgtgagccactgcacccggcctcagctatctttcttgaaatgaacaagtgatgtggcactgggggagtttgtcgagcctggtttgtgtgttttatccagaagtgctggacaaaatgtgttcccacattacgcagcaaacaggaacaccagaatgactgcctcctcatcagagcagactattcccaccaaacactcccctaatcttgtatacagacagaaaaaaaggacctgtcattagataccctgcttcccttccttcaaatgatccaatttcaccctctcaattttttcatagcagtttttcacctgtcctcttgtgattttcctaatacttttctttaaatcaactaaaaatatatatatatagaaaaaaatatatatatatagagagagagagatggtttcgctcttgttgcccaggctggagtgcaatggtgtggtcttggctcattgcaacctctgtctcccgggttcaagtgattctcctgcctcagcctcccaagtaactgggattacaggcatccaccaccacgcccagctaattttgtatttttcatagagatggggtttcaccacattggccaggctggtctcagactcctgacctcaggtgattggcctgtctcagcctcccaaaatgttgggattacaggtgtgagccaccatgcctgacctaaatcaactaaaatatttttatttaattatatcttaaaaggaaatgttacagaggtccacaatccctcatctgcaattccaaagtccaaaaataatctgaaaactgcaagttttcccccaaagtttaagtcaaacttatttatcagcaaaacttgacctgaagtaatgtgaggctatttatttatagtctctatttattccaattagtgtggcttgtcacagatttcttcacagaaatattaatgtgtttgcttacaaggtgcttccccagacactgctgggggtatgaagttacatacagtaaatgtacagggtgatcttttcaaaatctgagaaattctgaattctgaactatatctgtccccaggggttttcgtaagggtttatgaacctgtcataccaccataagtagaaaatcagtaccacctgctagaagaagagaagatgaccgtaaaaataaatataattaaactacatattgtaattttaagaaaaatgttttttaaaaacacctaaactcacacagatctcctctgaccccatcagcagagcctggtcacaagcctctaaattccaaggcccatcacctgtttccctgtgtgatttgaaatggggtcaagctcccatttctccttgaagaactgagcacctactttgaatatctcatcaggaaggcattttattgctgatggctggaaatatggcatcaaatccttgtcaagcatccggagctctgccttagttaatccagctggggagaaaaaggaatcacgggggtttagttcaagccatcagaactccgcttgttttattaatggtgctgcataatgttcagatctgagtgttctaggcaggcatcattccttacaaaaggccctggaaatcacactggggaatcaagttccttcatcaactcagaaaaaaaaaatgtgggtcacattagccctgattggcctcctacagtgaaacgcatgcccagaaggaacttcaatttacacactttcaaattttgtataaacctacttaggggccaattaaatcacattctaaactagcggttttccaaactttagtgtacacaagaatctccaaaagagcttgttttaaaagcagatttgcagacccaccctctgcaacttcaaatcatgaaatgtaggttctactgtaacgccactgatgtttgctacacatggccaaggataatgttttattttgtgtccccacatttaagtttggaaagagagagaaaggtatgctcagggtgagtcttacctgcaatggtcccaagctcctgcaagacagaactggtccactcagtgggatccccaaacacaacttcagccttcctcttaaactcggctaagacatgtgtgctgcagagcagggtcccaattctggccactaccaccctggtagtggttaaagagggagggatataatatgagcttggactcttcagccaaaaacaaacaaacacacacacacacacacacacatacacacacacactgcacagtaggctcagcagggacagcagatccagcttatcccattagcccagtgggattttagcccagaaaggtgccaagtgtcaggaggtggaatatctggatggatggatggatggatggatggatggatggatggatggatggatgaattaacccatttgccattttgcacattcatattttagttacctgaattctgagatctttataagtgggatttcagtgatgtttatagcacacagggttgcaccaagtcctaccaaatgaaagctcttcaggtcctggatactgtatcctgaatcatccaggtacccttgcaaaatggattcagcctaaaaaatagtaagaataaaagataaaccatccaggaatgatcaaggtccccaggcctgaggggataaataagctgttgctgtagtctcctgactgatctcccatcctactcctctggaatccccactccaaaccatcctggcctctgcatccaagttcatgtccttaagatactacttcaactgagtatgtcccctaatctatgaaagtgtcttcaggcaatgaatctcttacatccttcaccaatattaaaggcacttgtgtcctgtgtgtctgctgcttatttccttcagtcactcttatgaccctcagacagtttggacatacaacttcttctgcatcaggatccaaacttttccagcatctttttccacaacgtttcttctcccttttttttttttttttcgttttttggagacggagtcttgctctgtcacccaggctggagtgcagtggcacagctcgctgcaacctccacctccccaggttcaagcaattctcgtgctttagccacccaagtagctagaattacaggtgcacatcaccacatctggctttttgtattttttgtagagacggggtttcaccatgttgcccaggctggtatcaaactcctgacctcaagtgatccacccacctcggcctcccaaagtgctgggattataggcatgagccaccgcacccagcctttccacaacctccaacaaaaccttataatttcctgtctctttgcctttgttcaaaccagtcctttcatctgaaatgcccttctgcacttccaagtgcagacattcttttttttttttctttggttcaactcaaatgtcaccttcttcatgaggtttcagccagaatgattttttcttcctctatggtcctacagaaatatgtttaccccttaatgattttttcttcctctgtggtcctacagaaatatgttcaccccttcaattccaatgtttactcttcaattccaagagtagcacacaaaatgctttggtgttaaactattttaaactaagccttgatttaaggcggctgacacataagtctccataattctagcacagtggctattcatcattcataacttcctctggagaaccacctctcttgtattcctgcttcatgtggttcagtcaaagctaactgcaccaagttccaggagtgatgaatttcaattcataccttagcctagctgcagtcactggttctggattggacatgtgatttaaccagagtcaaccagaactttgagtggagcattaggggaagagctttctttactctggacttgaacttagaaggatatacacaaggatctgctggaacctaccacatgcaggcatagagcctgtctctcaatgaagccaacacaaagaaaagcaaagttcaaaaacatgataagagacagattcccacccagagtgttgaagatcctggatccagctgtatctgaacactacccctgaactttccagtaatgggagtcaataaattcctttttttttttttttttttgagacaaggtctcactctgtcacccagcctggagtgcagcggcatgatctctgctcactacaacctccctctcccaggttcaagcgattctcatgcctcagcctccccagtagctgggattacaggcaggcatgaacatgcctggctgatttttgtatttttagtagagaagtagtttcactgttgggccaggctggtctcaaactcctgacctcaagtgatctgcccgtttcagcctcccaaagttctaggactacaggtatgagccactgcacagtgcacccagcccctttatttgttcaagccagtttgtgttgggttttctgccacttgtaactaaacatgtgctgattcattttatctacctatgtaggacctgaggaggcatccaagccaatcctatgaagatcagctacaaaataaagtctgggctgggcacggtggctcacacctgtaatcccagcactttgggaggccgaagcaggaggatcacttaaagtcaggagtttgagaccagcctggctgacatggtgaaagcttgtctctaccaaaaaatacaaaaattagccaggcatggtggcacgtgcctgtggtcccagctacttgggaggctgaggtgggaggattgcttgagcctgggaggtggaagttgcagtgatccaagattgtgccactgcactccagcctgggtgacagagggagactctgtctcacaaaataaagtctggttccttcagtgctcatgggagcaagtaaaagagattataagacctcacaaggcaaagatgaggagacatccaaggagagacccctaagtggaagcgaaaatcacaggctatagtcaatcttcccaactctctttgcttttttttgttttttttttttgagacagcgtctcactgtgtcacccaggctggagtgcagtgacatgatcttggcttactgcaacctccatcccccagggttcaagtaattcttatgcctctgactcccaagtagctgggattacaggcgcccgccaccacacccaactaattttttgtgtttttagtagagacagggtttcaccattttggccaggctggtctcaaactcctggcctcaagtgatctacccacctcagcctcccaaagtgctgggatcacaggcatgagccaccatgcccgacccccatctccgtttaatgttagtcatccccatcacacaatatagatcattaaggtgttgagagaaagtgttgaggaagattgtgaaatgttgcaatgaaattccgtcttcatgggctggtgcttcccacccctcaggtgtgtttaaatgctacctactcagagaaaactgccctctggactctttcatctcaaacagcccttcttcccactattcccatgagcaccttgtccattttccgcatcacttatcgtgatttttcaaatttttcactttgattactcacttttttgtctgtatcaccaagtaggctcccaaaagaaaaggaccatatccaggtagtttaccaatgagtccccagcacctagcataatgcctgccacagagtaggagttcaataagtacttcttgagtaagtaaatgaatgagtgaatgaatgaatgaatgaaacaacatctgagtgagtgacttacaaaagtcgtgggcaatatatattatttgacatgcttcattttgctgccttcatgtaatagactggcttcaggaagatttctcatgtttctgaaaatcggactggtcagtgtcctcatcaagttgtcctccatgattacaaagctcacagctactatgggggcgggggatgcagtaacaccatgcttagacttatatgtctttgacttaatgggactgtatatccaaagtggtaatagctaacatttattgtagctaacatttcttgagtgcatactgtgtgctggcaccactttgaccactttacacatactatctcatctaatcctcctaaataacactatgaggtaagtattaatagtatccctagtttgcagatgagcaaactgaggcaaggagaggttaagtaactaggccaagatcacacagctagaaaatgatcgttctggccaggcgccgtagctcctgcccgtaatcccagcactttgggagacaaaggcgggcagatcacctgaggtcgggagttggagaccagcctgaccaacatggagaaaccccgtctctactaaaaatacaaaataagcagggtgtgatgatgcatgcctgtaatcccagttactcaggaggctgaggcaggagaattgcttgaacccgggaggcagaggttgcagtgagtggagattacactccagcctgggcaacaagagcaaaactcattctaaaaaaaaaaaagaagaagaagaaagagaaaagaaaatgatggttctaggatcaaaacccaggcagtctgattccagggcccatactcttagccagtgaaggtgtttggctatggagaaaagatggagattcaagttagttttcaaatttttcttattaagtcttcataatcaggtttcactagttgactcagagcaatttgggctcctcaactatcaggcacgctcactttaaaatgaaagtgcaaaaatacaatttaaataaaattactttgaagatatggtattaaattgtccttgccactgaaacccggaaaatgcaagctcagcctgcaaggtgataagttaaaataaatttccttgagtgacgagaccagtgtatatgtaatgattccaagacaattaataccaacacttttaggcaatattaactgttgaaaaatgaatagctttaagatttcaatctctctgttcatccctctggcttctataatagttttttcccttatattggtttttgagctgaactatctgttaatgtagttccgtcaggtctgactattaatctagaaacctgcatttaaggttgattgggagctaaagttgaagaactgactacaaatgatacatgcaaatgttaggttttcatatcctcttcaaacatattgataaatctcactgccatccatgagaattaaaatcggtgcgaagggaaggagataccctagtttctgcatgtatttttctgtttctccgagcctgcaaatagaatgattttaatagcacagtgtgatgtggcacttctcaaactagtcaaaaccagttttagagtcaaagaaccatgggattatagaatatcagactggaaggaacctcagattgttggggctaaccctctcatcttgcaaaggaggagactaacccagagaaaggggctgtaatgatgataatgttgatgatgacaatgcaatgatgtcaatatcaacataaaaacagctaatttttattgactactatgtaccagacattgttcaaaatgttttcaagtattaactcatgtaatcttcatggccaggatctgcatgttgcccagctccaggggtgcaccattcacagtgtattctgtttgaatggtgccactcagagttatgaaacatggccaccgtgattacaaaaagctcaaaagagtagggggacattcattggttttggctgctcagcgtccacactttttctttggaaaattaccctccattagcttcctattgctactctaccaaattaccccaaacatagtggctttaaaactacacgtttattctcttatagttctggagactgtatgtcctaaaatggactagaaggatgggttccttctgggagttctaagaagaattcattttcttgctttttctagcttcaagaggccacctgcatttccttggctcatagccccatcctccatctccaaaatcagcagcagagcaccttcagatctctttctaggtccctctgcttccattatcatgtcaccttctctatctatgaccctccttgctccttcttaaaaggacccttgtgattacattgggcccacccaaataatccaagataacctcctcatctcaagatccttaatcgcatcttcaaagtcccttttgctccataagttaacatagtcacagcttccaggggttcagacatggacatctttcggggaccttaatcagcccaccacatatccttccccactcggctcatgtggtcatgagatgctgataagatggactccgctccctgatgcaggcctcgccaaccaacatattcaacccctgaccagagtggttgctcaggggcgggcaactacatgagtggagtcaatgctgaggcttttccaaaaactaataaagaagaggcacatttttatgggcttgttaattaggttgtatataagcctagtgctgaaaatgaccactgagagagctgcctaagaacgaagataacatggaagggctaacacttccctttccagcagtacagtggattagacaccctaaactcctctcccaatcaaaacaattaaaattctgggaagaacttcttctttagcccatgaaggattaattaacataggaattggccacccaccataaacaactagaaaatttaacaaaatacatcagacaactgcatccggatatggaacaacagaatcataatcccagaagaagaaaaacaaacaagatgagccttaaaattaccctcacttactgccaaaaagcagtttccaagacatggatcaggaagaggaaccaaaacaagcccagtggcccagctgagtggaggatacagatatccaagttcagagaggccacggcacttatcacttgggcaaagtattggagaggaggaaactgcagagggttccagaaagctgcagagatgtctagtactgactgctattttgcacatgcaaagagtgaaactccacataggcaggaaaagagtcatcagtaatcagaaaggattaggctgagcaacttccagagctcatatggagctggaaatagttcacattctcaccagccaaagtagagagatcttgaaatacatggagcattaggtaacatcctcaaaggaatcatgtcacagtaatgatgataaattaacaacagaataaaggtcactctggttttaccctaacaaaactcaaaaggaagcatcaaaaggatcaagctgatttgaaagtaacttaagtgtatgacagaacaaagcccaatactcttcaaagaaatacaactaaatcaaatactcaacaatgtaaaatccacaatgctcatcacccaatcaaaattgctaggcttgcaaacaaaaaaagaaaatatgactcataactaagagaaaaatcagtcaacagaaacagactcaaaaatgaccatcatgaggaaattaacagtaaggatatgaaggcagctcttataaatatggaatagttaaaagacccagaaacatccacccacctgtcccggggtccattctgtttgccagcttagggaagccacagtgtctatggagctgaggtccagctgctccagctcactctcattaagagccagagcaatgcgccccagggagacgatatggtgttctctccagtaagatggcatgtcccaaacctttaggcaaaaaaagaaaatagattagactgaacactgtgatggtatttacaatgatttgaatgtttgtgcccctctaaaatgcatatgttaaaacctaatctccaatgtgatagtattggaaggttgggccttgggagatgattaggtcataagggtggagccctcatgagtgggattagtgcccttgtaaaagagaccccagagagctagctacaagctagtccctttccaccatgtgaggacacagtgagaaagcaccatctatgaaaaacaagtccttaccagatactggatctgccagtgcttcaatttttgatttctcaccccccagaactgtgagaaataaatttctgtttataagttaccagtctatgatattttgttatagcagcctgaatggacaaaggcagtatctaaaatagacataaggaggctaggtgcggtggctcacacttggaatctcagcatttttggaggccaaggcatgcagatcacatgaggccaggagttcaagaccagcctggccaacataacgaaaccctgcctctactaaaaatacaaaaaaaaaaaattagctgtgcatggtggcgcacatctgtaatcccagccacttgggtggctgaggcacaagactcacttgcagaagttgcagttagccaagatggtgccactgtactccagcctgggtgacagagcgagaccctgtctcaaagaaataaaaataaaacagacatagggaaacctattgtgcagcatggtgaccacagttgataataatgtgttgaatggttgaaaattgtgaagagagtagatgttaaatattctcacaacaaaaactgacaacggacaagtatgaaaggtgattgacatgttagcttgctttaaccattccccaatgtatacatacatcaaaacatcatattgtatatatgtatacaccataaatatatataatttgtatttgtcaactatcccttaataaaaaataaataaaaattaaaaagacatagggtaaatcttaatataaagctctcttttaaatcaataataaaaacatgactatatttgttttaaaaaaaagatagacaaaagacatcaacaaggaattcacaaaagaaaaattaaatgtatggggaaaactttattctcagtcataattaaagaaatatacattaaaaacaatcagaccatgttgattcatcacatgtcaagaagtataaagacttatcaccatttacgatactggaaagatacaaggtaagggataccctcatttgttgtagttgagaatataagttggtacatactcttgagagggcaatttggctatctgtatcaaaagcttttaaagtatttatattctttgacccagaaattccacctctgggaatttagcctgagtaaatgagacaagtacccaacagtatatgtataaaggcatacattgaggcatttttttaatgcaacctgaaagtctaacaattagctaaataaataagaactaaccataaataaaatgaagttgcccctgtggatacctccaccaaggattggttccaggacttcttaaagataccaaaatccatggatattcaagtcccttatataaaatagtgtagtatttgcatataatctatgcacatcctcatgtatactttaaatcatctctagattagttataatacctgatgcaatataagtgctatgtaaatatgtacaaaatgttatgtcaaaaatgctactgtatttttatgtgtattgttttttattgttatattgttatttggatcattttttctaaatacattctacctgaggttggttgaatctgcagatgtggaacccactgatatggaaggctaattgtattacaaagctatttaaaatactgatataggctagatgctgtggcttacgcctgtaatcccaacactttgggaggccaggcagacagatcacttgaggtcaggagttcgagaccagcctggccaacattgtgaaaccccatctctactaaaaatacaaaaattagccgggcatggtggcggatgcctgtaatctcagctacttgggaggatgaggcaggagaattgcttgaacctgggaggcagaagttgcagtaagccaagaccacactattgtactccagcctgggcaacaagaacaaaactccatctcaaaaaataaataaataaataaaatactgacatagatgtacattttggatatgaaaaaatatgcagtctgtactgttgggtgtagaaagcaagttattgaatagtaggtaagtataatatcatttttgtaaaacatgagatatatgtatgaaatatataaaataatattttttatatacatagttttggagtctggtaagcttcaagtgaatataccaaaatatcaacagtgcttatctgctgaacagtgcttatcaaaatatcaacagtgagtaaaagattatcactgattttcttctttattattttctgacttttctacaataaacttgaagtactcatataatacataaatacagttatatttataattttaagacattgaattgtttaacccttgagggtaactagatattccacaaccatgtaaagagctaaaacagggctgggtgcagtggctcatgcctgtaatcccagcactgtgggaggccaaggtgagcagatctcttgaggctaggggtttgagacaagcctggccaaacaaaccccgtctctactaaaatacaattattagccaggcatggtggcttgtgcctgtagtcccagctactcaggtggctgagacacaagaatcacttgaacccgggaggcagagtttgcagtgaacaacagatcgcgctgctgccctccagcctgtacgacagagcaagactctgtcttaaaagaaaaagaaaaaaaaaagaaagaaaagctaaaacaggccacaaagggaccttttccttttatttatttatttgagacagagtctcgctctatcaccaggctggagtgtagtgacgcaatctcggctcatggcagcctccgcctcccgggttcaagcaattctcctgcctcagcctcccgagtagctggaactacaggtgcatgccacctgtagagatggggtttcaccatgtgggccaggctggtctcgatctcttgacctcgtgatccgcctcccaaagtgctgggattataggcatgagccactgcacccagcctatttttatttatttttgagacaaggtatcagctctgacgcctaggctagagtgcactggcgcaatcttggcttactgcaacctccacctcccgggttcaagccattctcctgcctcagcctcctgagtagctggaactacaggcacatgacaccacgcctggctaatgtttgcattttgagtagagacagggtttcaccatgttggccagcctggtctcgaactcctgacctcccgtgatccgcccgcctcggcctcccaaagtgttgggattacaggcataagccactgcacccagccaacaaagggacctttttaaagatggaaagctacttccagtcctctttttactcctttctgttatattttcagacaaatttgcaaatgattctgagaaaacctgctgtgagcagcagctggggctctccaggtgaaggaataaagcctaattcctgcaaaccgccttggtcagaggcactgggacatccacagaacttgattcactgagcatctgctagatgccaagacacatctcatcccattctctgccacagccctaagagggaggaactggaaatttcctcctttctcagataaggagatcacaatatcactgagctgatgcagtaaaatttcaagatgatgtatgggaaaactgctcgaggagggttctatgtgcagaaatgctgaactggttttggtgtttttcttttttcctctgtttaatgtttcctccctgaggtgggcttcacctgtattgctttctccttcagggtcatcagctgaggccggctgaaaccctggacagctcccagcagttccacggtcctgatgaatgtgtcttcctccatgcaccgcaggtcctccagggcccagcaggcgttggcttcggccaacttgaagatgtcatcagaagagggggccacgactccatggcaacctgaagaaaggagagatgcagggaaggagaagaggaaagagaatttcaggaaatgttagtgtattgaaatgagtaacattctttgccagtcagatttatgcatggaagggactacagagtaaaagcagagagcaggaggcagggaccagtttgaatcccacttctgccatttcttgctaattactgggtaacaaagtcatggaaatgccagctttttgcacatccacatgcacaaactaggagcggtgcaacattacaattaaactacaaattccttgagggcagtaagagctggtggtgaacagtcctgactttcgattcaaatccaaggctatttaacctctctgtatttcagttttcttgtctataaaatggaaataaaatcgcctcataggatggttgtgaaaactaaatgtgttatatgtaaagcacttagaacagtgccacgcatacagtaagcactcgatacacaatttctgttgcctatcttaccccctatgcctttgagtttgcagcccagcataaagattccaaaattatgcagcagccttcctataaaaatgggaagatgggctgtgcggtggcgcatgtctttaatcccagcactttgggaggccaaggcagaaggatcacttgagcccaggaattcaagaccagcctgggcagcatggtaagacctcacctctaaaaaaaaaaaaactacaaaatattagctaggcatggtggcacatgcctgtagtcccagctacccagcaggctgaggtgggaggatcacctgagcctgggaggtcaagattgcagtgagccgtgatcacacctattctccagcctgggtaacggtgagaccctgtgtcaaaataaaataaaaaataaaaaccaggaagatgagttgctgtccatgaaaatcatagtgggggttgtggctttcccatcccaagagaaaagagaaatgagcctattgcatttctcttggcaacaggaaaaacatccatttcggtctttgaataatgtgaaccttacctactcaaaccagctaccaaccaacatgcaagtgagatattgtctttgctggatctgatgtgcccaacctgggagaaaactctaggatccttaagagcaaccctggatttcttatcttagccgcagaccaccactgatgctgacagatgcaccaaccccacccaaggccagggtatttcccgagccccatggcctctctcttctcattgcaccttcaatggaaattcactgcattccaatcacgaggcaaaagtagaccagatcaaagatgccagttgtggtcctcaaacatctttatcataagccacttggagaggactaaagacccatccctccctaggccaacccccagcccaccctctagtgaaaacaacatcagtgatagcacagaactagagagggcagaatggttgactagtatatcaggaacttggtcttatcactgtgtctgtccagggcttagaatagagcctggtacatcgtaggtgttcagcaaatatctgtggagtgagtgaacacacacacacaaattaatataaaaccaagcggtaattctgctaatccatcaatcactaaagcacagcagcaattaatacccagaaccaactaaagaacactcaaactatgcactgataccagagcttccttcccctgagcatggaaagccttattttgtgtaccacatgcacacagtaagagctctgcaatatgatgacatggactgctagaccttgagggcagtgggagccagtggtggtcaccaccaactttgggtacaaatccaagaactgccatttactaactctgtgactttagacaagtcccttagtctcctaagccccagtttccttcatttaaaaaacaagagaacacccaagcctggaaatctgcagccctaaatgggaataggcattcctgttttcacgcccaaatgttaggttttggcctgccacatcccactatcctgtacccatataaaccccaaaccccaggctccatgagcatacaagcagatgagcagatgaacagaagagaagaggagcagaagagcagcatggcagagaaaggagcatctgaaggccaagaagagtttggctggggacagttggagaggagattggtcacaggacagccaaactccaggggaagatcatcttcccactccatcccctttctagctccccatccatcccaccgagagccacctccattacccaataaaaccccacattcaccaagaaaaaaaaaaaacgggagaacaagataatgcatggagggataatcacccaaggtagacacaaatccaattgtgatacttctttgctcgatgtcacacgatgcctccacccagagtaaaagccaaagtcattagggtttcctttaacagtcactacatggctggccttgtctctcactcccctccaaacacactggcccttacctcccactcatggaagtggtcctgttgcactctctgcctggaagactctaattgaaaatatccacatggcccactctcccttgggtcttcattcaaatgtcaccttctcaataagatgttccttcatgattctcttttcacatgacagtcccctctcaacactcgtggtgcattcccttgctttattttccttcttggcacttttcatacaatataccatatattttacttatgtatggaaatttattgtcttgctaccctccatttgaaagtaagctctttatttacaaaattggtgcttaacgaatatttgttgggtggatgaaagcaagcactgactgtcaactactatcactgggggtgattaactttgtctcctcatgcctggccccagtctgcacttagtaggtgcatggtaataataataaaatatctaacacttggacaggcatggtagctcacatctataatcccagcactatgggagaccaaggcaggaggatcacttgaggctcagagttcaagatcagcctgggcaacacagtaagaccctatctctacaaaaaaataaaaaattatccagatgtggtggttcatgcctgtagtcccaactacttgtgaagctgaggtgggaggatcccttgagtccaggaggtcgaggctgtagtgaaccatgattgctgcactccagcctgggtggcagagcgaggccctgcctctataaaatcaaattttaggccgggggcagtggctcacgcctgtaatcccagtatttcgggaggccaaggcaggtggatcacctgaggccagcgttcaagaccagcctggccaacattgtgaaaccccgtctttactaataatacaaaacttagccaggcgtggtggcacatgcctataatcccagctagtcaggaggctgaggcaggagagttgctgtaatctgggaggtggaggttgcagtgggccgagatcatgccactatactccagcctgggtgacactccagcaagactccatctcaaaaaataaaaaaaatcaaattttaaaaatatataatacttattaaagatctgctacatgccaggcattccggtaaacatgtttctgggtttaaacccattaattctcacaataacccagtgaggtagagactttcattatccccatttgataaaggatgaaaactgaggcacacagaggttaaagagcttacccaaagccacacagccagtaagtggcagactcaggagtgaaatgtagccagcccggccctgtcactgctatgttaaaccactaatccatgttggtcctctaagtcaatcctactgaaatgtttgttacacatattcacgcattaaacctactagcctgggtatggagtatgggacatgattccaggtcactttacaaaagtgtaactcttttttttttgagacagagtctcgctgtcacccaggctggagtgcagtggtgccatctcagctcactgcaaccttcacctcctgggttcaagcaatcctcgtgtctcagcctcctgagtagctgagactacaggtatgcaccaccacacccggctaatttttgtgtttctagtagagacagggtttcgccatgttggccaggctggtctcgaactcctgacctcaagtgatctgcctgcctcaacctcacaaagtgctaggattacaggcttgagcaaccgcacctgaaccaaaagtgtaattcttttgcttatagattttgtcattctattgctttgcagacatttcatccagttccctgcataaggaggcctcttgatgtcagggaccctgcccaaaacattaattactatgtcagcaatggctcagataccctgaaaacactcaccaggcatagggtcatagctggggatcttatcactgctgttccgaacagactgaaagacagcccagaggaattctttagtgggcagggtcctggccatcttggaagctgcttgcagaaggatctcaggaaactgtgtgaaagaagatgaaagaagaggaataataattaaaaccccttaaatacagattgaaattagagttgaaactgtcacactacttactgccttcattctttgttatagcaacttctcatataaatctattccctagtcccactaatgtggtctctataaagaatcctataaaatgatactcaacttcaataaaaatccaataaagtctaaaaaacataagtaaatcacttttcacccatcagattgtcaaaaataaaatgaagtgctaatacttaatgctgggcacattcatttttccgtgggactgtcaaactgtgcactattagcagagcaatctggtaatatcgacaaaaaatgtaggttaggcgaggtgtggtggctcatacctgtaatcccagcattttgggaggtcgaggcaggcaaatcacttgaggtcaggagctcaagactggcctggccaacatggtgaaaccccatctctactaaaaatacaaaaattagtcagtcgtggtggcatgagcctgtcatcccagctacttaggaggctgagacagaaggatcaccagagcccaggaggctgaggttgtagtgagtcgagagcatgccactgcactccagcctgggtgatgggagtacaaccctgtctcagaaaaaaacaaaaaaaatgtatgtgtacctaccccttttttggtttggtgctatttctaagtttttactaaactttatatttgacatagtttggatgtgtaccccacccaaatcttattgaaatgtaatccccagtgttggaggtggggcccagtgagaggtgattggatcatgggggcagatttctcatgaatggttaaatacaatccccttggtactgtcctcacaatagtgcgtgcgttctctcgagatcttgttgtttaaatgcatgtagcacctcccccatcacgctcttgctcctgtgtcggccaagtaagatgtgtctgctcccccttcgccttccgccatgattgtaagtttcctgaggccttcccagaagctgggcagatgccagcatcatgcttcctgtacagtcggcagagccatgagccaatcaatcctcttttcttgtctctttttttttttttttttttcagagtcttgttctgttgctcaagctggaatacagtggtgtgatctccactcactgcaacctccacctcccaggctcaagtaattctcgtgcctgagcctcctgagtagctgggattacatgcatgcagcaccaagcaggctaatttttttttatttttattgtagacaaggtttcactatcttggcctggctggtctcaaactcctggcctcaagtgattcgcccacctcggcctcccaaagtgctgggattacagatgtgagccactgtgcctggcagtgtttgtttgtttgtttgtttagacggagtctcgctctatcacccaggctggagtgcagggcacgatctcggctcactgcaagctctgccccctgggttcacgccgttctcctacctcagcctcccgagtagctgggactacatgtgcccaccaccacgcccggctaattttttgtattttttagtagagacggggtttcaccctcttagccaggacggtctcaatctcctgacctcgtgatctgcccgcctcggcctcccaaactgctgggattgcaggcatgagccactgcacctggccctttttattttattttttttaatttaacttttattttaagttcaggggtacatggcaggtttgttatataggtaagcttgtgtcatggaggtttgttgtacaaattatttaatcacccaggtattaagcctagtacccattagttatttttgctgatcttctccctcctcccacctttcactctccaataggccccagtatctgctgttccattctatgtgacaacgtgttctcatcatttagctcccacttataagtaagaacatgcggtatttggttttctgtttctgcattagtttgctaaggatgatggcctccagctccatccatgtccctgcaaagaacacgatctcattcttttttatggccacatagtattccatgtaaacctcctttctttataaattacccagtctcaggtatttctttatagcaatgcaagaattgcctaacacaggtcaggtgcggtggctcacacctgtaatcctagcacttgggaggcccaagcaggcagatcacctgaggtcaagggttcaagaccagcctggccaacacggtgaaaccccgtttctacaaaaaatacaaaaattcactcggcatggtggtgtatgcctataatccctgctactcaggaggctgaggcaggagaatcacttgaacccaggaggtggaggttgcagtgagccgaaatcgtgccactgcattccggcctgggcaacacagctagactccatctcaaaaaagagaaagaaaaaaagaattgcctaacacaacatttgtcatgaaaaggaggtgaagaagggttgtctcaacatcctgggtggagactagcaatgaggggacttaggggactttgggttgccttggagaaagtgccatttctcatctagaacgaatacactttgtcaagacttgggattttattagaaggcctgcccatgggcccagataatccatcaggctctacaaacagctatgcctcactgggtcccggctcacccagagagcaagcctctcagcctatgtagctcccctttctagtctcatcttcagaattagagtcacagtctaagccagcagttcttcaaccctggctgtacctaagagtcacctgggacacccagagccagcccctaaagattctgactcagtcgggctgggctggataccatggattggtccctcttggaatctccccaggtgattctaatgtgcagctgaggctgaaggcaaccactctaagacagttaacttttagaaagcaatatgcatagcggagtgaatggcattttccattttgtattttctaaagaagagtgtgtatatgctcttgtgtttttagtagagacagggtttcgccatgttggcgaggcttgttatataggtatttttatacctatataaaacttgttatatagatattcaggatatttctgaaaggatgcagaagagattggggacagcaattgcctctgaagagggaggctagggaactagaagtctggggtgggagggagacttgcttctcatcctattacctttggtgcgattgggatttgttaaccaggagcatgtattacttgctttattaaacatttccagtatttaaaaaaagagtatacagaataatacaacaaaacacccatgtcctcacaacccaacttaagaaataaaacatcacaatataaataataagtccctccttcactctttgccttcctccttccccagaagtaactgtcactctgaatttggcattcacttttacacttctcttacatataaacttggcttacatttgctttaaattaattcagctactatttatttatttactttgtatcctactatagatattcttttgcaatctgggtttttttttaactctacactgtgtttttgagatttcacagtgttgatgcaagactttttcaatttcagaagttgatttttttaagattagatagatatatggatgttctcactgatatgtgggagctaaactatgaggacccaaaggcataagaatgatacaatgggctttggggacttggatggaagagtgggaggggccaagggataaaagactacaaatatgttgcagtgtatactgttcaggtgatgggtgcaccaaaatctcacaaatcaccactaaagaacttactcatgtaaccaaacaccacctgtaccccaataccttatggaaaaataaaaataataataaacatctaaacataagaaaaacaaggaaggaaaaaaaatagatggatatataacatttgtgcatcagggccgggcatggtggctcacgcctgtaatcccagcacttgggaggccgaggcgggcagatcacttgaggtcaggagttcaagaccaggctggccaacatggtgaagccccatatctactaaaaatataaaaattagctgggcatagtggcaggggcctgtaatcccagctactcgggaggctgaggcaggagaatcacttgaacccaggaagcagaggttgcagtgagccgagattgcatcactacactccagcctgggcaacagagggagactccatcgcaaaataaaataaaataataaaataaaaataaacatgtgtgcatcaactgcttataactatatattcagacatccagaatatgattttactgtgactggacttcagaatgtgctgcgtgtgatcctaggtgaagttgtgtgtgttcaggccctgctgagcatgtgtgaccatgtgcaccttgtgcctgcaagtgcaggtatgagagtgtgtggatgtgctttgtgggggatctgattgtcattcagcaaacattcactcctttcctgccctccacctccatggaaggagactccttcctgccccattaaagttggtcttggtcatgtaacttactttggccattggagtgtggcagaagtgacagcgtgccaacttccaacctaggacttaaggagaattgtacttatcccttcccttttttggtagtttcataccttcatggtaagaaaaacaagttctagagcagggctgccccagcgacccacaaatcaggcagcaaggaacatatgcgaattgttctatgcccttgagattgtgtggctttgttatgcagcacaaatgactaattcatgcttattttgcatgccccttggtctagactgtctaacttctggagccttactcctagaaaagcgtcatgcccaaatgtaatgatcaataaagacttgttattggattaaatgtgcttgcacatgtgattgcatacactggacatacatgtgcatgtgcatagggcaccagccagtgtgagagccagaacagacgtgcgtgtgagatgcacactaatagcagagtggtagctaagtaatatctgtctgcacacatctgcctggggggccacacaaaagggcctgagttcatttagctgtggactcactcccttttccagaaccttgcacatcctggaatggagctggaaacatcttagcccttggaggcagggaggaagcttccagaaccatagacagcagtaaacccaatgctgtataactgaccacactttctctccccttcaaactccttcagccttcttaagatggagcacaacattacctttgtggctatagagcttaattcatctcctgagaaaagtactagaaagggtcccaagtccttcgtggtctcggctgtccagtgctgagggagtctaaaaagagataataaccagtaaagtgaaaaaacatgctgtggtggacatctgttgccttttcccccagcacccttttctttcaggaatagattgtcttatactcatgtcaatcacatggtcccacttccttgaccaagaaaattggcatgtgatgcaggctgaccaatcagagtcatccctgggagttttgatggaactatcagagaagctctcctttctctggtatctctggcagtaggggaggatattggaggacattcatattaccaagtggaaaaagtaaagaccacaccaaggaacacagagctgagggatgggaggaacatattcctgaagatatcatttgagacacgggattcagccatgcctgaagaccaccagtggagtttcccgttacattccattcctgaattcaatacattcccgttctctttagtttgaattaaattattgaatttctgccatttacatcacagtgtgtccttcttctccctcctcaatagaagagtaattatatatttcttccttttactttaccataacagtcttctcttagaataagaaaaaacctttctcttgaacttggcaggataaaataaaggcactgacccagaatccactgttattcttgtatagatcataaatgcctacagtgaagagcattacactatctttggcgacatctctaaaggaggtctgcccaattagcagtgacagctggtgggaatgcaaaatcatacagccactttggaagacattttgttggtttcttacaaaagcaaacatgtttttgccatataacccagcaaacacactctttggtatttacacaaaggagttgaaaacttacgtctacatgaaaacctgtatatggatgttgatagcagctttatccataattgccaaaacttggaagcaaccaaatgtccttctgtaggtgaatggctaaataaactgtggttcattaagacaatgaaatatgattcagcactaaaaagaaatgagctatcaagccaaaaaaagacctggagaaaacttaagtgcatattactaagtgaaagaagtctatctgaaaaggctatctactgtatgattccaaatatatgatattctagaaaaggcaaaagtatcagtggttgccaggaattaggagtgagagaggaatgaacaggcaaagcccggaaggatttttagggcagtgaaaatactccgtatgatactataatggtgaatacatgttattatatacttgtctgaacccatagaatgtaaagcaccaagagtgaatcctaatgtaaaatatggactatggatgataatgagaatccaatgacgataatgtcaacataggttcatcagttctaacaaatgtacaactttggtgggggatattgatcatggggagcttatgcgtgtacggggtcagagagatatgggaaatctctatcttctccatttttctgagaacctaaaactagtattaaaaatagtctctagggtcaggcatggtggcccatacctataatcccaacactgtgggaggcttaggtgggtgaatcccttgagcccaggagttcaagacccacctaggcaacatggtgaaattccatcccttaaaaaaaaatacaaaaattagctgggtacagtgatgtgcacctgtggtcccagctacttgggaggctcaggtgggaggatcacctgagcccagggaggttgaggctgcagtgagccattactgtgccacagcactccagcctgggtgacagagcgagaccccatttaaaaaaaaaatagtctttaaactaataataataccactaccttgcatctgtaaagggccaccttttccaaatttccccttcatatgccaagctgtgtaagaaacaactctttgagatttttagggcagctactattgattccactttacagcaaatctgaagccaaggccaggcgcggtgactcacgcctgtaatcccagaactttgggaggccgaggtgggtggattacgaggtcaggagatcaagaccatcctggccaacatggtgaaacccagtctctactaaaaatgcaaaaaatagctgggcgtggtggcacatgcctgtaatcccagctactcgggaggctgaggcaggagaatcgcttgaaccagggagtcagaagttgcagtgagccaaggtcgtgccactgtactccagcctggccacagagcgagactccgtctaagaaaaaaaaaaaaaatctgaagccaaaagaagaaaggtcacatttccaaaataagcataagaattttatctcatcctaagccagagactctgtttgcagggcaggagagccaaggttgagtgtccctccacagagcataccacaccaccaaaagccaaacccagccgctcctcacccatactgtcccaggagcttgagcctcactgcagccttttgctcagggttgaggtctgggcagtctctgaggccgtgtagagcagtcgcccaagccctgggggagatccccctgtcgatgatggctgccggcaagtgacacagcaggttccccatgatgtccacagtgtactcatcagcaatggagtcgtcctgaacccaaaaagtggagccaatgggcagttccaacagagcataagatcgtgagccattagaaacacatcctgtggggtgggcactgtggcttatgcctataatcccagcactctgggaggccgaggcagacagatcatttgaggccaggagttcgaaatcagcctggccaacatggtgaaaccccatctatactaaaaatacaaaaattagccaggcattgtggcacatgcctgtagtcccagctactcgagaggctgaggcacaagaattgcttgaacccgggaggcagaggtcgcagtgagcctagatgatcatgccattgcactccagcctgggtgacagaggaaaactctgtctcaaaaaaaaaaaaaaaaaagttccgatcccaaacttcttaaagattccaggatcttaactgcctaccctgccatggattcctagagatggaacaggacctgatcttccccagggatgcaggcctcagaaatctggaaaaccctggcttggcacaccaggagcactcagtaattgagcgctgctgtcactgttattgctctgagaatattccagacttcaaccaccattatactattggcaccacaacaatgaaaacagcacttcaaagtttgtatgccacttgaaagccttcaaagctcttttgcactttcacaaaatgacaaaaatagttcacatttattgtgtgtttcctatgtgcgggcaccgtcctgagtatctgtgggtctcaatctagtcatcttcaaagtaactgtaagaagtaagtactactattatccattttttaaagaaaaagaaactgccagctagtggcaaggctaggattcacacccagacaatctggcctttgagcctttacattaaataatacattgcccatgtcatgttttcaatactctatgaggagactaggtatggtggctcatgtctgtaatcccaacactttgggaggccgaggtggatggatcacctgatgtcaggagtttgagaccatcctggccaacatggtgaaaccctgtctctactaaaaatacaaaaaataaccgggtgtagtggtacacacctgtagtcccagctacttgggaggctgaggcaggagaatcagttgaacctgggaggcagaggttgcagtgagctgagatcgtgctactgcactccagcctgggtgacagagtgagactctgtcccaaaaaaaaaaaaaaaaaaaaaactctaagggggatgtgctactccccccaccttacagatgagaagactgaggctcagggaggaaaggtgattcacccaaagccacataaccaatcactggcacccagtctctgggtactgtgctctttccattcctccagagtagcctcccctcctttgggccgtggggcagaagtctccagaaccaatggaaaagattgttccaaccagcaactttagagtaagcttcaagacccaccagtcagacttctggccatcacagcaggctcaagatcctagccagatccctcacagggctccccaagatggccacagatacccagagggaggggacacccaggaagagttttcttaccaggcactgctgcactttcctgaggactgaagtctttttgtgggaatctaaaaccaaggagtccaaccagctcttccccaggctgatcagaaagggcacacactgggaggctgggacagaagccaggtagcgggccctggagagagatggggagaacaagaggaggaacacatggctgctcctgttggcccctaaaatcagggttcgcctgcccaagagctcatcccaaagacattctttaaatgtttgttcttcccagtgatgctttggctccctgcagcaggtagcaagcttcccacccatcatgctcctcttctatgggtgggaacaaacttcccatcatgctcctcttccatgaatgggcaacaaacttcccatcatgctctcttctatgggtgggaacaaacttcccatcatgaccctcttctgtgggtgggaacaaacttcccatcatgctcctcttctatgccccagagcattgatcagagaatgggttctcgtaaatagacatgttgattgatggactggttgaggggagatgaaaaagaaaggaaaaagtagatgaaaaaagagacaacgaggagagagaataactagaaagtgagaaagaaagataagaaaaagagtggaaagagaaattctgtcatctccagtccagaactctgccttccactcttgatccttcagctcttgacaatgtattctcaacagggtcaaagtcaccctcaagaaggagaaactggtttggatggagataaaaaagactgttatgacaacgatctgtaacccactgaagctcattcctactcaaaatatttttcctcagtgtataatttctttcattcaggagaaattaaaattttctcctttggaaggcattaacaaaagttgagaaagtctgcatgaaacccatccggcctactcaaccccaccacttcactgttccaaaaccagatacaacgtcttcccccaaacatgttcttccgcctctatgctatcccacactcctttttttctttatcccaagcctgtcattgctatctccttagcattatcccaatctgttcttacttctggccaggtgtggtggctcacagctataatcccagcactttgggaggccaaggtgggtcacctgagatcaggagtttgagaccagcctgagcgacatggtgaaatcccatctctaccaaaaattcaaaaaattagctgggcatggtggcatgcacacctggggtttcaactactctagaggctgaagtggaagaatcacttgaacctgggagacagaggttgcagtgagctgagatcgcaccactgcactccaacctgggtgacagaatgggaccccgtctcaaaacaacaacaacaaaaaaatctattctaacttctccattcccagtaccactgtcttatttatttcttcattcatccagcaaatatctacaaagcaactacagtaagccaggcactgttctaggcaccaaatataacaatgaacaagacacaagtggtccttaattctagcagagagaagaaacacagaaaccagtgaacaaaacaagacatttgagctattaatagctggcatggtggctcacgcctgtaatcccagcactttgggaggccgaggcaagcagatcatctgaggtcaggagtttgagaccagcctggccatcacggtgaaaccctgtctctaccaaaaaatacaaaaattagccaggcgtggtggcgcacacctgtaatcccagctactggggaggctgaggtaggagaatctcttgaacccaggtggcggaggttgcaatgagccaagattacaccactgcactccagcctgggtgacagagggagactctgtctcaaaaataaataaattaattaattaattaattaatgtaaggaagaaaataaaatggctctgtaaaagagaatgacaatggggataatcaaacgagccccttctaaggcagttccatttgagctgagacatgaatgagtggtagaagtgccatgccaagatctgggttactgaaataatcttcagctgtctcccttctttccattcagcctccctgcattctatcctctatgtgggtagtggctatgatctttctgaaatataaatctgatggctgcacacagaatatgtgtgaaacacctcactttacaaagcccccatgatctgatgtcccttaactctctggccttctctcccctatctactctgcagatatacccccatgtgttcccagagtgattggcttttcttacccactgctgaaccggacatccccattccactgcctggaatgtccatcttaaactctctatccagcattagttcggtctcccctgtctctccagtctatgggtgatattccctcctctatgtccagtcctcattatccctcccctttgcctctcctttggaaggcaataaaaaagttgagaaaggctgcatgaaacccatctggcctagtcaaacccaccacttgactgttcaagaaccagattcaacatcttcccccaaacatgttcttccacctctgtcctaacagaggtgtgtaacagcacctggctttcttggaaagcacctagtttttcttggagaaccatctccctcactctaagtccttgtggtttctctccagctccaggggtgcaatacagattgaagtcagtcaaagttacccatcctcccaacctcactgattggttcaaggaaaacaaattcgcccaattaaggtcaatgagagtcaaacccaagatgtgtattgagaatgatgggaagagaacatttttctattttccctgaatatgaaactgggaaaatttaaccttagatcttccagggttcccacagaggaacaacttgcctgatagtgaagccaacagagaaaagctgggacaagagacaaatgacatcatttgcacccctagatcgagccatgcctgaagtccctctctgaactttccagttacctgaacaaaaaattcccttttattgcataagccagtttcagtttcagttctgttgctttcaaccaaatatcaacctgatataattggcttcatgtttgtctattccctctcccaccatgagattataaggtcttataaattaataggaatttctaaatcttcagatagaaaatttagctatctgagaactagcacacagcaagtactcaatgaacttttttttttttttgaatgaacgaagacaataagagcaaaaaaaggtagagggaaataaagaaggagagaaggagagaaacaatgtccagatcatgtttgaaaagcagggccaccctgcaggcccaaaagctcacacatgccaggagaaacgcctactgctcccctcaactctgattcccctggagcctggcacagccgcaaagccaggccagatgggacctgcctcactgacactcattcaggcttgggttgctttggcttggtttttagataacaggaaaagcaagaaggtctgtctcaaatgtctgtgtgatactcagaattgaaatcctggatctcaagggcttaactctctaaggcatcctccactctgcctctggttcctgaagaaaacccagtggggagagaatcattttgacttcagtgattcccaccgatctcactgatgagccagaaggtgggggctgatgttcacttacgggagtgcagccaagaggaaaggtggcatagacaatctggaaacttcccagtatttccacgccaaacaattaacctgaaagattaagcagttctggggatttaatgcagtaagaagaagaacaaacacagtgctttccatgtgccaggtactattctaagtgcttcatacgtgttcatttatgcaatagagcaccctatgatgtgagtaccactactgtgcccagtttatggatgagaacgctgaggtaataacaaacatgaacacggacatttaagtgtgagaatgctcatcgcaattttgcttatatctgcaagaaagggagatataattttaatgttcctcagcaggggattggttaagtaaattatggtatatacatacaatggaatagtgcacctactttgttttaataaggtagatttatatatatattaacactaaatgatgtctgtgctattttatgcatgtgcatatatatagttatattatatataatgtaaaaaccacaaatgggctcatattgtacacacaattctgttaaactatatatatagcttaactatacatatagttatatatagcttaactatacatatagttatatatagcttaactatatatatagttttatacatatatatatcttagctatatatataaacagaacagcatgtatagtatgagctcacttgtgttttttttttttgttttttttttgagacagtctcactctgtcacctaggctgtagtacagtggtgtaatctcagctcactgcaacctccgccttccaggttcaagcagttctcctgcgtcagcctcccgagtagctaggattacaggcaccccccaccatgcacggctaatttttttttttttttgtatttttagtgggtttttttggggttttttttttgtattttttaaaaatttttttaggttggccaggctggtttcgaactcctaacctcaagtaatccacccatctcggcctcctaaagtgctaagattactggcgtgagcactgtgcccggcccacttgtggtttttatattacatatagatacttttttaaaatttggtttagagatagagtctcactctgtcacccaggctgaaatgcagtgactccatcataactcactgccaccttgaactcccaggttcaagtgatcctcctgcctcagtttcccaagtagctaggactacaggcatgtgccaccatatttggctaatttttgtatttttttgtagagttagagtcttgctgtatgtttcccaggttggtttcaaagtcctatcctcaaatgaacctcccacttcagcctcccaaagtgctagaattacaggcatgagtcattgtgaccggcctatattttatatataaaagaaaactctcaaaagtaatagcaaactgttctttatagttacctctgaaacacagttgctcacctcagtattattgacattttcgaccaggtacctctgtgtagtggtgctgtcctacgcattgtaggatgttgagctccacccctggcctctacccactagaggccagtagctctgctccggttgtgacaaccaaaaatgtttgcagacatggccaaaagtcccctaagagggcaaaacagtactctgttgagaacaactgctctggggaaaatttggggaaattttactttctctgtattggttgtttgtgtgtgtgtgtgtgtgtgtgtgtgtgtgtgtgtgtgtgtgtgtaataattatgtgttttcagaatgggcgtggtggctcatgcctgtaatcccagcactttaggaggctgaggctggtggatcactggaggtcaggagttcaagaccagcctggccaacatgatgaaaccccgtccctactaaaaatacaaaaattagccaggtgtggtggtgcgcacctagaatcccagttagacaggagactgaggcaggagaatcacttgaacccaggggcggaggttgcagtgagattgaaccactgcattccagcctgggcaacagagcaaggtcctgtctcaataataataataatgataataataataataataataataatgtgttttagtaaaaatataaacgagaaaggcaaattttctaattaactggtatttgaaggctctgagagctggaagcctaggaaagcaccttcactggggcaactcttcctgctcgaacatgtaggtcttcctcaaagcaggtctagcttccatccatttgctcagttattggcttgcccacctgggcaggtcttttaatatagttcagtggtttgtaccagcaaactgattagaaatgcaaagtattaggcctcaccccttacctactatatgtaaaactctgggagtggggcccccaatttgtgtttttacagccttccacacaatgctgatgcaagctcaactttgagaatcactaacagaattaacagtccaagggaatgagagagcttcattaaaactttgcatattcctgtaatgatcttgaaggattatacaccaagcactctatgcttcctggttttctgggagataatttactctttggaaattcttcattctggtctgaaacacaaggccagagttgagaaggtgctttttaatatccattacaggagtctgtaagccagcggttcacaccaaaagttcaaatgctgtaaggcctgtgtttactagcttagacactgaaaaatcagtcactggctgggtgaagtggctcatgcctgtcatcccagcactttgagaggctgaggcaggaggatcacttgagcccaggaatttgagaccagcctgggcaacatatcaagaccctatctctgcaaaaaataaataaattagccaggcatggtggtgtgtgcctttagttccagctactcaagaagttgagacggaaggatctcttgagcccaggaggttgaggctgcaatgaaccatgattatgccactgtactccagcctgggtgacagggcaagactgtgtctcaaaaaaaaaaaaaaaagaaatcagttactgcttctagtctccccaaaggttaaatagcctccataaccttttagaaaatgaagttttaagtgagaaagaaaatgttctcagagtactgtttgcattcacagctactcctggaaagtgttggaatattagctgaaggagtaccttccatcccacgggaacataattatgaacacacaaaatactgtcccttatgaatatattttaaggcttaaatttgagtatggaataaccaatctgttgattctttggctgtagtgatgtgggtatttggagctgtttaaaaagaaactggaaaaaacctggctcaatctttggccctaagtcatcacttgcgtctgaatgattctggcataaagatctttagatgccacagccaatccaggcccagtgactggggtagggagagagaattagaagctggaaaatgctttaacggtacctgatagggtgaaagcagggcgaagttgttctgaaaatcctggaaatgggccagaaagaagtcagtgctcatggcatcaatgtgtgagcaggtcacgcctttgaccagctgcccagcactagaaaacaaaacaaggcactcgggagaggaaggaagtgaggaggagaggaaagaaagagcgagagaaggcagatatgatcagatacaagctacagatgtgactgcctaactctcccacctggaaaagtaggctgctctggcagaaggctctgaaaggcctgttcagcacccaggccgagttacattttcctacctcaaaagctcctcaggccttctggtggtctttaacagaagctcatacaggaacagagcctgcaagaagagaatgatcgttagcactgatggcacactcagcgtgtgcccaacactgtgaggccctatcaggaaggcattccacaatcccattttacagatggggaaatggaggcttaacaggctatgtgagttacctaggactagaccattgctaagtggcagtgcctggatttgaaccagtgtcaaggtaattccagagccccactctcacactccagatgattctctacaccccatgaggcagatgagaggcaaagacattggtggcagggctggagttttacttcctgtgctttaggaagctgctgaccagttgagaactccctgtgtctcctattcacatgtgcacctgccaggttctgaacaccaggcatcccagaataaaccaggatgggctagaaagcaccagcaataaagtacctgcagctagcagcatgcactgtcttcaaaccaccccccaatgagcactctagaacaatgcctagtagacaggggagtcatttgttttaaggaacataattttacagaatccctaacttgcagacgcaatcaagtactacatttgtctattttatagactctgaaactttgagtatcattccccagacagaataaattatgagctagcattagacatggtagctgagagccaaaaagcagcacaaaagcccaggcaaaaggaatggacgatggaggaaagttctggacactgtttgtggccccagagattttcctgggctcagggtcaaattctgggaccctcccatggtcttccccatctcaggaaacagcaatcccatcagctcaatggccgagacaaaagtatcttgaagtcttcggtggcagaatgtattttccaaaataaaactattggctgggtgccctggttcacgcctgtaatcccaggactttgggaggccaaggcgggcagatcacttgaggtcaagagttcgacaccagcctggccaacatggtgaaaccctgtctctataaaaaatacaaaaattagccgggcatgatggtgtgtgcctgtaatcccagctacttcaggaggtggagccatgaggattgcttgaactcaggaggaggaggttacagtgagccaagattgtgccactgcaccccagcctgggtgaccgagagagactctgtctcaaaaaataaaacagaataaaataaaacggttgccactgtcactaaatttagaggtggtttgtgacacagcaatagatcagcaggacatcatccagcctcatgagagaccctgagcaagagcgcctggaacagacagggatttgtgtcgtttcattcacttttgcacactcagcactgtgagcagtgcctggcgcactgttgggctgtgcttgataaacatttgctgggtgcatgtaatgaagactgactgaatacaaaggtagtcaacgatggtactaaggcagagcatccacttcaacagcaccatccccccaagcggcctcaatgctcacagcatttaataatacacataatagccgcgaatacttacagtacacagcccaggtggcatgtatcagtgaccctcattttattatcaaaattatccccatttcgcacatgagaaaactgaggccatataaggaacttctccaaagctaataaataagtgggagccagaattcatgctcaggtcttgtctaactttttttttaagagacagggtctcgctctgtcgcccagactggagtggagtagcatgaccataactcactgcattgcggaactcccatgctcatgtgatcctcctgtctcagcctcctgagtagctgggaccacagacatgcatcaccatgcctggctaattttttaactttttgtagagacaggatcttgcttgctatgttgcccaggctggtctcgaactcctggcgtcaagcgatcctcctgcctcagcctgtccaaattcttaacactatactattctgcctcctatactaatcccacagaaataaatttcttttatcaaattaaccttaaaacagaccattcattctcacaagacagatagtcagaaatacaggatcgatctgtgtttcatggtaatacctggctccttccaagttccttatccttcaggactgtagagttgaatccaggttgcctccttaaatcaaagagagacacttccttaaagaaagccccttgtatctccacgatgcctggggcagtgtcttccgcttggaccatctgccagaagcgagaagcaacaaaacaacattgtaaaaaatgcattgagctttgaggaagggccaggcactacatcacaggcaataaaatccatcagaaccgctcagcaaccctaggaagtggagagtagcatcatccccatttcacaggtgaggaaacagagacttaaagtgtgatgagttgaaagtcagtaagtgacattcaagcccaagtctgtctgattccaaaacccgtgctccttatttctttattttaactgtatgggctacttgctatctgcaaggtattggtgttttaggccaaaccccttaggttttagggtttttttctttttttgagatgggaatctccctctgtcgccaggctggaattcagtggcatgatattggctcgctgcagtctccgcctcctgggttcaagcaatttccctgcctcagcctcccgagtagctgggactacagaagtgcactaccacacccggctaatttttcgtatttttagtagagacggggtttcaccatgttggccaggctggtctcaatctcctaacctcatgatccgcccaccttggcctaccaaagtgctgggattacaggcgtgagccacctcacctggccaagttttggtttcttaacagattttgccattggacagaacggacctgatagagcaagatgtcaaaagactccctgacaagtaaaaaaggggccaggcatggtggctcacacctgtaatcccagcactgtaggaggccggggcaggtagatcacttgagcccaggagtttgagaccagcctgggcaacatggcaagaccccatctctagaaaaacaaaaattagtgagcagcacaggcctgtagtcccagctacttgggaggctgaggtgggaggatcccttgagccccagaggtggaggctgcagtgaaccaagatcacgccactgcatgctggctggggtaatagagcaagaccctatctaaacaaacaaacaaaaaaacagaatacggacatggctgtggaccatgaaaagggctttcagatgcaccctaggactttgggtttatttttagatttgaaaaacaaattttaggccaggcacagtggctcacacctataatcccagcactttgggaggccgaagcagaaggattatttgaggccaggagttggagaccagcctgggcaacatagcaatactccatctctacaaaaaattaataaattagccagacatggtggcttgaggccaggagtttgagactagcctgcgcaacatagcaagaccatgtctctacaaaaagttaaaaaaattagccaggcatggtgatgtatgcctgtggtcccagctactcaggaggttgaggcaggaggattgcttgagcccaggagactgtggtgagctatgatcataccactgtactccagcctgggcaacacagaaagactgtctcaaaaaaaaaaaaaaaacttgaacctgaagtgttataagatcaggaaacctgatttaagaaaagtcttccagattagactgtctggcagaacaaaggggtctgggaagaatgacagcatgactgaagggctccgtctggaaaggaaggaaggtgtgccaccaggagaaggcagagccaccccagacaccaacagctgagacaatcccagccctgggttcatggcccaaagtcacagcccactcaccaaccccaaaaacataccccctgtgacatgtggctgagcaccagacatcttcctctcaccttgctgaggataccttgctgctgggcaggtgacaagtcggatacatactgggagacggcacttctcaggacctgcgagatgtccttgcgtttcatgctgcagaaggcctgggtgctgaccccagccagcagtgcgcccatctggctgacattgtagaaagacagcacctggaagaggagcggctgcgcagtcaggctctgctcccgcctttcacctctccaacgtgcactcagcccatctatgtgccaagtatagggatgggtgacaccttaggggcacagcagtgagccagacagatgctgcctccacaggccttccttccttctatcaagaaagagagttggccaggcatggtggctcacgcctgtaatcccagcactttgagaggccaggcgggtggatcacctgaggtcaagagttcgagaccacctggccaacatggtgcaaccccatctctactaaaaatacaaaaattagccaggcatggtagcaggttcctgtaatcccagctacttgggaggctgaggcaggagaattgcttgaacccaggaggcagaggttgcagtgagctgagattgtaccattgcactccagactgggcaacaagagcaaaactctgtcagaaagaaaagaaagaaagaaagaaagaaagaaagagagagagagggagaggaaagaaagaaagaaagaaagaagagagagagagagaaagaaagaaagagagaaagaaaggaaagaaagaaagagaaagaaagagagaaagagagaaagagagagagaaagagagaaggaaagaaagaaagaaagaaaggaaggaaggaaggaaggaaggaaggaaggaaggaaggaaggaagaaaggaaaagaaagaaaggaaggaggaaagaaagaaagaaagaaaaaagaaagaaaggaaagaaagaaagagagagagagaaagaaagagaaagaaagaaagaaattatacttgggttttttttccttcctttagagtgaagatgctagatagttttccatataatcaaggacatgctttggttctatgaaaagacagctgagtgggttcctttagttattctgtgtataatgggtgatctcatgctgtcttcaaagaaggcaagaccttttgacctcttgccttctgagtaaaagtggcctcacccctcagtaggaagagttgggtattaggaaagagacaaaccactttgtcctgggctgggagggaacaaaaccgtctccctcaactccctaaaatcaaattcagagaggactgtcaaggtggacccatggagccccagtcaaggtccagaaacaaggattcaaagccttcaacataaagtcaccacgaggctagaagagaccagatgaatgggctggcctggtacctgagtcagaaagtgggagtgcgtgggcattggtcatggtgccataatggagacagtgagcacaggagttaaacaagatggctctgaggccaggtgccctgggttcaatcccagctgcgtaactttcacgtggccttttccagttcccttacacactctgtacctcacatgaatgaactggaaaatgaagactacagcactactgacttcagaggattgttggattaagttattaattcacttagaacacaacctggcacatagtaagtgttcagtaaatgtttgttattccacaccctccctcccttggccccgcgatggaggaagcaggctaggaccagccctcggagctgcagctgcccttcatccctccctcgccctctctaccgacatcctgctccagttcccacttggatttactttggggaatgtggttggaatatatctggctcagaaccatgacataccaaacccagctttaaaaacttgaagaaatttaaaaagacttggatgccaagaagaaattccctactccttctctttgggaggccaaggcgagtagattgcttgagcccaggagtttgagatctgcctgggcaacattgcaaaatcccatctctacaaaaaaatacaaaaatcagcccggcgtagtggcatgtgcctgtggtcccagctactcaggaggctgaggtgggaggatcacctgagcctggggggtcaaggctgaagtgagccaagatcagatcacttcactccagcctgggcaacatagtgagaacctgtctcaaaaaacaaaaaaagaagaagaaaccccccagttcctgaggccaactccagcactgccttctggatgcatggattctactctataactcttaaacccctttaccgcctgaatcaaaagcttttgttttcattttaccacctgaatcaaaagcttttgttttcatttccaacctcagtgatgcgatctcggctcactgcaagcaccgcctcccaggttcacgccattctcctgcctcagcctcccgagtagctgggactacaggcacccgccaccatgcccagctaattttttttttttttatttttagtagagacatggtttcaccgtgttagccaggatggtcttgatctcctgacctcgtgatctgcctgtctcggcctcccaaagtgctgggattacaggcgtgagccatcgtgcctggcctagtgattttcttccttgtgagacactgtggtattttgctttagaacaagcaaaatggggccccttacattttcaacatcttcacctcttcccatatcctccttcaagctgcatgggaggtactaacactagaattacggtgctaggttagtaaacatgacctttaaggagtagtctctcctttattctttgggattcctactacttttttcttttttttttttaagacagagtcttgctctgtagctcagcccagtctggagtacagtggcatgatctcggctcactgcaacctcctctgcctcctgggttcaagtaattcttctgcttcggccttccaagtagctgaaattacgggtgtgcaccatcatggccagctaatttttctatttttagtagagacagggtttcaccatgttgtccagctggtctcaaacccctagcctcaagtgatccattcatcttggcctcccaaagtgctgggattacaggcataagccaccatgcccagcctactaccttttgtcaaaataaaaattgatgagttttgtatagttggtcagacacagttaaaactaaattcacagtttagcaattataatgggtgcttgttaaacatctggtagtaaattatgttgtttcaaagtaattaaaattaatatccagaagccaaaaataaacaaatgtttgttattattatttgattggaatgggtcctaatccaatccttgtgagccagtttgcatcctgggaagtggccaggagtgtggactaaacgagaaggacaccaaaagccaggcacggtggctaatgcctgtaatcccagcactttgggaggctgaggcaggcggattacaaggtctggagttcgagaccagcctggccaacatggcgaaacctcgtctctactaaaaacacaaaaattcgctgggtgtggtggtgggtgcctataatcccagctactcggaaggctgaggcaggagaattgcttgaacccggaggcggaggttgcaatgagctgaggttgtgccattgcattccagcatgggcgacaagggcaagactccgtatcaaaaaaaaaattagatgacaccaaaatgcttagaactcaagtctcccagatttggagccctctggactggacccaacccagggacattaattgtccccaaaagaatgtccattttccaccccaggagcagcaagaacacagattaagactcctgtccccaccaaagaggactgtctgcctgacagttccctctctgcagcccaagattggaataaaatagcatcatccctaatcccaaagtaatggcaaaaaccgcaattactttttgcatcaacctaatagcagcttaacagcaggcaggaaaatcctgggatgagtcccacacgttgatcccactcagggatggctaatgccccttgagctcctactctgtggtgtcacctggtggtctagcttcttcatgtgtaccaactcacataatattcgcagcaatgctacaggataagtgctactgatttgttgttgttgttgtttaattttttttgaaatggagtcttgctctgtcgcccaggctggagtgtggtggcacaatctctgctcactgcaacctctgcctcccaggttcaagccatcctcctgcctcagcctcccaagtagctggaagtacaggcatgcaccaccacgcccagctaatttttgtatttttagtagagacgggatttcaccatgttgatcaggctggtcccaaactcctggcgtcaggtgatcctcccacctcagccttccaaagtgctgggattgcaggtgtgagccaccttgcccggcagtaagtgctattgttgtccacatattacaaatagagaaactgagtcaaagtgaaaatagcaaattgcctagtgtcacacagctagtaaatggcagagcaaggatttgaacccaggcagtcctcctcagcatcatcttcttactgattggttgccccctgctgtgtgtgtataactgattgttatggattaaatatttgtgtctcccaagaattcatatgtcaaagtcctaacccctaatgtgatggcattgggaggtgagacctttaagaggcctttaagggttagatgaggtcatgagataatgagggtcaggtggggccctgatccaatgggattagtttccttgtaagacgcaacaccagagagcttgctctcatgtggtcaggtgagcacacagcaagatggcggccacctacaggccaagagaagaggcctcagaatgaaacctacctttctgacacctcgatgttggacttcccagtctccagaactgtgagaaataaatttatgttgtttacgtcactcaatctacagtatatttttatggcagcctggcaggtctaatacacttaggtcatcttaggggaataaaaaagagtttaaaactccagctgaccttctcatgggccaagtatttggcagacaagatgatgacctggctcttggcccaacctgagacctggttgagggtggagatggctccgtgcacagcctcgggggagagggattccagttggctgtaggtcaaacctacaactgcatccgacaaagaacccagggtctcattgagggtctggttctccatggcaatgtccaggagttctgctttgagctggaaggagagcaaactggaatgagtgttgacaggaagcagggcggtggctccctctgaccatatccagggctattaggactccacagcacctcccttgaccctgttccatcaaagcttttgggctcagaaacgatgaaggaaagggactcctgccatgcccagacagtgagacaccacacggaggacacctcttctggccttagctttcatgtgccaaaaattccctttgtacttcatttaaatattaatttgttattaaatcaaccttaatccatgtcagaaagaaatgttccttttataactacaccacactcttatagcatctgttgtcctctggtgaccttgaactctatatccatatatacaggctgttttcctttcattaaatagccaggggccttttcacgataatttataatctgtgtaaaaataacttttttttttttcttaagacagagttttgctcttgtcacccaggctggagtataatggtgtggtcttggctcactgcaacactgcaacctccaccttccgggttcaagcgattctcctgcctcagtcttctgagtagctgggattacaggcacccaccaccacactaatgtttgtatttttagtagagacagggtttcaccatgttggccaggctggtctcaaactcctgacctaatgtaatccacatgcctcagcctcccaaagtgctgggattagaggcatgagccaccacatccagccaaaaataactcttcacagtacaagaaagtaaaagtctcctgaacatcctaccacataaaaataaccatgattagcaattgggtgtatatgcttccaagatttcttatacctttataaatatagagacactacatagtttttacagagttgtaatatgaacagaagcacaatttactcttcttaaaattatctcttgcacctctttgcacatttctatgatcattttaataattattttattgcaatatgcctgcctaatgatagtaaagtgtgcgttacagctgctttcggtaaggaatgtgataaagtcacctactatacaatgagctctgtaacaaaaaacaagaatggttcatattttaacacccgaatttacgtaataacgtagtcatttcaggcaggtgcacaaaacgggtttctggcaatattgaaatagccactggggggcagcagagtgaagtagaagaaacaactgtcaaagcgcctgggttctctaagttcggcaactgccttacctagaaatcagtttccacatctgtaaaacgaaggggtggactacagtggcagctcccaaagtgtggagcacacccagcggcatctgcaacacctgggaacttgttagaaacgcagattgccaggctgctcccggacctcctgaatcagagactgggtggggctccgaaatccagggatccccagactccgggtcacagatggggaccaccgggaccctggcctgttaggaaccagccacagcaggaggtgagcagcaggccagtgagcattaccgcctgagctctgcctcctgccagatcagaagcggcattagattctcctaagagcaaaccctattgtgcactgtgcatgcaagggacctagtctgtgcgctctttatgagaatctaatgcctgatgatctgtcactgtctctcatcacccccagatgggaccgtctagttgcaggaaaacaagctcagagctcccactgatttcacattatggtgagttctatagttatttcattatatattacaatgtaataatagaaataaagtgcacagaccgggcgtggtggctcacgcctgtagtcccagtactttaagaggccatggcaggcggatcacgaggttaagagaatgagatcatcctggccacatggtgaaaccccgtctctactacaaatacaataaatagctgggcgtggtggcgtgcacctgtagtcccagctactcaggaggctgaggcagagaattgcttgaacctgggaggcagaggttgcagtgagccaagatcgtgctaccgcactccagcctggcaacagagcgacactccatcaagaaaggagaggagaggggaaggggaaagggatgggggggggagggggagggagggagggaaggaggaaagaaagaaagagagagagtgagaaagagaagaaagaaaggaaggaaggagggaagaaagaaagagagagacagaaagagaaagaaagaaagaaagaaagaaagaaagaaagaaagaaagaaagaaagaaagaaagaaagaaaaggaaggagaaggaaggtagggagggagggagggagggaggaagggagggaaggaaggaagagcacaataaatgtcatgcacttgaatcatcctgaaacaatcccccggccccagtccacggaaaaaaattgtcttccacaaaaccggtccctgatgccaaaaaattgtcttccacaaaactggagcgctgccacaagctgtatttgaacaggccctccctgtgatttagatgcaccctccagtttgagaacaactgagctagatgatcccaaagggctccctcgtatctaattgcatgccaccttctccttggtcagggctgccacatgttgagacccctgggccttaaaatacatttatcttcttatttttgcttgagcttgcttaggacggattcttttccttggaaacaaaaatatgcattcacaggctaggctcggtggctgacgcctgtaatcccagtactttgggaggccaaggcaggtggatcacttgaggtcaggagttcaagaccagcctagtcaacatggtaaaaccccatctctactaaaaacacaaaaaattagccgggcatggtggcatgcacctgtagtcccagctactccagaggctgaggcaaggcgaaggttgcaatgagctgaaattgtgccacttgaacccggaaggtggaggttgcagtgagctgagattatgccactgcattccagcctgggtgacagaatgaaactcaaaaaaaaaaaaaaaatgaatcttataaaaaaaaaaaaaaagatgcgctgaccaaaatagatgaccacactctcaaatgtcaaatgtgtttggggactttatggtgatgtgtgggaaactgctgtgaaattattgatggctttatcaaaatttcatttaatattattttcatgtcatcgtttcgattttaagcaaattgaaaagggccacaaaaagtggaatggaagggggtagaattgagacttgactggaaggaagaagagagagatggagaggaaggaaaatgagagagagcgtgtttgctcatctggtcttgggaactcataaccaccctgcaatccagtgttctcaatcctggctgcattgaaatcacctgagaagcattaaaatcaggccatacctcaggccacaccccagaacaataacatcagattctctgagggtggcacctggcagggatgttactaatctagaggcaggtttgagaaccactgctttatctcctgccgctccaggtgtgctcaccagagcagcggttccagaatcacctggaagcttgttggaagtgcagcgtcagctgggcgcaatggctcacacctgtaatccaaacactttgggaggccaagggaagaaggaggaagaagaggaagatgggagtcctgggcctcaaccaagagctcctgaatcagaatctgcatttaagatccccaggtgatctgtatgcatatttaagttcgaagtagctgtccctttcttttcttttctgttcttttttttagatagggtcttgctctgtcatccaactagagtgcaaagtcactatcatagctcactgcaggctggcactcctgggctcaagcgatcctcctacctcagcctcccaagtagctggcactataggcacatggcttttttttttttcttttgagacagagtctcactctgtcacccaggctggagtgcagtggcacgatctcagctcgccacaacctctgcctcccaggctcaagcaattctcctgcctcagcctcctgagtagctgggattacaggcgcatgccaccacgcctgtttttgtatttttagtagagacagggttttaccatgttggccatgctggtctcaaactgacctcaggtgatctgcctgccttggcctcccaaagtgctgggattacaggtgtgaaccaccgtgcccagccatttttttgtattttttgcagagacaagggtctcatgatgttgaccaggctggtatcaaactcctggcctcaaatgatcctcccatcttggcctcccaaagtgctgggattacaggcatgagccaccgcgcctggctgagacagctttctgattaaattttgatctccccagggtctgttcctttgctattttctgtctgggcttcgatgttacacttacctcagtatttgagatgccatctgctgtgactggacttaaatcccgactaatgctgtgtgttttctcccaaaggctaagctaggctgttctccccatttctctggctagtgacactgaggcagaggcctcagggccttgcaccccagctgtaccttctggacgccagcctggaagcccctcaggtggctgcacttgatcatctggtaaagcaggacttgagccacagtggcatccagcaattccaggtcattgtagaaacaaaccagcagccccagcctgtgtgagaagaggagggatggtggtggaggtgtaaccgcagaaccagcccattctggttcaattttgtgtaataaaatggtgagttgtttttcagttgccacggactcccaggttgaaggtcacataacctgaacatcctcagatgaaccaaatgtgcaaccacaggcggaacctaactgctcagacgagaccaaggaatgggggctgaattatgaagtggacaccacatggcatggtccacgatccaatcagaatgagtcctggcatcacctcatggcatgatccaatcagatcacacctcccagcatcaccttgtagcaagacccaatcagatcaagtctcattaccctccgcctataaaacctgccccagtccccagctcagagacacagatttgagcactgtcttctgtctccttggcagttgattcacagtaaccgtttctctctacaaaaacctagtgcttcagtgtttggttttccattgcgcatgggcaaacagacccagtttggttccataacagagcgctgagagctggtggacacgacaccccctccccagtccaaaccaatggggtcttgattatattaatagcagctaccacttatcaggtcttgcactgagccgtttacctgcattatctcattacagcaaccctacaaggcaggtgctctcattagccccatttcacagatggggaaactgagatttgatttgtccaaggcaagtcggtggagctgggacttaaatgtagatccatccacctatgccatctctccacctgggatgaataaagggaataaggaaagaaaggtgccggtagccacagaaaactccccattttccacattacaatcactaaattctagagctgatctcaccctgagggattctctgaggcagagatctgtttctaggatgccttctggtacccacccagatgatgcaaagatttcagaaggaaaacaaaaaagactggaggaaaacaatggaagggcagaagtgagattggaaccctggtataactgagagcaatgtttctaatcattaagagcattgattcaggcctactgcagtttgaagcccagctctgtgagatgtttgctctccaccaagttacttaacttctctgtgaggccgctatacacagttctgcagtgcacactctgtactcccatactgtacagagacagggtgcctctccctctctgagcctctaataaatgggaggttttctttgttcatcgtagcacatcataataaacatgagtgaaagaaagaggatgattgtgaaagttaaatgagaagtggtataccaactgatttgcatacatcctggtggaaaccaagcactcattcattaatgaatgctcttagagttagaatgaagactacctagttgaccacagtcagcctggctccaccctctcagagcgatattgacatattcagacatgcccacaggagagctgccaggtccagggaaaagatttgaaagtgtgtctcatgaggaaaagagatctgcttaacttcgagtaaagaagactcaggggaagtgagggctgaacgttaataaccacgttgagaataactattcacaacagccaagacatggaatcatcctaagtgtccaacacgtcagtggatgaatggattttttttttaatttggtggatacacaatggaatgctcttcagctttgaaaaagaaggaaattctatcatatgggaccccatggatgaacttggaggatcttatgctaagtgcaataagctaggcacagaaagacaaatactgtatgacctcatttatattatgaatctgaaaaacaaataaacaaacgtcatactcatggaagcagagccttagaatggttggtaccaggggctggaggtgggaagggagttggagagacttggacaaaggacacaatgtttcagttaaataggaaaataggttctggagatctattgtacatgctgactacagttaataacaatgtatcatatactcaaaaattgctaagagtagattttaagtgttctcaccgcaaaaaaagtatgtgaggtaatgataacttaattagcttgatttagccattccacaatgaatatatatctcaaaacatgttgcacaccatagatataatttttatttatcaatttaaaaaaataaattataaaagaaaaataggcttggcgtggtagttcatgcctctaatcccagcactttgggaggccgaggcgggcggatcacaaggtcaagagatcaagaccatcctggccaacatggtgaaaccccgtctctactaaaaatacagaaattagctgggcgtggtggcgtgtgcctgtagtcccagctacttgggaggctgaggcaggagaatcacttgaacccaggaggcagaggttgcagtaagccgagattgtgctactgcactccagcctggcgacagagtgagactccgtctcaataaataaataaataaaaagaaaaaatattattcattgagcacttactatgtgtcaagtgtgcaaatccctacaaagaccttaatgcagtaggcatatttttatctcccattttacagatggggaagcggaagtatagagaggtgatgtaaaaatagcaaagatcacaccattagtaagcagcagggaaggattaagccacgaagtctggctcgaggtcccatgttcttaatcatgaggctatttttgtgtgtgtgaaaccatagaaaaatgagacacatgctaataagtgaaaattactaaaggtaggtgaaacagatgtaattgtttcctatgagtacattccctaagagtggctcatttgaaaaaccttaaggtaatatgggatgggagcagtgtggcaataataagaccttgagagacggacacaagaggattgaatgatggcccagcacagtggctcacgctgtaatctcagcactttggaagtccaagatgggtggatcacttgaggtcaggagtttgagaccagcctggccaacatggcgaaacccagtctccactaaaaatacaaaaattagctgggtgtggtggcacacgcctgtaatcccagctacttgggaggctgaggcaggaggatctcttgaatctggaggtggaggttgtagtgagctggtattgcaccactgccctccagcctggacaacaaagtgagactctgtctcaaaaaaaaaaaaaaaaaacaggattgaatgggccaggcgtggtggctcatgcctgtaatcccagcagtttgggaggccaaggcaggcagaccacctgaggtcagcagttcaagaggtcaggagtttgagaccagcctgaccaacatggcaaaaccccatctctactgaaaatacaaaaaatttagctgggcatggtggcacacatctgtagtcccagctactctggaggctgaggcatgaggattgcttgaacccaggaggtggaggttgcagtgagccaaggtcacatcactgcactccagcctgggtgacaggacaagactctgtctcaaaaaaataaataaataaaaggaaaaggagggagtattgagctgtactcaccacctccttcccattgcccactcccttcctgacacccctggaatgtagagttcctgtccctcctcccctgctgggaatggttttagggaggatcacattacagtcactaaattctagagctgatctcatccaggggctccccagctaagagtctgataacaagtggttctcatatatggggacccatgagttatttccaacacttgactcaacttaaacggaaatcacacattcactttggaacaggacccagtcctgagtatttaaaatgtttcatttctgtgctgagagacagaattagcacttgataaggttgcataaaatgcctggcacacaggagatgctcagaaagcatttatcctttcacccagcttcataacctcttcataaaaaaagttgcagacacctctcctcacatgcacagagaaatatgggactattcaaagagatggaccagccacctcccttccctccctgggtgttttgctgctcagagaattctgatgcttagatcacatcttgggaaagggctccaaggcccagagctcatgcgcttgcctgtggatggtggaggtattcctcatgttaaagttggaggagctgatcctctccagaaacgcctgggccagctcaggtgtgatgtcatagaccatgtccagctgcttggtggcgttgtcatagctgataaacagcccaatctagttggtggacaaggacgagaatatcagtgaggagggtggaagtggcccagtgtggccccaccctggtggtctgcactgtgccccatcatggacacttggatacacctcctggttctcattgtcattgatgtctttttttcttttctttttttttttttttttgagatggagtctcactctgtcgcccaggctggagtgcagtgacatgatctcagttcactgcaacctccacctcctgagttcaagcaattctcctgcctcagcctccggagtagctgggactacaggtgcccaccaccacgcttggctaatatttgtatttttagtagatatggggattcaccatgttgtccagggtggtctcgaactcccagcatcaagtgatccacccgcctcggcctcccaaagtgctgggattacaggcgtaagccaccatgcctggcctcattgtcattgatttcttagtggtctgtaactgctactttagtttcctcctcaacctaactattctttaggaaagaattattttttaatatctgagaaactgggctttttaaaagctaatctttgcacatttatttctagatttgttatatggaggtcagagaatgtggtccacaaactttctgctttgaagaatcagaatttttttaatagatgaatgagtttataaatggcccttgggtgatggaaaagagtatgtgttctctttgtaagacacaaagtttggtgcatatatattcaacttattaaatatactattcagattcccaacattcttgttcctttttggcccacatgtccaagggtgccacgtgcaagtcttctactactacaggactcatatctatctattcttccttgtatttctagcagattataaattatatatataattttggggggacactgactcactctttttattttatttttgtttttatttttatttttatttttatttttttgagatggagtctcgctctgtcacccaggctggagtgcagtggcacaatctcagctcactgcaacttcagcctcccgggttcaagtgatactcctgcctcagcctcctgagtagctgggattacaggcatgtgcaaacatgcccagctaattattgtatttttagtagagacggggtttcgccatgttggccaggctggtcttgaacacctgacctcacatgatccacccgcctcagcctcccaaagtgctgggagtagaggcgtgagccactgcgcccggccaagacacagtctcactcttttacccaggcttgggtgcagtggtgtgatcatggcttattgcagcctcgacctcctgggctcaagtgatcctcccacctcagcctcccaagtagctgagacctcaggcacacaccactacacctggctaatttttaaatttttttgtagagatgaggtttcactatgttgcccaggctggtcttgcacttctgggctcaagtgatcctcccgccttggcctcccaaagtgctgggattactggcatgggccacggcagccggcacattctatattttgagacagtattatgctgtgctcaaaggggctcataaagttgtctctcctttgtggatcgtactctttgtccatataaaattcctttttaggctgggtgcggtgtctcacacctgtaataccagcactttgggaggctgaggcaaggcagatcacctgaggtcaagagttcaagaccagcctggccaacatggtgaaacctcgtctctgctaaaaatataaaaattagccaggcatggtggcatgtgcctgtaatcccagctactcaagaggctgaggcaggagaatcaattgaatctgggagaaggaggttgcagtgagctgagatcacgccactgcactttagcctggacgacagaacgagactccatttcataaataaacaaataaaataaaataaaattcccttttagcagtgttattcttgacttctcccttctctacatttttagcgaataaaaagttgcaaatgagtgcttgaccttagaaatttcctctagggggctcagctttctaagaatttaagctgttacctactcaaagcaactctacattgagagagtaaatggcagtggggtgcggggttaattctaggactgcgggttataaaaatcggaagacacttgaaatactcccagccccctaggtaaaagagagaaagctcgttctgcctctggtttcataataaccacacattgctgggtcaatgggtccttgaacaggccagtcatttgatgcaaacacttctctcactggccagaagagtcagggcacatatgacttaaaaaagaagaagaagaagaagaaaaattggaagcaaggcccacagtctcttcatgaatccctttgtgttggcagcattctttggagaccgcattcaagaatcatgatgcccaagggattttagtgtcattgggtcaacatcaaaaggagagggacaggttgactagtgagttaagtaatcctagaatgtgtgtatttctccactgtaaactctagaattaaaatctaaccattgctcatgctcaagtagctattaacagaagtagctataaatagaagtagctattaatccattttgcttttgctttgagttcatgatcccaaggatgggaaactttatatcttttgtccttggccttctccagtacaaaaatacccttaaatgatttttggttcatagccaacaatccctttaaatcatgtttgctcacatcagagatgtgatcttagttgtccacaattctttttcttttcttagcccttccacactaaaacaatcccatgactgcccacttgaaactttcttccaatagttttcttcagaggcccaaggtctattgggctgaggagggtacacaatttccaaggcaccttgactggctggatgaacattagagtttgagaatacaccttaccccaagtgtattagtaatggtgacatataaatgtacttttccaacttacttctataggactaattttcgtaatttcttcaaacgataggtgaaccatgtatctgcccaaaacccataagtgttccgcactgacccatgaagcagagtcatctacaaaaaataaagaaatgaataaatgagtacatacataaataaaagcctttacactggtatgtgatagtcaacagcatctttgctgtcaagacctacttcttgcattcattaattcgttattttgtttttcggggttttttgagacagaatctcactctgttgcctaggttggagtgcagtggtgtgatctcagctcatctcaacctccgcctgccggttcaagcgattctcctgcctcagtctcccaagtagctgggattacaggcacccaccaccatgcccagctaatttttgtatttttagtagagacagagtttcaccatgttggtcaggctggtctcaaactcctgacctcaggtgatctgcccgcctcttgcctccccaagtgctgggattacaggcatgagccaccatgcccagccacattaatttgttattaactcattcaacaatcatatacgtagggcctacctgtgagatcacacgtaggatgtgaacacagattcgcaccacctcagaccttgttactcaatagtgtggtctttggaattgcaacatctgtgtcacctggccacttgttagaaatacagattattctttctggccaggcacctgtaatctcagcattttgggaggctgaggcaggaggattgcttgaggccaggagtttgagaccagcctgagcaatatatcaagaccctcatcgctacaaaaaaaaaaaaaaaaaaaaaaaaagccagctgtagtcccagctactcaggaaactgagaagggaggattgcttgaaaccaggaatttgaggctacagtgagctatgtgctcactctactgcactccagcctgggcaacagagtgagaccctgtctctttaaaaaagtaataataattaaaaataaataaataaataagaaatgcaggttatttttaatacttaaaacccacaaaatgtagttttattttttcattcgattggttactaaataatctggaaacatgaggccagatgtggtggctcacacctgtaatcacgaaatcccgtctctactaaaaatacaaagaaaaattagccaggtgtaatcccagctacttgggaggctgaggcaggagaatctcttgagcccgggaggtggaggtggcagtgagccgggatcacatcactgcattccagcctaggtgacagagcgagactctgtctaataataataataatgataataatctggcaacatgaaatatgtttgactgtcttgcatcactaccactagtaaggatcatcctcactgattatcaccatgttactatacttgaaccaaagcaaactacttgtacctccccaaaacattctccaaatcttggctggccttggccctgctcaccacctacaatgcttttccttaccccttctgcatccaaatcctactttaagatcaagctcaaattttcctctcccttaaagccttctcttatctccttcacgagaagtactttctccctccactgaattcccagaggactctctctgacaccccgttctggcttttgaaattccccatcactgagatgtcagtggaattttaaaaaccatcaagtcagccaggtgcggtggctcaagcctgtaatcccagaactttgggaggccgaggcaggtggatcacaaggtcaggagatcgagagcatactggtcaacatggcgaaaccccatctctaataaaactacaaaaattagccaggcgtggtagcgcacgcctgtagtcccagctactcaggaagctgaggcaggagaatcacttgaacccgggaggcagaagttgcagtgagctgagatcataccactgcactccagcctgagtgacagagccagactccatctcagaaaaaaaaaaaaaaaaaaaaaaaaatatatatatatatatatatatctcaagtctaggatcagacttcaagtttcactgagctggaagtggctgccaatgctccccagctctttagcaaaagacatttacacacgatattgtattggaggcatttggggaaaatgaaggaagtggggagcatttacagggtgcagtgactctaacatcaagagctatttgcagaagccgtgggcaatgacagatgccaaaacaagatggagaaatcaacttttatatagactgattcacaagaaaatatggagtgcctctcccaaaccaggaatcaaagatgttggggtgacacaggcagactcctacgatcctctagatggggaccctggacatttgccttgcctatatagagggctggaaacttttgaggctagaaccacacattcacaaagaaccaagcttagttgtttattttttaacttattactaagcataaactgtatttctgtagatcaatcatccccaagcttgggatttttttttttcttcctctgttgtccaggctggagtgcagttggatgatcatagctcactgaagcctcaaactcctgcctcagcctccctagtagctaggactacaggcacacatcaccacatcaggctaatcttttaatttttttgtatgggggggggggtctcactacattgcccaggctggccttgaactcctggcctcaagcaatcctccttcctcagccttccaaaatgctaggattagaggtgtaagcgaccacacctggccagcaaggttgggatatttttaacagccaaagtatttccagttccctcaagggccttcatgaaaaaacaatttaagtccaaacagaattaattttaactcactgtagtttaataatgaagcgcaccgtataagaattttagaaggaaagtctgtgcctaattaaactctggcaataaagacagagaagtctgaaggtagagaggctttctcatggttacccagtgtgagactctgattcctggagaccacaattatgcaccaggcagagggaattctactatgcatttgagactttgattatgatgttgtttaatgttcattatgcacaaatctcagagctgaattccaggaaaagattgattggcattccccatcctccagccccatctgctttccttatgttttccccacaccgagctcattcccgtctcagggcctttgtatttcctgggatttctctctgggatgccctttctccagagccttacgtgactggttccatctcctcattttggtcttgattcaaatgtcacccacttgagaggtcttccctgattccttagtccaagagttggcaaactacaatccatggggcaaactctgcccatcacctgtttttatacagcccatgagccaaaagtggtttttacatttattattttggacttttttttagagacaaggtctcgctctgtcacccaggctggagtccagtggctccatcacggctcactgcagtctcaaactcctaggttcaagggatcctccccccctcagcctccagggtagctgggactacaggcatgtaccaggacaccggctattttttaaaaaattttttaagaaatggggtcttgctatgttgcccaggctggtcttgaacttttggcttcaagtaatcctcctgcctgggcctcccaaagtgctgggattacaagcatgagccactgcaccctgcctgctttttaccttttttaatagctgaaaggaaaatcaaaagaagaatcctatttggtgacacatgaaaattatgcaaatttcagcatccattagtaaagctttactgggacacaggcatgctcattcatctattgtctacagctgtcttcaagctgcagcgtcagagctgaatagttgaggcagagatggtaggcttacaaagcctaaaatatttacctggtcctttacagaaaacatttgccaagcgctcttctagtctaaagtacctgtaatatcctttctgcctgggtgcagtggtttatgcctgtaatcccagcactttgggaggccaagccaggtggatctgttgaggtcaggagtttgagaccagcctggccaacatggaggaatcacatctctactaaaaatacaaaaatcagctggacatggtggcaggcacctatactctcagctattcaggagctgagagaatcacttgaactctggaggcagaggttgcagtgagccgagattgtgccactgcactccagcctgggtgacagagtaagactccgtctcaacaaactattttattttcttcatagccgctaccagtatctaaatttctaagttctctctctctctttatttacttacatgtttaaaaaaattgtctccaccaacactcccacaataaaacaatagggccgtaagagcagagactttgttttgtttccttctctatcttcagctattgatacataatgggcttttaaaaagtttattctgtttacattactgacattaaaggtttaacaaattgaagctatctgagaaattgtttgtattgctaatctatatagccattcttttctatcgctgttttgtttgtttgtttgtttgtttgcttgttttgcgacaaggtctcactacatcactcaggctggaatgcagtggcacaatcccagctcactgcaagttctgcctcccgggctcaagtgattctcccacctcagcctcctgagtagctgggaccacaggcgcacgccaccacacctggcttttgtttttttgtttgtttgttttggtaaagacagagttttgccatgttggccaggctggtctcaaactcctaacctcaagtgatctgcccgtctcagcctcccaaagtgctgggattacaggtgtaagccaccgcacttggccccaattttcttttcttttctttctttctttttttttttttttttttgagacagagtctcactctgtcacccaggatgcagtgcaattgcatgatctcagttcactgtaacctccacctcctgggttcaagtgattctcctgcctctgcctcccaaatagctgggattacagacatgaaccaccacgccaggctaattttttgtatttttagtagagaagggggttcaccatgttggccaggctggtctcgaactcctgacctcagttgatctgcctgactcagcctcccaaagtgctggattgtaggcatgagccaccatgcccggcccacctggccccatttttaagtgtagagttgagcgggagtaagtacattcacatccacactattgtgcaaccgtcactaccagccatatccagaatattttgaaccttgcaaaatcgaaactgtaccaccattctaatttctgtctctatgaatttgactgctctaggacacaaggaggctttaagcaacatccatcaaataaataaatcctgacctacctgctttggtagccaggacgctcagagttaaatttctgtgttttattgcctagatttcctaataaaattctcttctgtctctctctctttttttttttttcgagacggagtctcgctctgtcacccaggccggagtgcagtggtgcaatctcagctcactgcaatctctgcctccccggttgaagcgattctcctgcctcaacctcctgagtagctgggattacaggtgcccgccaccacgctcggttaatctttgtatttttagtagagatgtggtttcaccatgtcggtcaggctggtctcaaactcctgacctccggcaatcctcctgccttggcctcccaaagtgctgggattacaggcgtgagccaccacgcccagcctcttctctcattttaatatagatttttttatacagattttgaacttgtacccctactcaaggttcacattgaatctaagttaccatcattatttatattccactgaaaaactaggacacaacatagattatgaaatacaaacacatcattatttcagacttccacatgtgcgaattgcattgacgaaatggagtatgtgttttactccataagccacatcaaactgtttttcgaagcagtgggaggtggtaaaaatcagaagagaacaacatcaaatccatgttttcagagatgaaacacctcttctgaagatattcttcccccaaaaagtgaccccacctccaagtacaggctcaccagtggcattggaggatgtctgcagtattccagtcatccaggaatagagagctctctgggaatgagccgaggttttgtcgtagagatctttgaacactgcagatctaaatgacacaaacacaggcatcagctaataacaagcaggtgggggctgcattaaaaagggagtcacaacagcaaatgtgacccaaagctgtcttgtgttaaaacccttccctctcctggatgtggagaaatagaaacgcttttacactgttggttggaatgtaaattgattcaaccattgtggaagacagcgtggtgattcctcaagaatctaggactagaattaccatttgacccagcaatcccatttctgggtatgtacccaaaggattataaatcatgctactataaagacacatgcacacgtatgtttattgtggcactattcacaatagcaaagacttggaaccaacccaaatgtcctccaatgatggactggattaagaaaatgtgacacatatacaccatggaatactatgcagccctaaaaaaggatgagttcgtgtcctttgcagggacatggatgaagctggaaaccaccattctcagcaaactatcacaaggacagaaaaccaaacaccgcatgttctcactcataggtgggaattgaacaatgagatcacttgggcacagcaaggggaacatcacacaccggggcctgttggggggtggggggagggggtggggatagcattaggagatatacctaatgtaaatgatgagttgatgggtgcagcaaaccaacatggcacatgtatacctatgtatcaaacctgcacgttgtgcacatgtaccctagaacttaaagtatatttaaaaaaaaaaaaccttccctttcttgaatgtaaattggttcaaccattgtggaagacagtgtagcgattcctcagagatctagaactagaaataccatttgacccagcaatcccattatcgggtatatacccaaaaatatataaatcattctgtcacaaagataaatgcacacatgatcattgcagcactaatcacaatagtaaagacatgtagtcaacccaaatgcccatcaataatagactggataaagaaaatgtggtacatatataccatggaatactatgcagccataaaaatgaacaagattatgtcttttgcagggacatgaatggacctggaagccattatcctcagcaaactaacgcaggaacagaaaatgaaacaccccatgttctcacttgtaagtggaagctgaacgatgagatcacatggacacagggaggggaacaacacacactgggtcctattgtgggggtggggtgggggagggagagcattaggaaaaatatctaatgcatgctgggcttgatacctaggtggtgggttgataggtacagcaaaccaccatggtacacgtttacctatgtaacaaacctgcacatcctgcacgtgtaccccagaacttaaaaataaaaaatacccccaaacacactccttaggtatatgtaactatttttcccattttcctcttccccttcacagctaaacaccttccaaagaatattccatacatattgtctccacttcctcacctcttggtccttttttggggaagtgggggcagctctgttgagatataattcccacaccttacaattcacccatttaaagtacacaattgggccgggtgcagtggctcacacctgtaatcccagcactttgggaagccaaggcaggcggatcacctgaggtcaggagtttgagaccagcctgaccaatatgatgaaactctgtctctactaaaaatacaaaaagtagccaggcgtggtggcatgcacctgtaatcccagctacttgggaggctgagacaggagaatcacttgaacctgggaggtggaggttgcagtgagccgagatggggccattgcactccagcctgggcaacaagagtgaaactctgtctcaaaaaaaaaaaaattacataaataaagtgcacaattcagtggttttcagcatataagacaaagagccaaagatgtttctgatggcatcatttgagtctctggatccagctgcgtctgaagtcagccctacacctaaaactattcaattacatgttccaataattccctttttgcttcaagccagtttgaatgggtttttctgccacttgggcttactgcttactcaggtgagcagtaaccaataaactctctctcccaatgatgttctcttccacaatctgcatgaacggaggagggaagagatgctgaatttaaaaggaatagtataatagcctgcagagcaaaagggtttcatttatatacatcttagtgaaaaaaactggcaacctagagaaacgatgaggtcagcatactgtctgttttccttactgtcatttggctacaaaagataaaaggaatctcccacattgttatcatcatattggtcttttccttcttctaaatcatttttagatttagaaagagtgctttatggccataaagcactacgattagtaaaaggagggagttggcggtggttgcaatgcctatggcccttcaagtcctagacacaatcctcgaatttggtttacatcaaagcgcaatttcatgcagggctaggtgtaaccaatctaagctggcatggcatctcaaaaggagaagtcctcctttttcagtgctctgtcactccttctgttgtccctcaagaacaaagtctcagtttagtgccagtgaatctttaaaacacactcagattgtttttaaatcaatggaacaagacccaggctcagagcctttgtgctgtggactgaatgtttgcgtttccccaaatttcatgtgttgaaatcctaattcccaacatgaaggtattaagaggtggagttttgaggaggtgattaggtcatgaggctggatacctcatgaatgggattagtgcccttataaaagagatcccagagggcttgcccaccccttccaccacaaggggacacagcgagaaggtgccacctataaaccagaaagcgagccctcaccagacaccaagtctgtcagcaccttgatcttggacttcccagcctccagaaccatgagaaataaatgcttcttgtttactagccacacagtctatggtatttttgttacagcagcccaatctgactaacacacttaagcaagcaacatatctagctaggatttagtaacactttttttcccactgtatttatttatgtgggtttttttgtttttgttttttgtttgtttgtttttgtttttgagatggagtctcactctgtcgcccaggttggagtgcagtggcatgatcccagctcactgcaacctccacctcccaggttcaagcaattctcctgcctcaagcctcccaagtagctgagattacaggcgcccaccaccatgcccaactaatttttgtatttttagtagagacgtggtttcattatgttggccaggctggtctcgaactgctgacatcatgatccacccacctctgcttcccaaagtgctgggatcacaggtgtgagctaccacacccagcctaactctacagatgttaagcttttgctccaaaacgtgcatatcaacctgtccccacttgtcttctccctgctgccaggtgagatctctttgcattctcacggcagcccctgaaggatactgtgatgagttaagaaggtgggagtggctgggtgcagtggctcatgcccgtaatccaagcactttgggaggccaaggcaggtggatcacttgatgtcaggagttcgaaaccagcctggccaacatggtgaaactccatctgtacaaaaatacaaaaattagtcgggtgtggtaggcgcccctgtagtcccagctacttggaattgaggcaggagaatcacttgaacctgggaggcagaggttgcagtgagccgagactgcaccactgcactccaacctgggcgacagagtgaaactccatctcaaaaaaaaaaaaaaaaaagttcggaggaaccactcacaggttcttaaaggcatcctcgcgcaggtcccgagggagccgctctgtgatgtctggctggagaaagctccctgaggaccccctcagcaccttgcccaggatctccacacactccagggagttcagcatctggaaacacctctccagtgtgatgagaaacaggctgcggttcacgccagggctctgcaaggctcgttctcgaatctctcctaagtcgatgatgatgtctttcaggtcctaaagcaatgacagaagccagggtcagtttgagagctgcctctcaatttggaagccctgggagggaccacacattgcaacagtggggaaagtcaggatcactgtttaacaccatgctgctatgttgaaacttctagaactttccattccctgcagaacaaagtcaaggcctttacctgctccactcactgtggagcctccccactctccagatacacctgaccagcatacatcaggcccttggtctacctggaattctgcatctttcctttttctggtgaaatctagctcatcctttaagtctgggctccattgccatcaccttcgtgaagtctctgatccctccaacttcctacttcagatcagatttccctcccttatgtggccccagggcatcttgaaccaaactctataatgcattaaagggactcatgcgtctttcctccaagcccgtggttctcatctctgggtgcacatcagaatcacctggggagcttttttaaagttttattttatttttgagacagggtctcgctctgtcacccagtccgcagtgcagtggtgtgattatagctcactgcagcctcaacctcctgggattgagcgatcctcccacctcagcctcccaagtagctgggactacaggcacacaccaccatgctcagctgttttttgtagagatggagttttgccatgttgtccaggctggtctcaaactactgggctcaagcaatctgcccacaacctcccaaagtgctagggttacaggtatgagccaccatgcccagcccatctggggagattttgaaaaggactgattggcctggaacagtggctcacatctataaccccagcatgttgggaggctgaggcaggttgatcgcttgagcctaggagtttgagaccagcctaggcaacacagggagaccccgtccctaaaaaataaattttttaaaattagctgggtgtgctaatgcgcacctgtagtcctagctgcttgagaggctgaggtgggaggatcacttgggctcaggatatcaaggctgcagtgagctataattataccactgcactccagcctggatgacagagcaagcccctgtctctaagaaaaaaaaaaaaagcctccattttactgagcattgactctactgtgccaagcttggagcaaacgcatcatctgattaattctcatgacaatcctaccggttaagccccatgggtactagggtcaggcattcccaggttccagtcccagggcctccattatctgtgtgtcctgagcaaatgacttaccttttttaagtcccagtttttaaggtctcagaggccttgctttgtgatattcttggatcactaaactcattttgtggaaattaactgttcctgttttttgtctcccctccagaccgtgagctccttgagtgcaggaggatctctgtgtccactggtgcatagtaatttttactagtggatgtttattgacccaaacaaccaggagattctcagatccttaattaggacccgtcatttcttagtactgacaaaaatatgaattaggccaggcatggtggctcacacctgtaatcccagcactttgggatgccgaggcaggcaaatcacttgaggccaggagtttgagaccagcctggcaaaatctcgtctctactaaaaatacaaaaattagccaggcatggtggtgcacacttgtaatcccagctacttgggaggctgaggcacaagaatcacttaaacccaggaggcagaagttacagtgagctgagattgtgccactgcactccagcctgagtgacagagtgagactctaaaacaaaaataaaataaaaaattgtaaggctcttcattttaactttggagaaagtaagtgagaaaacttaggtcctaaagcccccagggggttggaggaggcagaaggagagaggaagagacaatgaagtgccaaggcctaagtcaggctccatttattcaattcaacaaacaccacgtggtgctggacttcctctatccccgggatgccaagggctcctcaccaagccgtccttcttgtcttctaagaggcatttcatggcagtgcggaactgctgggcgtctgtcttcctcaggtcctccagcagcttctggggctggtgcagacgctgctccaggtggttttccacggctgcctgtatttggggtgttgggagagtcggtgagaacagaaagagcaggagcacatggtcagcaccatggccagcggcccagcccggccccctcattccctgcagagaccacaactagtggccagcagtctggtttggccacctccaaatttcctcattagccgccaacatttaaaaatgaggggccgggcgcggtggctcatgcctgtaatcccagcactttgggaggccgaggcgggtggatcacgaggtgaagagatcaagaccatcctggccaacatggtgaaaccccgtctctactaaaaatacaaaaattagctgggtgtggtggcgcacgcctgtagtcccagctacttgggaggctgaggcaggagaatcgcttaaacccgggaggcggaggttgcagtgagccgagatcacgccactgcactccagcctggtgacagagcgagactccacctaaataataataataataataataataataataataaggggattcctcacaagatcaagatttccaacttctcttgaaaaagtaaaggtttggcaacaccgggcgcacatccccttttaggcaataattggcaagccaagggacagctgcttgttgaagtaactctcaggcccagccagcccgattcccccattccatggcctgcctggtccctgtaggtgattgagtttcccatcagcagcagctggcctttgtccaggaagtttagaaccccagatggcactgtgcccaaacccaagctaccagcgcaggagcaaaatacaggcgaaatgaacatactctgtggcgccccctacagggcaaggagcagaagccatcagcctctcctgaaagagatttcctctgctgtctggggaattgttctctaaatctccgttaaacagacatgcccagtgtgatgtcatgggcaaaaggtgaactctagacccagacaagccacatgcaaatccccaccttgacacttactagctgtgtggcttggacgagttacttaactagggttgctgggtaaaatacaggatgctcagttaaatttgaatttcgggaaaaactacacatttttttttttttagtataagtatgtctcaaatgttgcataggacatacttaacttaaaaaatcactcattgtttgccaggcaaggtggcccacacctgtaatcccagcactttgggaggccaagacaggcggatcatgaggtcaggagtttgagaccagcctggccaacatagtgaaaccccatctctactaaaaatacacaaattagccaggcatgatggcatgcacctgtagtcccagctactcaggaggctgaggcaggagaattgcttgaacctgggaggaggaggttgtggtgagctgaggtggcaccactgcactccagcctgggcaacagagcaagactctgtctcaaaaaaaaaaaaatcactcattatttatctgaaattcaaacgaaattggacatctgttttttgtttttgtttttgttttagagacggggtcttgctctgttgcccaggctgaaatgcagtggtatgatcatagctcaccgtaacctcaaactcctgggctcaagcaatcctcctgcctcagccttccaagtagctgggactacaggccacaccaccacactcagctaattttttgtttgtttgtttgtttgtttttgtagagacaaggtctcgctatgttggcaaggcttgtctccaactcctggtatcaagcaatcctcccacctatgcctctcaaagtgctgggattacaggcatgagccaccacgcagagtcacatctgtatttttatttgctaaatcagcaacatttcttctgggcctcagttttcttatctgcaaaatggaggcaataaaaggacttaccacatagagttattacgaggattcagtgtgtaaagtgttttcaatagttgctagcacacagtaagtgctcaatagtgccagctattattctctatcttgaccaaggtgaataatggcttaattaaagcctcacccctaaaatgaaaggtccttgtgggtagtatcatgactgccttgttccctactgagtcctcagtgtctagagctatgcctggcagaaagcagatgttaactcagtagatattaaatgaataaataaatatggtatatacacaggaagaaacgtatcagattaaagtggatacatctcatgagtatccattactgctagagagaaaagtggcaagcattgcttctctttcagtttccctgactgatgaagaagaaaagaaaagcagatgccatgtggggccaacagccacctccagctagtggctgtcctgggacctggcagacaacaagctctctgagtatatttgtgatatcaatgttggttgatgtttccattttcattcaaacataagtaaaaatggaaacaatgaagacatatgtcagaactttattcattaattaatgacataaatggtcttttgctaaactgaataactttctcaatactgaaagaatttttctccaattttctgtgttatccacagtgtgatgataaagacacaacatgccacactaggctaacagtcaggcaggtgctgtgcagttcaccgcagtcaccaccacaggcacgagccctattgccccttaaggcaaatatcctttgccacctatcaacacgcttgtgctattgttttcattacagtagtacatttcttggacccatggtagaaagactgatcactatatccaaatatccactctctcactgctctttatcaattgaacccttcaacttttagttgggtacatggctcctttgcagtaagatgtgactatggtgtgtgagccaatctaatgtgtgcagagggaattgtgtaacttgcgtcatatcctcagaagagaagatgctacccttgtcttcttcttcccacccatcccctggattggagaaaggaagaggtgtttccaccacatgggcaataactccccaggagatgacaaagcaaaaagataaaaggaacctgggtccttggataacctcatggaatagagctgccttttctctgtggctttcccacttagctctggactattgtacgagaaagaaataaactaaattgtttgagctgctgtcatctgggggtctttttgttatagcagctcagcctatatcctaatataccatgtctccatcaaaggtgggaaaatgaaagaaagacaaaatagcttatatcatgtttcaagaaaaactggacagaacccttttccttgcagaagcaaagactatctctacatccagcccacttctccaacttacctggcccctgagtttgcaatccctgagcactgagatgggaacatatagatgggtctcaggtacacacctgcaggctggggatggtgaaggcaacattccgggaattcagataggccaggactctgtgggacaggtcatccgtccacacgtgggagcttcagttgaagacagacaggaaaagatcacaatgacagattctcctacaagcactactgtactagctaagtgcccaggggacaggtagggatggaccaggggtgttaggactttgtacttggaagtgggaggtttctcttttcttttctttccttttttcttttctctttttttgaaacagggtcttgctctgttgcgcgatcacggctcactgcagcctcaatctccccagcccaagtgatcttccaacctcagccacccaagcagctgggatcacaggtgcatgccacaacacccagctaattttttgtagagatggggtctcactatgttgcccaggctggtctcaaactcctgggctcaagcaatcctcccacctctgcctcccaaagtgctgggattacaggagtgagctgctgcacccagcctgaagtaaaaaatttcttaaccaggcacagtgataggatagtttccaattctaggaatctgcctggatcccattctctcaaagccaattcccaaatttctaagctgtatgcaatattctaattcccgtaacaatctgcttagattgactacaatccaaactgattgtgttagaaagatgtacatttaaaagcagacgctaggtacaccatgagaggctggaatagcatagttaggagtgtgggctccaaactggatttgaatcctagttccatcacttagttgtgtggcttgagacaatttgataaattttcttgtgcctcagtttccctttatatgaaatatggttaacaactgtgagattaaaatttgttcacacatgaaaattgcgtaagactgtgcccaacacacagtaaatgcccatgaatagccttttctcatttttttttttttttttggagacagagtctcactctgttacccaggctggagtgcagtggtgcaatctcagctcactgcaacctccgcctcccaggttcaagcgattctcctgcctcagccttccaagtagctggaattacaggcgtgcaccaccacatccagctcatttttctatttttagtagatactgagttttgccatgttggccgggctgggctggaactcctggccttaagcgatcctcctaccttggcctcccaaagtgctgggattacaggataagccaccatgcccagcctatgaaaagccttttgtaatcttacgtttgcttctttgtttgtttgtttgtttgttttgcgatggagtctcactctgttgcccaggctggagtgcagtggctcaatcttggcttatcacaacctcagcctcccgcgttcaagtgatcctcctgcctcagcctcctgagtagctgggactacaggtatgcaccaccatgcctagctaattcttttgtacttttagtagggacagggtttcactatgttggccaggctggtcccgaactcctgacttcatgatccgcccaccttggcctctcaaagtcctggaattataggcatgagccaccgcgcccggcctgtaatcttataaagagatggatggatggatggatggatggatggatggataaattaataaacaaataaaatacttagactgaaagaatatatccaaaagtacccattggtgttatcttagggaaaggagtggttatgggagtctttcactttaacataactgggtatccctgatatgaggccccaagacccctatttcttatcgatcatagtactcatcatattagaattgtttattaatattggcgtttccacactacctagttccctgccccatgtccctggtatctgtctgtatgcagcttataatgcaacagtaaagggaatgggtcctggagccaggccactggttcacatcccagttgtgtgtccttgatttacccttcctggacctcagtttcaccattgtgtgcaatgggataacaatgggaactacattgtgtggcacttgtgaggattagattgtttctataccaagcgcttagtagaatacccactacatagaaagcactcaataaatttcagctcttaggccggttgcggtggctcacacctgtaatcccagcactttgggaggctgaggccggcagatcacctgaggtcaggagtttgagaccagcctggccaacatggtgaaaacccatctctactaaaaatacaaaaattagccgggcatggtgacaggtacctgttaccccagctactcaggaggctgaggcaggaaaattgcttgaaccccggaggcagaggttgcagtagtgagccaagatagcaccactgcactccatcctgggcaacaagagcgaaactccattcaaaaaaagaaaaaaaattggcccttatgctagagtgaggaggaatgatgcccgtggagcaagggtgtttgttgacttttaacacattgtgcattgattgacattttttgtaatgagcacatattactttttacaattaagagacttccattaagaatttttattaaaaatctataccatcccttcccctctcctctagggcattttacctttgaaactgtatgagatccagcagtgctacaatgggagtaaaatgaaaagacatctttaaaaggaatcattccagagagatcacagctacattctgtttgaaattcttagaagaagcaagtggagatcaagggggcacattaccattcagatagcttccatctattatttcttcctaaaaggcaaagagataacatcaagtataacaaccaaaagttggcaattccagtagaggggactctgattaacagaaaatagaatactcaccgccatgttttgcaacaatggatgcaaatctgtaagaaagccattgaatgtataactagaattgaattgtgtgaccttcaaaactacaaatatgggttagaggaaaagccccagaaagccactcatgaaggaaatttttagtgaattttaattataactgtccactctgagaaaaccgtggcttgaaaaatcctcttagaataatcctgaaaaatcatacatgcacacacacacagcccaaaaacaacttacagcatcatgtacctaatgtatctatcccacctcattccctaaactgtgtggaaaccacaaagggtgattcttccctcaggacttaccctgcctggaatttggcactgtataactcgacactccatggctcagaaaaaggaataggaaaagggagtatgtcgtaggttcctgagacattctcctgtagttaatttaagcatagaagtgattcatgttttttttatatatatatatatacacacacacacatatatatacatgtatataaacatatatatatatatggacattcagaagacaaaaggaaaaaccaagtacacccagaatcccttgaagtcttttttaaaaataattttaaaactcacctactttcctccatctaaacccccattcttgtctaagacactatcatctctctccttggtgactacaatggcctgtttatcttaccactacccccaactcctctcaatccagcaaaaaggtatagtcaaaagtagactctgggccaagcatggtggctcacacctgtaatcccagcactttgcatggccaaggtgggtggatctcttgaggtcaggagttcgagaccagcctggccaacatagtgaaaccctgtctctactaaaaataaaaaattaactgggtatggtggtgcatgtctgtagtcccagctacttgggaggctaagacaggagaagtgcctgaatctgtgaggtagaggttgcagtgagccaagattgtgccactgcactccagcctgggcaacagagcgagactctggaaaaaaaaaagtatagactctattttatctgcaagaaatttatatccagaatacaaaaactactcttataattcaataagaagatagacaagccaaggggaaaaaatgttaaaaagatatgaagttggccaggcgcagtggctcacgcctgtaatcccagcactttgggaggctgaggtgggcggatcatgaggtcaggagatccagaccatcctggctaacacggtgaaaccccgtctctaataaaaatacaaaaaaattagacgggcgtggtggcgggcgcctgtagtcccagctactcaggaggctgaggaagaagaatggcgtgaaccctggatgcggagcttgcagtgagccgagatcacgccactgcactccagcctgggcgacagagcaagactccgtctcaaaaaaaaaaaaaaaaaaaagatatgaagctaaacttcataaaaacagatgtaaaaatggccaataagcacacaaatgattatcacattagctaacaagaactagcaaattaaactcacaacgagaccactacaaagtcactagaatgggcaaaaatttcaaagactgactatcaaatgttggtgatgatatggaacaacaggaactctgatacagctgcttggaaaaactgtttgacagtgtcttacagagttaaatatacaataccctatgatcaagaaatgccacccctaggtatttaagcaagagaaatgaaaacatgtctacacaaagatttatatagcttcattcatagtagccagatattggaaacaatcacatatccaataacaggtgaaaggttaaacatggatctccattcagtgggataccattcagtaataacaataaaaagaaattcatcataattttattctagctatgatggagtaattggcaatggataaaccgtcctgccttaaacaattaaaaagctggctgaaatatgtgcatccaaaggtttttcagacattgggcagcaggtagcacaagactgagatccctgagcaaggaaggcaaacaagctgagctctataattgctcctgctcactgcctggagtctccatccaacagcacaaggagggagaatccaaacagagcctacaggtctcactgagcagaagagacggagtggaacttcagggaaatcaaggcagctagaatccataagaagaatacttggagaggaggaagttgcacagagagaaagttctggagatctgaagaagggtcttttgagactttggctgaatatttatctacctttgcatgtaagaaacctcctgaggctggagaatgaaccaccagtaggcagaacaatccttggacctcacaggggaatgagaatagctcctgcaagctgcaatggaaaaacctccaaacacattgggcatcagggtcaatcatcaaagaacaattgcctccatgataggccaaattagccctaggctaaagtttattccagatctgccctaacaaatctcaaaagcaagttctggaaggatctaattgattccaagtaactgaattgcattccagaacaaaacccaacaatatttaaagcagtatcataaaacccagaaacttcagaaaaccagaagatggagaaagaaaaaaaagaaaaaaaaaatccagcaatgtaaaatccgaagtgtccagatctaatcaaaaattatcaggcaggccaagcacagtggctcatgcctataatcccagccctttgggaggccaaggaaggtgaattgtctgagctcaggagtttgtgaccagcctgggcaacatggtgaaactctgtctttaccaaaaatgcaaaactttgccagggatgtggcaggtgcccaactactcaggaggctgaggtgggagggtcactgaagcctggaaagttgaggctgcaatgagccatgattatgccactgcactccagccagggtgacagagcgagaacctgtctcaaagaaattttttaataaataaataaaaattaccaggcatactaggaagcaggaaaatatggctgattatcaggagaaaaatccaatcaatagaacagatccagaaatgacacagaagatccaattaatggacaaggatgtttattataaatacactctggaagttcaagaaagtagggaaaactgcgagcatgttaaagggagagacatggtaggtgcgaaaaaaaccccaagaggaacttctagagataaaaatacaatatctgaaatggaaaatacacgggatagagttaacagcatattagacactgcagaagaaaaattagtgaacttgaagtcatagcaaaataaattatccaaagtaaaatgcagggggaaaaagactgaaaaaattaagagggctctggagcaatatgaaactatttgaaatatgtgtaactggagtcccaggaaaggagggggtggcacaaaaatatctgaataaatcattagcagggcacagtggctcacgcctgtaatcccacattttggaaggttgaggtgggcaaatcacttgaggccaagagttcaaaaccagcctggccaacatggtgaagccccatctctataaaaaaaaaaaaaaaaaaaaaaaaaaaaatagccaggtgtggtggctgtaatcccagctactcaggaggctgggcacaaaaatcacttgaacctgggaggcggaagttgtagtgagccaagattgcaccactgcactccagcctaggtgacagaatgagactgaaaaaaaaaagagagagagagaaagaaaaagaaagaaggaaagaaagagagaaagaaaaagaaagaaagaaagaaagaaagaaagaaagaaagaaagacagaaagaaaacatgacctcatgaccaaatttgtgtaaagtttgatagaactacagatccacagatccaagaagctcaaccaaccccaagcagaggaatcatgaataaagccacgccaaggcatataataatcaaattgctaaaaaaaaaaaaaaaaaaaaaaaaaagatttttaaaaaaaatccagcaaaaaggagttgcatctagactatattgaaaaactcttaaaactaaaaaataaataagcaatccaatcagaaaacgagcagaagatatgtgcagatatatcatcaaagtatatatacagatagcaaataaacacatgggaagatactcagcactgttagccattaggaaaatgaccactgaaaccacagtataattataggcctatcagatggctaaaataaaaaaaaaaaaaccataatgaagccaggcgcagtgaatcatgcctgtaatcccagcactttggaaggccgaggtgggtgaatcacctgagatcaggagtttgagaccagcctggccaacatggtgaaaccccatctctactaaaaatacaaatattagccgggcatggtggctcatgcctgtagtcccagctactcaggaggctgaggcaggagaattgcttgaaccgaggaggcggaggttgcagtgactgggaggcagaggttgcagtgagccaagattgcaccactgcactccagcctgggcaacagagtgagacttcaactcaaaaataaataaataaatgtaaccataatgccattaactagaaagaatgaggagaaactggatcactcacacattgctgatgagaatataaggtggtacagctattctggaaaatagttttgttgtttcttataaaattaaatgtgtacttaacgtacagcccaacaattacactcttgggcatttatcccagaaaaatgaaaatttatgttcacataaaaacctgtacacaggctgggcatggtggctcacgcctataatcccagcactttgggaggctgaggcgggtggatcacgaggtcaggagatcaagaccatcctggctaacatggtgaaaccccatctctactaaaaatacaaaaaattagctaggcgtggtggtgggcgcctgtagtcccagctacttgggaggctgaggcaggagaatggcatgaacccaggagacagagcttgcagtgatgcagtgagccaagatcacgccactgcattccagcctgggtgacagtgtgagactccaactcaaaaaacaaacaaacaaacaaacaaaacctgtacacaaatgttcatagcatctttattcatagtagctgaaaaattgaaccaatctaaatgtcattccgtaggtgaatggttaaactcactgtgtgtggtatgtccacgccattgaatactactcagcaatgaaaaggaatgaactattgatacaggcaacaactttgatgcacctaaagggaattatgcagagggaagaaagccaatcactgaaagttatatactaatgattccaattataaagcattcttgaaataaaattatagaaatgcagactagattggtatttgccatggggagagggagataaggctgtcaagggatgcatgaaagaggcttgtgatcaccaggagagttctgggtcctgattagggtagctgatacattagtctacacatgtcacaaaattgaagaaaacgatataaaatatacacacaaaggagtgcatgtaaaactagtgaaatctgaataagccctgaatttgtaccatgatttcctgaattttatatggtactattgttacataacatgtaaccaatgggggaaactgggtgaagagtacacaggatatttcttttgcaacttcctatgaatctgtagttatttcaaattcttttttattttcttgagagacagagtctttctctgtcacccagcctgcagtgcagtggcatgatcatggctcactgcagccttgaactcctgtgttcaagtgatcctctggtctcagcctcctgagaagctgagactacaggcatgcactaccatgcccagctaattcttttagttcttgtagaaatgggttcttgctatgtttcccaggctgatctcaaactcctggcctcaagcaatcctcccatctcggcctcccaaagtgctaggaatacaggcatgggccaccatccctggccacacaattgttttttaatttagttatagtagtctgtaccactgtaggatgacaatagttaacaataatatatagtttcaaatagctagaaggaagatactgaacagaaagaaatgagaaatgtttgagatggtagacatgctaattaccctgactgatcaccatacattatacacatcaaaacatctttatgtaccccataaatatgtacaattattatatgtcaatttttttttttttttttgagatggagtctcactctgtcacccaggctggagtgtagtggcgcaatctcggctcactgcaacctccgcctcccaggtgcaagcgattctcctgcctcagcctcctgagtagctgggataacaggcatgcgccaccacacccagctaatttttgtatttttagtagagatggggtttcgccatgttggtcaggctggtctggaactcctgacctctggtgatcagcccacttcagcctcccaaagtgctgggattgctggcgtgagccaccgtgcccggcgtatatgtcaattttttaaataaataaaataataaatttttaagcacccaaagggaaaaaacccatattatatgcataggaaaaggaatacactactgatataggcaacaacacagatgaacctcagaaacattatgcttatgctgagcaaaagaagccagtcaaaaaagaccacataacatatgattccatttctgtgaaactctaagttatctagaatatctagattctaggttatctaggttacctagaataatcttcagtgacaggaaacaaattaggattggtctggggcattattagggtacaatgggaaaagtttattgcaacagggcacaagggaacttcttggagtgatggaaaaattttttttgtagatatagggtctcactatagtgcccaagctggtctcaaactcctggtttcaagcaatcctcccttcgagcttcccaaagtactgggattacaggtggatgccattgcacccagcccccattttttgtatcttgattgtggtggttgtcacacagatacatacatttatcagatctcactgaactgtacacttagtgtgcattgtattgcctgtaaatttatacctcacaaaagtcagtatcaaacagaaatggaaaagcatggactgtggagccagtcacctgttctgaatcacgtctccgccacttactagctgtgcaatgttggacatatttctttctttttttttttcttttttcttttttcttttttttttttttgatatggagtcttactctcttgcccaggctggagtgcagtggtgcaatgtaggctcactgcaacctctgcttcccaggttcaagcgattcttctgcctcagcctcctgagtagctaggattacaggtacccaccaccacaccaggctaagttttgtatttttagtagagacaggctcccaccatgttggccaggctgatctcgaactcctgacctcaagtgatccacccactttggcctcccaaagtgctgagattacaggtgtgagccactgagcccggccttggacgagtttcttaatctctctgcactccaattttctcgtctgtaaaatgagattaatgatggtaccagcatcactgtgtcgtatgagggtaaaatgagttgttaatagtgaatcacttcaaacagggcttgacacaaagtcagctctgtatacaagtttattaaataagcaaagaaaccagattgctgttttcaaacagatgctgttttgaaaaacaccatctcaataaactcatgtcaccctccgcctctccccattaccctcaagataaagcccaaagcttttccagtggctgtatcaccctcctctttgcccctcattccccaaggttcctgcccgggccatctgtctgtccctccaaggtgctatcctccccagattaagaccctctcacaggctgttcctcggcccagaggattcttccccttcctctttgcccggctaagatcaattcattgtaaatttcaagtcttcaatgaagccttccctgaattctctgaccaggccaaagcaattagccagaagaaaaggccaatacttctcttttatagcaccaagcacagctataatacatatttatgtttgtgtaattatttcatgctttcattcttcaaatatttgttgagtagtgctgtgagccaggcattattctatgctctgagaatacagcagtttacaaaactaactccctgctggtgtgttggaatgaaccaggcagaaaatagataatcaaataactatattatgtcagggagtgatgagtgccatggagaaaaatacaacagggtaggaaaaaggagccaaagaaggagcaggtactattatagacagtttggtcagggaagacctgaggaggtgacatttaagcagagacctcaggaagtgaaggaatgaaccacgggaatatctggagaaagaaagttccagaaagaaaagaaaatgacagtgcaaaggctttgatattaggagcataatttgcatgttcttcagtaaattgcacgaagaggatataacagtaggacatggggttggagaggtatcagggccagagtgagtagggtctttcaggccattgtgagcgctctgacttttacttggagggaggtgggagccactgaagggatattagccaggatggatgctctgacttgtttttgtttttgttttgttttgtttgagatggtgtctcactctgtcgcccaggctagagtgcagtggcacaatctcagctcactgcaacctctgcctccccggctcaagtgattctcctgcctcagcctctcaagtagctgggattataggtgcccaccaccacacccagctaatttttatatttttggtagagacgagatttcaccctgttggccaggctggtctcgaactcctaagctcaagtgatccacccacctcagcctcccaaagctctgggatgacaggtgtgagccactgtgcctggccctgacttttattttaactggatcaagcacattgattgctgtgttgagaataccctggagtggaacaatggctgtagcaggcagaccaatctaggaaagagatgacaataacttggtcattgatagcagtgaaggtggtgagaagtggttggattttggaggtatttggaaggcggaaccaaaagatcgaatgtagggaataaaagaaatagacaggtcaaagacaacgccaaggatttggcctgagcatgtagagagggagatgggaaaaccataggagttccaagtttagggaaggatccctggagctcagttttggacatactaagcttgagaagccttttagagtgcagatagcatagcaagtaggcagtttgatgtttgagctgcaggttaggggagaggtgctgtcagaagcagctatctaaacagagatagtcagtggttttttgtgtttgcttttttgtttgttcttctgttttgggggtttttttggggtgggggacagagtctcactctgtcacgcacgctggagtgcagtggcacgatctcagctcactgcaacctctgcctcccaggttcaagtgattcttgtgtctcaacctcccgagtagctgggattacaggcttgtgccaccgcaccggctaatttttgtatttttagtagagacggggtttcaccatgttggccaggctggtctcaaactcatgacctcaagggatccacctgacacaggctcccaaagtgctgggattacaggcatgtaatcccactgcacccggccccagatagtcagcatgtatttaaaccctgaatgatatcaccaagaaagtaaatttacggagaaattaggtgagttttaaggactgagacccatggcacccccattgccaagacgttgagtagactaggtgcagccagagaaatcaagactgtggaaaactccccaaaacaaacaacctagtttctttagccaatatattacagagagagagagaaagagaacctattaactcaaaaacaaaagattgatcacttgtagaatctagactttatttggatcctgattcaaacacgtcatgtctgggcacggtggctcacacctgtaatcccagcactttgggaggccgaggcgggcagatcacttgaggtcaggagtttgagaccagcctggccaacatggtgaaaacccatctctactaaagatacaaaaacttagctgggcatggtggcgcatgctaataatcccagctactcgggaggctgaggcaggagaatctcttgaacccgggaggcagaggttgcagtgacccgagatcatcccattgcactccaacctggatgacaagagtgaaactctgtctccaaaaaaaaaaaaaaaaagaaagaaacaaagcaaacaaacaaaaaataccataaaataaaataaaaaattaggccaggcacagtggctcacacctaatcccagtgttttgggaggccaaggcggaggattgtttgaggccaggagtttgagaccagcctgggcaacacagcatgactccatctctataaaaacatttaaaatttagcctggcgtggtgatgcacacctgtagtcctagccactcaggaggctgagatgggaggatctcttgagctccagaattcaaggctgcagtgagctatgatcatgccacagcactccagcctgagtgacagaacaagatcctgtctcaaaaaaacccaaaaaattgtgagacaattgaggaaatttgaacccctgattagatatttgaagattttaaagaattattgttaatagttttaagtttgataatgaaactgtggttatatttttaaaggagtccttattgtttcaaggaggggttagtaaagtaccacccacggaccaaatggaaatggccacctgattttgcgtggctcatgagcttaaaatgattttttaggttgtttaaatagttttttaaaaccaaaagaataatatttattgacatatgaaaatttcctgtgtgaaatttaaatttaagtgtctataaataaagttttattggcacatgtccatgcccattcattggcctattgtccatggcagcttcctgctaccatagcagagttgagtacttgggacagggaccacacggcctccaaagctcaaaatatgtattccatgttcccttactgaaaaagcttgccagcccttcttttattttatgtatttatttaattgtttttttttttttaagacagagtctctctctgttacccacgctggggttaccatgcctcagcctcccaagtagctgggactacaggcgcccgccactacgcccggctaattttttgtatttttagtagagacagggtttcgccgtgttggccaggatggtctcgatctcctgatcttgtgatctgcccgcctcggcctcccaaagtgctgggattacaggcgtgagccaccgcgcccagccgccatcccagcattctttaatacagatttccctcgtattgtctaacttgttatgcaaatggattatttttcccccaaggggacctccacaaccagctttgaaaacaattatgcttactttactgcccaggacaaaaacagtaaaacttttgaagttcccagacaccaaaagccctgcattctaacaggttttgagcaaacctgatcccaaagcactttgtaggtgtgttggcccaccatcaccatcctgtaactggaatgctcaactccaattagaagtcttcctggtttgggtcatttggggctttggggaacctttgacccttttttctcccttccctttgggcagctgccctgggacattggccccattcacaattctccagtttcccagacaatgtggcttcttgtgctctttgctcgggcccctctattaaccctacagagaggtttcaggttactgaaacagacgccttgctttctgctgtgcataatgtcctttttccagcgagaaactgcagcccatcaggatctggttttcattaaaggcactttgggtcacttttttagcagattggtcaaaaggactgaaaggactgggcagggcgatgatgatttggaggtcaatagctttctctatgggccataccccttccccactgaaagattcccccactgcagactggaggaaatcagtcaggcaaggatccctgtggtgaaaactactcgaaaacagcaacaataaccacaaacctgaaatgaagacaatttcctgagaaactatgaatgtttggatgtgcaaggtgatcaactttcccagttgcccgggaccaaaaggtttcccaggactcaaaactttccattttgaaaccagaaagtcacaggcaaacccggaggagttggtcaacccatggatcagagtcacttcatcttctgtgaaatttgcaaagatgctaggaggttcccctcctgctgggacaccccagcccagacacaaaccattaattcacaattacatggagtttcactgtctgcaaggctgctccatttaagctctgggtcatgaacacataactctaggcatactgacactagctgggagattttccaccaaaaaaaaaaaaaaaaaatgccatttcatgactattaatccaaaataggtaaatgtgtctggcttatagaataccagcctgattacaaatgcttggtgttggaatggcccagctcacagtggttgtagaagtccagtaggcccaggctttgtggctcactcctgtaaccccagcactttgggaggccaaggtgggaggatcacttgagtccagaagttcaaaaccagcctgggcaacatagggagactccatttctacaaataatttaaaaattagctgggtgcatgcttgtagccctagctactcaggaggttgaggcaggagaattgcctgagcctgggaggtcgaggccacagtgagccgcgatcacatgtcactacattccagcctgggtgacagagcaaaaccctggctggaaaaaaaaaaaattcaagcagtcaaggggatgggagcttggtggactagagatgcccagacttgagttcctgtcccagccctgccactaatagtgagattttaggcacagctattccccttcttctctcctaagtctccggagaacaaatggctttggattcaatgaggaaaagaaggaaagaaaggaagaaactggtctcactgagcatcctctatgagccgggcatcacaccaaaccctcctccctgccatttactctttccaccagccttgaagggttaccgtactcagtttcacagatgagttcagtgcacatgaacgatcacacagcaagcgaatggcagggagggggttatgatgcagggctgtggggctccctagtaaccttaatcccagcagtcatttaatgagacagaactaagtacacgcctctctaacatacattgtctcgcttaattcttgtagaatctttgaggcaagcattaccattatctctacttcacagatgagcaaatgggttctgagaggtcaagtgacctgcccaaggtcacacagctatgcatgcacagccaggtggcaaacccagagctaaatgattccaacgctcactggctttgctctccagtgtcagctcagcagccctgaacctaaaactccaggtggcaggtgccaccccagggataagaccccagctttaacctgaaccaaaccttaaccagctgtgtgctattgggcaaattccttaacctctataggcttgcatttcctcctctgcatagtaggcatggcactcatacctccctggcaaaaaactatcaactttatttgtttatttatttatttatttatttatttatttatttattcattcatgagacagagtctccctgtgtcgcccagaatggagggcagtggcctgatctcggctcactgcaacctccacctgccggattgatgcgattctcctgcctcagcctccggagtagctgagattacaggcgcccgccaccacacctggctaatatttgtatttttagtagagatggaatttcaccactttggccaggctggtctcgaactcctggcctcaagtgattctcctgcctcagcctcccaaagtgctgggattacaggtgtgagccccggcctttacctgcatttttccatttcttccacacaactgttatgaggtaggcattactttatacataaaatacataaagaactaagcccagcactggcacatataagaatgcaatgaatcgtagctattattactaatgctattgtttttactgctgctatattattataacaaataatcattgtgatagttattgcaacagttgtcatcgctgttattatattactaagtagttttggatcccttttctcgaagtttcatcgtcccccccctccaagtgccctccaatcccaggagcccttaaagccgctgcacccacactttgcccaccctctttctctcggtctgtcccccacgccatccatcacctgcaccccttccctagggaggcccagcggtgggcgcccacccgctcccccagcgctttgccccgtgagtcctccgcccaggcccccgcgcgcgcctcacctgcggagcccggcttggccgcactgagtcccacgggcgggcgggtggcgcagggcggggccgcggggctcatgcggggagcgggcaggcaggagagcggcggggcggcgccagccggcagctctgcgacctcctccctgcagcggcccaggtgggaactcagccagggcagcggcgggggtcacagtccccgcctgggacttcctatctgtcgaagctt

SEQ ID NO: 41 - DNA, 3762 bp, Homo sapiens
acagaggctgtggctggaaggagctgggcatccggcctgaggcgcagcggtcgcgttagttcggcccaatggcggcaccgctgcttcacacgcgtttgccgggagatgcggccgcttcgtcctctgcagttaagaagctgggcgcgtcgaggactgggatttcaaatatgcgtgcattagagaatgactttttcaattctcccccaagaaaaactgttcggtttggtggaactgtgacagaagtcttgctgaagtacaaaaagggtgaaacaaatgactttgagttgttgaagaaccagctgttagatccagacataaaggatgaccagatcatcaactggctgctagaattccgttcttctatcatgtacttgacaaaagactttgagcaacttatcagtattatattaagattgccttggttgaatagaagtcaaacagtagtggaagagtatttggcttttcttggtaatcttgtatcagcacagactgttttcctcagaccgtgtctcagcatgattgcttcccattttgtgcctccccgagtgatcattaaggaaggcgatgtagatgtttcagattctgatgatgaagatgataatcttcctgcaaattttgacacatgtcacagagccttgcaaataatagcaagatatgtaccatcgacaccgtggtttctcatgccaatactggtggaaaaatttccatttgttcgaaaatcagagagaacactggaatgttacgttcataacttactaaggattagtgtatattttccaaccttgaggcatgaaattctggagcttattattgaaaaactactcaagttggatgtgaatgcatcccggcagggtattgaagatgctgaagaaacagcaactcaaacttgtggtgggacagattccacggaaggattgtttaatatggatgaagatgaagaaactgaacatgaaacaaaggctggtcctgaacggctcgaccagatggtgcatcctgtagccgagcgcctggacatcctgatgtctttggttttgtcctacatgaaggatgtctgctatgtagatggtaaggttgataacggcaaaacaaaggatctatatcgcgacctgataaacatctttgacaaactcctgttgcccacccatgcctcctgccatgtacagtttttcatgttttacctctgtagtttcaaattgggattcgcagaggcatttttggaacatctctggaaaaaattgcaggacccaagtaatcctgccatcatcaggcaggctgctggaaattatattggaagctttttggcaagagctaaatttattcctcttattactgtaaaatcatgcctagatcttttggttaactggctgcacatataccttaataaccaggattcgggaacaaaggcattctgcgatgttgctctccatggaccattttactcagcctgccaagctgtgttctacacctttgtttttagacacaagcagcttttgagcggaaacctgaaagaaggtttgcagtatcttcagagtctgaattttgagcggatagtgatgagccagctaaatcccctgaagatttgcctgccctcagtggttaacttttttgctgcaatcacaaataagtaccagctcgtcttctgctacaccatcattgagaggaacaatcgccagatgctgccagtcattaggagtaccgctggaggagactcagtgcagatctgcacaaacccgctggacaccttcttcccctttgatccctgtgtgctgaagaggtcaaagaaattcattgatcctatttatcaggtgtgggaagacatgagtgctgaagagctacaggagttcaagaaacccatgaaaaaggacatagtggaagatgaagatgatgactttctgaaaggcgaagtgccccagaatgataccgtgattgggatcacaccaagctcctttgacacgcatttccgaagtccttcaagtagtgtgggctccccacccgtgttgtacatgcaacccagtcccctctgacggcagaaatttgtgactgagatgtgacatttgggattccccatcacttgtcatgccctcagcacccagcttgtgccattgggcattgatggcattgaactagagcgagtgcctgcctcggctgtggcacttccaggttcgactgaatcaagcatctgaagactgggtttttttgttgttgttgttccccttacagacaaaatgaagactatcatgtgcaatcttttacagtggggttgatgatacatttggaaggatttgcttgtttaatatgtacattttttgtgttaacagctttttgacacaattactgggtaatttctaatataggcagcagactgttttacgggttgctgttttaacatgggtttttgtcagatccatggtcttaggacttgactgatgagctttcagtgaagaatcctctaagataaaacttctatttaaagactttaactagaaagtgtttattttggctacattgttcaccttctgctgtattggtatttgtctgttgggatttcaagggagtgtagagaagacagaaggaaagctgagagctggcccgacatggtctgggacacagagttggagctggcactgaagatctccagggacttcagagaccaataaaagcccatagggaagagagagaggatatagggaaacagaatcagatgtgtaatatacttggcacagcgaaaaaatggatttaaaagacaaaaatggaggtccaggtagatgtaattcacacagactgaaagtgagttcgggcttgtgtaaaacacatgagattggatttgaccccttggctctcaagtgtccccttagatctagaactgctccttggtggccattagatcgagtcagttttgatctgcatcacttagttattgggaatttctttgttggaaacaggaaaatttttttagattatttggtgtacggttttgctcacaacaataggtggaagttgctagtgcagtcttggtctgatggctgtgtgcatcgcacattcggcttggtgaaatccttctctaaagcctctttttgtatttttataactaaacagaggaagtcttcagaagacctcgctttaaaacaaatttgtgcaaacactgctagagtcattttgaagctcaagcattttcactttgtttcttacatgtgtacttttttgtttacttgtgaaaatggccatctttaagcatatttattttctgccactttatttaaaggcaagcaatattttcttgatcataaatattttgtaatgaaatacttcctcttttccagggctttgtatgcacttgtataattacattgatggcaatgtagagtttgaatttcagtctgtaaatacttttttggaaaatagaaatttttattgctttaaagttttggatatgggtggttttcttttccgggtttggtggaaagtaatttgagaactttaaggttgtctttttaactgctggcaaaatgttgattttttaatattagataaaacgagtaaacgaaattccccagaaattagtagtaagtggggtctttgtgggttgggaagtagttttaatgtagaaagacatttacatataagtctgtttaatttcaaaggagtttgtgaaaaaaaatccatggtgaaaatgaaacaatgacatggttaatctggaacttacgttcttataccaataaaaggtacctcaatacatgttctttcaaaaaaaaaaaaaaaaaa

SEQ ID NO: 42 - DNA, 719 bp, Homo sapiens
  misc_feature (520)..(520): n is a, c, g, or t
acataaagaaaattttcatttatttttccctcagatatacttcaaaataacatgtagacacagaatcacaaatatatacaaaacaaagtatcgcctggttatttcaagaagagttttccctcaattttcttagctgctgtgcataaaattcaaagctttcagggggttcaatgaaagggacaggctctccagcatacttcagcaaagatgcgtaacgctttagaaacagatcatccaattttgtattctttgcagtcaaatgttcatacagtgctttagcagatgtgacatctttctctgagacatagcttttcatgagggaattgtatgcttcttccttttcatttaattcaggaatcaattctaacacagatttcacagttgatgcctttccttgtttcctagaattcctaaggaggaacaacaacaaaatcggggtttgttcagcaattgcaccacatctctgtaggagagctctggcatcatccaccttgcctgcatccacaagttgaaggaaaanatcagtgacaggtttataaattgcaaactgattggccaatctctccgccatgatggcttatctttcaaactgctggtccaactgctcctctattactttctggataagatgccaaggccgagtantcgggtcaatgactttatcctctgaagtaagctattntcatggtttctattgcggcatctatgtaatcattctt

SEQ ID NO: 43 - DNA, 5095 bp, Homo sapiens
acatgctcctcctgtccttctggcggagcgtgcttcccgctgcggggacgttcgagcaatggcagccctgctgagatccgcgcgttggttgctgcgtgccggggcggccccgcgcctcccgctctccctgcgcctcctccctggcggcccgggccggctgcatgccgcctcctatctgcccgccgctcgcgccgggcccgtggccggaggactactgagcccagccaggctgtatgccattgctgccaaagaaaaagatattcaagaggagtccactttttcttctaggaagatttccaatcagtttgattgggctctaatgagactagatctttctgttcgaagaactggccgcattccaaagaagcttctacaaaaagtttttaatgatacctgccgctcaggtggcctaggtggtagtcatgccttgcttctactacgtagttgtggttctctcttgcctgaactaaagcttgaagagagaacagaatttgctcataggatatgggacacacttcagaaattaggtgctgtgtatgatgtgagtcactataatgctttacttaaagtctatcttcaaaatgaatataaattctcaccaactgatttcctggcaaaaatggaggaagcaaacattcaaccaaatcgagtgacataccagagattgattgcttcttattgtaatgtaggagatattgaaggtgccagcaagattcttggatttatgaaaactaaggatctcccagttacagaggcagtattcagtgcccttgtgacagggcatgccagagctggtgatatggagaatgcagaaaacattctcacagtgatgagagatgccggaattgagcctggtccagacacatacctcgcattattgaatgcatatgctgagaagggcgacattgaccatgttaagcagactctggagaaggtggagaagtccgagcttcaccttatggaccgtgatttactgcaaattatttttagcttcagtaaagctgggtatcctcagtatgtctcagaaattttggaaaaagttacatgtgaaagaagatatattccagatgcaatgaacctcattttacttttagtcactgaaaaattggaagatgtagcgttgcaaattttactagcatgccccgtatcaaaggaagatggcccaagtgtctttggcagtttctttttacaacactgtgtgactatgaatacgcctgtggagaagctaacagactactgtaagaagttaaaggaagtccagatgcactcctttcctctgcagttcaccctccattgtgctttactcgccaataaaactgatttggcaaaagccttaatgaaggctgtgaaggaggaaggttttcctatcagacctcactatttctggccattgctagttggacgtcggaaggaaaaaaatgttcaaggtataattgaaatcctcaaaggaatgcaagaattgggagtacatcctgatcaggaaacatatacagattatgtgattccatgctttgatagtgtaaactcagcacgagccattttgcaggaaaatggatgtctgtctgatagtgatatgttttctcaagctggattgagaagtgaagcagcaaatgggaacttagactttgtattatcatttttgaaatcaaatacattgcccatctcgctgcagtctataagaagtagcctactgctaggcttcaggaggtctatgaatataaatctttggagcgagataacagaattgttgtacaaggatggacgttattgccaggagcctcgaggaccgacggaagctgttggctattttctttataacttgattgacagcatgagtgactcagaagtacaggccaaggaggagcatttgagacaatacttccatcagctggagaagatgaatgtaaaaattcctgaaaatatctacagaggcattcgtaatctcctggaaagctaccatgttcctgaattgattaaggatgctcacttgttggttgagagtaagaatttagactttcaaaaaactgtgcaacttacatcatctgaattggagtccacacttgaaacactaaaagctgaaaatcaacctataagagatgtcctaaagcaactcatattagtgctttgttcagaagagaatatgcaaaaagcccttgaattgaaagcaaaatatgaatccgacatggttactggtggctatgcagctttaataaatttatgctgtcgacatgataaagtagaagatgccttgaacttgaaagaagaatttgaccgcttagattcatctgctgtccttgacaccggcaagtatgtaggccttgtaagagtattggcaaagcatggcaagctccaagatgctattaacattctgaaggagatgaaagagaaggatgttcttatcaaagatacaacagccttgtcctttttccacatgctaaatggcgcagctttaagaggtgaaattgaaacagtaaaacagttgcatgaagccatcgtgactctagggttagcagaaccatccaccaacataagtttcccattggtcactgtacacttggaaaagggcgacctatctactgctcttgaggtcgccattgactgctatgaaaagtataaagtattaccaaggattcatgatgtcttgtgtaaactggtagagaaaggcgagactgatctaattcagaaagcaatggactttgtgagccaagaacaaggtgaaatggtgatgctctatgatctcttctttgccttcctacaaacaggaaattacaaagaggccaagaagatcattgagactccagggattagagctcgatctgcaaggcttcagtggttttgtgacagatgtgttgcaaataatcaggttgaaactctggaaaaattagtggagctgacacagaagctatttgaatgtgatagagaccagatgtactacaatctgctaaaactgtataaaataaacggtgactggcaaagagctgatgcagtctggaataaaatccaagaagaaaatgttattcctcgtgaaaagacattaagattattagcagaaatccttagagagggtaaccaggaagttccgtttgacgtacctgagttgtggtatgaagatgaaaaacattccctgaattcttcgtcagcctcaaccacagaacctgatttccagaaagatatattgattgcctgccgattgaaccaaaaaaaaggggcatatgatattttcctgaatgcaaaagagcaaaacattgtgtttaatgctgaaacctacagcaatctcattaaattactgatgtcagaagattattttacacaagcaatggaagtgaaagcattcgcggagacccacatcaagggcttcacactgaacgatgctgccaacagccgcctcatcataacgcaagttaggcgggattatttgaaagaggctgtgacaacactgaaaacagtattggatcagcagcagaccccttctaggttagcagtgacccgtgtcatccaggcattggccatgaagggtgatgttgaaaacatagaagtagttcagaagatgttaaatggactcgaagactccattggactttcaaaaatggttttcatcaataacattgctttggctcaaataaagaataataacatagatgccgcaatagaaaacattgaaaatatgcttacttcagagaataaagtcattgaaccccaatacttcggcttggcatacttattcagaaaagtaatagaggagcagttggaaccagcagttgaaaagataagcatcatggcggagagattggccaatcagtttgcaatttataaacctgtcactgattttttccttcaacttgtggatgcaggcaaggtggatgatgccagagctctcctacagagatgtggtgcaattgctgaacaaaccccgattttgttgttgttcctccttaggaattctaggaaacaaggaaaggcatcaactgtgaaatctgtgttagaattgattcctgaattaaatgaaaaggaagaagcatacaattccctcatgaaaagctatgtctcagagaaagatgtcacatctgctaaagcactgtatgaacatttgactgcaaagaatacaaaattggatgatctgtttctaaagcgttacgcatctttgctgaagtatgctggagagcctgtccctttcattgaaccccctgaaagctttgaattttatgcacagcagctaagaaaattgagggaaaactcttcttgaaataaccaggcgatactttgttttgtatatatttgtgattctgtgtctacatgttattttgaagtatatctgagggaaaaataaatgaaaattttctttatgtacttatgtatgtgtgatgcatgttcaaagtcttattgaccataactctgtgcacttggttattggacatttttggagtttttttctctgggaaaaatcgatagtgttttcttcaatgctgctgctgtgtgaagccatactttttcaggattcttcccctaattggctctttggtttccctgctctgtttcatttatttcattaaaatgttattcctttatttaagattcacttattagtctgctgtttctctgaaaaattttagagctaggtatagtgaccgtgaactttctaacgcataatattctgtgatacagccattccgtacatgtgtgaagtcctgcataactttcgaactttgttaaatgttggcactaggagtcatcagatctaggcttcatcattttccagtgagaagcagagacccaaagggcctgttacttgtgcttggtcaggggactgtctgtcatgcctggaggctcttcggcacacttccccatctttcccttctgccactgtggcttcaagcacctctgttcatagagcgtctctgaaattgagtctcggtcatgacttatcccgaagtagagcaatgtgtttcctctcattgtagtttcaggactttgtcagtacaagctctgccctaggcttgttactttatactcatatcctgaaaagatgtgatttcatctatgaaggggtaaaatattggtttgtatttaattgtttgaaataaaagtgatccctata

SEQ ID NO: 44 - DNA, 5427 bp, Homo sapiens
agatgcgacgaggttagcggggcccgaccactccttggcttcccaggggtgagcctcgcgagttaggagttgggtagagagtcagcccggggcccggcatccgctttttcgttgaagcaacgactttggccggatgactccccagggtcggcatcagcgtgggactgggaacaggtgaagggaaacagaagagagcctgagaagaccgctctcctccgatcctaattgactgagccaatgagagccaaagaggtcacatgctcaactgggcggggagcgggtttccacctgcggcatctttcgcgagcggggagatgagtggggcggaatatgggagaaagggagggcccgccacgctctggctggacacggccttaatcggcccgttcactcgacgtttttggttctacgttgaccccgagaaaccgaaaccgcagggcctagggcgggtggaacgagagggaactacatttcccagcaggctgcggaaacgggactgcggccactacttccggcgtgtaccgagagactggcgtccggcgtgtaccgagagactggcgtccggtgtgcaggtggccacatggatcctggcagccggtggcggaacctgcccagcgggcctagcctaaagcacttgactgacccctcttatggaatcccgcgggaacagcaaaaggcagcgttgcaggagctgacgcgggcgcacgtggagtccttcaactacgctgtgcacgagggtctcggcctcgcggtgcaggctatacctccctttgaatttgctttcaaagatgagcgtatctcttttactattctggatgctgttatcagtccacctacagttccaaaagggaccatctgcaaagaggccaatgtttatccagcagaatgccggggccgaaggagtacctaccgtgggaagttgacagctgatatcaactgggcagtgaatggaatctcaaaaggaatcattaagcagtttcttggctatgttcccatcatggtgaaatccaagctttgcaacttacgtaaccttcccccacaagccctcattgagcaccatgaggaggcagaggaaatggggggctattttataatcaatggcattgaaaaagtcatccgaatgttgattatgcctcggagaaattttcccattgcaatgataagaccaaaatggaaaaccagagggcctggttatactcagtatggagtttcaatgcactgtgtgagggaagaacattccgctgtcaatatgaacctccactacttggaaaatggcactgttatgttgaactttatttaccgaaaagaactgttctttcttcctttgggatttgcacttaaggcacttgtcagcttttctgattatcagatctttcaggagctcatcaaaggaaaagaggatgattctttccttaggaactctgtttctcagatgttaaggattgtaatggaagagggttgttcgacacaaaaacaggtccttaactacctaggtgaatgcttcagagtaaaactcaatgttcctgactggtacccaaatgagcaagctgcggagttcctgtttaaccagtgcatctgtatccacttgaaatccaatactgaaaagttttatatgctttgtctcatgacgcgaaagctctttgctttagccaaaggagagtgcatggaggacaatcctgatagtttggtgaaccaggaagtcctcacaccgggtcagctcttccttatgttcctgaaggaaaaactggaaggttggttagtgtctattaaaatagcttttgataagaaggctcagaagaccagtgtttccatgaacactgacaatttgatgaggatttttacaatgggcatagaccttacaaaaccatttgaatacctttttgctactgggaatctgcgttctaaaacaggtcttggcctcctacaagattctggactttgtgttgtggctgacaagctgaacttcatacgctacctctcccatttccgctgcgtgcacagaggggctgattttgccaagatgaggaccaccacagtacgcaggctgctgccagagtcctggggcttcctttgtcccgtgcataccccagacggggagccctgtggcctgatgaaccacctaactgccgtatgtgaggttgtcacacagtttgtgtatacggcatctattccagctttactgtgcaacttgggggtcactcccattgatggagctccccaccgatcatacagtgagtgctaccctgtcctgctggacggtgtcatggttggctgggtggataaggatcttgctccaggcatcgcagattctcttcgtcattttaaggtgttgagagagaaaagaattcctccctggatggaagtggtccttatacccatgacaggaaaaccaagtctgtacccaggattgttcctttttaccactccttgtagactggtacggcctgtgcagaacttagcattgggcaaagaagagctaattggaactatggaacagatcttcatgaatgtcgctatctttgaggatgaagtttttgctggagttaccacacaccaggaactctttccacacagcctgctgagtgtgattgccaacttcatccctttctctgatcacaaccagagtccacggaacatgtaccaatgccagatgggtaagcaaactatgggctttccacttctcacttatcaagaccgatcggataacaaactgtatcgtcttcagactcctcagagtcccttggtgagaccctccatgtatgattattatgacatggataactatccaattgggaccaatgccatcgttgctgtgatttcttacactggctatgatatggaagatgccatgattgtgaataaggcctcttgggaacgaggctttgcccatggaagtgtctacaagtctgagttcatagacctctctgaaaaaattaaacaaggagatagtagcctggtgtttggcatcaaacctggtgacccacgcgttctgcagaagttagatgacgatggattgccgtttataggagcaaaactgcagtacggagatccgtattacagctacctcaacctcaacaccggggaaagttttgtgatgtactataagagtaaagaaaattgtgttgtggataacatcaaagtgtgcagtaatgacactgggagtggaaaattcaagtgtgtttgcatcactatgagagtgcctcggaacccaactatcggagataaatttgccagtcgccatgggcagaagggcattttaagcagattgtggccggctgaggacatgccttttactgagagtgggatggtcccagacattctgttcaatccccatggttttccatcccgcatgaccattgggatgttaattgagagtatggccgggaagtctgcagctttgcatggtctctgccatgatgctacacccttcatcttctcagaggagaactcggccttagaatactttggtgagatgttaaaggctgctggctacaatttctatggcaccgagaggttatatagtggcatcagtgggctagaactggaagcagacatcttcataggagtggtttattatcagcgcttacgccatatggtctcagacaaatttcaagtaaggacaactggagcccgagacagagtcaccaaccagcctattgggggaagaaatgtccagggtggaatccgttttggggagatggaacgggatgcgcttttagctcatggtacatcttttctccttcatgaccgcctcttcaactgctcagatcggtcggtagcccatgtgtgtgtgaagtgtggcagtttactctctccactgttggagaagccacccccttcttggtctgccatgcgcaacagaaaatacaactgtactctgtgtagtcgcagtgacactatcgatactgtttctgtgccttatgtttttcggtattttgtagctgaactggcagctatgaacatcaaagtgaaactggatgttgtttaacttgatgttgaccttttggattaagaggactatcagattaaagcaaaatgtaattttaattcaatgaagatatcattaccaggttactcttgagatttttcaacggtgttagaactctcaaccaagacctgaaaaccaagtatgcaaggtttctgaatctctctggtagattaactattgacaatgattttctgttatctttgttcaaaaagttcatgtcttctcaaaatatgaaatattgataaatggaagagcatacggtgacaagtctcctttccaaccccaggttccctacaccctgctctcagcaggcagtgagtgtcacacacctgttaatccatcttgagcaggacagtactatacaaatagaatgcaagctgtaatgtaattttatattttcttatagccacgttgaagtaaaaacaaacaggtacagtgttttttaccagctttatagaagtacagttgttacatatttaatgaatacaatttgatgggtctgactatatgcacacacctttgataccatcaccacaatcagggtaataaacatacctgtcatctccacaagtttcctcctgcccctttgttttttgctttttggttgctgttgagtttttgttttgtcttctgtggtaagaacacttaactcaagacctaccctcttaacaaatctttaagtgcacgatatagtattgttaattccaggcaccatgttgtacaacagatctttagaccttacttgtcttgcataactgaagctttatacctgttgaacaactctccatttccctggcccctagcaaccacccttctaccctgtttctatgagtttgactattacagatatctcatatagtgggatcatgcaatatttgtcctgtgactggcttatttcacttagcatagtgaaataagattcatccattttggaagccaggcatggtgctgtgcatctatagtccctgctatttgagaggctgaggtgggaggatcatttgagtgcaggagttcaaggacagcctgggtaatataggaagaccctgtcttgaagaccctgacctcaagtgatccacccacctcggcctccgaaagtgctaggattacaggtgtgagccactgtgcctggcctccggtgagtattttatatttagtctacacttccatacttggcttttttctgcttttatattgatctgctttcatagcagtgtgtagagtgccacttatgttttctttcttgtgtacagtattttattgtatggatttaccatcccctgtgtatttaagttgttccattctttggccattataacttttttctgcaaatattctggtgacttatctttggccattataaactgttgataatagatcatcttgtatatacttctgcaattataagatgttttttgatgatgtaaaaaaaaaaaaaaaaa

SEQ ID NO: 45 - DNA, 1138 bp, Homo sapiens
agggctactaggacgcacgcgcgagatagaacctctagtctcgtggagagattgaagatggcggcttctcaggcggtggaggaaatgcggagccgcgtggttctgggggagtttggggttcgcaatgtccatactactgactttcccggtaactattccggttatgatgatgcctgggaccaggaccgcttcgagaagaatttccgtgtggatgtagtacacatggatgaaaactcactggagtttgacatggtgggaattgacgcagccattgccaatgcttttcgacgaattctgctagctgaggtgccaactatggctgtggagaaggtcctggtgtacaataatacatccattgttcaggatgagattcttgctcaccgtctggggctcattcccattcatgctgatccccgtctttttgagtatcggaaccaaggagatgaagaaggcacagagatagatactctacagtttcgtctccaggtcagatgcactcggaacccccatgctgctaaagattcctctgaccccaacgaactgtacgtgaaccacaaagtgtataccaggcatatgacatggatccccctggggaaccaggctgatctctttccagagggcactatccgaccagtgcatgatgatatcctcatcgctcagctgcggcctggccaagaaattgacctgctcatgcactgtgtcaagggcattggcaaagatcatgccaagttttcaccagtggcaacagccagttacaggctcctgccagacatcaccctgcttgagcccgtggaaggggaggcagctgaggagttgagcaggtgcttctcacctggtgttattgaggtgcaggaagtccaaggtaaaaaggtggccagagttgccaacccccggctggataccttcagcagagaaatcttccggaatgagaagctaaagaaggttgtgaggcttgcccgggttcgagatcattatatctgtaagaaagatttgctggctgcggtggctcacacctgtaatcccagcactttgggaggccaaggcgagtggatcacggggtcaagagagcgagaccatcctggctaacatggtgaaaccccgtctctataaaaaaaaaaaaaaaaaaaaaaaaaaaaaa

SEQ ID NO: 46 - DNA, 1277 bp, Homo sapiens
gagagagagaagatggcggcttctcaggcggtggaggaaatgcggagccgcgtggttctgggggagtttggggttcgcaatgtccatactactgactttcccggtaactattccggttatgatgatgcctgggaccaggaccgcttcgagaagaatttccgtgtggatgtagtacacatggatgaaaactcactggagtttgacatggtgggaattgacgcagccattgccaatgcttttcgacgaattctgctagctgaggtgccaactatggctgtggagaaggtcctggtgtacaataatacatccattgttcaggatgagattcttgctcaccgtctggggctcattcccattcatgctgatccccgtctttttgagtatcggaaccaaggagatgaagaaggcacagagatagatactctacagtttcgtctccaggtcagatgcactcggaacccccatgctgctaaagattcctctgaccccaacgaactgtacgtgaaccacaaagtgtataccaggcatatgacatggatccccctggggaaccaggctgatctctttccagagggcactatccgaccagtgcatgatgatatcctcatcgctcagctgcggcctggccaagaaattgacctgctcatgcactgtgtcaagggcattggcaaagatcatgccaagttttcaccagtggcaacagccagttacaggctcctgccagacatcaccctgcttgagcccgtggaaggggaggcagctgaggagttgagcaggtgcttctcacctggtgttattgaggtgcaggaagtccaaggtaaaaaggtggccagagttgccaacccccggctggataccttcagcagagaaatcttccggaatgagaagctaaagaaggttgtgaggcttgcccgggttcgagatcattatatcttctctgttgagtcaacgggggtgttgccaccagatgtgctggtgagtgaagccatcaaagtactgatggggaagtgccggcgcttcttggatgaactagatgcggttcagatggactgagcttggatgcttctgaggcaagctgaagctttgggttctgactgacccaccctacaggactgctgaacagagagcccagtgtgactagggatcctgagttttctgggacaattccagctttaatcaatacattttgttaaatgtgccataaaatgagactttttacgcctttataaggccttagatgtaaataaactcacccaaacaaaaaaaaaaaaaaaaaa

SEQ ID NO: 47 - DNA, 725 bp, Homo sapiens
ccttccttcctgcttcgcctccgcgcctcgcgctatgggacagagcccccgatccgccagcaccacctgaggatccagaaaccgccccagcgatggaagaggatcaggagctggagagaaaaatatctggattgaagacctcaatggctgaaggcgagaggaagacagccctggaaatggtccaggcagctggaacagatagacactgtgtgacatttgtattgcacgaggaagaccataccctaggaaattctctacgttacatgatcatgaagaacccggaagtggaattttgtggttacactacgacccatccttcagagagcaaaattaatttacgcattcagactcgaggtacccttccagctgttgagccatttcagagaggcctgaatgagctcatgaatgtctgccaacatgtgcttgacaagtttgaggccagcataaaggactataaggatcaaaaagcaagcagaaatgaatccacattctagtcctttatgcagtatacaaggagaactgtcctgtaggatattctcttcctgatggtgcagaacccagaattagaagtttgtggttacagcatactctgtccttcagaaaggcgtgattctagctgttgaccccttgcagctgttggaatctctgcaagaacctctgtattcttctaataaattccctcttttatttaaaaaaaaaaaaaaaaaaaaaaaaaaaaaaaaaaa

SEQ ID NO: 48 - DNA, 6584 bp, Homo sapiens
tttttttttcctttttgggagctgaaaaatttccggtaagggaaagaagggctcctttcgctccttatttccccgcctccttccctcccccaccttcccctcctccggctttttcctcccaactcggggaggtccttcccggtggccgccctgacgaggtctgagcacctaggcggaggcggcgcaggctttttgtagtgaggtttgcgcctgcgcagcgcgcctgcctccgccatgcacgggggtggccccccctcgggggacagcgcatgcccgctgcgcaccatcaagagagtccagttcggagtcctgagtccggatgaactgaagcgaatgtctgtgacggagggtggcatcaaatacccagagacgactgagggaggccgccccaagcttggggggctgatggacccgaggcagggggtgattgagcggactggccgctgccaaacatgtgcaggaaacatgacagagtgtcctggccactttggccacattgaactggccaagcctgtgtttcacgtgggcttcctggtgaagacaatgaaagttttgcgctgtgtctgcttcttctgctccaaactgcttgtggactctaacaacccaaagatcaaggatatcctggctaagtccaagggacagcccaagaagcggctcacacatgtctacgacctttgcaagggcaaaaacatatgcgagggtggggaggagatggacaacaagttcggtgtggaacaacctgagggtgacgaggatctgaccaaagaaaagggccatggtggctgtgggcggtaccagcccaggatccggcgttctggcctagagttgtatgcggaatggaagcacgttaatgaggactctcaggagaagaagatcctgctgagtccagagcgagtgcatgagatcttcaaacgcatctcagatgaggagtgttttgtgctgggcatggagccccgctatgcacggccagagtggatgattgtcacagtgctgcctgtgcccccgctctccgtgcggcctgctgttgtgatgcagggctctgcccgtaaccaggatgacctgactcacaaactggctgacatcgtgaagatcaacaatcagctgcggcgcaatgagcagaacggcgcagcggcccatgtcattgcagaggatgtgaagctcctccagttccatgtggccaccatggtggacaatgagctgcctggcttgccccgtgccatgcagaagtctgggcgtcccctcaagtccctgaagcagcggttgaagggcaaggaaggccgggtgcgagggaacctgatgggcaaaagagtggacttctcggcccgtactgtcatcacccccgaccccaacctctccattgaccaggttggcgtgccccgctccattgctgccaacatgacctttgcggagattgtcacccccttcaacattgacagacttcaagaactagtgcgcagggggaacagccagtacccaggcgccaagtacatcatccgagacaatggtgatcgcattgacttgcgtttccaccccaagcccagtgaccttcacctgcagaccggctataaggtggaacggcacatgtgtgatggggacattgttatcttcaaccggcagccaactctgcacaaaatgtccatgatggggcatcgggtccgcattctcccatggtctacctttcgcttgaatcttagtgtgacaactccgtacaatgcagactttgacggggatgagatgaacttgcacctgccacagtctctggagacgcgagcagagatccaggagctggccatggttcctcgcatgattgtcaccccccagagcaatcggcctgtcatgggtattgtgcaggacacactcacagcagtgcgcaaattcaccaagagagacgtcttcctggagcggggtgaagtgatgaacctcctgatgttcctgtcgacgtgggatgggaaggtcccacagccggccatcctaaagccccggcccctgtggacaggcaagcaaatcttctccctcatcatacctggtcacatcaattgtatccgtacccacagcacccatcccgatgatgaagacagtggcccttacaagcacatctctcctggggacaccaaggtggtggtggagaatggggagctgatcatgggcatcctgtgtaagaagtctctgggcacgtcagctggctccctggtccacatctcctacctagagatgggtcatgacatcactcgcctcttctactccaacattcagactgtcattaacaactggctcctcatcgagggtcatactattggcattggggactccattgctgattctaagacttaccaggacattcagaacactattaagaaggccaagcaggacgtaatagaggtcatcgagaaggcacacaacaatgagctggagcccaccccagggaacactctgcggcagacgtttgagaatcaggtgaaccgcattcttaacgatgcccgagacaagactggctcctctgctcagaaatccctgtctgaatacaacaacttcaagtctatggtcgtgtccggagctaaaggttccaagattaacatctcccaggtcattgctgtcgttggacagcagaacgtcgagggcaagcggattccatttggcttcaagcaccggactctgcctcacttcatcaaggatgactacgggcctgagagccgtggctttgtggagaactcctacctagccggcctcacacccactgagttctttttccacgccatggggggtcgtgaggggctcattgacacggctgtcaagactgctgagactggatacatccagcggcggctgatcaagtccatggagtcagtgatggtgaagtacgacgcgactgtgcggaactccatcaaccaggtggtgcagctgcgctacggcgaagacggcctggcaggcgagagcgttgagttccagaacctggctacgcttaagccttccaacaaggcttttgagaagaagttccgctttgattataccaatgagagggccctgcggcgcactctgcaggaggacctggtgaaggacgtgctgagcaacgcacacatccagaacgagttggagcgggaatttgagcggatgcgggaggatcgggaggtgctcagggtcatcttcccaactggagacagcaaggtcgtcctcccctgtaacctgctgcggatgatctggaatgctcagaaaatcttccacatcaacccacgccttccctccgacctgcaccccatcaaagtggtggagggagtcaaggaattgagcaagaagctggtgattgtgaatggggatgacccactaagtcgacaggcccaggaaaatgccacgctgctcttcaacatccacctgcggtccacgttgtgttcccgccgcatggcagaggagtttcggctcagtggggaggccttcgactggctgcttggggagattgagtccaagttcaaccaagccattgcgcatcccggggaaatggtgggggctctggctgcgcagtcccttggagaacctgccacccagatgaccttgaataccttccactatgctggtgtgtctgccaagaatgtgacgctgggtgtgccccgacttaaggagctcatcaacatttccaagaagccaaagactccttcgcttactgtcttcctgttgggccagtccgctcgagatgctgagagagccaaggatattctgtgccgtctggagcatacaacgttgaggaaggtgactgccaacacagccatctactatgaccccaacccccagagcacggtggtggcagaggatcaggaatgggtgaatgtctactatgaaatgcctgactttgatgtggcccgaatctccccctggctgttgcgggtggagctggatcggaagcacatgactgaccggaagctcaccatggagcagattgctgaaaagatcaatgctggttttggtgacgacttgaactgcatctttaatgatgacaatgcagagaagctggtgctccgtattcgcatcatgaacagcgatgagaacaagatgcaagaggaggaagaggtggtggacaagatggatgatgatgtcttcctgcgctgcatcgagtccaacatgctgacagatatgaccctgcagggcatcgagcagatcagcaaggtgtacatgcacttgccacagacagacaacaagaagaagatcatcatcacggaggatggggaattcaaggccctgcaggagtggatcctggagacggacggcgtgagcttgatgcgggtgctgagtgagaaggacgtggaccccgtacgcaccacgtccaatgacattgtggagatcttcacggtgctgggcattgaagccgtgcggaaggccctggagcgggagctgtaccacgtcatctcctttgatggctcctatgtcaattaccgacacttggctctcttgtgtgataccatgacctgtcgtggccacttgatggccatcacccgacacggagtcaaccgccaggacacaggaccactcatgaagtgttcctttgaggaaacggtggacgtgcttatggaagcagccgcacacggtgagagtgaccccatgaagggggtctctgagaatatcatgctgggccagctggctccggccggcactggctgctttgacctcctgcttgatgcagagaagtgcaagtatggcatggagatccccaccaatatccccggcctgggggctgctggacccaccggcatgttctttggttcagcacccagtcccatgggtggaatctctcctgccatgacaccttggaaccagggtgcaacccctgcctatggcgcctggtcccccagtgttgggagtggaatgaccccaggggcagccggcttctctcccagtgctgcgtcagatgccagcggcttcagcccaggttactcccctgcctggtctcccacaccgggctccccggggtccccaggtccctcaagcccctacatcccttcaccaggtggtgccatgtctcccagctactcgccaacgtcacctgcctacgagccccgctctcctgggggctacacaccccagagtccctcttattcccccacttcaccctcctactcccctacctctccatcctattctccaaccagtcccaactatagtcccacatcacccagctattcgccaacgtcacccagctactcaccgacctctcccagctactcacccacctctcccagctactcgcccacctctcccagctactcgcccacctctcccagctactcacccacttcccctagctactcgcccacttcccctagctactcgccaacgtctcccagctactcaccgacatctcccagctactcgccaacttcacccagctattctcccacttctcccagctactcacctacctctccaagctattcacccacctcccccagctactcacccacttccccaagttactcacccaccagcccgaactattctccaaccagtcccaattacaccccaacatcacccagctacagcccgacatcacccagctattcacctactagtcccaactacacacctaccagccctaactacagcccaacctctccaagctactctccaacatcacccagctattccccgacctcaccaagttactccccttccagcccacgatacacaccacagtctccaacctataccccaagctcacccagctacagccccagctcgcccagctacagcccaacctcacccaagtacaccccaaccagtccttcttacagtcccagctccccagagtataccccaacctctcccaagtactcacctaccagtcccaaatattcacccacctctcccaagtactcgcctaccagtcccacctattcacccaccaccccaaaatactccccaacatctcctacttattccccaacctctccagtctacaccccaacctctcccaagtactcacctactagccccacttactcgcccacttcccccaagtactcgcccaccagccccacctactcgcccacctcccccaaaggctcaacctactctcccacttcccctggttactcgcccaccagccccacctacagtctcacaagcccggctatcagcccggatgacagtgacgaggagaactgagggcacgtggggtgcggcagcgggctagggcccagggcagcttgcccgtgctgctgtgcagttcttgcctccctcacggggcgtcacccccagcccagctccgttgtacataaatgccttgtggcagagctcccggtgaacttctggatcccgtttctgatgcagactcttgtcttgttctccacttgtgctgttagaactcactggcccagtggtgttctcactcctaccccacccaccccctgcctgtccccaaattgaagatccttccttgcctgtggcttgatgcggggcgggtaaagggtattttaacttaggggtagttcctgctgtgagtggttacagctgatcctcgggaagaacaaagctaaagctgccttttgtctgttattttatttttttgaagtttaaataaagtttactaattttgaccaaaa

SEQ ID NO: 49 - DNA, 342 bp, Homo sapiens
atccggcgttctggcctagagctgtatgcggaatggaagcacgttaatgaggactctcaggagaagaagatcctgctgagtccagagcgagtgcatgagatcttcaaacgcatctcagatgaggagtgttttgtgctgggcatcgagccccgctatgcacggccagagtcgatgattgtcacagtgctgcccgtgcccccgctgtccgtgcggcctgctgttgtgatgcagggctctgcccgtaaccaggtcagtggctccagggctctgcctcttagctggagggcgaggtgtctaaagggaggaagacctgtaggtcgggatggtgatgacctggtttga

SEQ ID NO: 50 - DNA, 3748 bp, Homo sapiens
cttttgatttcaagagttaggagctcgagaaccgtttggcaatatgtacgacgcggatgaggatatgcaatatgatgaggatgatgatgaaatcaccccggatttgtggcaagaagcatgctggattgtaatcagttcctattttgacgagaaaggcttggttagacaacagctggattcttttgatgagtttattcagatgtctgttcaaagaattgtggaagacgctcctcctatagacctacaggctgaagctcagcatgctagtggagaagttgaagaaccgccacgatatttgctgaagtttgaacaaatttatctttccaagcctacccattgggaaagagatggtgctccttcaccaatgatgcccaatgaagctagattaaggaatctcacgtattctgctccgctttatgttgatataacaaaaacagtcattaaagaaggtgaagaacaacttcagactcagcatcagaaaacttttataggaaaaattccaattatgttgcggtcaacttactgccttttgaatggcttgacagatcgtgatctttgtgagttaaatgaatgccctttggatcctggtggctatttcattattaatggatcagaaaaggttctgattgcccaagagaaaatggcaacaaacacagtttatgtgtttgccaaaaaggattctaaatatgcctacacaggagagtgtagatcatgtcttgagaattcttcccgacccaccagtactatatgggttagcatgctggcaagaggaggacagggtgccaagaagagtgctattggtcagcgcattgtggcaactctaccatatatcaagcaagaagttcccatcattattgtgttcagagcattaggttttgtgtccgacagagatattttagaacatattatttatgattttgaagatccagagatgatggaaatggttaaaccttctctcgatgaagcttttgtcatccaagaacagaatgttgcactaaatttcattggttcacgaggagcaaagcctggtgttactaaagagaaaagaattaaatatgcaaaggaagttttacaaaaagaaatgctccctcatgttggtgtcagtgatttttgtgagaccaaaaaagcctatttcttgggatacatggttcataggttacttctggcagctttgggtagaagagaactagatgacagagatcactatggaaacaagagattggatcttgctgggccgctgcttgcattcttatttagaggtatgtttaagaatttgcttaaagaagtgcggatctatgcacagaaatttattgatcgaggaaaggattttaacttggagttggcaattaaaacacggatcatatctgatggcctaaaatactctttagctactggaaactggggtgatcaaaagaaagctcatcaagccagagctggagtatctcaggtgttaaaccgcctgacttttgcgtctactctttctcacctgcgtcgtttaaattctcctattggtagagacggcaagctagcaaaaccaagacagttgcataatacgttgtggggaatggtgtgtcctgccgagaccccagagggccatgctgtaggacttgtgaagaatttagccttgatggcgtatatttcagttggatctcaaccatctccaattctggaatttttagaagaatggagtatggaaaatttagaagaaatttctcctgcagctattgctgatgcaaccaagatttttgttaatggctgctgggttggaatacataaagatcccgaacaacttatgaacaccctaaggaaattgagacgtcagatggacatcattgtgtctgaagtttctatgatcagagatattcgagagagggagattcggatctatacggatgcaggccgtatttgtagaccacttctgattgtggaaaaacaaaagctacttttgaagaagaggcatattgaccaattgaaagagagagaatataacaactatagttggcaggatcttgtggccagtggggtagtggagtatattgataccctggaagaagaaacagtgatgcttgcaatgactccagatgatttacaggagaaagaagtagcttattgttccacatatacacactgtgagattcatccctcaatgatccttggtgtctgtgcatctattattccctttcctgatcataaccagtcccctagaaacacataccagtctgctatgggtaagcaggctatgggagtttacatcaccaacttccatgttcgcatggacacattggcccatgttctctattatcctcaaaagccacttgtgactacacggtctatggaatatctacgatttagagagctgccagcaggcatcaactcaattgtggccattgcatcatacactggatataatcaggaagactctgttatcatgaatcgttcagctgtagaccgcggcttcttcaggtctgttttctatcgctcatacaaagaacaggagtctaaaaaaggatttgatcaagaagaagtttttgagaagcctacacgtgaaacatgccagggcatgaggcatgccatttacgacaagctggatgatgatggtttgatagctccaggggttcgtgtatcaggagatgatgttattataggcaaaacagtcaccttgcctgaaaatgaagatgaattggagagcaccaatagacgctataccaagagagactgtagcacttttctcagaactagtgagacgggcattgtggatcaggttatggtaactctcaatcaggaaggatataaattttgtaaaataagggtacgctctgttaggattccacagattggagacaaatttgctagtcgacatggtcaaaagggtacttgtggtattcagtatagacaagaggatatgcctttcacctgtgaaggtatcacccctgatatcatcatcaatccccatgccatcccctctcgtatgactattggtcacttaattgaatgccttcaagggaaggtatcggctaacaagggtgaaattggtgatgccactccatttaatgatgctgttaacgtgcagaagatttctaatcttttatctgattatggctatcatctcagaggaaatgaggtcctgtacaatgggttcactggtcgaaaaatcacatcacaaatatttattggccccacttattaccagcgtttgaagcatatggtggatgataagattcactctcgtgctaggggacctattcagatcctcaatagacagcccatggagggtagatctcgtgatggtggcctgcgttttggagaaatggaacgagattgtcagattgcccatggagcagcccagtttttaagggaaagattgtttgaggcatcagatccatatcaggttcatgtttgcaatctttgtggaataatggcgattgccaacaccaggacccatacatatgaatgcaggggctgccgcaataaaacccagatttctttggtgcgaatgccttacgcatgcaaactattgtttcaggaacttatgtctatgagtattgcaccgcgaatgatgagtgtttagctattttacaggagtcaacaagataattaaatatcttggtgtcttgtttctattgtgtggctttataaaaatgacaaatatgtactgtgttgtgataaaaagtattttatttgtttaatgatatgcatgcttttcttctgtaaatatataataaatttttgtagatagtcttgatgtgtg

SEQ ID NO: 51 - DNA, 1422 bp, Homo sapiens
ggtggcccctgggcgagatgccgtacgccaaccagcctaccgtgcggatcacggagctcactgacgagaatgtcaagttcatcatcgagaacaccgacctggcggtggccaattcgattcggagggtcttcatcgctgaggttcccataatagccattgactgggttcagattgatgccaattcctcagtccttcatgatgaattcattgctcacaggcttggattaattcccctcattagtgatgacattgtggacaagctgcagtactctcgggactgcacatgtgaggagttctgccccgagtgctcggtggagttcaccctcgatgtgcggtgcaatgaagaccagacgcgacatgtcacgtctcgagacctcatctccaacagcccccgggtcattccggtgacatcccggaaccgagataatgaccccaatgactacgtggagcaggatgacatcctcatcgtcaagttgagaaagggccaggagctgagacttcgagcctatgccaaaaagggctttggcaaggagcatgccaagtggaaccctactgcaggggtggcttttgaatacgatccagacaatgccctgaggcacacagtgtaccccaagcccgaggaatggccaaagagtgagtactcggagctggatgaggatgagtcgcaggctccctatgaccccaacggcaagccagaaaggttttactacaatgtggagtcctgtggctctctgcgtcctgaaaccattgtcctgtcagccctctcaggattgaagaagaaactgagtgatttacaaactcaattaagccacgagatccagagtgatgtgctaaccataaattaactgcagcttgcctgcttcagcaaaaacggagattcaggccagcagctggatatgggggtctctcttcagactcttctcgtttctgagaatctagtctactgttggttgagcttcttggcaggacatcagtaccaactagaagtgggtcatagatagattaccagggatgcagtggtgtttaggcaggataggtctttactggccctgactgctgttaataattggcagcagtgctccccagatcccagaaggtccctgctggagtgtttccagtgcacctgtagggaaccaactagacttctctcctggttagtccagctctttactctaaaccctttctgtccaaaatgagtcattttcagttgtaccttagatgtctggtgttgaggatcaagtgccatagcctttattcagggggcctataaacccttccagttcttgccccagggctggcctgctagcgcttcaaattcccaggtgtccctaatttgagaagtaaccttttggaatagcattagaccctggctgtcccctccccaccaaataaacatgatatttcaaaaaaaaaaaaaaaaaa

SEQ ID NO: 52 - DNA, 778 bp, Homo sapiens
tatgttgtgctctttattgccaaaaataaacacttttaaaaagacaacttctgttaatcaataaatatcctttcctttctgtactggatctctggttttctccaggctgcctaattaactgctttgtgcatgttcctatctggagacagtgctgaggggttagggccagagccttcattaaggagaaatgaaatactcctcctgtgtatgtgggaggggcccttgctcctgcggctgcttcacccccacccccacccccacccccaccaaactaaatgatcacggcattggaggtgaaggggctcatgactgctggacagacaatgaaatatcatgtttatttggtggcgagggcacagccactgtctaatgctattccaaaaggttactgatcacactacggacacctgcgaatgctaagcgctagcatgccacccctgggtcaagaactgcaagggcttataggcccctgaataaaggctatggcacttgatcctgaacaccagacatctaaggtacaactgacaatgactcattccggacagaaatggcttagactaaagagctggactaaccagcgaaagaagtctagttgatcccttcaggtgcactggaaacacttcagccgggacctttctggatctggggaccactgctgcccaacataaactgcagaccaggccatttaagacctatgctttgctaacacaactggtgccctgatatccatttatgacccactttttattgccactgtgaccttgccagaaagctaacc

SEQ ID NO: 53 - DNA, 1616 bp, Homo sapiens
accgcggagcagacgcggaggctggtggccctgggcgagatgccgtacgccaaccagcctaccgtgcggatcacggagctcactgacgagaatgtcaagttcatcatcgagaacaccgacctggcggtggccaattcgattcggagggtcttcatcgctgaggttcccataatagccattgactgggttcagattgatgccaattcctcagtccttcatgatgaattcattgctcacaggcttggattaattcccctcattagtgatgacattgtggacaagctgcagtactctcgggactgcacatgtgaggagttctgccccgagtgctcggtggagttcaccctcgatgtgcggtgcaatgaagaccagacgcgacatgtcacgtctcgagacctcatctccaacagcccccgggtcattccggtgacatcccggaaccgagataatgaccccaatgactacgtggagcaggatgacatcctcatcgtcaagttgagaaagggccaggagctgagacttcgagcctatgccaaaaagggctttggcaaggagcatgccaagtggaaccctactgcaggggtggcttttgaatacgatccagacaatgccctgaggaccacagtgtaccccaagcccgaggaatggccaaagagtgagtactcggagctggatgaggatgagtcgcaggctccctatgaccccaacggcaagccagaaaggttttactacaatgtggagtcctgtggctctctgcgtcctgaaaccattgtcctgtcagccctctcaggattgaagaagaaactgagtgatttacaaactcaattaagccacgagatccagagtgatgtgctaaccataaattaactgcagcttgcctgcttcagcaaaaacggagattcaggccagcagctggatatgggggtctctcttcagactcttctcgtttctgagaatctagtctactgttggttgagcttcttggcaggacatcagtaccaactagaagtgggtcatagatagattaccagggatgcagtggtgtttaggcaggataggtctttactggccctgactgctgttaataattggcagcagtgctccccagatcccagaaggtccctgctggagtgtttccagtgcacctgtaggaacaactagacttctctcctggttagtccagctctttactctaaaccctttctgtccaaaatgagtcattttcagttgtaccttagatgtctggtgttgaggatcaagtgccatagcctttattcagggggcctataaacccttccagttcttgccccagggctggcctgctagcgcttcaaattcccaggtgtccctaatttgagaagtaaccttttggaatagcaggagcaagggcccctcccacatacacaggaggagtatttcatttctccttaatgaaggctctggccctaacccctcagcactgtctccagataggaacatgcacaaagcagttaattaggcagcctggagaaaaccagagatccagtacagaaaggaaaggatatttattgattaacagaagttgtctttttaaaagtgtttatttttgcaataaagagcacaacataaaaaaaaaa

SEQ ID NO: 54 - DNA, 2338 bp, Homo sapiens
gctccgcgcttctgcttccggcggcgctggtggtggcgcgcggcgcggcgcggcgatggcggcgggtggcagcgatccgcgggctggcgacgtagaggaggacgcctcacagctcatctttcctaaagagtttgaaacagctgagacacttctaaattcagaagttcatatgcttctggaacatcgaaagcagcagaatgagagtgcagaggacgaacaggagctctcagaagtcttcatgaaaacattaaactacacagcccgtttcagtcgtttcaaaaacagagagaccattgccagtgttcgtagcttgctactccagaaaaagcttcataagtttgagttggcctgtttggccaacctttgcccagagactgctgaggagtccaaggctctaatcccaagcttggagggacggtttgaagatgaggagctgcagcagattcttgatgatatccagacaaagcgcagctttcagtattaatctccaaacatcactgctgctcggagaaaccacatccccaggcataacaccaccttcccactgtctggggctgacttgcacagaaattctgttgaagacagttgagaattcctttggagaaaacagcccagcttggcgtggggttaggttgctgtttcaaataactcacaggcccaggtgacatggaatcttggagcagccttgtgcagtggcagccagtggcttcctgaacgtgcctctgcgaagtgtgagatgaggggtcacataaccacactgttgactacctcattcctggtttttggcctccacatcatcttttttcttaatatttcatgttttaatttcagggtgtttatactttttgaaactagaccagaagatagtagactttatagagaaagaccagttttacctagatactaaaggaagaattaaaccgctgttagtttgaaatgctttttttttttttttttaaatggagatagggtcttaactcttgtccaggctggaggagtgcagtcgtacagtcatggctcactgaagtcttgaccccgctgcctcagcctcccaaataactggggccacaggtgtgcaccacaactctcagctaatttttaaaattttttatagaggtggggttttactatgctgtccagactggtcttaaactcctgggctcaagtgatccccctgccttggcctcccaaactggtgagattacaggcatgagccaccacaactggcctgaaattcttaaaggatgggagtgtcgatgacagcaccttggcatcgttgtgcctaacctgggagacggaagaagcacgccatgggaagtgtttacacttggggacaagtgctaagtattgtggagcccatagccccttgagatagatggctactttgcctttcttcttgaactgtcttgcagaatgtggatttggggtaagtggtcttgaaggattcatttagtcaccctcaaattaagatttttacttcatctttcttgggcctgcacctccaagataacaaagaagaagcaatggtcgtgccaaagaggtccacaaccaggtgtgcactgttcactgcagcccatttgctgtatgaactgtggttgttgtgtgcccaatgacaaggctactaagaaattcatcatttgaaacgtagaggccgcagcagtcagcgatgtttctgaaatgagcatccttgacgcctgtgtacttcccaggctggatgtgaagctacattaccatgtgagttgtgccattcacagcacagtggtgaggaattgagctcatgaagcaggcaaggaccgaacacctccaccccaacgtagacctgcaggtgctgccccatgacctccaccaaagcccatataaggagcggagttgttaaggactgaagaaaaacttctctggagaaaaataaaattgcaattctacttaaaaaaatttttttttttttttacttcataggccaggcttgaagttctgaacactttgaagtctccaattatgagagatccagtctaagcctctggcctgctaattagcaataagtgctttatttggaaggagggagtcatccactcttgagccactgcagtgaagtcacttgatctcagtctgggggaaaacacttcaatagctaaacattctagctttgatttttctgaagggaatacacttgttttcaattttggggtttttctttggggcacttgcttgactctgtatgaacttgtgatccaaggaaaaaggagaaagaacagtgttggcttttaaaatcaggatggttttatgtttgctacgaaataaggcaagaataaaaaattcttatttttaaaaaaaaaaaaaa

SEQ ID NO: 55 - DNA, 566 bp, Homo sapiens
aaaatacgaattttttattcttgccttatttcgtagcaaacataaaaccatcctgattttaaaagccaacactgttctttctcctttttccttggatcacaagttcatacagagtcaagcaagtgccccaaagaaaaaccccaaaattgaaaacaagtgtattcccttcagaaaaatcaaagctagaatgtttagctattgaagtgttttcccccagactgagatcaagtgacttcactgcagtggctcaagagtggatgactccctccttccaaataaagcacttattgctaattagcaggccagaggcttagactggatctctcataattggagacttcaaagtgttcagaacttcaagcctggcctatgaagtaaaaaaaaaaaaaaaatttttttaagtagaattgcaattttatttttctccagagaagtttttcttcagtccttaacaactccgctccttatatgggctttggtggaggtcatggggcagcacctgcaggtctacgttggggtggaggtgttcggtccttgcctgcttcatgagctcaatttctcaccac

SEQ ID NO: 56 - DNA, 903 bp, Homo sapiens
  misc_feature (359)..(359): n is a, c, g, or t
cctcaccgtgctggtgtcccatacgatgaccccaccgaccagatgtttgtgttctttccagaggagcccaaggtgggcatcaagaccatcaaggtgtactgccagcgcatgcaggaggagaacatcacacgggctctcatcgtggtgcagcagggcatgacaccctccgccaagcagtccctggtcgacatggcccccaagtacatcctggagcagtttctgcagcaggagctgctcatcaacatcacggagcacgagctagtccctgagcacgtcgtcatgaccaaggaggaggtgacagagctgctggcccgatacagtaagctccgagagaaccagctgcccaggatccaggcggnggaccctgtggcgcgctactttgggataaagcgtgggcaggtggtgaagatcatccggcccagtgagacggctggcaggtacatcacctaccggctggtgcagtagctaccgcctgacagcccctagaggcggacacacagcgacccccatccctgcangacaaacgcccctgccctgccagaatccggcccccacagctctcacggctgctgctcctctggactccccaggcangtggnctncaccccacgtctcccgtcctggngtgagggcttctgtgggccagnccngccccaatcacctnngtgattgtgcgagatgcagcctcagaaggaaacaagcccccagagggaggtcacctggnggcagctggtgccgggtctcacccaaaccacgcttggtcccctctgtgggggtttgggggcggggctccaccaagcactgttttctcctggcccttggctttcaccctttttttccttctcgggggcctgatggtgggggccccgcccgctcgggttttattccttcaaaccggcccgc

SEQ ID NO: 57 - DNA, 1248 bp, Homo sapiens
ggcggcggcggcggaggctgccatggacgacgaggaggagacgtaccggctctggaaaatccgcaagaccatcatgcagctgtgccacgaccgtggctatctggtgacccaggacgagcttgaccagaccctggaggagttcaaagcccaatttggggacaagccgagtgaggggcggccgcggcgcacggacctcaccgtgctggtggcccacaacgatgaccccaccgaccagatgtttgtgttctttccagaggagcccaaggtgggcatcaagaccatcaaggtgtactgccagcgcatgcaggaggagaacatcacacgggctctcatcgtggtgcagcagggcatgacaccctccgccaagcagtccctggtcgacatggcccccaagtacatcctggagcagtttctgcagcaggagctgctcatcaacatcacggagcacgagctagtccctgagcacgtcgtcatgaccaaggaggaggtgacagagctgctggcccgatataagctccgagagaaccagctgcccaggatccaggcgggggaccctgtggcgcgctactttgggataaagcgtgggcaggtggtgaagatcatccggcccagtgagacggctggcaggtacatcacctaccggctggtgcagtagctaccgcctgacagcccctagaggcggacacacagcgacccccatccctgcaggacaaacgcccctgccctgccagaatccggcccccacagctctcacggctgctgctcctctggactccccaaggcaggtggcctccacccacgttctcccgtcctggggtgaggcttcctgtggcccagcccgccccattcacctgtggatttgtgcgagatgcagcctcagaaggaacaaggcccccagagggaggtcacctgggggcagctggtgccgggtcttcacccagaccacgctgggtcccctctgttgggggtttggggtccgggtctcccaccagccactgcttcctcctgggccctcggccttccacccctcgtcttccctccctcgggggccctgatgcgtggcggcccccacccggcctcggctctttactccattcacagccgtgcacgcgctcaagccaccagggtgcgagatgccagctctggagttctcggttgttgtaggaggttgggtgttttcaaatggtaaagatgttttgagcaaataaatttgcttgatacagaaaaaaaaaaaaaaaaaaaaaaaaa

SEQ ID NO: 58 - DNA, 588 bp, Homo sapiens
cgcaggcgcaagataagctaggagccgcgcgagtcgtagtgtcgctgtttgcgggtctccgcgcgggaccggggcgcagcggggtcgctgaggcgagggtgtcatgtcagacaacgaggacaattttgatggcgacgactttgatgatgtggaggaggatgaagggctagatgacttggagaatgccgaagaggaaggccaggagaatgtcgagatcctcccctctggggagcgaccgcaggccaaccagaagcgaatcaccacaccatacatgaccaagtacgagcgagcccgcgtgctgggcacccgagcgctccagattgcgatgtgtgcccctgtgatggtggagctggagggggagacagatcctctgctcattgccatgaaggaactcaaggcccgaaagatccccatcatcattcgccgttacctgccagatgggagctatgaagactggggggtggacgagctcatcatcaccgactgagctggagtcatcttcctgcccttgccccatgcccaattttcattctcactttatatgtgtaaataataaaatattcaactttcaaaaaaaaaaaaaaaaaa

SEQ ID NO: 59 - DNA, 852 bp, Homo sapiens
ctcctagtcgcagcaagcgcggaactggggttgcggcgtctaagtgtttccggtggattcccagggactgtcggaggtgtggactctgcctgcctacctggtctgggaagatgttctaccatatctccctagagcacgaaatcctgctgcacccgcgctacttcggccccaacttgctcaacacggtgaagcagaagctcttcaccgaggtggaggggacctgcacagggaagtatggctttgtaattgctgtcaccaccattgacaatattggtgctggtgtgatccagccaggccgaggctttgtcctttatccagttaagtacaaggccattgttttccggccatttaaaggggaggtcgtggatgctgttgtcactcaggtcaacaaggttggactcttcacagaaattgggcccatgtcttgcttcatctctcgacattccatcccttcagagatggagtttgatcctaactccaacccaccatgttacaagacaatggatgaggatattgtgattcagcaggacgatgagatccgcttaaagattgtggggacccgtgtggacaagaatgacatttttgctattggctccctgatggacgattacttggggcttgtaagctgagcctggtggcctcctacccttggtcctactctaggaagtgtgattgtcacacttatcatgttgtccagaggtccagtctggctgctgttgtggaggcaaggaaggcaactcatcccagaaggcatctggtgcttcttgtagcttaactactgcctcctcatttttcagtatgtgttctaagtataaaaagtccttggttctcatggaaaaaaaaaaaaaaaaa

SEQ ID NO: 60 - DNA, 867 bp, Homo sapiens
ccctcactaaagggacaaaagctggagctccaccgcggtgcggccgctctagaactagtggatcccccgggctgcaggtccggtcttgtccacgctagggggtgcacgtactcccaactgtggtcgcgctctcaccccttctgctgctctcgtggccccctcgcgatggcgggcatcctgtttgaggatattttcgatgtgaaggatattgacccggaggccaagaagtttgaccgagtgtctcgactgcattgtgagagtgaatctttcaagatggatctaatcttagatgtaaacattcaaatttaccctgtagacttgggtgacaagtttcggttggtcatagctagtaccttgtatgaagatggtaccctggatgatggtgaatacaaccccactgatgataggccttccagggctgaccagtttgagtatgtaatgtatggaaaagtgtacaggattgagggagatgaaacttctactgaagcagcaacacgcctctctgcgtacgtgtcctatgggggcctgctcatgaggctgcagggggatgccaacaacctgcatggattcgaggtggactccagagtttatctcctgatgaagaagctagccttctgaacctcgcctgaagccagcctctctgccaagtcactcaggtcatgggcattgttcaagcctgagtggcagccgctcttgctcacctgttgaggaagggctggctcactgtccaccgtggcggcatctttaactggcctccactcaatgggaaactgactcgcctgtgaaagacacagtgggagagctgaaaatgaatcagaagctttatgtatatgatttttaaattaaactttactttttcagactgcc

SEQ ID NO: 61 - DNA, 960 bp, Homo sapiens
  misc_feature (631)..(631): n is a, c, g, or t
gggggcactggccttcaaccaagccaagcgccttggcttctgtcctggatgctcagtgttttgcggtttacgatgcgcatcgttttcccggctctggcaccagatccatactattctgtaaactgaggcggcgatgcccaaaaattggaagcaattactctaagatatcaaaagattgaagcaagccatgaaacctctgcgcggagagagccgagcccgaggcggcggggagccagcacgtgtgcgcagctccgccctcttttggcgaagcacctaggcctggcccctcccgcgacctgtagcgcggcggagcaagcgcagaaggctgggagggctgcgcgggctgcgcgtcgccatggagcccgacgggacttacgagccgggcttcgtgggtattcgcttctgccaggaatgtaacaacatgctgtaccccaaggaagacaaggagaaccgcattctgctctacgcgtgccggaactgtgatttccagcaggaggccgacaacagctgcatctatgtcaacaagatcacgcacgaagtggacgaactgacccagattatcgccgacgtgtcccaggaccccacgttgccgcggaccgaggaccacccgtgccaaaagtgcggccacaangaggctgtgttcttncagtcacacagtgcgcnggccgangacccatgcgcctttactacgtgtgcacaagccccacacttgcgggcaccgctggaccgaatgacctctntnttccccgatgtaataaacaccaaattcatgcgtgaaaaaaaaaaaaaaaactttcatccgncanctcatgatatattcnaccggaattccgaccgnacctgagngtacactttctttatggcgataanctgngatatggctactgtctgngaatgttcntcatcccanatcaccgactaaggaaactgggcatagacnatnatatggtggtatcnttc

SEQ ID NO: 62 - DNA, 652 bp, Homo sapiens
ggtggcggcggcggcggacccttggggtctggacgcaacggcggcgggagcatgaacgcccctccagccttcgagtcgttcttgctcttcgagggcgagaagaagatcaccattaacaaggacaccaaggtacccaatgcctgtttattcaccatcaacaaagaagaccacacactgggaaacatcattaaatcacaactcctaaaagacccgcaagtgctatttgctggctacaaagtcccccaccccttggagcacaagatcatcatccgagtgcagaccacgccggactacagcccccaggaagcctttaccaacgccatcaccgacctcatcagtgagctgtccctgctggaggagcgctttcgggtggccataaaagacaagcaggaaggaattgagtaggggccagagggggctctgctcggcctgtgagccccgttcctacctgtgcctgaccctccgctccaggtaccacaccgaggagagcggcggtcccagccatggcccgccttgtggcacccctcaccctgacaccgacgtgttggcacccctcaccctgacaccgacgtgtcctgtacatagattaggttttatattcctaataaagtatagcgggagagacctggaaaaaacaacaacaaaaaaacct

SEQ ID NO: 63 - DNA, 433 bp, Homo sapiens
cacgaggcgagtcgttcttgctcttcgagggcgagaagatcaccattaacaaggacaccaaggtacccaatgcctgtttattcaccatcaacaaagaagaccacacactgggaaacatcattaaatcacgtgcctgcttccccttcgccttctgccgtgattgtcagtttcctgaggcctccccagccacgcttcctgtacagcctgcagaactctgccccagagcacatcagctatgtgccccagctctcaaacgacaccttgtccggggaggctcaccctgtccaccttcacgctggagcagcctctaggccagttcagcagccacaacatctctgacttggataccatctggctggtggtggccctcagcaacgccacccagagcttcacggccccacggacaaaccaggacatccctgctcctgccaac

SEQ ID NO: 64 - DNA, 494 bp, Homo sapiens
tgggggggcagggtctcactctgtcacccaggctggagtgcagtggtgcgatcatagctcactgcggcctcgacctcccaggctcaaggctctcagattctcagaccccaccctggggaaggatggaggatgaggacaagcttcccaggtccacatgacagagggactcaggattggcaatttggcctgtgtccagctttgctgctcagagagctcagatccccacccctcaattcactcccttgtgtggaagggagtgaccctcactttctagcacacacagggaagagggcttggctaagagcacccaggaggtcagggcaggactggggattagatcacacccaaatgcaagctctgtggcactcctggtatagccctgctgtccccctgtagcctggccctgggaagggggctcccagacttcacaccccctcacctctttatccaggggaccggctgtctcttcctccttcatccccagcctctgtcag

SEQ ID NO: 65 - DNA, 959 bp, Homo sapiens
gtaccggtccggaattcccgggatctcgacacctggactagccgggttgtatttggaaacgcggagtgagtttttccgtgctgtgtaggggctaacaatggacacccagaaggacgttcaacctccaaagcagcaaccaatgatatatatctgtggagagtgtcacacagaaaatgaaataaaatctagggatccaatcagatgcagagaatgtggatacagaataatgtacaagaaaaggactaaaagattggtcgtttttgatgctcgatgaatgctgggaattcagaggaatgtcttcacttatacttggatttgctctcttcccatttctgattgttgtatagctttcgattttgcttacagtagttcccccttatcttcgggagatacattccaaggcccccagtgaactcctgaaacctcaaacagtaccaaacctttatacactgttttttccatatatatatacctatgataaagtataatgtataaattaagcatagcaagagattaataataatgtaatagaacaatgataacatactataataaaagttatgtgaatgtggttggtctctcttgctttcaaaatatcttcttgtacagtactcacctattttagaatgtggttgactacaggtaaccaaaaccacagaaagggaaactttggatgmggggggcactactgtacttaggaatacaactatatacatatgattttatttttaagaccatattatatttgggtatctactaatattttgtataaagcaattttttgttccattacgtgactttttgttttattgtatatgtaatttaacacacaataaagggtaaagttgcttcccmaaccacagttttaatcaaaacctagaatcatctgcagtccttgttaaaaatgcaggtttctagaaccctctgaagttctgattaaataaatttwttgcaaacca

SEQ ID NO: 66 - DNA, 971 bp, Homo sapiens
gcactgtgcgtcatgtaccagcgccggaagttggtctcgacacctggactagccgggttgtatttggaaacgcggagtgagtttttccgtgctgtgtaggggctaacaatggacacccagaaggacgttcaacctccaaagcagcaaccaatgatatatatctgtggagagtgtcacacagaaaatgaaataaaatctagggatccaatcagatgcagagaatgtggatacagaataatgtacaagaaaaggactaaaagattggtcgtttttgatgctcgatgaatgctgggaattcagaggaatgtcttcacttatacttggatttgctctcttcccatttctgattgttgtatagctttcgattttgcttacagtagttcccccttatcttcgggagatacattccaaggcccccagtgaactcctgaaacctcaaacagtaccaaacctttatacactgttttttccatatatatatacctatgataaagtataatgtataaattaagcatagcaagagattaataataatgtaatagaacaatgataacatactataataaaagttatgtgaatgtggttggtctctcttgctttcaaaatatcttcttgtacagtactcacctattttagaatgtggttgactacaggtaaccaaaaccacagaaagggaaactttggatgaggggggcactactgtacttaggaatacaactatatacatatgattttatttttaagaccatattatatttgggtatctactaatattttgtataaagcaattttttgttccattacgtgactttttgttttattgtatatgtaatttaacacacaataaagggtaaagttgcttccccaaaccacacttttaatcaaaacctagaatcatctgcagtccttgttaaaaatgcaggtttctagaaccctctgaagttctgattaaataaatttattgcaaacca

SEQ ID NO: 67 - DNA, 469 bp, Homo sapiens
ttttttttttgaacactgtgcccagctaatttattaaattttgttgtagagatggggtctcattatgatgcccaggcgggtcttgaatttctggactcaagtgatcctcctgccttggcctcccaaagtcctgggattataggggtgagccctgttgcccggcctaaatctggctcttcagattccgagagagagggtacaggagcctctctgctcagcctgaagaacacccagaaggagggaacctcaaagagccccgagatggaaggctctcctggcagcgctcctgcaggggtgcctgtggaacagtgtgccccagggctaggagagttcatggcccagtctgcttcactgggctgccacagtgtgaaagctgggcctagacctggctcctgacatcctagtgagggctgggacttagtggttctgaaagacctttactggatggttcttccagagtggggtatgg

SEQ ID NO: 68 - DNA, 423 bp, Homo sapiens
gagtctgggacgcgccgccgccatgatcatccctgtacgctgcttcacttgtggcaagatcgtcggcaacaagtgggaggcttacctggggctgctgcaggccgagtacaccgagggggatgcgctggatgccctgggcctgaagcgctactgctgccgccggatgctgctggcccacgtggacctgatcgagaagctgctcaattatgcacccctggagaagtgaccacgctggaacccacccacccgctgtgctgaccatgggccctgagcgtcctgccccgaattcacgaggctgaggcatccgggagctggcgtaatgcctggccgcagtgtgtgtgtatcccataccccactctggaaggaaccatccagtaaaggtctttcagaaacaaaaaaaaaaaaaaaaaaaaaaaaaaaaaa

SEQ ID NO: 69 - DNA, 4237 bp, Homo sapiens
agattcgggcagggagcgggcggaacctttctaccgcgtctctagctaacacgcacggcggggacagtttaggcctccgcgcaccgttcgccgggagtcttgcagtttgcttggtgcagggaaggcgggcgcggaggttctatctgtttcttcctccttcgtgagcagcatggacgtgctagcggaggagtttgggaacctgactccggagcagctggcggcgccgatcccgactgtagaggaaaaatggaggctgcttccagcatttttaaaggtgaaaggccttgtgaaacagcatatagattcatttaactatttcattaatgtagagataaagaagataatgaaagccaatgaaaaggttacaagtgacgctgaccctatgtggtacttaaaatatcttaatatctatgttgggcttcctgatgttgaagaaagcttcaatgtaactagaccagtgtcccctcatgagtgccgtttgagagacatgacatactctgcccctattacagtggatattgaatatacccgaggcagccagaggatcatccgcaatgccttacctatcggcagaatgcccataatgctacgtagttcaaactgtgttcttacaggaaaaacgccagcagaatttgccaaactgaacgaatgtcccttagatccaggtggctacttcattgttaaaggagtagaaaaagttattcttatccaagagcagctgtctaagaacaggatcatcgtggaggctgatagaaaaggggctgttggagcttcagttaccagctctacccatgagaaaaaaagcagaaccaatatggctgtgaaacaaggacgattttatttgaggcataatactttgtcagaagatatacccattgtcatcatatttaaggccatgggtgttgagagtgaccaggaaattgtgcagatgattggaacagaggagcacgtgatggctgcatttgggcccagtctggaagagtgccagaaagctcagattttcacacagatgcaggcattaaaatatatagggaacaaagtaagaaggcaaaggatgtggggaggtggaccaaagaaaaccaaaatagaagaagcaagagagctcctggcttccaccattctgacccatgtcccagttaaggaattcaatttccgagccaaatgtatctatactgcagtgatggtgcgaagagttattctggcccaaggagataataaagttgacgacagagattattatggtaacaagcgactggaattggcaggacagcttttatctcttctttttgaagacttgttcaaaaaatttaattctgaaatgaaaaagattgccgaccaggtgattcctaagcaaagagcagcccagtttgatgttgtcaaacacatgcgccaagaccagatcaccaatggcatggtgaatgctatttctaccggaaattggtctttaaagagatttaaaatggaccgccagggtgtaacccaagtgctgtctcgcttgtcatatatatccgcactgggcatgatgacaagaatctcttcccagtttgaaaaaacgagaaaagtgagtggtcctcgctccctccagccatctcagtggggaatgctgtgtccttcggacactcctgaaggagaggcatgtggtttggttaaaaacttggcccttatgacacacatcacaactgatatggaagatggacccattgttaaattagccagtaacttgggagtagaagatgtgaatttattatgtggggaagagctctcttacccaaatgtgtttcttgtctttcttaatggtaacatcttaggtgtcattcgagaccacaaaaagctagtgaatacatttcgactcatgagaagagcaggatatatcaatgaatttgtttccatctcaacaaatcttacagatcgatgtgtctatatttcttctgatgggggaaggctatgcagaccctacataattgtcaagaaacagaagccagcagtcacaaataaacatatggaagagctggcccaagggtacaggaattttgaagatttcttacatgagagtctggttgaatatttagatgtgaatgaagaaaatgattgtaacattgcactgtacgaacacacaattaataaagacaccacccacttggagattgaacccttcactcttctcggcgtgtgtgctggacttatcccataccctcaccataaccagtcaccgagaaacacttatcagtgtgccatggggaaacaagccatgggtactataggatacaaccagcgaaacagaattgatactctcatgtatctactagcatatccacaaaaacccatggttaagacaaaaaccattgaattgatagaatttgagaaactgccagctggacagaatgcaacagttgctgtgatgagctatagtggctatgatattgaagatgctcttgttttaaacaaggcctctttagacagaggctttgggcgttgccttgtatataaaaatgctaaatgtacgttgaaacgatacaccaatcagacttttgataaagtgatggggcccatgttggatgctgctacaaggaaacctatctggcgacatgaaatcttagatgcagatggtatttgttctccaggtgagaaagtagaaaacaaacaagtgcttgtaaataagtccatgcccacagtgactcagattcctttggaaggaagtaatgtaccacagcaaccacagtacaaagatgtacccataacctacaaaggagcaacagactcatatattgaaaaagtgatgatatcttcaaatgctgaagatgcttttctgatcaaaatgctgctgagacagacaaggcgtccagaaattggagacaaattcagcagtcgtcatgggcaaaaaggtgtttgtggcttgatcgtcccccaggaagacatgccattttgtgattctggcatctgtccggacatcatcatgaacccacacggcttcccatcacgaatgacggtggggaagctcattgagctgctggctggcaaggccggtgtgctggacggcagattccactacggcactgcgtttggaggcagtaaagtgaaggatgtgtgtgaggacctcgttcgccatggttataactacttggggaaagactatgttacatccggcatcacaggtgagcccttcgaagcatacatctattttggccccgtgtactatcagaagctgaaacacatggtgctagataaaatgcatgcccgggcccggggcccacgagccgtccttaccaggcaacccactgaaggacggtctcgtgatggtggcttgcgtctcggggaaatggaacgtgactgtttaatcggttatggagccagtatgcttttgctagagagactaatgatttcaagtgatgcctttgaggttgatgtctgtgggcagtgtggacttctggggtattctggctggtgccattactgcaagtcatcctgccacgtgtcttccctccgtattccgtatgcctgcaagctgctcttccaggaactacagtctatgaacatcatccccaggttaaaactgtccaagtacaatgaatgaggatggaaaaaatgattattaaagagaacaagtgatacatccaatgcaacggaaagcagaagggatttaggactacgtctcctcctgtgaagaattcccttgcgtattctctctctaaaacaaccaaaaaaaaatggagaggctttttatatactctaagactggctaaacaaccttgatcattgagcctcgagccatgggagagatgctgaccatgtggactgcaaggctgcttgattcacagatggatgtgacctaaaggataaataagctattacttatgtgctgatctcttgacattcactcattagaagaccttactccttcaagcaaatgtttggggtcaaatttaccatatcttctggctaaccatattcaagattcttctgaaacttggaggatgtaaagaatccatttgatttggtcagcctggcttttgtcgtggtggctggctcggataaattttcccaacaattaaatcttgcctttacacacccaaactttgtaattttagtcttggtgaaatataatgaatttgttcctaccttgtcaagcaagaatgtcgtcttctcctatggactcaattgctattattttaaacctgcatgattgtaccatgcaatactattcgttaaatatctgaatctagaaaaaaaaaaaaaaaaa

SEQ ID NO: 70 - DNA, 1843 bp, Homo sapiens
  misc_feature (578)..(578): n is a, c, g, or t
tcttcggtggcgatccggtcctagaaagggcggtgggctccacctcggcctagaagcagacgggaccgtaggaagccgtcgcgggaagctcagccgaattggagttggagcccccggattgcgctgaccctgagctctcagactccccagtacaatgactcaagcagaaattaagctctgttctttgttgctgcaagagcattttggagagattgtagaaaaaattggagtccatctgataagaaccggcagccagccactaagagtaattgcccatgacacaggaacatcactggatcaggtgaagaaagccctgtgtgtcctcgtccaacataacctggtgagttatcaagtgcacaaacgtggtgtggtggagtatgaagcccagtgcagccgggtattgcgaatcgttagatatccccggtacatctatactaccaaaactctgtacagtgacactggagagctgattgttgaggagcttctgttgaacggcaaactgacaatgtgcactgttgtgaagaaagtggcagaccggctcacagagaccatggaggatggcaagaccatggactatnctgaagtatcaaacacatttntncgactggcagacacacactttgtacaacnnnncccttcggtacctaccactgagaattcagaccctgggccaccaccacctgcncccacacttgtcattaatgaaaaggacatgtacctggttcctaaactcagcttgatagggaaaggtaaaaggaggagatcatctgatgaagatgctgctggggagcccaaggcaagagaccaaaatatactacagataacaaggngcccattccagatgatgggatttattggcaggcccaaccttgacagattccgccaacacttccgtgaccaagccattgtgagcgcagttgctaacaggatggaccagacaagcagcgagattgtgcgaaccatgctccgaatgagtgagattaccacttcctctagtgctcccttcacccagccattgttttccaatgagatcttcagatccctacctgttggctataacatctctaagcaagttcttgatcagtatctcactctgctggcagatgatccactagagtttgttggaaagtctggcgacagtggtggaggaatgtatgtcatcaacctccataaggcattacgatccctagccacagccactctggagtccgtcgtacaggagagatttgggtctcgctgtgctagaatatgccgtctagttttgcagaagaaacacatagagcagaagcaagtggaagactttgcaatgattctgcaagaggcaaaggatatgctatataagatgctctcaggaaatttcatgtcactccaggaaattcccaaaacaccagaccatgccccatccaggaccttctatttatatactgtgaacatcctgtcagctgcccgaatgttgttcgacaggtgctacaagagcatagccaacttgatagaaaggaggcaatttgaaaccaaagagaataagcgtctactagaaaaatctcagagggtagaagccatcattgcatctatgcaggctactggtgcagaggaagcacagttacaagaaatagaggagatgatcacagctcctgaacgtcagcagctagagaccctgaaacgtaatgtcaacaagttggatgccagtgagatccaggtggnngaaaccatcttcctgctggagtcttacattgagtgcaccatgaagagacagtgatccagaagaagcatcttcctcagaagatctggggggatggaaagcaaaataaaggaggtgcctggatgnaaaaaaaaaaaaaaaaaa

SEQ ID NO: 71 - DNA, 1621 bp, Homo sapiens
cggaagccgccgaaactctggggtagagtggcacgcgctttttgcttttccgcgtcttcttcggtggcgatccgcgtcctagaaagggcggtgggctccacctcggcctagaaggccagcgggagccgtaggaagccgtcgcgggaagctcagccgaattggagttggagcccccggattgcgctgaccctgagctctcagactccccagtacaatgactcaagcagaaattaagctctgttctttgttgctgcaagagcattttggagagattgtagaaaaaattggagtccatctgataagaaccggcagccagccactaagagtaattgcccatgacacaggaacatcactggatcaggtgaagaaagccctgtgtgtcctcgtccaacataacctggtgagttatcaagtgcacaaacgtggtgtggtggagtatgaagcccagtgcagccgggtattgcgaatgcttagatatccccggtacatctatactaccaaaactctgtacagtgacactggagagctgattgttgaggagcttctgttgaacggcaaactgacaatgtcagctgttgtgaagaaagtggcagaccggctcacagagaccatggaggatggcaagaccatggactatgctgaagtatcaaacacatttgtgcgactggcagacacacactttgtacaacgctgcccttcggtacctaccactgagaattcagaccctgggccaccaccacctgcccccacacttgtcattaatgaaaaggacatgtacctggttcctaaactcagcttgatagggaaaggtaaaaggaggagatcatctgatgaagatgctgctggggagcccaaggccaagagaccaaaatatactacagataacaaggagcccattccagatgatgggatttattggcaggccaaccttgacagattccaccaacacttccgtgaccaagccattgtgagcgcagttgctaacaggatggaccagacaagcagcgagattgtgcgaaccatgctccgaatgagtgagattaccacttcctctagtgctcccttcacccagccattgtcttccaatgagatcttcagatccctacctgttggctataacatctctaagcaagttcttgatcagtatctcactctgctggcagatgatccactagagtttgttggaaagtctggcgacagtggtggaggaatgtatgtcatcaacctccataaggcattagcatccctagccacagccactctgaagtccgtcgtacaggagagatttgggtctcgctgtgctagaatattccgtctagttttgcagaagaaacacatagagcagaagcaagtggaagactttgcaatgattcctgcaaaggaggcaaaggatatgctatataagatgctctcagaaaatttcatgtcactccaggttggatgccagtgagatccaggtggacgaaaccatcttcctgctggagtcttacattgagtgcaccatgaagagacagtgatccagaagaagcatcttcctcagaagatctggggggatggaaagcaaaataaaggaggtgcctggatgcattaaaaaaaaaaaaaaaaaaaaaaaaaaaaaaaa

SEQ ID NO: 72 - DNA, 1946 bp, Homo sapiens
gatggcggcgcccaggccctgccacgcagacttccgcccggcgcggagaccgaaggctggcggctggtcgcgttgcaggcaacatgtcggaaggaaacgccgccggcgagcccagcacgccgggagggccccgacctctcctgactggggcccgggggctcatcgggcggcggccggcgcctcccctcacccccggccgccttccctccatccgttccagggacctcaccctcgggggagtcaagaagaaaaccttcaccccaaatatcatcagtcggaagatcaaggaagagcccaaggaagaagtaactgtcaagaaggagaagcgtgaaagggacagagaccgacaacgagaggggcatggacgagggcgaggccgtccagaagtgatccagtctcactccatctttgagcagggcccagctgaaatgatgaagaaaaaagggaactgggataagacagtggatgtgtcagacatgggaccttctcatatcatcaacatcaaaaaagagaagagagagacagacgaagaaactaaacagatcttgcgtatgctggagaaggacgatttcctcgatgaccccggcctgaggaacgacactcgaaatatgcctgtgcagctgccgctggctcactcaggatggctttttaaggaagaaaatgacgaaccagatgttaaaccttggctggctggccccaaggaagaggacatggaggtggacatacctgctgtgaaagtgaaagaggagccacgagatgaggaggaagaggccaagatgaaggctcctcccaaagcagccaggaagactccaggcctcccgaaggatgtatctgtggcagagctgctgagggagctgagcctcaccaaggaagaggaactgctgtttctgcagctgccagacaccctccctggccagccacccacccaggacatcaagcctatcaagacagaggtgcagggcgaggacggacaggtggtgctcatcaagcaggagaaagaccgagaagccaaattggcagagaatgcttgtaccctggctgacctgacagagggtcaggttggcaagctactcatccgcaagtctggaagggtgcaactcctcttgggcaaggtgactctggacgtgaccatgggaactgcctgctccttcctgcaggagctggtgtccgtgggccttggagacagtaggacaggggagatgacagtcctgggacacgtgaagcacaaacttgtatgttcccctgattttgaatccctcttggatcacaaacaccggtaaaatgagcaggtggaggaggacggcgcctgtgcccacggctgctgcctgctccagacattttgttcttgaatctgtgagacccagaaggggcccactgagcccactcactccagcctttggcaaccattgttccaggtcccccagggcttcctcccacagcagctgtgaatggcacagtgaccttcctgcagcgtggagatggcacatccttgctgctggggacttggccctgctatttatttttgtatttatgtcttaatctcttccactgatgcatcctccaagggtagatggggagggtctgtgtgaaggggccggcttctcttggtgcctgctgggttgcaggggcaggaagcgtgtggactgcagcttctgctggtgctccccccgtcctcctggaggcagtataggagagagagcaaggattgagtctgagacttaagcactcggtcccagcttgccagttcctggttctgtgtccttggacaaactacctaacctttctgagcctcctatacctcatccgacacaaatggggatgatacctacctccagggttggcgtgaggattcatgggctattatagatgaaaactgcacaaggccagaaccagcaggcactcaataaacgttcatgtcctttttctctaaaaaaaaaaaaaaaaaa

SEQ ID NO: 73 - DNA, 2640 bp, Homo sapiens
ggagtttctccaccagcaacatggccgccgcctgagaggagagccgggccgccgccgtctctgcagcccgcgggtaactgggccgttgccgccgtccgcgctcggcccccgcggagagatcgagctgaaggactgcgcggctggctctcctctagtatggccaatgaagaggatgacccagttgtacaggagatcgatgtgtacttggccaagagtctggcggaaaagctgtatctatttcagtaccctgtgcgtccagcctcgatgacctacgatgacattccgcacctctcagccaagatcaagcccaagcagcagaaggtagagcttgagatggccatcgacaccctgaaccccaactattgccgcagcaaaggggagcagattgcgctgaacgtggacggggcctgcgccgacgagaccagcacgtattcctcgaagctgatggacaagcagaccttctgctcttcccagaccaccagtaacacatcccgttatgccgctgcactctacaggcaaggtgagctccacctgacacctttacatggcatcctgcagctgcggcccagcttctcctacctggataaggctgacgccaagcaccgggagagggaggcggccaacgaggcaggggactcttcacaggatgaggcggaagacgatgttaagcagatcacggtgcggttctcccggccggagtcagagcaggcccgccagcgccgtgtgcagtcctatgagttcctgcagaagaagcacgcagaggagccctgggtccacctgcattactatggcctgagggacagtcgctctgagcatgagcgtcagtacctgctgtgccccggctcaagcggggtggagaacacggagctcgtcaagtcacccagtgagtacctgatgatgctgatgccacccagccaggaggaggagaaagacaagcctgtggcccccagcaacgtcctgtcgatggcccagctgcgcacgctgcccctggccgatcagatcaagatcctgatgaagaatgtgaaggtcatgccttttgccaacttgatgagcctcctgggcccctccatcgattccgtggctgttctgcggggcatccagaaggtggcgatgttggtccaagggaactgggtggtgaagagtgacatcctataccccaaggactcgtccagccctcacagcggcgtgcctgctgaggtgctctgcaggggccgagacttcgttatgtggaagttcacgcagagccgctgggtggttaggaaagaggtggcaaccgtgaccaaactctgcgccgaggatgtgaaggacttcctggagcacatggccgtggtgaggatcaacaaaggctgggagttcattctgccttatgatggggagttcatcaagaagcacccggatgtggtccagcggcagcacatgctgtggacgggtatccaggccaaactggaaaaagtctataatcttgtaaaggaaaccatgccaaagaagccggatgcacaatcagggcctgccgggctggtctgtggggaccagcggatccaagtagccaaaaccaaggcccagcagaaccacgcgttgctggagcgggagctgcagcggcggaaggagcagctgcgggtgcctgcggtcccgcccggtgtgcggatcaaggaggagcccgtgagcgaggagggcgaggaggacgaggagcaggaggcggaggaggagcccatggacacttcccccagcggcctccacagcaagctggccaacgggctgcctctcgggcgggctgcgggcacagacagcttcaacgggcacccgccccagggctgcgccagcacccctgtggctcgggaactgaaggccttcgtggaggccacctttcagagacagtttgtgctcacgctgagcgaactcaagcgcctcttcaatctgcacttggccagcctgccccccggccacacactcttcagcggcatctcggaccgcatgctacaggacacggtgctggccgccggttgcaagcagatactggtgccttttcccccccagactgctgcttccccggatgagcagaaggtgtttgccctctgggagtctggagacatgagtgatcagcatcgacaggttttgcttgaaattttttccaaaaattaccgggtacgccgaaacatgatccagtctcggttgactcaagagtgtggagaagatctcagtaaacaggaggtggataaagtactaaaggactgctgtgtaagctatggtggcatgtggtaccttaaagggacagtacagtcttgacaatagtagcaaactactaacccagcaaatctaagcccaaggaagaagggcggaaccagaagtagggcctcgacttgcttcagacgacacagagcaagaggaactgaccatctcatgacctgtggcattgcacggtgcagtggacagaagggattatcctcagccagtcgcagggtcagcttaagttagttagatcactcccagaagagaccagctgggaccttctttgcagtacaatttgaaattcctgatgtattttgcttattatttggtttcattctcataataaagagagtgtatactgacatgggcaggatgataaaaaaaaaaaaaaaaaaaaaaaaaaaaaaaaaaaaa

SEQ ID NO: 74 - DNA, 2177 bp, Homo sapiens
ggaagaaggcccctcggcctcagcagagcgctatcctccactggttacccgggttccccggcttgctaccgggctgctccgtgcatctttccccccaggcgtcaggaactgcgccctcatggcggaggtgaaggtgaaggtgcagccgcctgacgcggatccggtcgaaatagaaaacaggattatagaattatgtcaccagttccctcatggaatcacagaccaagtaattcagaatgaaatgcctcatatagaagcccagcagcgggcagtagccatcaataggttgttgtctatgggtcagttggatctcttaaggagcaatacgggccttttatatagaataaaggactctcagaatgctggtaaaatgaagggatccgataaccaagaaaaactagtatatcaaatcatagaggatgcaggaaataaaggaatatggagcagagatatccgctataaaagtaatttgccattaacagaaatcaacaaaattctgaagaatctggaaagtaaaaagcttatcaaagctgttaagtctgtagcagcctcaaaaaagaaggtgtatatgctctataacctgcagccagaccggtctgtgactggtggagcctggtacagtgaccaggattttgaatctgaatttgtagaggtgcttaaccaacagtgttttaaattcctacagtccaaggcagaaacagcacgagaaagcaaacagaacccaatgatacaaagaaatagttcatttgcctcatcacatgaagtgtggaaatatatctgcgaattgggaatcagtaaggtagagttatccatggaagacattgaaaccatcctgaatacactcatttatgatggaaaagtggagatgacgattattgctgcaaaagaaggcacagttggcagtgtagatggacacatgaaactgtacagggcagtcaatccaatcatccctcccacaggtttggtccgggcaccctgtggactctgcccggtttttgatgactgccacgaaggtggtgagatttcaccatctaactgtatttacatgacagagtggctcgaattttaatagagagctatgaactttattgacattttgcaaatgaagttacttagggagcagataatttaattcatgatggaacacgaaatctccttgaaagcaaacttcacaataatggacgtagacttgctgctatgaaaacatattttttttatttatgaagactaaatttatattggtaaaatagccagtagaatatgaaagaaataaggttagtagtgaaattcattcttcaataaataaaacactttgaaactccggaggaccacatctttcaagacttctgatgggtcaaggaaaaagatgccaacatacccgtatttaccaagtactatgataatggctagagtataaaaatgttctttttaaagttatttattaagttcttcattggaagtttttttttatatctggttcactaccaccattttctgtttcctactttctcagtggtttcattgaaaagaaattagaaggggttaaaggcaggaatagcaaagagtgcaaacttggggtatgactgggggagagtggaacatgccttttccgcacaatattaattcctttttgtatcagaaatgttcttttaggagattatgctaccatacttacttcaaacccaatgactactgtcaaggacatattttcagtacataaatactatcattttcattctaaagaatattttcactgttccttctttcttaaagtcttatgtttcactctttaactcaaatgtattctttgttagaatttaccctagattcttatttaatgtctgcagtagactgaatgtttgtgtgcccccagaattctaatgttgaaatctcatttccaatgtgatggtatttggaggtggggcttttggtaagtgataggtcaggagagtaacagcgctcatgaatgggattagtgcccttatataaagagacccagagagctccatcaccccttctgccatgtgaaagggagaagacaaacatccacgaaccaggaagtgggtcctcaccagaaaacaaatctgtaagcaccttgatcttggacttcccagcctccagaattgtgagaaataaatttctgttgttgataaaaaaaaaaaaaaaaaaaaaaaaaa

SEQ ID NO: 75 - DNA, 414 bp, Homo sapiens
  misc_feature (336)..(336): n is a, c, g, or t
gtttgtggtgattgtttattcaagaatatttggaacaaacatccctgcttaatagttactagttaagatctatattaacaaattagttttctctaacaaataaacatttaaataagttcaaaataaaacatcttactataaaacaaacgaacacattttgttttgtaagaatttaatgtgtaaataaatacagaaaaacaagcattacagcaaccagttttgctcatgatagcaattttaaagacacttcaacaaatacttttatgtaagaggctaattctgcaaaataatattcactttcctatggaataatttcacagtcccagaattttaagnccccaaggggaagagaaaagaaagtgggaaagggaaaccgtttcccaatttggggccctttttggggggnttttttaa

SEQ ID NO: 76 - DNA, 3285 bp, Homo sapiens
actccgcggtcctggtgccgcgtgcaggtcggtgcgcgcttctcccgaggtggaacgggcggcagtcaagcgccggcgttctctgccgtcaccctttccttgccggccggcacttcggctgcagagttttgcccacgcttcgagacttagggagcagtgcctttcagaatttgcccactcatctggtataactggttctgatggctgggaataaaggaagaggacgtgctgcttatacctttaatattgaggctgttggatttagcaaaggtgaaaagttacctgatgtagtgttgaaaccacccccactatttcctgatacagattataaaccagtgccactgaaaacaggagaaggtgaagaatatatgctggctttgaaacaggagttgagagaaacaatgaaaagaatgccttattttattgaaacacctgaagaaagacaagatattgaaaggtatagtaaaagatacatgaaggtatacaaggaagaatggataccagattggagaagacttccaagagagatgatgccaagaaataaatgtaaaaaagcaggcccaaaacccaaaaaggcaaaagacgcaggcaaaggcacaccactcactaatactgaagatgtgttgaaaaaaatggaggaattggaaaaaagaggtgatggtgaaaaatcagatgaggaaaatgaagagaaagaaggaagcaaagagaaaagtaaagaaggtgatgatgacgatgacgatgatgccgcagaacaggaggaatatgatgaagaagagcaagaagaggaaaatgactacattaattcatactttgaagatggagatgattttggcgcagacagtgatgacaacatggatgaggcaacctattaggcatgaaatttttcaaaaaatatttttatgatgcagcttctgaacatttggacagacttgatttgtattttatttctgataaggaataagtacttgtttctgttgttttggacaaatagttgttaccaaaatattcaaaaccactttgagtttacatactagttaccttaaaaattattccctgacactctgacattccttctaaacacctctgccatctctcttatgtactctcatgggtttttttgtatgatttgaatatgaatgtgcctaaagaatttttgctctcttaatctatgtatacatacttgaacaaatcattcttgcttaactgctgatcttttgtaaaactattgctagtcataatggatttctataacctgaaagttgatataatacagcacgtggagcactttaaaaacaaagaaacttgaaaataattttattttttacctttacatggtgtgttctagactactgcattatgactacataaacctacctgagctcttacaagtacagaattgtgagacagaggttttgtgatggaaaacatacacatagggaaaaataccaaactgagcaatataaatttcacagctcacatcttagccagtacaaagaaactttcacatggggtgaatattataatacttgaatttgagacttaatactgtatatgaattaatcatcccactgtaataactgacatcttcaaatagttgggcttatgaaaaatttgactaagacattttattttatactttatgtaaatgtgaaataagttctggtcatataaatatgtagaatgcatgtgtgtgaactctatgaagtgaagtgtggtggccttttatgtttcagagtactcaagtgtacaattgatttatataaaaatgcaaaatgaactcaggtcattttgaaattaatattaatatttttgttgtttaactttaaagctagctcccctaactttatatattttttgggaaaaaatacctaaaaacctcagcctacaaagactctcagaaatgccaaagattttcaaggaaattcatattatatatttcaaaaatgatttatcaatgttatctaccaaaagaaataattttatttttcccctttggggagatattatcctctaatatgagaatcagatccctagattctatttctacctatactattaaactcaaccttgaacctgagcttggttgtttctgtgccttagtttctccactcgtaaacaatttcctataaaaattgtttaaacaccaaccaaaatttaccatatgtattcattctcttgccttaaagatcacttcttaacttacatttggctctcaccctctaaaaatttgtagtttggagagtaactgaaagcacaattttaagaaacatgttttcctcacaaaatatattgtaatctgattttcctatcttgtattcatgcagaaaattggagaaaatgtgtattgtcttgtgccattctgcaatatgaatttctcaggaaaaagatgtttcaatggcaatactttcttaaataattcaagatgggtgtgaggacctatcttattagtttagtgctgtgttaaccatttgtgaattacttcttgtttaaaggaagttgtcaaaaaattttaagattctaaatggaatacacgaataaagtaagccttagaatagaaacctcacaaaaagttgtcatttgcggttgataattaacataggtgttttattctctcaactccttagtaccagagccaaatacaatttagttttcagaaatcttatcaatattgttctatttacctacttataaatctgcagataaccacttgaaccaggcaaatacactgaagataaaggttatttctttttttagctttgaatttgtcatgaccattttagtcttgcagatagcagggcagccccttggggcaaggatttactctgggaggtaccgttaagagccttctttccccctttgaaagatccttttacaatgttaaagtatactagttgcaagaacaagcaggatttgcaggttgctttaccagcatgagtctcatttttctggcttaaaatctgggactgtgaaattattccataggaaagtgaatgttattttgcagaattagcctcttacataaaagtatttgttgaagtgtctttaaaattgctatcatgagcaaaactggttgctgtaatgcttgtttttctgtatttatttacacattaaattcttacaaaacaaaatgtgttcgtttgttttatagtaagatgttttattttgaacttatttaaatgtttatttgttagagaaaactaatttgttaatatagatcttaactagtaactattaagcagggatgtttgttccaaatattcttgaataaacaatcaccacaaacaa

SEQ ID NO: 77 - DNA, 1377 bp, Homo sapiens
gggagcctgcggagttcgagaccatgctgctgttctgccccggctgcgggaacgggctgatcgtggaggagggacaacgctgccaccgcttcgcctgcaacacgtgcccctacgtgcacaacatcacccgcaaggtaacaaatcggaagtacccaaaactgaaagaagtggatgatgtgcttggtggagcagctgcctgggagaatgttgactctactgcagagtcgtgtcccaaatgcgaacatcctcgtgcttacttcatgcagcttcagacccgctctgcagatgagccgatgaccaccttctacaagtgctgcaatgctcagtgtggacaccgctggagggattagggccaggatggcccagctgccctagtgtgtgcttgccttgtccctcggggtagatgcttagctggcagtatgagttgtgtgtcctgagggtctttgctagtgtggtggaaagataaaccttttgaggtgaagagccagggggtcaggaaatatggcctatctgccaggcagggtggatgaagtcatgaatgtctgggagtttttctgtgtggggaggagacagagacccataactaaatatgctctgtgtaaagtcctattctttcatcttccactttattggcagttgacattcccttactcccgatcaacactcttaaatatttgtactgtttgtaaaacttagtacatgtccctaaaatatttaactgttacttgtaaacttgtgtaatttattatttattttaatcaaaattctgaatatttcatttaaatgaaagttggaatattgccttctcagtgttttaaaatagcttaagaggtagaaagcagaccccaaatcacagtgatcccagaatagactacatacttacatttgaacaggctacgaaaatgttaatcaccgggatttcaatttctttctttattcctattcttactcccttattcatttttttgaacctcccctgatttgcttcctcgggttgctttaaatgtataagatactatttatttatttatttattttatttatttatttatttatttaagacagagtcttgttctgtcgcccaggctggagtgcagtggtacgaattcagctcactgcaacctctgccttccgggttcaagggattctcctgtctcagcctcccaagcagctgggactacaggcatgcaccaccacacctgggtaattttgcatttttagtagagatggggtttctccatgttggtcgggctggtctccaactcctgacctcagataatctgtctgcctcggcctcctaaagtgctgggattacaggcgtgagccactgtgtcccaccaatctttacatttaatatgactattttcttattgttcg

SEQ ID NO: 78 - DNA, 3800 bp, Homo sapiens
ggcgcgggcgcatgcgcaggcgcgggccggtggggtggcctggagcggcgtgcgtaatgtcggcactttgctggggccgcggagcggcggggctcaaacgagccctacggccttgcggccgcccgggactccccggcaaagaagggaccgccggtggcgtctgcggccccaggaggagctcgtccgccagcccccaggagcaagaccaagaccgcaggaaggactggggccacgtggagctgctggaggtgctccaggcgcgggtgcggcagctgcaggctgagagcgtgtcggaggtggtggtgaacagggtggatgtggcgcggctcccagaatgtggcagtggagatggtagcctccagccacccaggaaggtccagatgggggccaaggatgccaccccggtgccctgtggccgctgggcaaagatactggagaaggataagcggacccagcagatgcgtatgcagcggttgaaggcgaagctgcagatgccattccagagcggggagttcaaggcgctgaccaggcgcctgcaggtggagccccggctcctgagcaagcagatggccgggtgcctggaggactgcacgcgccaggcccccgagagcccctgggaggagcagctggcccggctgctgcaggaggcccctgggaagctgagcctcgatgtggagcaggccccgtcggggcagcactcgcaggcccagctctcaggtcagcagcagaggctcctggccttcttcaagtgctgcctgctcactgaccagctgcccctcgcccaccacctgctggtcgtccaccacggccagcggcagaagcggaagctgctcacgctggacatgtacaacgccgtgatgcttggctgggcgcggcagggtgccttcaaggagctggtatatgtgttattcatggtgaaggatgccggcttgactccggacctgctgtcctatgcggctgccctccagtgcatggggaggcaggaccaggacgccgggaccatcgaaaggtgtctggaacagatgagccaggaggggctgaagctgcaggcactcttcaccgccgttctgctgtctgaggaggatcgggccactgttctgaaggccgtgcacaaggtgaagcccaccttcagcctcccgccgcagctgccgcccccggtcaacacctccaagctgctcagggacgtgtatgccaaggatgggcgtgtgtcctacccgaagctgcacctgcccttgaagaccctgcagtgcctctttgagaagcagctccacatggagctggccagcagggtgtgcgtggtgtccgtggagaagcccacgttgccaagcaaggaggtcaagcacgcgcggaagaccctgaagaccctgcgggaccaatgggagaaagcactgtgccgggcgctgcgggagaccaagaaccgcctagagcgcgaggtgtacgagggccggttctcactttaccccttcctgtgcctgctggacgagcgcgaggtggtgcggatgctcctgcaggtcctgcaggcgctgcccgcccaaggtgagtccttcaccaccctggcccgggagctgagtgcgcgcactttcagccggcacgtggtgcagaggcagcgggtcagtggccaggtgcaggcgctgcagaaccactacaggaagtacctctgcttgctggcctccgacgccgaggtgcccgagccctgcctgccgcggcagtactgggaggagctgggggcgcccgaggccctgcgggagcagccctggcccctgccagtgcagatggagctgggcaagctgctggcggagatgctggtgcaggctacgcagatgccatgcagcctggacaagccgcatcgttcctctcggcttgtccccgtgctctaccacgtgtattccttccgcaacgtccagcagatcggcatcctgaagccgcacccggcctacgtgcagctgctggagaaggccgcggagcccacgctgaccttcgaggcggtggatgtacccatgctttgccccccgctgccctggacatcgccgcactctggtgctttcctgctcagccccaccaagctgatgcgcacggtggaaggcgccacgcagcaccaggagctgctggaaacctgcccgcccaccgcgctgcatggcgcactggacgccctcacccaactgggcaactgcgcctggcgcgtcaacgggcgcgtgctggacctggtgctgcagctcttccaggccaagggctgcccccagctaggcgtgccggccccgccctccgaggcgccccagccgcccgaggcccacctgccgcacagcgccgcgcccgcccgcaaggccgagctgcgccgtgagctggcgcactgccagaaggtggcccgggagatgcacagcctgcgggcggaggcgctgtaccgcctctcgctggcgcagcacctgcgggaccgcgtcttctggctgccgcacaacatggacttccgcggccgcacctacccctgcccgccgcacttcaaccacctgggcagcgacgtggcgcgggccctgctggagttcgcccagggccgcccgctcggcccgcacggcctggattggctcaagatccacctggtcaatctcacggggttgaagaagcgggagccgctgcggaagcgcctggcctttgcggaggaggtgatggatgacatcctggactccgcggaccaacccttgacgggccgaaagtggtggatgggcgcggaggaaccctggcagacgctggcctgctgtatggaggtggcgaacgctgtgcgcgcctccgaccctgccgcctatgtctcccacctccccgtccatcaggacggctcttgcaacggcctgcagcattatgctgctctgggccgcgacagcgtgggcgccgcctccgtcaacctggagccctcggatgtgccgcaggacgtgtacagcggcgtggccgcgcaggtggaggtgttccgtaggcaggacgcccagcggggcatgcgggtggcacaggtgctggaaggtttcatcacccgcaaggtggtgaagcagacggtgatgacggtggtgtacggggtcacgcgctatggcgggcgcctgcagattgagaagcgcctccgggagctgagcgactttccccaggagttcgtgtgggaggcctctcactatctcgtacgccaggtcttcaagagtctacaggagatgttctcggggacccgggccatccagcactggctgaccgagagtgcccgcctcatctcccacatgggctctgtggtggagtgggtcacacccctgggcgtccccgtcatccagccctatcgcctggactccaaggtcaagcaaataggaggtggaattcagagcatcacctacacccacaacggagacatcagccgaaagcccaacacacgtaagcagaagaacggcttcccgcccaacttcatccactcgctggactcctcccacatgatgctcaccgccctgcactgctacaggaagggcctgaccttcgtctctgtgcacgactgttactggactcacgcagctgatgtctccgtcatgaaccaggtgtgccgggagcagtttgtccgcttgcacagcgagcccatcctgcaggacctgtccagattcctggtcaagcggttctgctctgagccccagaagatcttggaggccagccagctgaaggagacactgcaggcggtgcccaagccaggggccttcgacctggagcaggtgaagcgttccacctacttcttcagctgacaccccgtgagccttgtcagtgtgtaaataaagctcttttgccacccccag

SEQ ID NO: 79 - DNA, 698 bp, Homo sapiens
cggcagccagaagacgcggtcccgcaggtgctgcgccagcgagaggcggtacagcgcctccgcccgcaggctgtgcatctcccgggccaccttctggcagtgcgccagctcacggcgcagctcggccttgcgggcgggcgcggcgctgtgcggcaggtgggcctcgggcggctggggcgcctcggagggcggggccggcacgcctagctgggggcagcccttggcctggaagagctgcagcaccaggtccagcacgcgcccgttgacgcgccaggcgcagttgcccagttgggtgagggcgtccagtgcgccatgcagcgcggtgggcgggcaggtttccagcagctcctggtgctgcgtggcgccttccaccgtgcgcatcagcttggtggggctgagcaggaaggcaccagagtgcggcgatgtccagggcagcggggggcaaagcatgggtacatccaccgcctcgaaggtcagcgtgggctccgcggccttctccagcagctgcacgtaagccgggtgcggcttcatgatgccgatctgctggacgtagcggtacgaatacacgaggtagagcacggtgacaagccgagaggaacgatgcggcttgtccacgctgcatggcatctgcgtagcctgcaccatcatgtacgccagcatcttggtcacatcatcagcactgacagaggccatggctg

SEQ ID NO: 80 - DNA, 3861 bp, Homo sapiens
gcggccgctctcagcggagggggcggccccggggcccaggcgccgcggcccggcaccccgtggaagagccgcgcgtacagcccgggcatccagggactacatgaggaaataattgacttttataacttcatgtccccttgtcctgaagaagcagctatgagaagagaggtggtgaaacggatcgaaactgtggtgaaagacctttggccgacggctgatgtacagatatttggcagctttagtacaggtctttatcttccaactagcgacatagacctggtggtcttcgggaaatgggagcgtcctcctttacagctgctggagcaagccctgcggaagcacaacgtggctgagccgtgttccatcaaagtccttgacaaggctacggtaccaataataaagctcacagatcaggagactgaagtgaaagttgacatcagctttaacatggagacgggcgtccgggcagcggagttcatcaagaattacatgaagaaatattcattgctgccttacttgattttagtattgaaacagttccttctgcagagggacctgaatgaagtttttacaggtggaattagctcatacagcctaattttaatggccattagctttctacagttgcatccaagaattgatgcccggagagctgatgaaaaccttggaatgcttcttgtagaattttttgaactctatgggagaaattttaattacttgaaaaccggtattagaatcaaagaaggaggtgcctatatcgccaaagaggagatcatgaaagccatgaccagcgggtacagaccgtcgatgctgtgcattgaggaccccctgctgccagggaatgacgttggccggagctcctatggcgccatgcaggtgaagcaggtcttcgattatgcctacatagtgctcagccatgctgtgtcaccgctggccaggtcctatccaaacagagacgccgaaagtactttaggaagaatcatcaaagtaactcaggaggtgattgactaccggaggtggatcaaagagaagtggggcagcaaagcccacccgtcgccaggcatggacagcaggatcaagatcaaagagcgaatagccacatgcaatggggagcagacgcagaaccgagagcccgagtctccctatggccagcgcttgactttgtcgctgtccagcccccagctcctgtcttcaggctcctcggcctcttctgtgtcttcactttctgggagtgacgttgattcagacacaccgccctgcacaacgcccagtgtttaccagttcagtctgcaagcgccagctcctctcatggccggcttacccaccgccttgccaatgcccagtggcaaacctcagcccaccacttccagaacactgatcatgacaaccaacaatcagaccaggtttactatacctccaccgaccctaggggttgctcctgttccttgcagacaagctggtgtagaaggaactgcgtctttgaaagccgtccaccacatgtcttccccggccattccctcagcgtcccccaacccgctctcgagccctcatctgtatcataagcagcacaacggcatgaaactgtccatgaagggctctcacggccacacccaaggcggcggctacagctctgtgggtagcggaggtgtgcggccccctgtgggcaacaggggacaccaccagtataaccgcaccggctggaggaggaaaaaacacacacacacacgggacagtctgcccgtgagcctcagcagataatggctcctggctgcgtcagcctcccccacccctctgcagactgccccgcggcctcggccaccggcaggggaaccgagaccagcaccccgcacgtcagccgggctcgcggcacgcccgccgctgatcactctgcatgtttcttcgtgtggtggtcgcgtccatcttcaagaacagctcgttgtgctcatctgtgaagccttattaaacgtggacgttgttttctgccttcccaggattcttccttcagtgctgaggcaggtcgggctcaggaactgcagggacgtgaacatgcgcttgcggtttgaggtagccgtgtctgttccttcgcggtttgctattttcatttcctgttcgtcaaagcagcagaggagatcaaaccccgttcgtgtgtctttcctccacggataagcttgggaggtcattgttttactgccctcacattttgtttgaaatttcagaactgtttttctatgtaaatattgaaaacttatgatttgtgcaataactcagatattttttatttaatttcctattttcacataagttatatttaagggaggagggaattttttttaaacaagcttaggtcctttcccgagctgcattttctaagttgggtcatcgtgtcggctggttgtctgacgagcatcgttacaaacaccatgatgaggggtttggggttttattttgatgtcttttcttttggtcggaagtgagtgaaggagccaggtcgccctgaaggttttccaaagggcttggctccagagccacctggcagactgcccgtggccctgctgtcgggccccaggccgttgtcctgctctgaccacagagttttaatgttttggttttcacttcttttaaactggacaacaaatccagcatttcaagtgccagaagtataactttctaaggagagaagggttgtcacattataaaatctttaggaaaatgtgaactggaaaacgcttcggtcagttttagtgacatagcctgtgatgatgggtctggtgactattattgcggaccgtggtacccagttttaggaatgtggagaaaggaattctgttgattccgttgaggaatctgtagcgtatgcattcgttctgttaagagcaaatctaggagaagtgcttcagctgcccagtgcgccgtggggagtgttttaacggatcgtgtcgcaggagagcacagcccagcgttggggccgggaccgctggcgcccgacgtcggaagcatacaggtatactatgcaagtgtattctgccacaacaaccactgtctttgttacctttttttgaacaagaatatatccatcctgcctaaccctgagtttttggagcaccacagttgtcctgggagttggttgcatcttgtaggccatctgacttcctgtttttaaaacgggggtctggtcttgctaaacactacaggtaggttggtctttgaagtccactagtggagaatgtcaagacaagatacttattaccatgacatctgatgcatgtgcagcagtggggagttctagattgatctctgaatgtgatcgacgcccagcaaggacaagctttaaaatgtctgcggtctgcccttttgaagcaggactggctcactctgtcattgggagctgtcagctgcgactgcaggttctctaggaggcattccagaatagagtagcacactgtgtctgcagttctcgatgaccgaaagttatcaaaaatatttaaaatatttaaattgtgaacctattgataaagaatatttataaaaactgatctgtaggcctgtactaatctctacgcattagcaatattgactgtaaacccacattaaggaaaccactacgggtctggcagtgcgtgtcccgtggggtgtgcattttaaaactcgattcatagacacaggtaccatgttccatttccgtcatggtgaagcaaatgaattggcctggctaccactgtggtcgcgtgctacaggtttgacaaaaagatatcatgtttcgatttttttgtgtgtggacaacaatatggaagctaaaattgacatatttttatgtaaagtttttctattctttgatttttaataaactttggaaaccagttt

SEQ ID NO: 81 - protein, 651 aa, Homo sapiens
MAAPLLHTRLPGDAAASSSAVKKLGASRTGISNMRALENDFFNSPPRKTVRFGGTVTEVLLKYKKGETNDFELLKNQLLDPDIKDDQIINWLLEFRSSIMYLTKDFEQLISIILRLPWLNRSQTVVEEYLAFLGNLVSAQTVFLRPCLSMIASHFVPPRVIIKEGDVDVSDSDDEDDNLPANFDTCHRALQIIARYVPSTPWFLMPILVEKFPFVRKSERTLECYVHNLLRISVYFPTLRHEILELIIEKLLKLDVNASRQGIEDAEETATQTCGGTDSTEGLFNMDEDEETEHETKAGPERLDQMVHPVAERLDILMSLVLSYMKDVCYVDGKVDNGKTKDLYRDLINIFDKLLLPTHASCHVQFFMFYLCSFKLGFAEAFLEHLWKKLQDPSNPAIIRQAAGNYIGSFLARAKFIPLITVKSCLDLLVNWLHIYLNNQDSGTKAFCDVALHGPFYSACQAVFYTFVFRHKQLLSGNLKEGLQYLQSLNFERIVMSQLNPLKICLPSVVNFFAAITNKYQLVFCYTIIERNNRQMLPVIRSTAGGDSVQICTNPLDTFFPFDPCVLKRSKKFIDPIYQVWEDMSAEELQEFKKPMKKDIVEDEDDDFLKGEVPQNDTVIGITPSSFDTHFRSPSSSVGSPPVLYMQPSPL

SEQ ID NO: 82 - protein, 1394 aa, Homo sapiens
MAALLRSARWLLRAGAAPRLPLSLRLLPGGPGRLHAASYLPAARAGPVAGGLLSPARLYAIAAKEKDIQEESTFSSRKISNQFDWALMRLDLSVRRTGRIPKKLLQKVFNDTCRSGGLGGSHALLLLRSCGSLLPELKLEERTEFAHRIWDTLQKLGAVYDVSHYNALLKVYLQNEYKFSPTDFLAKMEEANIQPNRVTYQRLIASYCNVGDIEGASKILGFMKTKDLPVTEAVFSALVTGHARAGDMENAENILTVMRDAGIEPGPDTYLALLNAYAEKGDIDHVKQTLEKVEKSELHLMDRDLLQIIFSFSKAGYPQYVSEILEKVTCERRYIPDAMNLILLLVTEKLEDVALQILLACPVSKEDGPSVFGSFFLQHCVTMNTPVEKLTDYCKKLKEVQMHSFPLQFTLHCALLANKTDLAKALMKAVKEEGFPIRPHYFWPLLVGRRKEKNVQGIIEILKGMQELGVHPDQETYTDYVIPCFDSVNSARAILQENGCLSDSDMFSQAGLRSEAANGNLDFVLSFLKSNTLPISLQSIRSSLLLGFRRSMNINLWSEITELLYKDGRYCQEPRGPTEAVGYFLYNLIDSMSDSEVQAKEEHLRQYFHQLEKMNVKIPENIYRGIRNLLESYHVPELIKDAHLLVESKNLDFQKTVQLTSSELESTLETLKAENQPIRDVLKQLILVLCSEENMQKALELKAKYESDMVTGGYAALINLCCRHDKVEDALNLKEEFDRLDSSAVLDTGKYVGLVRVLAKHGKLQDAINILKEMKEKDVLIKDTTALSFFHMLNGAALRGEIETVKQLHEAIVTLGLAEPSTNISFPLVTVHLEKGDLSTALEVAIDCYEKYKVLPRIHDVLCKLVEKGETDLIQKAMDFVSQEQGEMVMLYDLFFAFLQTGNYKEAKKIIETPGIRARSARLQWFCDRCVANNQVETLEKLVELTQKLFECDRDQMYYNLLKLYKINGDWQRADAVWNKIQEENVIPREKTLRLLAEILREGNQEVPFDVPELWYEDEKHSLNSSSASTTEPDFQKDILIACRLNQKKGAYDIFLNAKEQNIVFNAETYSNLIKLLMSEDYFTQAMEVKAFAETHIKGFTLNDAANSRLIITQVRRDYLKEAVTTLKTVLDQQQTPSRLAVTRVIQALAMKGDVENIEVVQKMLNGLEDSIGLSKMVFINNIALAQIKNNNIDAAIENIENMLTSENKVIEPQYFGLAYLFRKVIEEQLEPAVEKISIMAERLANQFAIYKPVTDFFLQLVDAGKVDDARALLQRCGAIAEQTPILLLFLLRNSRKQGKASTVKSVLELIPELNEKEEAYNSLMKSYVSEKDVTSAKALYEHLTAKNTKLDDLFLKRYASLLKYAGEPVPFIEPPESFEFYAQQLRKLRENSS

SEQ ID NO: 83 - protein, 1079 aa, Homo sapiens
MDPGSRWRNLPSGPSLKHLTDPSYGIPREQQKAALQELTRAHVESFNYAVHEGLGLAVQADINWAVNGISKGIIKQFLGYVPIMVKSKLCNLRNLPPQALIEHHEEAEEMGGYFIINGIEKVIRMLIMPRRNFPIAMIRPKWKTRGPGYTQYGVSMHCVREEHSAVNMNLHYLENGTVMLNFIYRKELFFLPLGFALKALVSFSDYQIFQELIKGKEDDSFLRNSVSQMLRIVMEEGCSTQKQVLNYLGECFRVKLNVPDWYPNEQAAEFLFNQCICIHLKSNTEKFYMLCLMTRKLFALAKGECMEDNPDSLVNQEVLTPGQLFLMFLKEKLEGWLVSIKIAFDKKAQKTSVSMNTDNLMRIFTMGIDLTKPFEYLFATGNLRSKTGLGLLQDSGLCVVADKLNFIRYLSHFRCVHRGADFAKMRTTTVRRLLPESWGFLCPVHTPDGEPCGLMNHLTAVCEVVTQFVYTASIPALLCNLGVTPIDGAPHRSYSECYPVLLDGVMVGWVDKDLAPGIADSLRHFKVLREKRIPPWMEVVLIPMTGKPSLYPGLFLFTTPCRLVRPVQNLALGKEELIGTMEQIFMNVAIFEDEVFAGVTTHQELFPHSLLSVIANFIPFSDHNQSPRNMYQCQMGKQTMGFPLLTYQDRSDNKLYRLQTPQSPLVRPSMYDYYDMDNYPIGTNAIVAVISYTGYDMEDAMIVNKASWERGFAHGSVYKSEFIDLSEKIKQGDSSLVFGIKPGDPRVLQKLDDDGLPFIGAKLQYGDPYYSYLNLNTGESFVMYYKSKENCVVDNIKVCSNDTGSGKFKCVCITMRVPRNPTIGDKFASRHGQKGILSRLWPAEDMPFTESGMVPDILFNPHGFPSRMTIGMLIESMAGKSAALHGLCHDATPFIFSEENSALEYFGEMLKAAGYNFYGTERLYSGISGLELEADIFIGVVYYQRLRHMVSDKFQVRTTGARDRVTNQPIGGRNVQGGIRFGEMERDALLAHGTSFLLHDRLFNCSDRSVAHVCVKCGSLLSPLLEKPPPSWSAMRNRKYNCTLCSRSDTIDTVSVPYVFRYFVAELAAMNIKVKLDVV

SEQ ID NO: 84 - protein, 346 aa, Homo sapiens
MAASQAVEEMRSRVVLGEFGVRNVHTTDFPGNYSGYDDAWDQDRFEKNFRVDVVHMDENSLEFDMVGIDAAIANAFRRILLAEVPTMAVEKVLVYNNTSIVQDEILAHRLGLIPIHADPRLFEYRNQGDEEGTEIDTLQFRLQVRCTRNPHAAKDSSDPNELYVNHKVYTRHMTWIPLGNQADLFPEGTIRPVHDDILIAQLRPGQEIDLLMHCVKGIGKDHAKFSPVATASYRLLPDITLLEPVEGEAAEELSRCFSPGVIEVQEVQGKKVARVANPRLDTFSREIFRNEKLKKVVRLARVRDHYIFSVESTGVLPPDVLVSEAIKVLMGKCRRFLDELDAVQMD

SEQ ID NO: 85 - protein, 346 aa, Homo sapiens; identical to SEQ ID NO: 84

SEQ ID NO: 86 - protein, 133 aa, Homo sapiens
MEEDQELERKISGLKTSMAEGERKTALEMVQAAGTDRHCVTFVLHEEDHTLGNSLRYMIMKNPEVEFCGYTTTHPSESKINLRIQTRGTLPAVEPFQRGLNELMNVCQHVLDKFEASIKDYKDQKASRNESTF

SEQ ID NO: 87 - protein, 1970 aa, Homo sapiens
MHGGGPPSGDSACPLRTIKRVQFGVLSPDELKRMSVTEGGIKYPETTEGGRPKLGGLMDPRQGVIERTGRCQTCAGNMTECPGHFGHIELAKPVFHVGFLVKTMKVLRCVCFFCSKLLVDSNNPKIKDILAKSKGQPKKRLTHVYDLCKGKNICEGGEEMDNKFGVEQPEGDEDLTKEKGHGGCGRYQPRIRRSGLELYAEWKHVNEDSQEKKILLSPERVHEIFKRISDEECFVLGMEPRYARPEWMIVTVLPVPPLSVRPAVVMQGSARNQDDLTHKLADIVKINNQLRRNEQNGAAAHVIAEDVKLLQFHVATMVDNELPGLPRAMQKSGRPLKSLKQRLKGKEGRVRGNLMGKRVDFSARTVITPDPNLSIDQVGVPRSIAANMTFAEIVTPFNIDRLQELVRRGNSQYPGAKYIIRDNGDRIDLRFHPKPSDLHLQTGYKVERHMCDGDIVIFNRQPTLHKMSMMGHRVRILPWSTFRLNLSVTTPYNADFDGDEMNLHLPQSLETRAEIQELAMVPRMIVTPQSNRPVMGIVQDTLTAVRKFTKRDVFLERGEVMNLLMFLSTWDGKVPQPAILKPRPLWTGKQIFSLIIPGHINCIRTHSTHPDDEDSGPYKHISPGDTKVVVENGELIMGILCKKSLGTSAGSLVHISYLEMGHDITRLFYSNIQTVINNWLLIEGHTIGIGDSIADSKTYQDIQNTIKKAKQDVIEVIEKAHNNELEPTPGNTLRQTFENQVNRILNDARDKTGSSAQKSLSEYNNFKSMVVSGAKGSKINISQVIAVVGQQNVEGKRIPFGFKHRTLPHFIKDDYGPESRGFVENSYLAGLTPTEFFFHAMGGREGLIDTAVKTAETGYIQRRLIKSMESVMVKYDATVRNSINQVVQLRYGEDGLAGESVEFQNLATLKPSNKAFEKKFRFDYTNERALRRTLQEDLVKDVLSNAHIQNELEREFERMREDREVLRVIFPTGDSKVVLPCNLLRMIWNAQKIFHINPRLPSDLHPIKVVEGVKELSKKLVIVNGDDPLSRQAQENATLLFNIHLRSTLCSRRMAEEFRLSGEAFDWLLGEIESKFNQAIAHPGEMVGALAAQSLGEPATQMTLNTFHYAGVSAKNVTLGVPRLKELINISKKPKTPSLTVFLLGQSARDAERAKDILCRLEHTTLRKVTANTAIYYDPNPQSTVVAEDQEWVNVYYEMPDFDVARISPWLLRVELDRKHMTDRKLTMEQIAEKINAGFGDDLNCIFNDDNAEKLVLRIRIMNSDENKMQEEEEVVDKMDDDVFLRCIESNMLTDMTLQGIEQISKVYMHLPQTDNKKKIIITEDGEFKALQEWILETDGVSLMRVLSEKDVDPVRTTSNDIVEIFTVLGIEAVRKALERELYHVISFDGSYVNYRHLALLCDTMTCRGHLMAITRHGVNRQDTGPLMKCSFEETVDVLMEAAAHGESDPMKGVSENIMLGQLAPAGTGCFDLLLDAEKCKYGMEIPTNIPGLGAAGPTGMFFGSAPSPMGGISPAMTPWNQGATPAYGAWSPSVGSGMTPGAAGFSPSAASDASGFSPGYSPAWSPTPGSPGSPGPSSPYIPSPGGAMSPSYSPTSPAYEPRSPGGYTPQSPSYSPTSPSYSPTSPSYSPTSPNYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPNYSPTSPNYTPTSPSYSPTSPSYSPTSPNYTPTSPNYSPTSPSYSPTSPSYSPTSPSYSPSSPRYTPQSPTYTPSSPSYSPSSPSYSPTSPKYTPTSPSYSPSSPEYTPTSPKYSPTSPKYSPTSPKYSPTSPTYSPTTPKYSPTSPTYSPTSPVYTPTSPKYSPTSPTYSPTSPKYSPTSPTYSPTSPKGSTYSPTSPGYSPTSPTYSLTSPAISPDDSDEEN

SEQ ID NO: 88 - protein, 1970 aa, Homo sapiens; identical to SEQ ID NO: 87

SEQ ID NO: 89 - protein, 1174 aa, Homo sapiens
MYDADEDMQYDEDDDEITPDLWQEACWIVISSYFDEKGLVRQQLDSFDEFIQMSVQRIVEDAPPIDLQAEAQHASGEVEEPPRYLLKFEQIYLSKPTHWERDGAPSPMMPNEARLRNLTYSAPLYVDITKTVIKEGEEQLQTQHQKTFIGKIPIMLRSTYCLLNGLTDRDLCELNECPLDPGGYFIINGSEKVLIAQEKMATNTVYVFAKKDSKYAYTGECRSCLENSSRPTSTIWVSMLARGGQGAKKSAIGQRIVATLPYIKQEVPIIIVFRALGFVSDRDILEHIIYDFEDPEMMEMVKPSLDEAFVIQEQNVALNFIGSRGAKPGVTKEKRIKYAKEVLQKEMLPHVGVSDFCETKKAYFLGYMVHRLLLAALGRRELDDRDHYGNKRLDLAGPLLAFLFRGMFKNLLKEVRIYAQKFIDRGKDFNLELAIKTRIISDGLKYSLATGNWGDQKKAHQARAGVSQVLNRLTFASTLSHLRRLNSPIGRDGKLAKPRQLHNTLWGMVCPAETPEGHAVGLVKNLALMAYISVGSQPSPILEFLEEWSMENLEEISPAAIADATKIFVNGCWVGIHKDPEQLMNTLRKLRRQMDIIVSEVSMIRDIREREIRIYTDAGRICRPLLIVEKQKLLLKKRHIDQLKEREYNNYSWQDLVASGVVEYIDTLEEETVMLAMTPDDLQEKEVAYCSTYTHCEIHPSMILGVCASIIPFPDHNQSPRNTYQSAMGKQAMGVYITNFHVRMDTLAHVLYYPQKPLVTTRSMEYLRFRELPAGINSIVAIASYTGYNQEDSVIMNRSAVDRGFFRSVFYRSYKEQESKKGFDQEEVFEKPTRETCQGMRHAIYDKLDDDGLIAPGVRVSGDDVIIGKTVTLPENEDELESTNRRYTKRDCSTFLRTSETGIVDQVMVTLNQEGYKFCKIRVRSVRIPQIGDKFASRHGQKGTCGIQYRQEDMPFTCEGITPDIIINPHAIPSRMTIGHLIECLQGKVSANKGEIGDATPFNDAVNVQKISNLLSDYGYHLRGNEVLYNGFTGRKITSQIFIGPTYYQRLKHMVDDKIHSRARGPIQILNRQPMEGRSRDGGLRFGEMERDCQIAHGAAQFLRERLFEASDPYQVHVCNLCGIMAIANTRTHTYECRGCRNKTQISLVRMPYACKLLFQELMSMSIAPRMMSV

SEQ ID NO: 90 - protein, 275 aa, Homo sapiens
MPYANQPTVRITELTDENVKFIIENTDLAVANSIRRVFIAEVPIIAIDWVQIDANSSVLHDEFIAHRLGLIPLISDDIVDKLQYSRDCTCEEFCPECSVEFTLDVRCNEDQTRHVTSRDLISNSPRVIPVTSRNRDNDPNDYVEQDDILIVKLRKGQELRLRAYAKKGFGKEHAKWNPTAGVAFEYDPDNALRHTVYPKPEEWPKSEYSELDEDESQAPYDPNGKPERFYYNVESCGSLRPETIVLSALSGLKKKLSDLQTQLSHEIQSDVLTIN

SEQ ID NO: 91 - protein, 275 aa, Homo sapiens; identical to SEQ ID NO: 90

SEQ ID NO: 92 - protein, 275 aa, Homo sapiens; identical to SEQ ID NO: 90

SEQ ID NO: 93 - protein, 142 aa, Homo sapiens
MAAGGSDPRAGDVEEDASQLIFPKEFETAETLLNSEVHMLLEHRKQQNESAEDEQELSEVFMKTLNYTARFSRFKNRETIASVRSLLLQKKLHKFELACLANLCPETAEESKALIPSLEGRFEDEELQQILDDIQTKRSFQY

SEQ ID NO: 94 - protein, 142 aa, Homo sapiens; identical to SEQ ID NO: 93

SEQ ID NO: 95 - protein, 210 aa, Homo sapiens
MDDEEETYRLWKIRKTIMQLCHDRGYLVTQDELDQTLEEFKAQFGDKPSEGRPRRTDLTVLVAHNDDPTDQMFVFFPEEPKVGIKTIKVYCQRMQEENITRALIVVQQGMTPSAKQSLVDMAPKYILEQFLQQELLINITEHELVPEHVVMTKEEVTELLARYKLRENQLPRIQAGDPVARYFGIKRGQVVKIIRPSETAGRYITYRLVQ

SEQ ID NO: 96 - protein, 210 aa, Homo sapiens; identical to SEQ ID NO: 95

SEQ ID NO: 97 - protein, 127 aa, Homo sapiens
MSDNEDNFDGDDFDDVEEDEGLDDLENAEEEGQENVEILPSGERPQANQKRITTPYMTKYERARVLGTRALQIAMCAPVMVELEGETDPLLIAMKELKARKIPIIIRRYLPDGSYEDWGVDELIITD

SEQ ID NO: 98 - protein, 172 aa, Homo sapiens
MFYHISLEHEILLHPRYFGPNLLNTVKQKLFTEVEGTCTGKYGFVIAVTTIDNIGAGVIQPGRGFVLYPVKYKAIVFRPFKGEVVDAVVTQVNKVGLFTEIGPMSCFISRHSIPSEMEFDPNSNPPCYKTMDEDIVIQQDDEIRLKIVGTRVDKNDIFAIGSLMDDYLGLVS

SEQ ID NO: 99 - protein, 150 aa, Homo sapiens
MAGILFEDIFDVKDIDPEGKKFDRVSRLHCESESFKMDLILDVNIQIYPVDLGDKFRLVIASTLYEDGTLDDGEYNPTDDRPSRADQFEYVMYGKVYRIEGDETSTEAATRLSAYVSYGGLLMRLQGDANNLHGFEVDSRVYLLMKKLAF

SEQ ID NO: 100 - protein, 125 aa, Homo sapiens
MEPDGTYEPGFVGIRFCQECNNMLYPKEDKENRILLYACRNCDYQQEADNSCIYVNKITHEVDELTQIIADVSQDPTLPRTEDHPCQKCGHKEAVFFQSHSARAEDAMRLYYVCTAPHCGHRWTE

SEQ ID NO: 101 - protein, 117 aa, Homo sapiens
MNAPPAFESFLLFEGEKKITINKDTKVPNACLFTINKEDHTLGNIIKSQLLKDPQVLFAGYKVPHPLEHKIIIRVQTTPDYSPQEAFTNAITDLISELSLLEERFRVAIKDKQEGIE

SEQ ID NO: 102 - protein, 115 aa, Homo sapiens
MNAPPAFESFLLFEGEKITINKDTKVPKACLFTINKEDHTLGNIIKSQLLKDPQVLFAGYKVPHPLEHKIIIRVQTTPDYSPQEAFTNAITDLISELSLLEERFRTCLLPLRLLP

SEQ ID NO: 103 - protein, 115 aa, Homo sapiens
MNAPPAFESFLLFEGEKITINKDTKVPNACLFTINKEDHTLGNIIKSQLLKDPQVLFAGYKVPHPLEHKIIIRVQTTPDYSPQEAFTNAITDLISELSLLEERFRTCLLPLRLLP

SEQ ID NO: 104 - protein, 58 aa, Homo sapiens
MDTQKDVQPPKQQPMIYICGECHTENEIKSRDPIRCRECGYRIMYKKRTKRLVVFDAR

SEQ ID NO: 105 - protein, 58 aa, Homo sapiens; identical to SEQ ID NO: 104

SEQ ID NO: 106 - protein, 67 aa, Homo sapiens
MIIPVRCFTCGKIVGNKWEAYLGLLQAEYTEGDALDALGLKRYCCRRMLLAHVDLIEKLLNYAPLEK

SEQ ID NO: 107 - protein, 67 aa, Homo sapiens; identical to SEQ ID NO: 106

SEQ ID NO: 108 - protein, 1075 aa, Homo sapiens
MKANEKVTSDADPMWYLKYLNIYVGLPDVEESFNVTRPVSPHECRLRDMTYSAPITVDIEYTRGSQRIIRNALPIGRMPIMLRSSNCVLTGKTPAEFAKLNECPLDPGGYFIVKGVEKVILIQEQLSKNRIIVEADRKGAVGASVTSSTHEKKSRTNMAVKQGRFYLRHNTLSEDIPIVIIFKAMGVESDQEIVQMIGTEEHVMAAFGPSLEECQKAQIFTQMQALKYIGNKVRRQRMWGGGPKKTKIEEARELLASTILTHVPVKEFNFRAKCIYTAVMVRRVILAQGDNKVDDRDYYGNKRLELAGQLLSLLFEDLFKKFNSEMKKIADQVIPKQRAAQFDVVKHMRQDQITNGMVNAISTGNWSLKRFKMDRQGVTQVLSRLSYISALGMMTRISSQFEKTRKVSGPRSLQPSQWGMLCPSDTPEGEACGLVKNLALMTHITTDMEDGPIVKLASNLGVEDVNLLCGEELSYPNVFLVFLNGNILGVIRDHKKLVNTFRLMRRAGYINEFVSISTNLTDRCVYISSDGGRLCRPYIIVKKQKPAVTNKHMEELAQGYRNFEDFLHESLVEYLDVNEENDCNIALYEHTINKDTTHLEIEPFTLLGVCAGLIPYPHHNQSPRNTYQCAMGKQAMGTIGYNQRNRIDTLMYLLAYPQKPMVKTKTIELIEFEKLPAGQNATVAVMSYSGYDIEDALVLNKASLDRGFGRCLVYKNAKCTLKRYTNQTFDKVMGPMLDAATRKPIWRHEILDADGICSPGEKVENKQVLVNKSMPTVTQIPLEGSNVPQQPQYKDVPITYKGATDSYIEKVMISSNAEDAFLIKMLLRQTRRPEIGDKFSSRHGQKGVCGLIVPQEDMPFCDSGICPDIIMNPHGFPSRMTVGKLIELLAGKAGVLDGRFHYGTAFGGSKVKDVCEDLVRHGYNYLGKDYVTSGITGEPLEAYIYFGPVYYQKLKHMVLDKMHARARGPRAVLTRQPTEGRSRDGGLRLGEMERDCLIGYGASMLLLERLMISSDAFEVDVCGQCGLLGYSGWCHYCKSSCHVSSLRIPYACKLLFQELQSMNIIPRLKLSKYNE

SEQ ID NO: 109 - protein, 534 aa, Homo sapiens
MTQAEIKLCSLLLQEHFGEIVEKIGVHLIRTGSQPLRVIAHDTGTSLDQVKKALCVLVQHNLVSYQVHKRGVVEYEAQCSRVLRMLRYPRYIYTTKTLYSDTGELIVEELLLNGKLTMSAVVKKVADRLTETMEDGKTMDYAEVSNTFVRLADTHFVQRCPSVPTTENSDPGPPPPAPTLVINEKDMYLVPKLSLIGKGKRRRSSDEDAAGEPKAKRPKYTTDNKEPIPDDGIYWQANLDRFHQHFRDQAIVSAVANRMDQTSSEIVRTMLRMSEITTSSSAPFTQPLSSNEIFRSLPVGYNISKQVLDQYLTLLADDPLEFVGKSGDSGGGMYVINLHKALASLATATLESVVQERFGSRCARIFRLVLQKKHIEQKQVEDFAMIPAKEAKDMLYKMLSENFMSLQEIPKTPDHAPSRTFYLYTVNILSAARMLLHRCYKSIANLIERRQFETKENKRLLEKSQRVEAIIASMQATGAEEAQLQEIEEMITAPERQQLETLKRNVNKLDASEIQVDETIFLLESYIECTMKRQ

SEQ ID NO: 110 - protein, 534 aa, Homo sapiens; identical to SEQ ID NO: 109

SEQ ID NO: 111 - protein, 398 aa, Homo sapiens
MSEGNAAGEPSTPGGPRPLLTGARGLIGRRPAPPLTPGRLPSIRSRDLTLGGVKKKTFTPNIISRKIKEEPKEEVTVKKEKRERDRDRQREGHGRGRGRPEVIQSHSIFEQGPAEMMKKKGNWDKTVDVSDMGPSHIINIKKEKRETDEETKQILRMLEKDDFLDDPGLRNDTRNMPVQLPLAHSGWLFKEENDEPDVKPWLAGPKEEDMEVDIPAVKVKEEPRDEEEEAKMKAPPKAARKTPGLPKDVSVAELLRELSLTKEEELLFLQLPDTLPGQPPTQDIKPIKTEVQGEDGQVVLIKQEKDREAKLAENACTLADLTEGQVGKLLIRKSGRVQLLLGKVTLDVTMGTACSFLQELVSVGLGDSRTGEMTVLGHVKHKLVCSPDFESLLDHKHR

SEQ ID NO: 112 - protein, 708 aa, Homo sapiens
MANEEDDPVVQEIDVYLAKSLAEKLYLFQYPVRPASMTYDDIPHLSAKIKPKQQKVELEMAIDTLNPNYCRSKGEQIALNVDGACADETSTYSSKLMDKQTFCSSQTTSNTSRYAAALYRQGELHLTPLHGILQLRPSFSYLDKADAKHREREAANEAGDSSQDEAEDDVKQITVRFSRPESEQARQRRVQSYEFLQKKHAEEPWVHLHYYGLRDSRSEHERQYLLCPGSSGVENTELVKSPSEYLMMLMPPSQEEEKDKPVAPSNVLSMAQLRTLPLADQIKILMKNVKVMPFANLMSLLGPSIDSVAVLRGIQKVAMLVQGNWVVKSDILYPKDSSSPHSGVPAEVLCRGRDFVMWKFTQSRWVVRKEVATVTKLCAEDVKDFLEHMAVVRINKGWEFILPYDGEFIKKHPDVVQRQHMLWTGIQAKLEKVYNLVKETMPKKPDAQSGPAGLVCGDQRIQVAKTKAQQNHALLERELQRRKEQLRVPAVPPGVRIKEEPVSEEGEEDEEQEAEEEPMDTSPSGLHSKLANGLPLGRAAGTDSFNGHPPQGCASTPVARELKAFVEATFQRQFVLTLSELKRLFNLHLASLPPGHTLFSGISDRMLQDTVLAAGCKQILVPFPPQTAASPDEQKVFALWESGDMSDQHRQVLLEIFSKNYRVRRNMIQSRLTQECGEDLSKQEVDKVLKDCCVSYGGMWYLKGTVQS

SEQ ID NO: 113 - protein, 316 aa, Homo sapiens
MAEVKVKVQPPDADPVEIENRIIELCHQFPHGITDQVIQNEMPHIEAQQRAVAINRLLSMGQLDLLRSNTGLLYRIKDSQNAGKMKGSDNQEKLVYQIIEDAGNKGIWSRDIRYKSNLPLTEINKILKNLESKKLIKAVKSVAASKKKVYMLYNLQPDRSVTGGAWYSDQDFESEFVEVLNQQCFKFLQSKAETARESKQNPMIQRNSSFASSHEVWKYICELGISKVELSMEDIETILNTLIYDGKVEMTIIAAKEGTVGSVDGHMKLYRAVNPIIPPTGLVRAPCGLCPVFDDCHEGGEISPSNCIYMTEWLEF

SEQ ID NO: 114 - protein, 223 aa, Homo sapiens
MAGNKGRGRAAYTFNIEAVGFSKGEKLPDVVLKPPPLFPDTDYKPVPLKTGEGEEYMLALKQELRETMKRMPYFIETPEERQDIERYSKRYMKVYKEEWIPDWRRLPREMMPRNKCKKAGPKPKKAKDAGKGTPLTNTEDVLKKMEELEKRGDGEKSDEENEEKEGSKEKSKEGDDDDDDDAAEQEEYDEEEQEEENDYINSYFEDGDDFGADSDDNMDEATY

SEQ ID NO: 115 - protein, 223 aa, Homo sapiens; identical to SEQ ID NO: 114

SEQ ID NO: 116 - protein, 108 aa, Homo sapiens
MLLFCPGCGNGLIVEEGQRCHRFSCNTCPYVHNITRKVTNRKYPKLKEVDDVLGGAAAWENVDSTAESCPKCEHPRAYFMQLQTRSADEPMTTFYKCCNAQCGHRWRD

SEQ ID NO: 117 - protein, 1230 aa, Homo sapiens
MSALCWGRGAAGLKRALRPCGRPGLPGKEGTAGGVCGPRRSSSASPQEQDQDRRKDWGHVELLEVLQARVRQLQAESVSEVVVNRVDVARLPECGSGDGSLQPPRKVQMGAKDATPVPCGRWAKILEKDKRTQQMRMQRLKAKLQMPFQSGEFKALTRRLQVEPRLLSKQMAGCLEDCTRQAPESPWEEQLARLLQEAPGKLSLDVEQAPSGQHSQAQLSGQQQRLLAFFKCCLLTDQLPLAHHLLVVHHGQRQKRKLLTLDMYNAVMLGWARQGAFKELVYVLFMVKDAGLTPDLLSYAAALQCMGRQDQDAGTIERCLEQMSQEGLKLQALFTAVLLSEEDRATVLKAVHKVKPTFSLPPQLPPPVNTSKLLRDVYAKDGRVSYPKLHLPLKTLQCLFEKQLHMELASRVCVVSVEKPTLPSKEVKHARKTLKTLRDQWEKALCRALRETKNRLEREVYEGRFSLYPFLCLLDEREVVRMLLQVLQALPAQGESFTTLARELSARTFSRHVVQRQRVSGQVQALQNHYRKYLCLLASDAEVPEPCLPRQYWEELGAPEALREQPWPLPVQMELGKLLAEMLVQATQMPCSLDKPHRSSRLVPVLYHVYSFRNVQQIGILKPHPAYVQLLEKAAEPTLTFEAVDVPMLCPPLPWTSPHSGAFLLSPTKLMRTVEGATQHQELLETCPPTALHGALDALTQLGNCAWRVNGRVLDLVLQLFQAKGCPQLGVPAPPSEAPQPPEAHLPHSAAPARKAELRRELAHCQKVAREMHSLRAEALYRLSLAQHLRDRVFWLPHNMDFRGRTYPCPPHFNHLGSDVARALLEFAQGRPLGPHGLDWLKIHLVNLTGLKKREPLRKRLAFAEEVMDDILDSADQPLTGRKWWMGAEEPWQTLACCMEVANAVRASDPAAYVSHLPVHQDGSCNGLQHYAALGRDSVGAASVNLEPSDVPQDVYSGVAAQVEVFRRQDAQRGMRVAQVLEGFITRKVVKQTVMTVVYGVTRYGGRLQIEKRLRELSDFPQEFVWEASHYLVRQVFKSLQEMFSGTRAIQHWLTESARLISHMGSVVEWVTPLGVPVIQPYRLDSKVKQIGGGIQSITYTHNGDISRKPNTRKQKNGFPPNFIHSLDSSHMMLTALHCYRKGLTFVSVHDCYWTHAADVSVMNQVCREQFVRLHSEPILQDLSRFLVKRFCSEPQKILEASQLKETLQAVPKPGAFDLEQVKRSTYFFS

SEQ ID NO: 118 - protein, 1230 aa, Homo sapiens; identical to SEQ ID NO: 117

SEQ ID NO: 119 - protein, 541 aa, Homo sapiens
MSPCPEEAAMRREVVKRIETVVKDLWPTADVQIFGSFSTGLYLPTSDIDLVVFGKWERPPLQLLEQALRKHNVAEPCSIKVLDKATVPIIKLTDQETEVKVDISFNMETGVRAAEFIKNYMKKYSLLPYLILVLKQFLLQRDLNEVFTGGISSYSLILMAISFLQLHPRIDARRADENLGMLLVEFFELYGRNFNYLKTGIRIKEGGAYIAKEEIMKAMTSGYRPSMLCIEDPLLPGNDVGRSSYGAMQVKQVFDYAYIVLSHAVSPLARSYPNRDAESTLGRIIKVTQEVIDYRRWIKEKWGSKAHPSPGMDSRIKIKERIATCNGEQTQNREPESPYGQRLTLSLSSPQLLSSGSSASSVSSLSGSDVDSDTPPCTTPSVYQFSLQAPAPLMAGLPTALPMPSGKPQPTTSRTLIMTTNNQTRFTIPPPTLGVAPVPCRQAGVEGTASLKAVHHMSSPAIPSASPNPLSSPHLYHKHNGMKLSMKGSHGHTQGGGYSSVGSGGVRPPVGNRGHHQYNRTGWRRKKHTHTRDSLPVSLSR

SEQ ID NO: 120 - protein, 1135 aa, Homo sapiens
MDPGSRWRNLPSGPSLKHLTDPSYGIPREQQKAALQELTRAHVESFNYAVHEGLGLAVQAIPPFEFAFKDERISFTILDAVISPPTVPKGTICKEANVYPAECRGRRSTYRGKLTADINWAVNGISKGIIKQFLGYVPIMVKSKLCNLRNLPPQALIEHHEEAEEMGGYFIINGIEKVIRMLIMPRRNFPIAMIRPKWKTRGPGYTQYGVSMHCVREEHSAVNMNLHYLENGTVMLNFIYRKELFFLPLGFALKALVSFSDYQIFQELIKGKEDDSFLRNSVSQMLRIVMEEGCSTQKQVLNYLGECFRVKLNVPDWYPNEQAAEFLFNQCICIHLKSNTEKFYMLCLMTRKLFALAKGECMEDNPDSLVNQEVLTPGQLFLMFLKEKLEGWLVSIKIAFDKKAQKTSVSMNTDNLMRIFTMGIDLTKPFEYLFATGNLRSKTGLGLLQDSGLCVVADKLNFIRYLSHFRCVHRGADFAKMRTTTVRRLLPESWGFLCPVHTPDGEPCGLMNHLTAVCEVVTQFVYTASIPALLCNLGVTPIDGAPHRSYSECYPVLLDGVMVGWVDKDLAPGIADSLRHFKVLREKRIPPWMEVVLIPMTGKPSLYPGLFLFTTPCRLVRPVQNLALGKEELIGTMEQIFMNVAIFEDEVFAGVTTHQELFPHSLLSVIANFIPFSDHNQSPRNMYQCQMGKQTMGFPLLTYQDRSDNKLYRLQTPQSPLVRPSMYDYYDMDNYPIGTNAIVAVISYTGYDMEDAMIVNKASWERGFAHGSVYKSEFIDLSEKIKQGDSSLVFGIKPGDPRVLQKLDDDGLPFIGAKLQYGDPYYSYLNLNTGESFVMYYKSKENCVVDNIKVCSNDTGSGKFKCVCITMRVPRNPTIGDKFASRHGQKGILSRLWPAEDMPFTESGMVPDILFNPHGFPSRMTIGMLIESMAGKSAALHGLCHDATPFIFSEENSALEYFGEMLKAAGYNFYGTERLYSGISGLELEADIFIGVVYYQRLRHMVSDKFQVRTTGARDRVTNQPIGGRNVQGGIRFGEMERDALLAHGTSFLLHDRLFNCSDRSVAHVCVKCGSLLSPLLEKPPPSWSAMRNRKYNCTLCSRSDTIDTVSVPYVFRYFVAELAAMNIKVKLDVV

SEQ ID NO: 121 - protein, 122 aa, Homo sapiens
MEEDQELERKAIEELLKEAKRGKTRAETMGPMGWMKCPLASTNKRFLINTIKNTLPSHKEQDHEQKEGDKEPAKSQAQKEENPKKHRSHPYKHSFRARGSASYSPPRKRSSQDKYEKRSNRR

SEQ ID NO: 122 - protein, 117 aa, Homo sapiens; identical to SEQ ID NO: 101

SEQ ID NO: 123 - protein, 1133 aa, Homo sapiens
MDVLAEEFGNLTPEQLAAPIPTVEEKWRLLPAFLKVKGLVKQHIDSFNYFINVEIKKIMKANEKVTSDADPMWYLKYLNIYVGLPDVEESFNVTRPVSPHECRLRDMTYSAPITVDIEYTRGSQRIIRNALPIGRMPIMLRSSNCVLTGKTPAEFAKLNECPLDPGGYFIVKGVEKVILIQEQLSKNRIIVEADRKGAVGASVTSSTHEKKSRTNMAVKQGRFYLRHNTLSEDIPIVIIFKAMGVESDQEIVQMIGTEEHVMAAFGPSLEECQKAQIFTQMQALKYIGNKVRRQRMWGGGPKKTKIEEARELLASTILTHVPVKEFNFRAKCIYTAVMVRRVILAQGDNKVDDRDYYGNKRLELAGQLLSLLFEDLFKKFNSEMKKIADQVIPKQRAAQFDVVKHMRQDQITNGMVNAISTGNWSLKRFKMDRQGVTQVLSRLSYISALGMMTRISSQFEKTRKVSGPRSLQPSQWGMLCPSDTPEGEACGLVKNLALMTHITTDMEDGPIVKLASNLGVEDVNLLCGEELSYPNVFLVFLNGNILGVIRDHKKLVNTFRLMRRAGYINEFVSISTNLTDRCVYISSDGGRLCRPYIIVKKQKPAVTNKHMEELAQGYRNFEDFLHESLVEYLDVNEENDCNIALYEHTINKDTTHLEIEPFTLLGVCAGLIPYPHHNQSPRNTYQCAMGKQAMGTIGYNQRNRIDTLMYLLAYPQKPMVKTKTIELIEFEKLPAGQNATVAVMSYSGYDIEDALVLNKASLDRGFGRCLVYKNAKCTLKRYTNQTFDKVMGPMLDAATRKPIWRHEILDADGICSPGEKVENKQVLVNKSMPTVTQIPLEGSNVPQQPQYKDVPITYKGATDSYIEKVMISSNAEDAFLIKMLLRQTRRPEIGDKFSSRHGQKGVCGLIVPQEDMPFCDSGICPDIIMNPHGFPSRMTVGKLIELLAGKAGVLDGRFHYGTAFGGSKVKDVCEDLVRHGYNYLGKDYVTSGITGEPLEAYIYFGPVYYQKLKHMVLDKMHARARGPRAVLTRQPTEGRSRDGGLRLGEMERDCLIGYGASMLLLERLMISSDAFEVDVCGQCGLLGYSGWCHYCKSSCHVSSLRIPYACKLLFQELQSMNIIPRLKLSKYNE

SEQ ID NO: 124 - protein, 362 aa, Homo sapiens
MLLVEFFELYGRNFNYLKTGIRIKEGGAYIAKEEIMKAMTSGYRPSMLCIEDPLLPGNDVGRSSYGAMQVKQVFDYAYIVLSHAVSPLARSYPNRDAESTLGRIIKVTQEVIDYRRWIKEKWGSKAHPSPGMDSRIKIKERIATCNGEQTQNREPESPYGQRLTLSLSSPQLLSSGSSASSVSSLSGSDVDSDTPPCTTPSVYQFSLQAPAPLMAGLPTALPMPSGKPQPTTSRTLIMTTNNQTRFTIPPPTLGVAPVPCRQAGVEGTASLKAVHHMSSPAIPSASPNPLSSPHLYHKQHNGMKLSMKGSHGHTQGGGYSSVGSGGVRPPVGNRGHHQYNRTGWRRKKHTHTRDSLPVSLSR

SEQ ID NO: 125 - protein, 115 aa, Homo sapiens; identical to SEQ ID NO: 102

SEQ ID NO: 126 - protein, 542 aa, Homo sapiens
MSPCPEEAAMRREVVKRIETVVKDLWPTADVQIFGSFSTGLYLPTSDIDLVVFGKWERPPLQLLEQALRKHNVAEPCSIKVLDKATVPIIKLTDQETEVKVDISFNMETGVRAAEFIKNYMKKYSLLPYLILVLKQFLLQRDLNEVFTGGISSYSLILMAISFLQLHPRIDARRADENLGMLLVEFFELYGRNFNYLKTGIRIKEGGAYIAKEEIMKAMTSGYRPSMLCIEDPLLPGNDVGRSSYGAMQVKQVFDYAYIVLSHAVSPLARSYPNRDAESTLGRIIKVTQEVIDYRRWIKEKWGSKAHPSPGMDSRIKIKERIATCNGEQTQNREPESPYGQRLTLSLSSPQLLSSGSSASSVSSLSGSDVDSDTPPCTTPSVYQFSLQAPAPLMAGLPTALPMPSGKPQPTTSRTLIMTTNNQTRFTIPPPTLGVAPVPCRQAGVEGTASLKAVHHMSSPAIPSASPNPLSSPHLYHKQHNGMKLSMKGSHGHTQGGGYSSVGSGGVRPPVGNRGHHQYNRTGWRRKKHTHTRDSLPVSLSR